(19) United States
(12) Patent Application Publication
(10) Pub. No.: US 2025/0311945 A1
(43) Pub. Date: Oct. 9, 2025

(54) SYSTEMS, DEVICES, AND METHODS FOR TIME-IN-RANGE AND MEAL-RELATED ANALYTE MONITORING

(71) Applicant: ABBOTT DIABETES CARE INC., Alameda, CA (US)

(72) Inventors: Vyshnnavi Parthasarathy, Fremont, CA (US); Prarthana Prashanth, Foster City, CA (US); Panganamala Ashwin Kumar, Oakland, CA (US); Rene S. Shabastari, Winnetka, CA (US); Jonathan Wood, Seattle, WA (US); Jose D. Vega, Santa Ana (CR); Shreya Gupta, San Jose, CA (US); Jeffrey Nishida-Boucher, Redwood City, CA (US)

(21) Appl. No.: 18/963,253

(22) Filed: Nov. 27, 2024

Related U.S. Application Data
(60) Provisional application No. 63/603,593, filed on Nov. 28, 2023.
(60) Provisional application No. 63/722,692, filed on Nov. 20, 2024.

(51) Int. Cl. A61B 5/145; A61B 5/00

(52) U.S. Cl. CPC A61B 5/14532; A61B 5/002; A61B 5/7246; A61B 5/742

(57) ABSTRACT

Systems, devices, and methods for detecting and measuring meal impact and/or an amount of time an individual is within a predetermined analyte range based on analyte measurements. These results and related information are presented to the individual to show the individual an analyte response associated with consumed meals, or change in an analyte level within a predetermined time period after meals are consumed. These results can be organized based on a ranking or scoring system so as to allow the individual to visualize analyte responses and range impact associated with the meals. Various embodiments disclosed herein relate to methods, systems, and software applications intended to engage an individual by providing direct and timely feedback regarding the individual's meal-related analyte response.

### CROSS-REFERENCE TO RELATED APPLICATIONS

This application claims priority to U.S. Provisional Application No. 63/722,692, filed Nov. 20, 2024, and U.S. Provisional Application No. 63/603,593, filed Nov. 28, 2023, both of which are herein expressly incorporated by reference in their entireties for all purposes.

The subject matter described herein relates generally to computing interfaces for analyte monitoring systems, as well as systems, methods, and devices related thereto. In particular, disclosed herein are various embodiments of Time-In-Range (TIR) and meal-related graphical user interfaces and methods related thereto for analyte monitoring systems.

### BACKGROUND

The increased prevalence of Type 2 diabetes and metabolic syndrome over the past few decades has been attributed to changing diet and activity levels. For example, consumption of more readily available high glycemic index foods can cause rapid post-prandial increases of blood glucose and insulin levels, which has a positive association with weight gain and obesity. These conditions can be further traced to an increased risk of developing these and other diseases.

Most people generally understand the importance of their diet. However, in practice, many people struggle with translating this general awareness to their specific food choices. These problems exist primarily because people cannot directly see the impact of their choices. This can lead to misconceptions around food portion size, misunderstandings about which foods are relatively healthy, and a general lack of awareness regarding the necessary duration and intensity of activity to maintain good health. These problems are further exacerbated by advertisements, habits, peer pressure, food preferences, and recommendations based on generalizations.

To address these issues, an individual's physiological responses can be tracked and better understood by analyte monitoring systems. Because high glucose levels are primarily driven by the consumption of food, the level of post-prandial glucose can relate to the amount of carbohydrates and other meal components consumed by the individual, as well as to the individual's physiological response to meals. However, a challenge for analysis of this influx of data is to represent the data in a meaningful manner that enables efficient action. Data relating to meal selection, and the subsequent impact, should be understood on a clinical basis, as well as a personal basis for the individual, the meal administrator, and/or the medical professional in order to understand and moderate glucose excursions, such as episodes of hyperglycemia.

It has been shown that individuals who maintain their analyte levels within a target analyte range are more likely to experience positive health outcomes. Thus, it would be advantageous to provide individuals with user-friendly and actionable information about the amount of time spent within a target analyte range; the impact of meals consumed by the individuals on their respective analyte levels; and a means by which to motivate and encourage the individual to make beneficial food choices.

Prior systems for tracking meal consumption and correlating consumed meals with an individual's analyte data suffer from numerous deficiencies. For example, some systems require that an individual perform numerous inconvenient and uncomfortable discrete blood glucose measurements (e.g., finger stick blood glucose tests). These solutions can suffer from an insufficient number of data points to adequately determine a glycemic response to a meal. For example, an individual may perform a discrete blood glucose measurement at a time before or after the time when the user's glycemic response peaks, making it difficult to accurately ascertain the glycemic response, and to meaningfully compare meals based on the glycemic response. A deficiency in data points can also make it difficult to detect the occurrence of a meal event in the user's analyte data. Thus, some prior systems place significant reliance upon manual logging of meals by the user. Moreover, many prior systems that seek to detect meal events based simply on the existence of a rise in glucose levels are inadequate because they fail to take into account the user's prior meal history, and thus can overestimate the number of meals the user has consumed.

Thus, improved systems, devices, and methods for meal information collection, meal assessment and detection, and correlation to analyte levels are needed. In particular, needs exist for improved graphical user interfaces for analyte monitoring systems, as well as methods and devices relating thereto, that are robust, user-friendly, and allow the individual to understand analyte responses and analyte range impacts associated with consumed meals.

### SUMMARY

Aspects of the invention are set out in the independent claims and preferred features are set out in the dependent claims. Features associated with one aspect may be applied to other aspects alone or in combination. Provided herein are example embodiments of systems, devices, and methods for detecting and measuring an amount of time an individual is within a predetermined analyte range based on analyte measurements. Also provided herein are example embodiments of systems, devices, and methods for detecting, measuring, and ranking meals for the individual in relation to that individual's analyte measurements. In many embodiments, these results and related information are presented to the individual to show the individual an analyte response associated with consumed meals, or a change in an analyte level within a predetermined time period after meals are consumed. These individuals can be those exhibiting or diagnosed with a diabetic condition, those considered as pre-diabetic, those with metabolic syndrome, and even those without diabetes, pre-diabetic, or metabolic syndrome conditions. These individuals can be any person motivated to improve his or her health by adjustment to his or her diet and/or activity practices. Resulting information can be presented to the individual to show which meals or aspects of the meals are causing the most impact on analyte levels.

In many embodiments, the individual's meal-related analyte responses (e.g., glucose responses) are based on analyte data (e.g., glucose data) collected by an analyte monitoring system (e.g., a glucose monitoring system), such as an in vivo analyte monitoring system (e.g., an in vivo glucose monitoring system). These responses can be compared with or linked to meal information to discover common consistencies (or inconsistencies), along with trends therein based on related historical glucose readings and associated algorithms, and comparisons.

Many embodiments disclosed herein are intended to engage the individual by providing direct and timely feedback regarding the individual's meal-related analyte response. In some embodiments, this analyte response can be provided to the individual in an easy-to-understand format to characterize the effects of meal consumption.

Many of the embodiments can be immediately informative to the individual, thereby encouraging the individual to take actions to better understand how their own diet impacts their body's analyte response. Many of the embodiments can also organize data, e.g., rank meals, according to changes detected in the individual's analyte level within a predetermined time period after consuming a meal. The individual can compare and contrast their current and historical analyte data to see their how their own efforts are related to better diet and meal selection, and how these choices directly affect their health. The individual can also better understand how a particular food choice can help them stay in a target range of analyte values, and visualize the analyte response and range impacts correlating with particular foods. In this manner, the individual is motivated to stay within a target analyte range.

Many of the embodiments provided herein are improved graphical user interfaces (GUIs) or GUI features for analyte monitoring systems that are highly intuitive, user-friendly, and provide for rapid access to physiological information of an individual. More specifically, these embodiments allow an individual to easily navigate through and between different user interfaces that can quickly indicate to the user various physiological conditions and/or actionable responses and correlate analyte data with meals, exercise, stress, or other factors, without requiring the user (or an HCP) to go through the arduous task of examining large volumes of analyte data.

In many embodiments, some of the GUIs and GUI features allow for individuals (and their caregivers) to better understand and improve their diet, eating habits, and manage other stressors as they see the correlations with these activities and their glucose levels. Likewise, in many embodiments, improved digital interfaces and/or features for TIR and meal-related systems may improve upon the visualization of the impact of food choices on analyte (glucose) levels and the amount of time spent in a target analyte range, the visualization of the good foods and bad foods that exist in the individual's current diet and their impact on glucose levels and TIR, the correlation of meal information to detected meal events, and the motivation for individuals to maintain and/or increase TIR by informing individuals of options of foods to eat while still maintaining a TIR goal, to name only a few. Other improvements and advantages are provided as well. The various configurations of these devices are described in detail by way of the embodiments which are only examples.

The improvements to the GUIs in the various aspects described and claimed herein produce a technical effect at least in that they assist the user of the device to operate the device more accurately, more efficiently, and more safely. It will be appreciated that the information that is provided to the individual on the GUIs, the order in which that information is provided, and the clarity with which that information is structured can have a significant effect on the way the individual interacts with the system and the way the system operates. The GUIs therefore guide the individual in the technical task of operating the system to take the necessary readings and/or obtain information accurately and efficiently.

Other systems, devices, methods, features, and advantages of the subject matter described herein will be or will become apparent to one with skill in the art upon examination of the following figures and detailed description. It is intended that all such additional systems, devices, methods, features and advantages be included within this description, be within the scope of the subject matter described herein, and be protected by the accompanying claims. In no way should the features of the example embodiments be construed as limiting the appended claims, absent express recitation of those features in the claims.

### BRIEF DESCRIPTION OF THE FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be depicted schematically rather than literally or precisely.

FIG. 1 is a system overview of an analyte monitoring system comprising a sensor applicator, a sensor control device, a reader device, a network, a trusted computer system, and a local computer system.

FIG. 2A is a block diagram depicting an example embodiment of a reader device.

FIGS. 2B and 2C are block diagrams depicting example embodiments of sensor control devices.

FIG. 3 is a block diagram depicting an example embodiment of an analyte monitoring system for use with an analyte monitoring application and/or a meal monitoring application.

FIG. 4A-1 is a flow diagram depicting an example embodiment of a method for associating analyte data with meal information, and ranking the meal information.

FIGS. 4A-2 and 4A-3 are graphs depicting post-prandial glucose traces.

FIG. 4A-4 is a flow diagram depicting an example embodiment of a method for associating analyte data with meal information, and ranking the meal information.

FIG. 4A-5 is a flow diagram depicting an example embodiment of a method for displaying an interface comprising information related to time-in-range.

FIG. 4A-6 is a flow diagram depicting an example embodiment of a method for calculating a user's glycemic control over a predetermined time period.

FIG. 4B is a block diagram depicting an example embodiment of a home GUI.

FIG. 4C is a block diagram depicting an example embodiment of a time-in-range informational screen.

FIGS. 4D to 4N are block diagrams depicting example embodiments of home GUIs, modals, and features related thereto.

FIGS. 5A-5N are block diagrams depicting example embodiments of meal logging GUIs and modals.

FIG. 6A is a flow diagram depicting an example embodiment of a method for displaying a report GUI.

FIGS. 6B-1 and 6B-2 are block diagrams depicting example embodiments of a report GUI.

FIG. 7 is a block diagram depicting an example embodiment of a meals impact GUI.

FIGS. 8A to 8E are block diagrams depicting example embodiments of meal review GUIs.

FIGS. 9A to 9Q-4 are block diagrams depicting example embodiments of onboarding GUIs and features relating thereto.

FIGS. 10A-10M are block diagrams depicting example embodiments of account and settings GUI and features relating thereto.

FIGS. 11A-11G are block diagrams depicting example embodiments of data loss GUIs and features.

FIGS. 12A-1 to 12D-2 are block diagrams depicting example embodiments of interfaces comprising activity cards and features.

FIG. 13A is a flow diagram depicting an example embodiment of a method for meal detection.

FIG. 13B is a flow diagram depicting an example embodiment of a method for determining bespoke time windows for when a user is expected to eat one or more meals, based on historical data.

FIGS. 13C-1 to 13E-5 are block diagrams depicting example embodiments of meal detection interfaces and features.

FIG. 14 is a flow diagram depicting an example embodiment of a method for identifying high analyte level impact and low analyte level impact time periods based on meal excursions of the user.

FIGS. 14A-1 to 15B are block diagrams depicting example embodiments of insights interfaces and features.

### DETAILED DESCRIPTION

Provided herein are example embodiments of systems, devices, and methods for monitoring and measuring analyte responses to meals for an individual. In particular, based on the analyte data collected, meal-related events and their impact on the individual's analyte levels can be further understood by a user, and eventually used to modify future meal selection and dietary habits.

Before describing this subject matter in greater detail, it is worthwhile to describe example embodiments of systems, devices, and methods with which the subject matter can be implemented.

A number of systems have been developed for the automatic monitoring of the analyte(s), like glucose, in bodily fluid such as in the blood stream, in interstitial fluid (“ISF”), dermal fluid of the dermal layer, or in other biological fluid. Some of these systems are configured so that at least a portion of a sensor is positioned below a skin surface of a user, e.g., in a blood vessel or in the subcutaneous tissue of a user, to obtain information about at least one analyte of the body.

As such, these systems can be referred to as “in vivo” monitoring systems. In vivo analyte monitoring systems include “Continuous Analyte Monitoring” systems (or “Continuous Glucose Monitoring” systems) that can transmit data from a sensor control device to a reader device continuously without prompting, e.g., automatically according to a schedule. In vivo analyte monitoring systems also include “Flash Analyte Monitoring” systems (or “Flash Glucose Monitoring” systems or simply “Flash” systems) that can transfer data from a sensor control device in response to a scan or request for data by a reader device, such as with a Near Field Communication (NFC) or Radio Frequency Identification (RFID) protocol. In vivo analyte monitoring systems can also operate without the need for finger stick calibration.

The in vivo analyte monitoring systems can be differentiated from “in vitro” systems that contact a biological sample outside of the body (or rather “ex vivo”) and that typically include a meter device that has a port for receiving an analyte test strip carrying bodily fluid of the user, which can be analyzed to determine the user's blood sugar level. While in many of the present embodiments the monitoring is accomplished in vivo, the embodiments disclosed herein can be used with in vivo analyte monitoring systems that incorporate in vitro capability, as well has purely in vitro or ex vivo analyte monitoring systems.

The sensor can be part of the sensor control device that resides on the body of the user and contains the electronics and power supply that enable and control the analyte sensing. The sensor control device, and variations thereof, can also be referred to as a “sensor control unit,” an “on-body electronics” device or unit, an “on-body” device or unit, or a “sensor data communication” device or unit, to name a few.

In vivo monitoring systems can also include a device that receives sensed analyte data from the sensor control device and processes and/or displays that sensed analyte data, in any number of forms, to the user. This device, and variations thereof, can be referred to as a “reader device” (or simply a “reader”), “handheld electronics” (or a handheld), a “portable data processing” device or unit, a “data receiver,” a “receiver” device or unit (or simply a receiver), or a “remote” device or unit, to name a few. Other devices such as personal computers have also been utilized with or incorporated into in vivo and in vitro monitoring systems.

### Embodiments of In Vivo Analyte Monitoring Systems

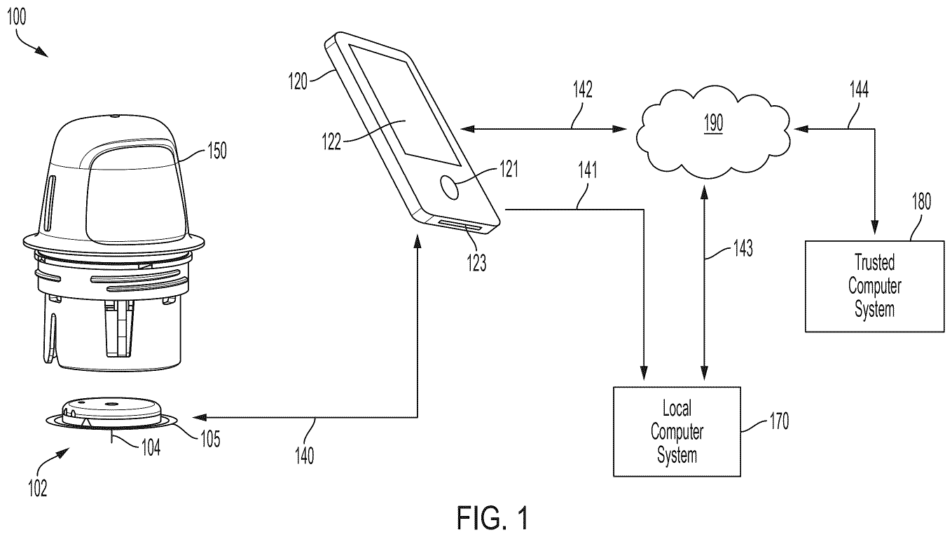

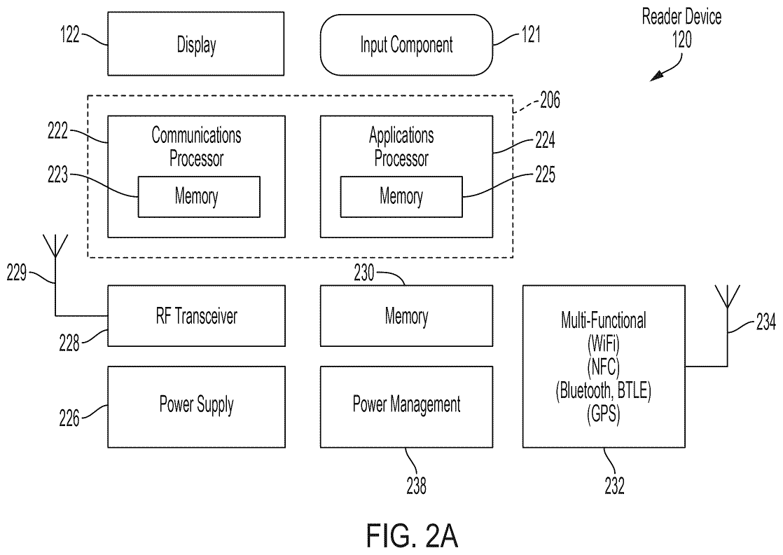

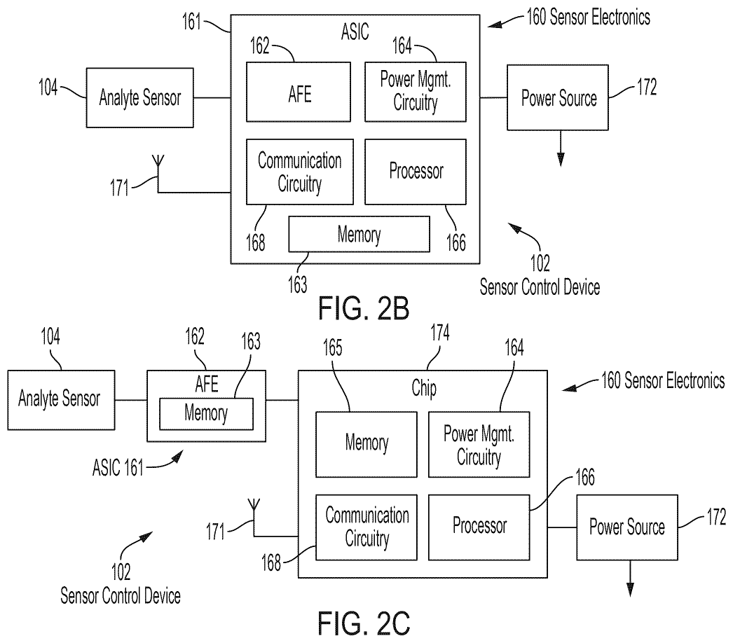

FIG. 1 is a conceptual diagram depicting an example embodiment of an analyte monitoring system 100 (e.g., a glucose monitoring system) that includes a sensor applicator 150, a sensor control device 102, and a reader device 120. Here, sensor applicator 150 can be used to deliver sensor control device 102 to a monitoring location on a user's skin where a sensor 104 is maintained in position for a period of time by an adhesive patch 105. Sensor control device 102 is further described in FIGS. 2B and 2C, and can communicate with reader device 120 via a communication path 140 using a wired or wireless technique. Example wireless protocols include Bluetooth, Bluetooth Low Energy (BLE, BTLE, Bluetooth SMART, etc.), Near Field Communication (NFC) and others. Users can view and use applications installed in memory on reader device 120 using screen 122 (which, in many embodiments, can comprise a touchscreen), and input 121. A device battery of reader device 120 can be recharged using power port 123. While only one reader device 120 is shown, sensor control device 102 can communicate with multiple reader devices 120. Each of the reader devices 120 can communicate and share data with one another. More details about reader device 120 is set forth with respect to FIG. 2A below. Reader device 120 can communicate with local computer system 170 via a communication path 141 using a wired or wireless communication protocol. Local computer system 170 can include one or more of a laptop, desktop, tablet, phablet, smartphone, set-top box, video game console, or other computing device and wireless communication can include any of a number of applicable wireless networking protocols including Bluetooth, Bluetooth Low Energy (BTLE), Wi-Fi or others. Local computer system 170 can communicate via communications path 143 with a network 190 similar to how reader device 120 can communicate via a communications path 142 with network 190, by a wired or wireless communication protocol as described previously. Network 190 can be any of a number of networks, such as private networks and public networks, local area or wide area networks, and so forth. A trusted computer system 180 can include a server and can provide authentication services and secured data storage and can communicate via communications path 144 with network 190 by wired or wireless technique.

### Example Embodiment of Reader Device

FIG. 2A is a block diagram depicting an example embodiment of a reader device 120, which, in some embodiments, can comprise a smartphone. Here, reader device 120 can include a display 122, input component 121, and a processing core 206 including a communications processor 222 coupled with memory 223 and an applications processor 224 coupled with memory 225. Also included can be separate memory 230, RF transceiver 228 with antenna 229, and power supply 226 with power management module 238. Further, reader device 120 can also include a multi-functional transceiver 232 which can communicate over Wi-Fi, NFC, Bluetooth, BTLE, and GPS with an antenna 234. As understood by one of skill in the art, these components are electrically and communicatively coupled in a manner to make a functional device.

### Example Embodiments of Sensor Control Devices

FIGS. 2B and 2C are block diagrams depicting example embodiments of sensor control devices 102 having an analyte sensor 104 and sensor electronics 160 (including analyte monitoring circuitry) that can have the majority of the processing capability for rendering end-result data suitable for display to the user. In FIG. 2B, a single semiconductor chip 161 is depicted that can be a custom application specific integrated circuit (ASIC). Shown within ASIC 161 are certain high-level functional units, including an analog front end (AFE) 162, power management (or control) circuitry 164, processor 166, and communication circuitry 168 (which can be implemented as a transmitter, receiver, transceiver, passive circuit, or otherwise according to the communication protocol). In this embodiment, both AFE 162 and processor 166 are used as analyte monitoring circuitry, but in other embodiments either circuit can perform the analyte monitoring function. Processor 166 can include one or more processors, microprocessors, controllers, and/or microcontrollers, each of which can be a discrete chip or distributed amongst (and a portion of) a number of different chips.

A memory 163 is also included within ASIC 161 and can be shared by the various functional units present within ASIC 161, or can be distributed amongst two or more of them. Memory 163 can also be a separate chip. Memory 163 can be volatile and/or non-volatile memory. In this embodiment, ASIC 161 is coupled with power source 172, which can be a coin cell battery, or the like. AFE 162 interfaces with in vivo analyte sensor 104 and receives measurement data therefrom and outputs the data to processor 166 in digital form, which in turn processes the data to arrive at the end-result glucose discrete and trend values, etc. This data can then be provided to communication circuitry 168 for sending, by way of antenna 171, to reader device 120 (not shown), for example, where minimal further processing is needed by the resident software application to display the data.

FIG. 2C is similar to FIG. 2B but instead includes two discrete semiconductor chips 162 and 174, which can be packaged together or separately. Here, AFE 162 is resident on ASIC 161. Processor 166 is integrated with power management circuitry 164 and communication circuitry 168 on chip 174. AFE 162 includes memory 163 and chip 174 includes memory 165, which can be isolated or distributed within. In one example embodiment, AFE 162 is combined with power management circuitry 164 and processor 166 on one chip, while communication circuitry 168 is on a separate chip. In another example embodiment, both AFE 162 and communication circuitry 168 are on one chip, and processor 166 and power management circuitry 164 are on another chip. It should be noted that other chip combinations are possible, including three or more chips, each bearing responsibility for the separate functions described, or sharing one or more functions for fail-safe redundancy.

### Example Embodiments of a Time-In-Range Software Application

In many embodiments, the subject matter described herein is implemented by a software application program that is stored in a memory of and executed by a processor-based device, such as any one of the reader devices (e.g., a smart phone), drug delivery devices, trusted computer system, local computer system, or any of the other computing devices described herein. In certain embodiments, the software is implemented as one or more downloadable software applications (“an App”) on a reader device such as a mobile communication device or a smartphone. In certain embodiments, the software, and its associated features and functionalities, can be implemented on a single centralized device or, in the alternative, can be distributed across multiple discrete devices in geographically dispersed locations. Likewise, those of skill in the art will recognize that the representations of various computer systems in the embodiments disclosed herein, as shown in FIG. 1, are intended to cover both physical computing devices and virtual computing devices (e.g., virtual servers or virtual machines).

Generally, the software can provide a mechanism for a user to define consumables (e.g., a type of food, type of drink, or a portion thereof), in a fashion that is convenient to the user. These consumables will be referred to generally herein as a meal or meals, and these terms are used broadly to denote all types of food and drink.

According to an aspect of many embodiments, the software can perform a number of functions related to the collection of meal information and association of that meal information with analyte information collected by in vivo analyte sensor 104 or by in vitro test strip and meter, or from trusted computer system 180. The software will be generally referred to, hereinafter, as the “time-in-range application,” “TIR application,” or “meal monitoring application.”

According to another aspect of many embodiments, the TIR application can be used by diabetic patients, including patients having Type 2 diabetes that are administering basal insulin, or Type 1 or Type 2 patients on multiple dose insulin therapy that have an underlying motivation to change their diet. It can also be used by patients with pre-diabetes or non-diabetic people who want to minimize their glucose excursions by controlling their diet. Users of the TIR application may have uncontrolled diabetes and a desire to bring their diabetes under control, but have found that following a prescriptive “diabetes diet” for an extended period of time is not sustainable because they do not want to give up the foods that they enjoy. Users of the TIR application may also have their diabetes under control, but their usual diet is no longer working because they are, e.g., on a new medication, on a new exercise plan, pregnant, or experiencing other life changes.

The TIR application can allow an individual to log information about each meal that the individual consumes (i.e., each “meal event”). The TIR application can associate analyte data from the pertinent time period where the user's log entry indicated that a meal was consumed.

In some embodiments, the TIR application classifies food choices according to glycemic (or other analyte) response. It can be used in conjunction with analyte monitoring system 100 to help people better understand the impact of their diet on their glucose levels. The TIR application can associate a measured analyte response with a meal event and store the results in a non-transitory memory or a database. In particular, the TIR application can display each meal with its associated analyte (e.g., glucose or other analyte) response to the user, for example, as a list where each meal is ranked and sorted by descending degree of, using glucose as an example, glycemic response magnitude. Users can see directly how food choices, along with portion sizes, affect their glucose levels. Users can learn which foods have the biggest impact. The TIR application also helps dispel myths about healthy eating (e.g., problematic high carbohydrate foods such as orange juice and breakfast cereal that can be incorrectly viewed as healthy).

In many embodiments, the TIR application can enable a user to see the good and bad foods (or good and bad eating behavior) that exist within their current diet in order to assist the user in determining which modifications they can make to achieve improved glucose control and improve the time that their glucose levels remain in a target range. The TIR application can assist the user by providing data visualization of glucose responses and the TIR impact of different food choices. The TIR application can also provide easy-to-understand scores for each of the logged meals according to changes detected in the user's glucose levels within a predetermined time period (e.g., three hours) after eating. In some embodiments, for example, a higher scoring meal can correspond to a meal associated with a lower glycemic response. Further, a lower scoring meal can correspond to a meal associated with a higher glycemic response. In this manner, a higher scoring meal indicates to the user that a particular food choice can help them stay within a target range of analyte values. In some embodiments, the TIR application can gamify TIR in order to motivate users to make beneficial food choices and increase a TIR metric.

The TIR application can utilize and evaluate the amount of time the user is within a range of values to assess glucose impact of meals (and other events). For example, the TIR application can evaluate the amount of time a user is within a range of values, or TIR (e.g., in a target glucose range of about 70 mg/dL to about 180 mg/dL). In some embodiments, a target range can be set by the user. For example, the target range could be set from about 80 mg/dL to about 170 mg/dL. The target range may have a lower bound of at least around 65 mg/dL, for example around 70 mg/dL or 80 mg/dL. The target range may have an upper bound of no more than around 180 mg/dL, for example around 170 mg/dL or 120 mg/dL The target can also be incrementally and automatically adjusted (without requiring user intervention) by the TIR application if the user is meeting the currently assigned target or goal after some predetermined period of time. For example, if the user has consistently reached a target of 30% of their glucose measurements in the target range, e.g., for the past week, the TIR application can set a new target of maintaining 35% of measured analyte levels in the target range for the user. Similarly, if the user has consistently failed to reach a target of 30% of their glucose measurements in the target range, the TIR application can set a new target of maintaining 25% of measured analyte levels in the target range for the user. However, those of skill in the art will understand that other target ranges can be utilized besides those listed or described herein, and that these numbers are not meant to be limiting.

TIR can be determined as percentage value by dividing time the analyte level has been within a range, over a total period of time. For example, if the glucose is within the TIR threshold for 6 hours, over a total period of 24 hours, then the TIR metric is 25%. Alternatively, the metric can be displayed in terms of time increments instead of percentage; using the example above, the TIR would be 6 hours (in a total period of 24 hours).

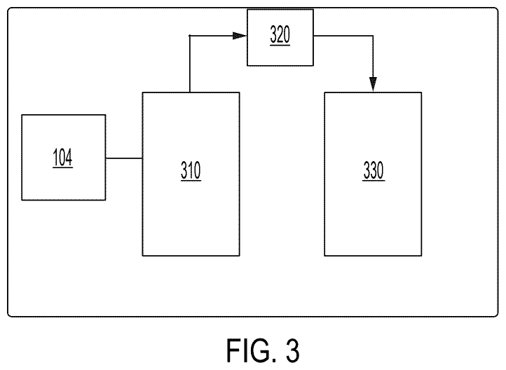

In many embodiments, the user of the TIR application can have their glucose continuously monitored, such as with sensor control device 102 of system 100. As seen in FIG. 3, the analyte levels can be transmitted: (i) from the sensor 104 to an analyte monitoring application 310; (ii) from the analyte monitoring application 310 to a cloud comprising server 320; (iii) from server 320 to a TIR application 330. In some embodiments, cloud 320 can include one or more servers having the same or different functions. For example, analyte data can be uploaded to a first server or group of servers responsible for collecting analyte data, and then downloaded to the TIR application by a second server or group of servers responsible for downloading the data for use by the time-in-range application. Those of skill in the art will also appreciate that the analyte monitoring application 310 and TIR application 330 can reside on a single device such as, e.g., a single smart phone, or, in the alternative, can reside on two different devices such as, e.g., two smart phones, or a smart phone and a dedicated receiver. Those of skill in the art will further appreciate that, in some embodiments, the numerous features and functions of the TIR application, as described herein, can be incorporated in analyte monitoring application 310. In such cases, the analyte data received from sensor 104 by analyte monitoring application 310 need not be transmitted to a cloud or server to perform the features and functions of the TIR application.

Additional details regarding features and interfaces of Time-in-Range software applications, any of which can be implemented and/or used in combination with the embodiments described herein, can be found in U.S. Patent Publication Nos. 2017/0128007, 2021/0030323, and 2022/0000399, all of which are hereby incorporated by reference in their entireties for all purposes.

### Example Embodiments of GUIs and Related Features for a Time-In-Range (“TIR”) Application

Example embodiments of various GUIs and related software features for TIR application will now be described. Those of skill in the art will understand that these various interfaces can be displayed on any of the embodiments of reader device 120 (e.g., a smart phone), drug delivery device 160, trusted computer system 180, or local computer system 170 described herein. These interfaces, and their associated features and functionalities, can be implemented on a single centralized device or, in the alternative, can be distributed across multiple discrete devices in geographically dispersed locations. It will be understood by those of skill in the art that any one or more of the example embodiments of the methods, interfaces, and systems described herein can either be implemented independently, or in combination with any of the other embodiments described in the present application. Further, although many of the embodiments described herein relate to glucose monitoring, those of skill in the art will appreciate that these same embodiments can be implemented for purposes of monitoring other analytes, such as, for example, lactate and ketones. In addition, those of skill in the art will appreciate that the embodiments described herein are not limited to the monitoring of one analyte at a time, although each embodiment described herein is capable of doing so.

### Example Embodiments of Home GUIs and Related Methods

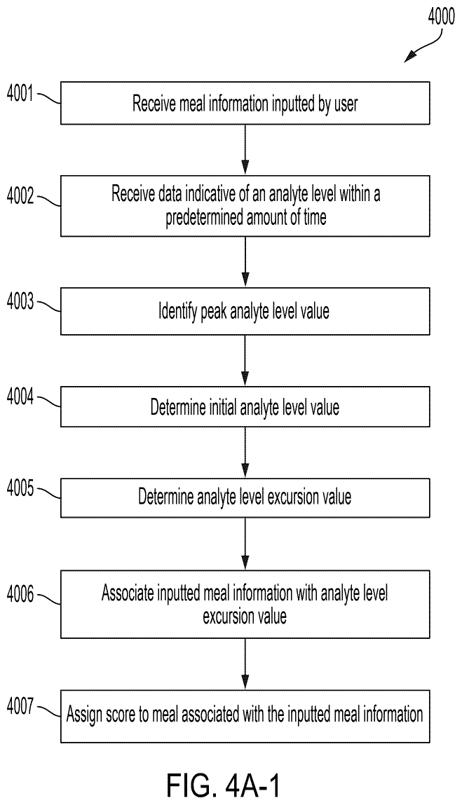

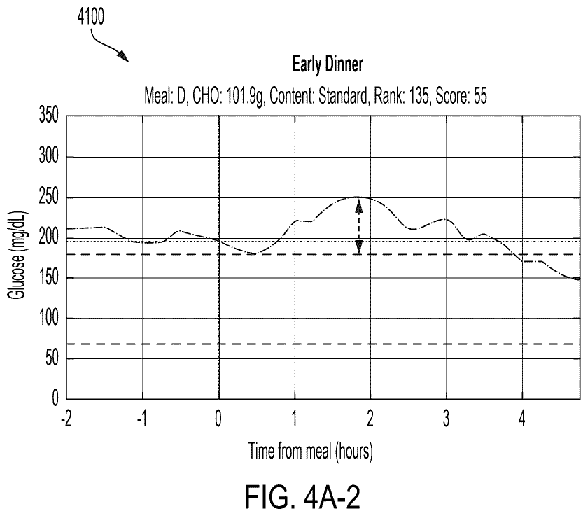

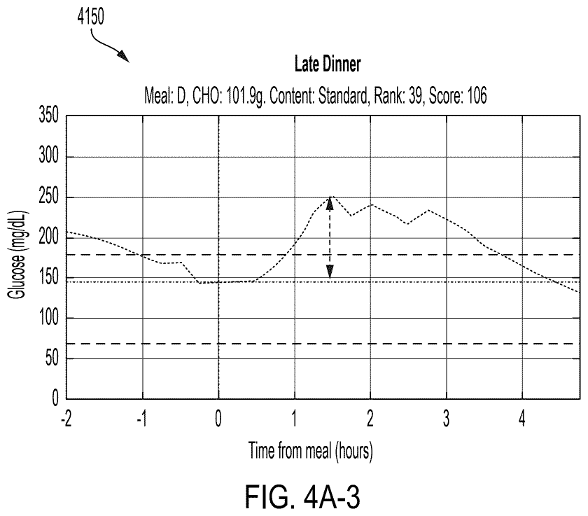

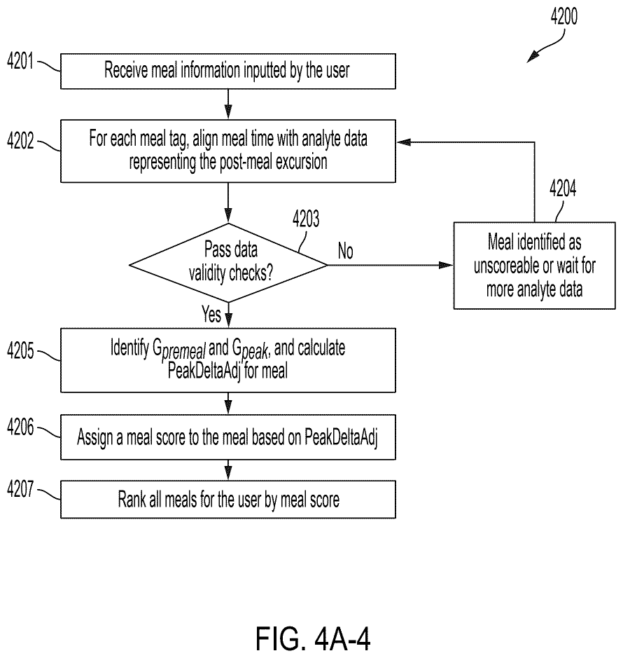

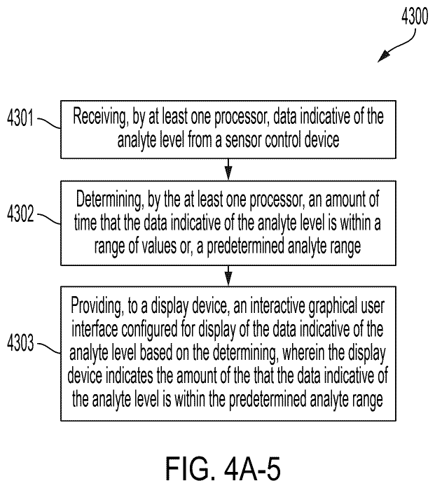

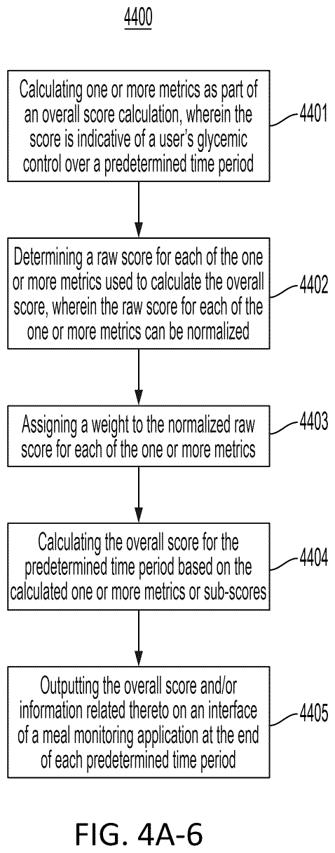

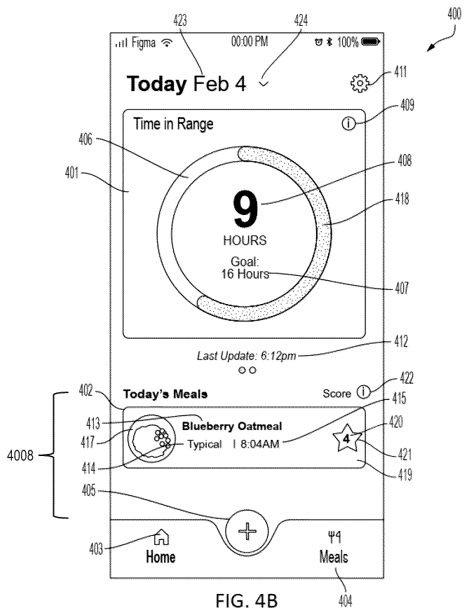

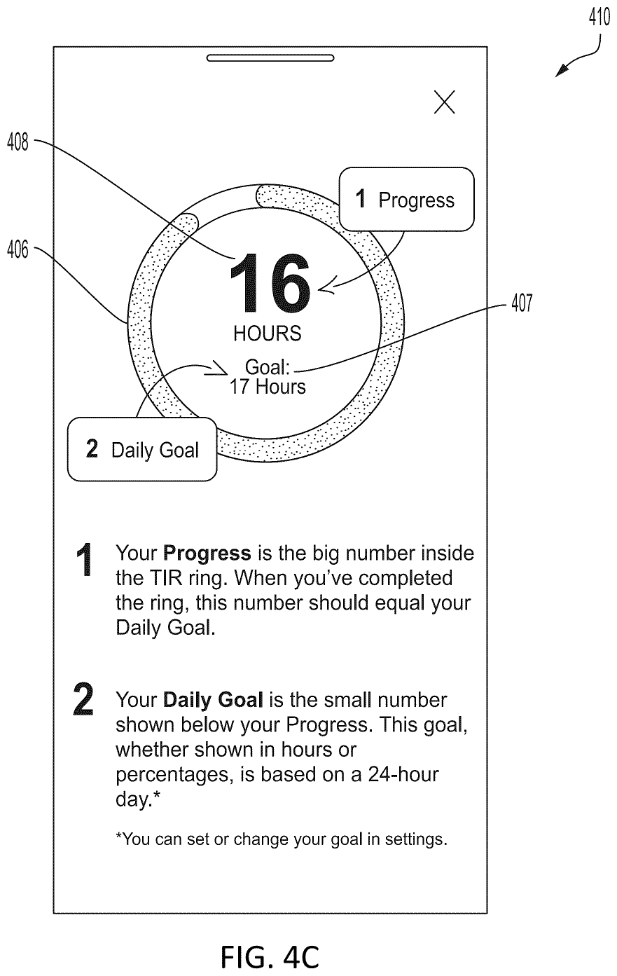

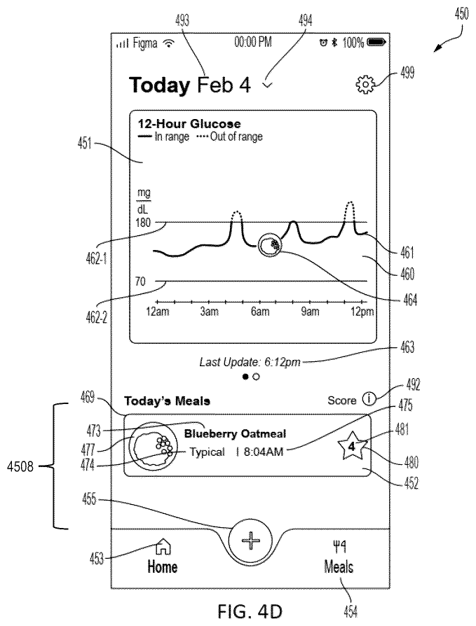

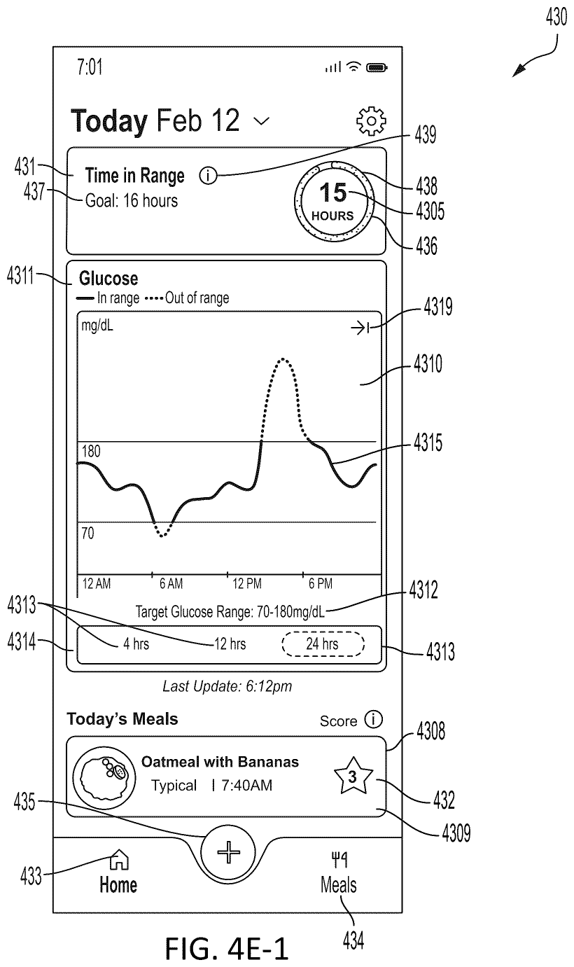

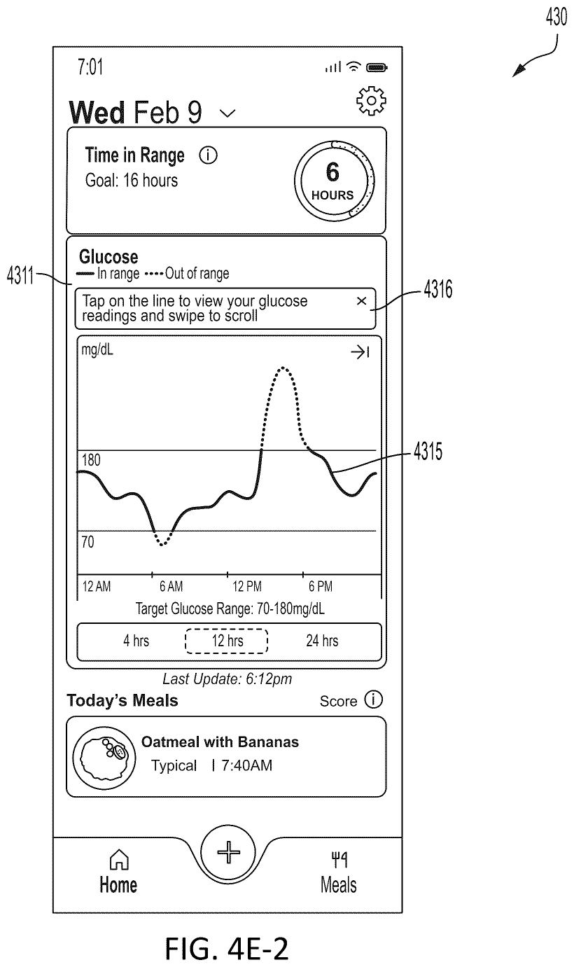

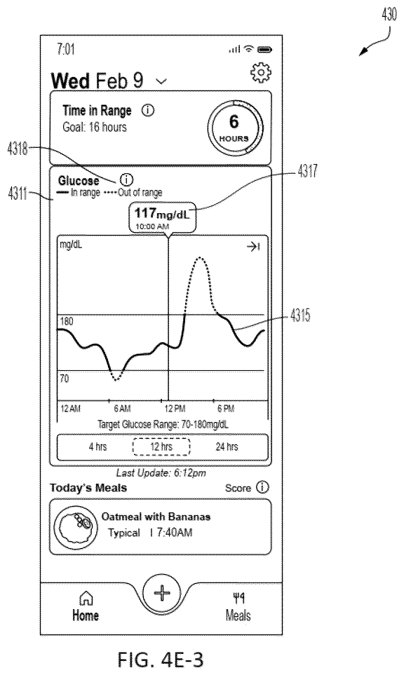

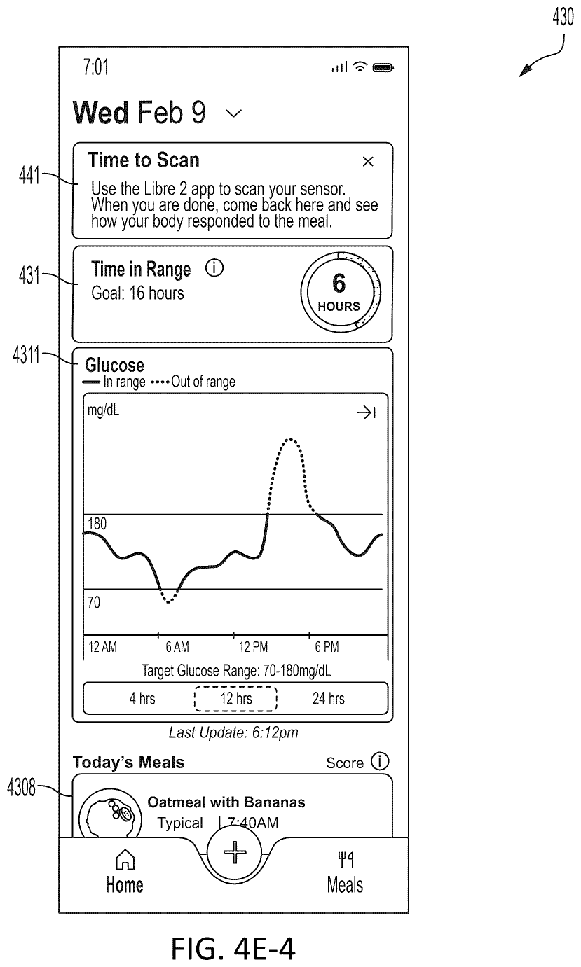

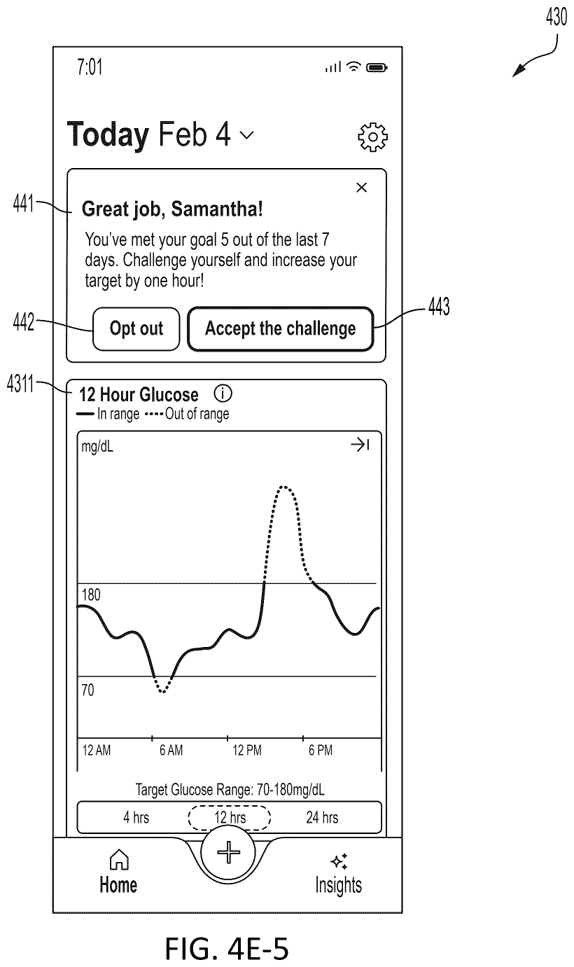

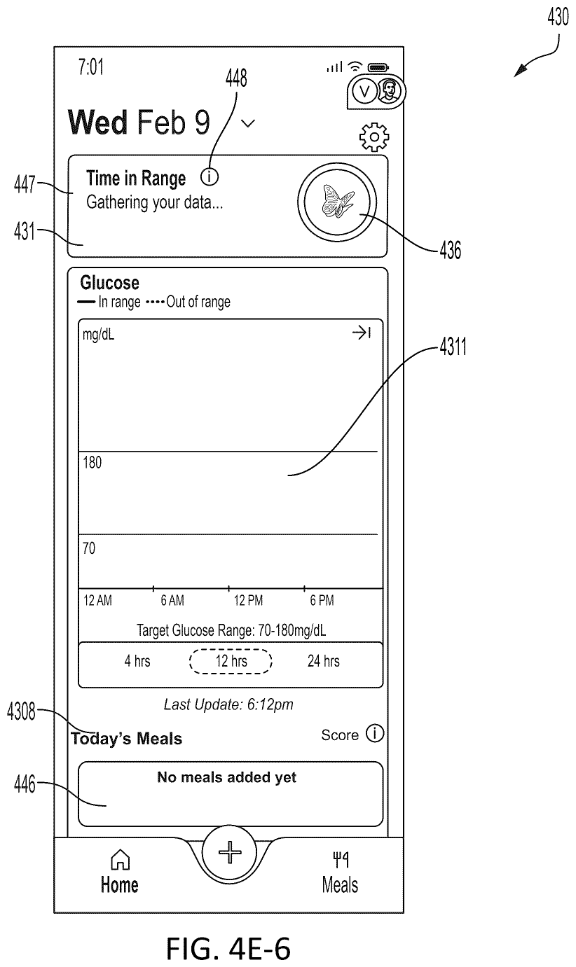

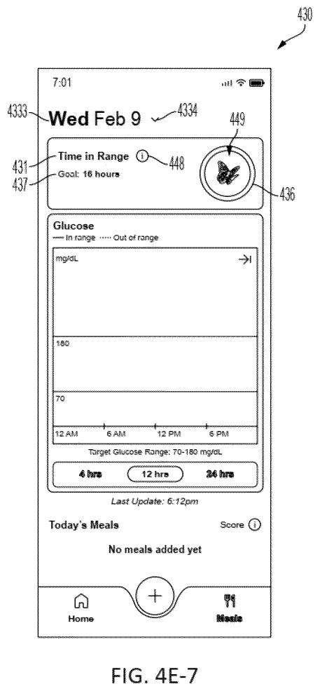

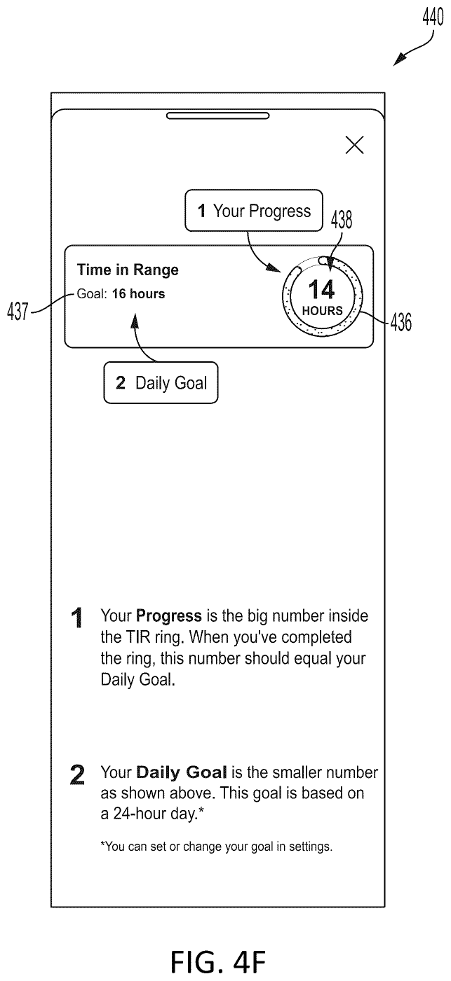

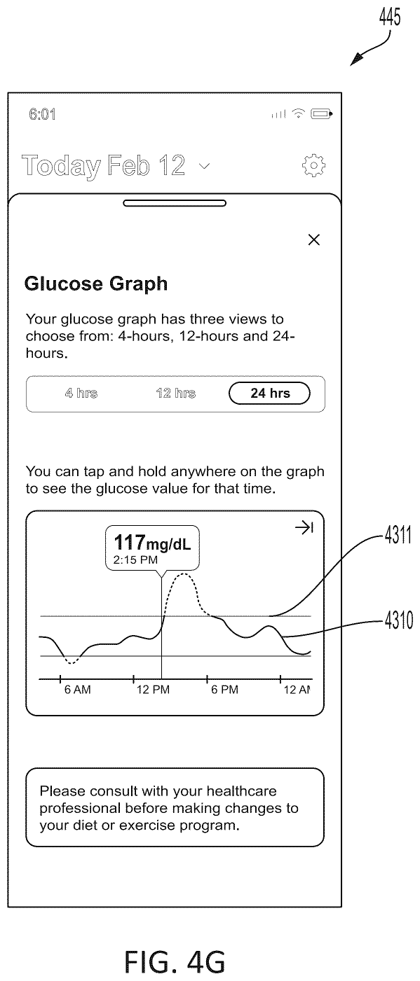

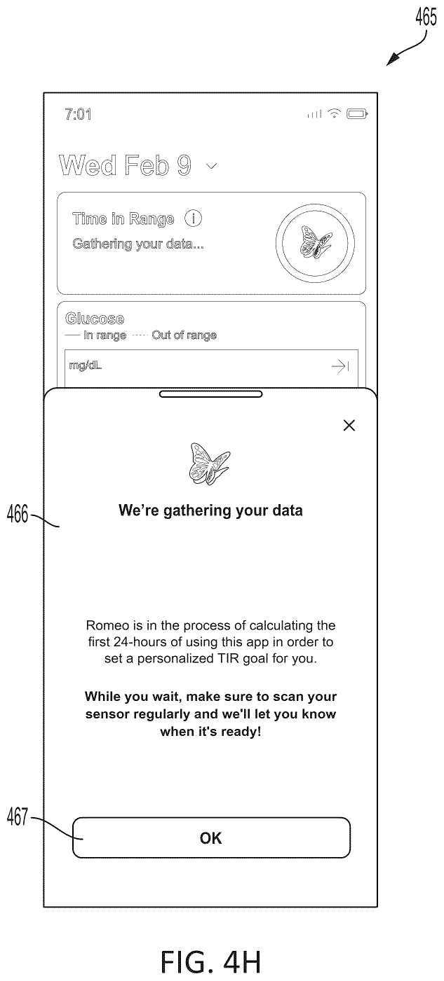

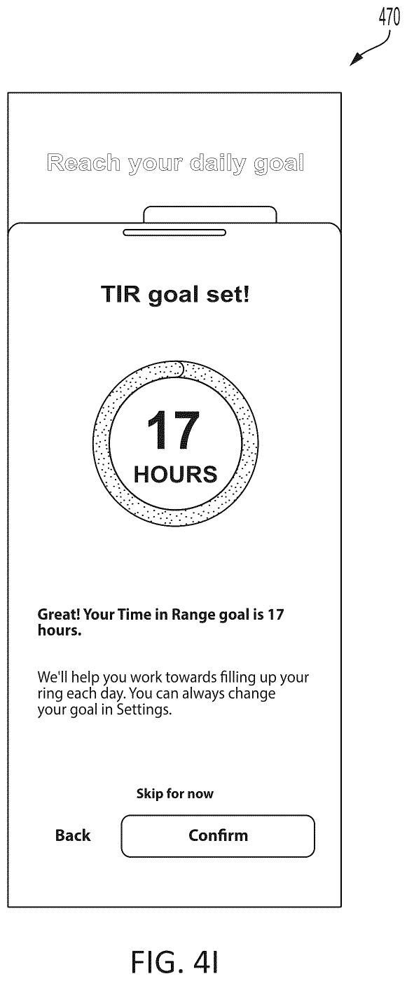

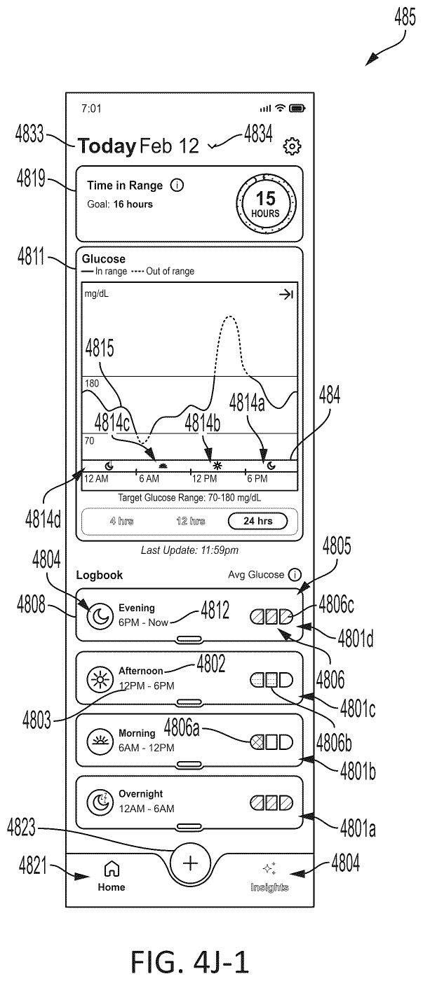

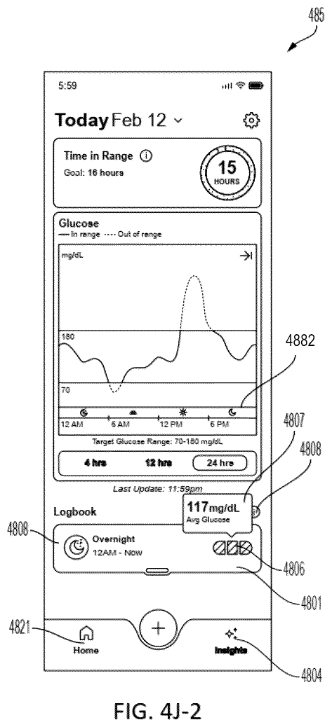

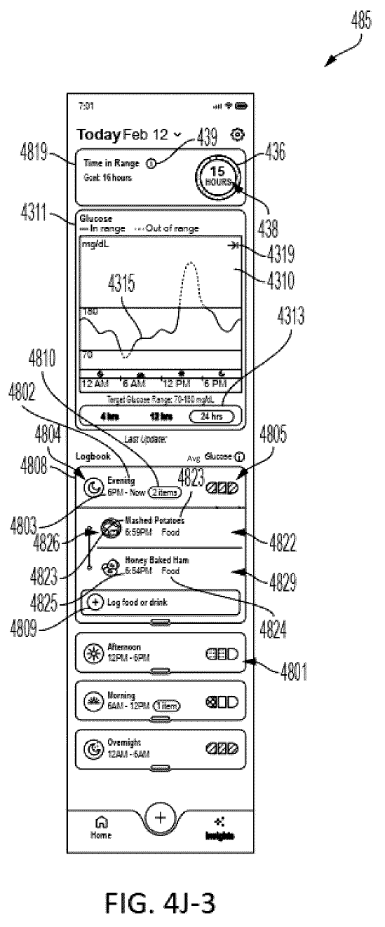

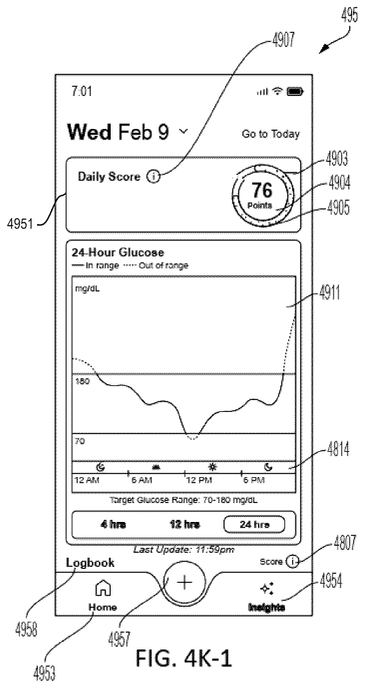

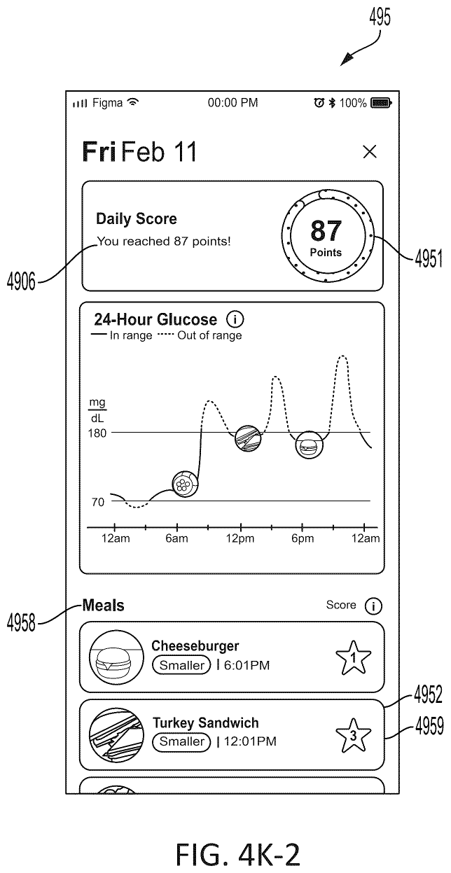

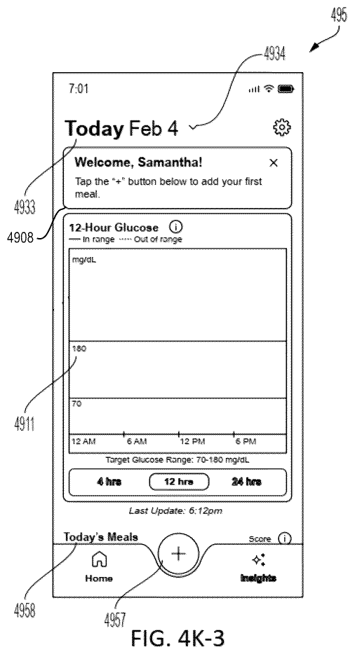

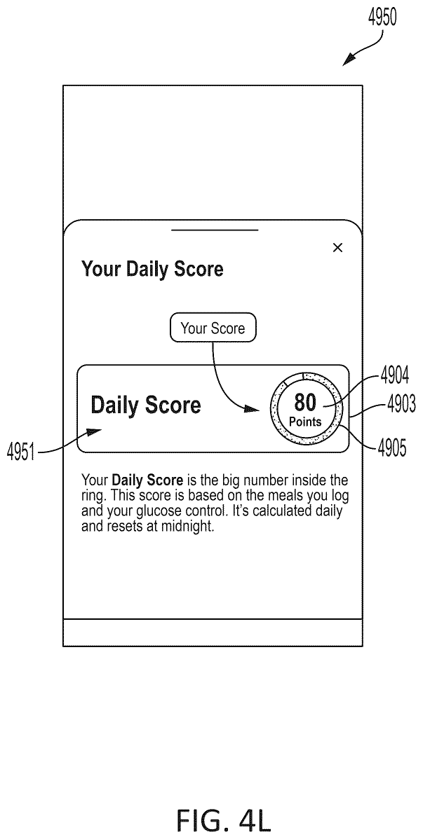

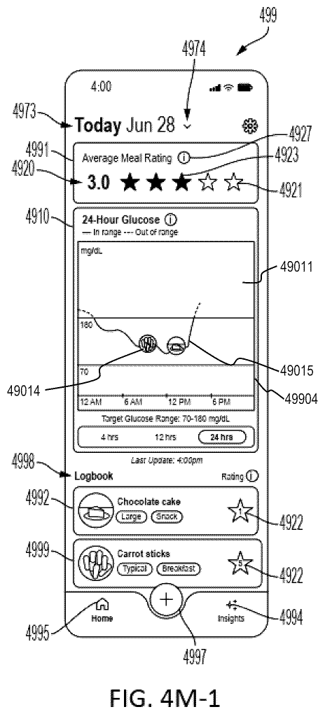

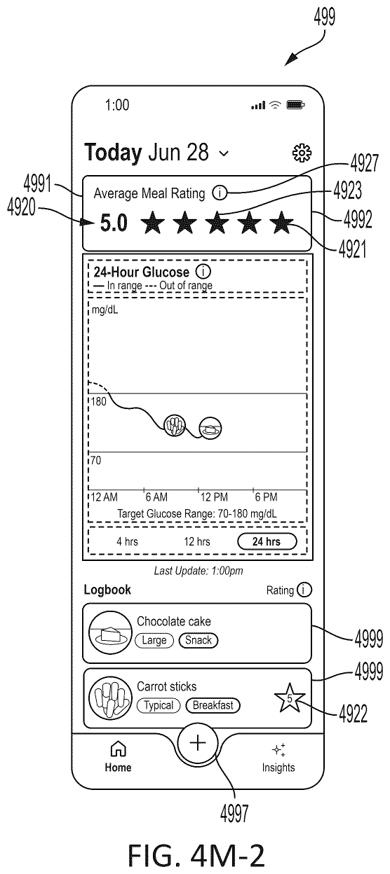

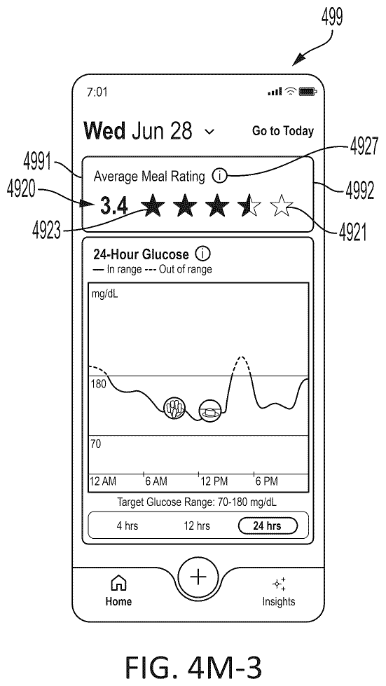

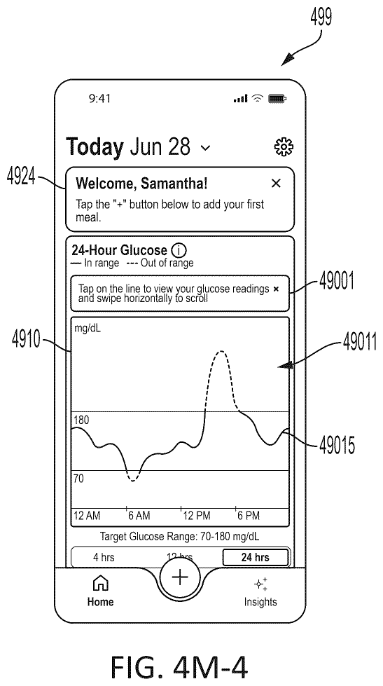

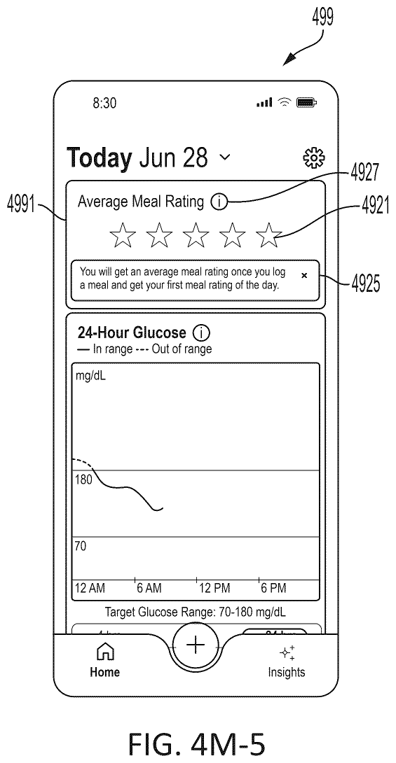

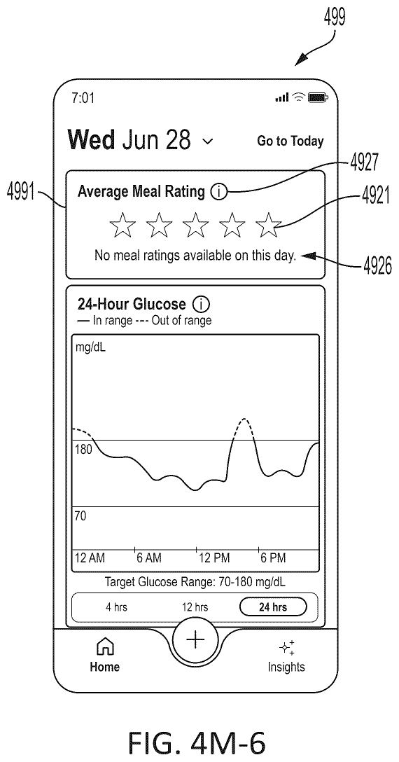

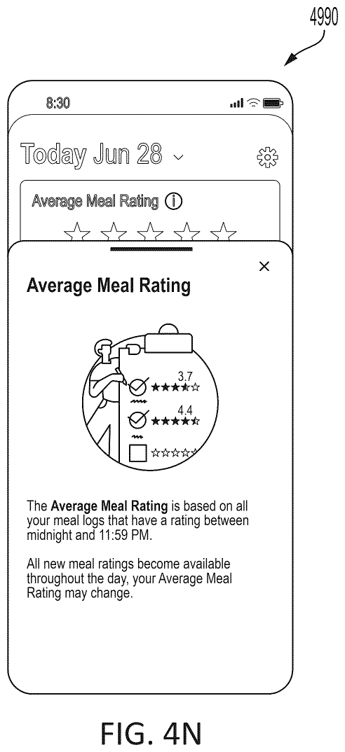

Example embodiments of methods for associating analyte data with meal information will now be described. As an initial matter, those of skill in the art will recognize that the method steps described herein can comprise software instructions stored in a memory of a computing device of system 100 (e.g., a reader 120, a local computer system 170, a trusted computer system 180), such that the instructions, when executed by one or more processors of the computing device, cause the one or more processors to perform any or all of the method steps described herein. Turning to FIG. 4A-1, a flow diagram depicts an example embodiment of a method 4000 for associating analyte data with meal information, where the meal information has already been entered by the user. As seen at the top of FIG. 4A-1, method 4000 begins at 4001, where meal information is inputted by the user. In some embodiments, for example, this can be a meal entry or in an entry in a meal log proactively entered by the user without any prompting from the TIR application. In other embodiments, the user can input meal information in response to a prompt displayed by the TIR application. For example, according to some embodiments, the TIR application can be configured to display a reminder notification to the user if a meal entry has not been entered after a predetermined reminder time period (e.g., no meal entry in the past week, no meal entry in the past three days, no meal entry in the past day, etc.).

Subsequently, at 4002, data indicative of an analyte level of the user is received by the TIR application within a predetermined amount of time. In some embodiments, this can entail the user scanning their sensor control device within a predetermined amount of time from the time of the meal entry (e.g., within three hours of the meal entry, four hours of the meal entry, within eight hours of the meal entry, within twelve hours of the meal entry, etc.). In other embodiments, this can occur if the sensor control device is configured to autonomously and wirelessly transmit analyte data to the reader device.

Referring still to FIG. 4A-1, at 4003, a peak analyte value in the analyte data is identified. In some embodiments, this can entail identifying the highest glucose value over a predetermined analyte level threshold (e.g., 170 mg/dL, 180 mg/dL, 190 mg/dL, etc.). In some embodiments, if a newly identified highest glucose value is higher than a previous highest glucose value, then the peak analyte value will be updated to the newly identified highest glucose value. The peak analyte value can also be a glucose value over the predetermined analyte level threshold during a time window after the meal entry (e.g., during a one-hour window after meal entry, during a two-hour window after meal entry, during a three-hour window after meal entry, during a four-hour window after meal entry). In some embodiments, the peak analyte value can also be a glucose value over the predetermined analyte level threshold prior to the time of the meal entry (e.g., three hours prior to the meal entry). When the peak analyte value is determined prior to the time of the meal entry, the post-meal period ends early so as to allow the user to receive a score (as will be described below) before the full time window subsequent to the time the peak analyte value was determined (e.g., the full three hour window).

According to an aspect of the embodiments, if a percent difference between a most recent analyte value relative to the peak analyte value is less than a predetermined threshold, then detection of the peak analyte value will cease. In some embodiments, detection of the peak analyte value will cease after a predetermined amount of time has elapsed (e.g., three hours) after a meal entry.

Then, at 4004, the initial analyte level value is determined. According to some embodiments, the initial analyte level value can be determined by ascertaining the analyte level value at or near the time of the meal entry (e.g., within fifteen minutes either before or after meal entry).

In some embodiments, a historical analyte data range is defined as a post-meal period, wherein the post-meal period ranges from the time of the initial analyte level value (e.g., fifteen minutes prior to or after a meal entry, thirty minutes prior to or after a meal entry, or one hour prior to or after a meal entry) to an end time of the peak analyte value (e.g., at two-hours after the meal entry, at three-hours after the meal entry, at four-hours after the meal entry). In some embodiments, the end of the post-meal period may be one of the two following events which occurs first: (1) a first analyte (e.g., glucose) reading within three hours from the initial analyte level value, or (2) a last analyte (e.g., glucose) reading prior to a new meal entry being inputted.

Subsequently, in some embodiments, at 4005, an analyte level excursion value can be determined, for example, by subtracting the initial analyte level value from the peak analyte level value. Then, at 4006, the analyte level excursion value can be associated and stored together in memory with the meal entry inputted by the user. Then, at 4007, and based on the analyte level variance, a score or rating is assigned to a particular meal associated with the inputted meal information. In some embodiments, a particular meal is scored only after the end of the post-meal period. In some embodiments, a score for each of one or more meal events is calculated based on a glucose response based on data indicative of a glucose level associated with each meal event.

In some embodiments, the meal is not scored if certain thresholds conditions are met. A first threshold condition, for example, can be less than a minimum number of analyte readings (e.g., eight glucose readings) within the post-meal period and/or no peak analyte value was detected before a predetermined time window (e.g., a three-hour time window) elapsed from the time of the meal entry. Further, in some embodiments, a second threshold condition can be when the post-meal period is less than a predetermined time period (e.g., two hours). In some embodiments, if a new meal entry is inputted within a predetermined time period following a previously inputted meal entry (e.g., within three hours following an existing logged meal entry), then a peak analyte value cannot be detected, and the meal cannot be scored. Further, in some embodiments, if a new meal entry is inputted prior to a peak analyte value being detected, then the new meal cannot be scored.

According to some embodiments, assigning or calculating the meal score at 4007 can further take into account certain physiological conditions present in the user before the meal is consumed. More specifically, certain individuals with diabetes can have a high initial analyte level (e.g., glucose level) value prior to consuming a meal. For example, some individuals with Type 2 diabetes, who still have the ability to manufacture insulin to metabolize glucose, may frequently exhibit a high pre-meal glucose level. Endogenous insulin present in those individuals prior to consuming a meal can thus impact the response to the meal and may attenuate the analyte level excursion value (also referred to as “PeakDelta”). To illustrate, FIGS. 4A-2 and 4A-3 show post-prandial glucose traces 4100 and 4150, respectively, of the same meal at different times for an individual. The second plot 4150, on the right, depicts an initial analyte level value (also referred to as the pre-meal glucose level) of about 150 mg/dL and a PeakDelta value of about 100 mg/dL. The first plot 4100, on the left, depicts a pre-meal glucose level of about 180 mg/dL and a PeakDelta value of about 70 mg/dL for the same meal consumed. Thus, the PeakDelta value, or analyte level excursion value, can be attenuated for individuals with high pre-meal glucose levels.

To account for a higher initial analyte level for certain users, according to some embodiments, an adjustment can be applied to the PeakDelta value. For example, the Peak Delta value can be adjusted according to the following example equation:

$$PeakDeltaAdj = PeakDelta + f ( {G}_{premeal} ) \qquad ( 1 )$$

As explained above, PeakDelta is the analyte level excursion value, or the difference between the peak analyte level value following a meal and the initial analyte level. PeakDeltaAdj is the adjusted analyte excursion value, and Gₚᵣₑₘₑₐₗ is the initial analyte level. In some embodiments, Gₚᵣₑₘₑₐₗ is the glucose level at the timestamp prior to the meal tag timestamp. In other embodiments Gₚᵣₑₘₑₐₗ is the minimum glucose level prior to the meal tag time stamp within a predetermined duration (e.g., one hour, ninety minutes, two hours, three hours, etc.).

In addition, according to some embodiments, f(Gₚᵣₑₘₑₐₗ) can be represented by the following linear function:

$$f ( {G}_{premeal} ) = a * {G}_{premeal} + b \qquad ( 2 )$$

Variables, a and b, can be constants determined based on simulations of a population of virtual Type 2 diabetic patients that consumed a variety of meals including varying carbohydrate amounts, fat content, and protein content. In other embodiments, variables, a and b, can be constants determined from in vivo data. Those of skill in the art will appreciate that other methods for determining variables, a and b, using one or more of simulated data, in vivo data, population data, or other test data can be utilized. The first variable, a, can be multiplied with Gₚᵣₑₘₑₐₗ, while the second variable, b, can be added to the product of Gₚᵣₑₘₑₐₗ and a. In some embodiments, for example, a=0.4 and b=−50 mg/dL.

According to some embodiments, if data is available for multiple instances of the same meal (or same type of meal) eaten at different times/days, a weighted average of the variables, a and b, can utilized in calculating the adjusted analyte level excursion value, PeakDeltaAdj. In particular, the Gₚᵣₑₘₑₐₗ and Gₚₑₐₖ values can be associated for multiple instances of the same meal. Additionally, in some embodiments, the values for multiple instances of the same meal can be further grouped by meal period (e.g., breakfast, lunch, or dinner). For each meal with more than one instance, variables, a and b, can be estimated as aₑₛₜ and bₑₛₜ. For meals with associated aₑₛₜ and bₑₛₜ values, a weighted average calculation for each parameter can be performed, wherein the weighting is more for meals with more instances. Subsequently, the PeakDeltaAdj value can be determined based on the latest weighted average parameters for a particular user.

Those of skill in the art will understand that other variables and/or constants can be utilized in the equations described herein without departing from the scope of the present disclosure.

According to other embodiments, a logistic function can be utilized to minimize the influence of Gₚᵣₑₘₑₐₗ when PeakDelta is low. This can serve to reduce improper scoring for small meals with minimal analyte level excursion values. One example embodiment of a logistic function is shown by the following equations:

$$k = \frac{1}{1 + {e}^{- \text{.5} * ( d - 5 )}} \qquad ( 3 )$$

$$PeakDeltaAdj = k ( a * Gpremeal + d - b ) + ( 1 - k ) d \qquad ( 4 )$$

In the above equations, d is PeakDelta, and a and b are constants.

Furthermore, according to some embodiments, one or more data validity checks can be performed before calculating a PeakDeltaAdj value. For example, in some embodiments, a first data validity check can be performed to ascertain whether the predetermined time window after the meal tag timestamp (also referred to as the excursion period) contains at least two (2) hours of glucose data. As another example, a second data validity check can be performed to ascertain whether there is another meal tag within the excursion period, and if so, whether the excursion period contains at least two (2) hours of glucose data prior to the meal tag.

According to another aspect of some embodiments, the scoring metric itself can be a function of Gₚᵣₑₘₑₐₗ and/or Gₚₑₐₖ.

FIG. 4A-4 is a flow diagram depicting an example embodiment of a method 4200 for associating analyte data with meal information that accounts for a high pre-meal glucose level of the user. As seen at the top of FIG. 4A-4, method 4200 begins at 4201, where the software receives meal information inputted by the user. In many embodiments, this can be meal tags entered by the user with time stamps that can be correlated with glucose data received from a sensor control device worn by the user. At 4202, for each meal tag, the meal time is aligned with the glucose data to reflect the post-meal excursion. That is, from the analyte data and the meal tag, the initial analyte level value and the peak analyte level value for each meal should be ascertainable. Subsequently, at 4203, one or more data validity checks can optionally be performed, as described above. If the one or more data validity checks are not passed, then the software can indicate that the meal is unscoreable. In some embodiments, the software can prompt the user to correct an unscoreable meal either by inputting more analyte data, by correcting the meal tag information, or by other means. In some embodiments, the software can prompt the user to delete an unscoreable meal tag.

Referring still to FIG. 4A-4, if the one or more data validity checks are passed, then at 4205, the initial analyte value (Gpremeal) and peak analyte level value (Gpeak) are identified. An adjusted analyte level excursion value (PeakDeltaAdj) can then be calculated using any of the methods described above. Subsequently, at 4206, a meal score can be calculated or assigned to the meal based on the adjusted analyte level excursion value (PeakDeltaAdj). Optionally, at 4207, all meals for the user can be ranked by their assigned scores.

In some embodiments, the adjusted analyte level excursion value assigned to each meal within a predetermined period of elapsed time (e.g., the last 24 hours) is averaged together and used to determine a combined meal score for a predetermined time period (e.g., the previous day or last 24 hours). In some embodiments, the combined meal score can be displayed on an interactive graphical user interface as a numerical value or a representation thereof. Specifically, in some embodiments, the combined meal score can be a maximum analyte level value. For example, in some embodiments, the maximum analyte level value of the combined meal score can be 150 mg/dL. According to an aspect of the embodiments, a determined analyte level value that exceeds the maximum analyte level value will be set to the maximum analyte level value for the purposes of determining a numerical range of the combined meal score (e.g., if the determined analyte level value exceeds 150 mg/dL, then the analyte level value will be considered as 150 mg/dL for purposes of determining the numerical range of the combined meal score). According to another aspect of the embodiments, the analyte level value associated with the combined meal score is divided by a predetermined value (e.g., 1.5 mg/dL) to achieve a unitless range of the combined meal score. For example, in some embodiments, the range of the combined meal score can be a numerical number between one and 100. As such, in some exemplar embodiments, if the combined meal score is associated with an analyte level value of 150 mg/dL, then the combined meal score is calculated as 100. Those of skill in the art will appreciate that other predetermined values, maximum analyte level values, and ranges can be utilized for the combined meal score without departing from the scope of the present disclosure.

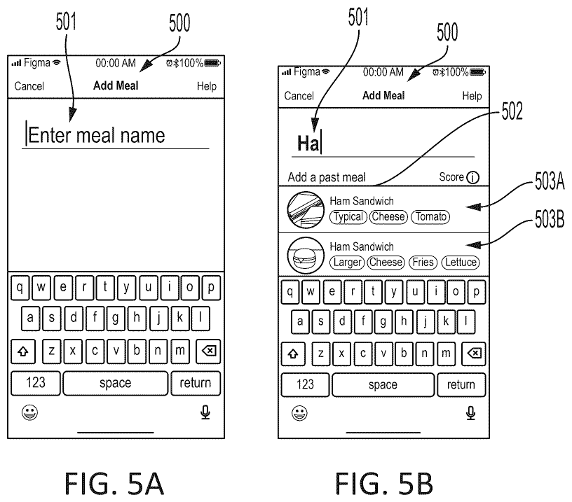

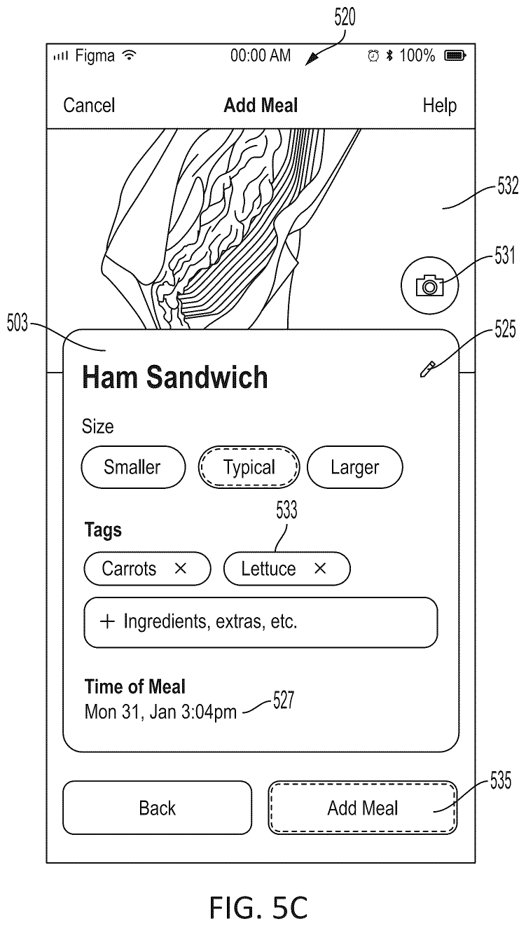

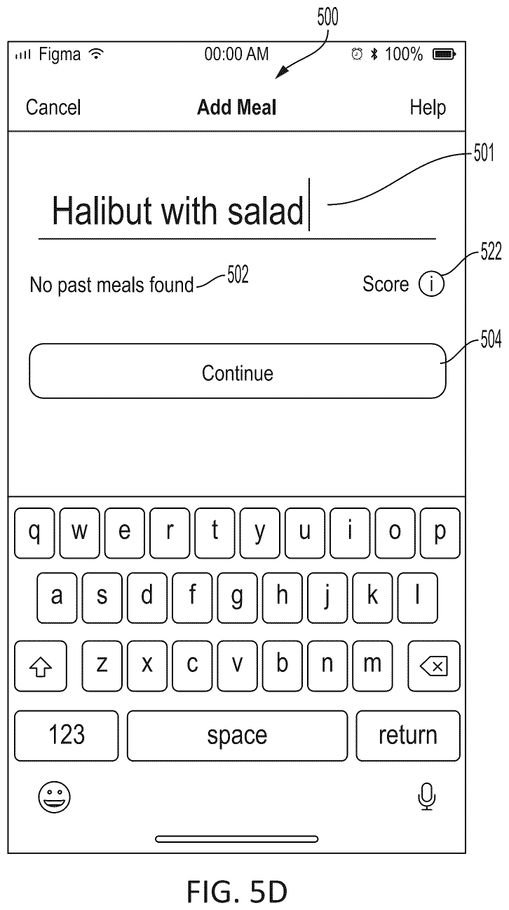

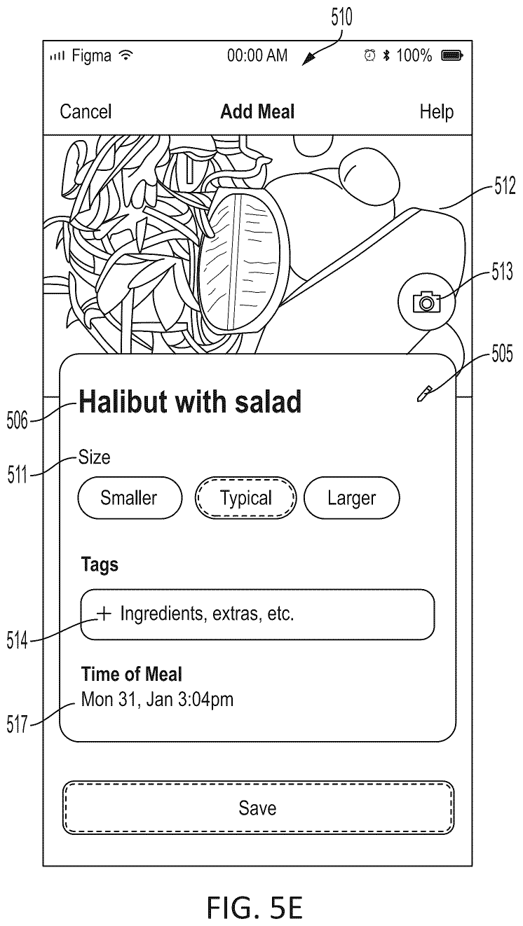

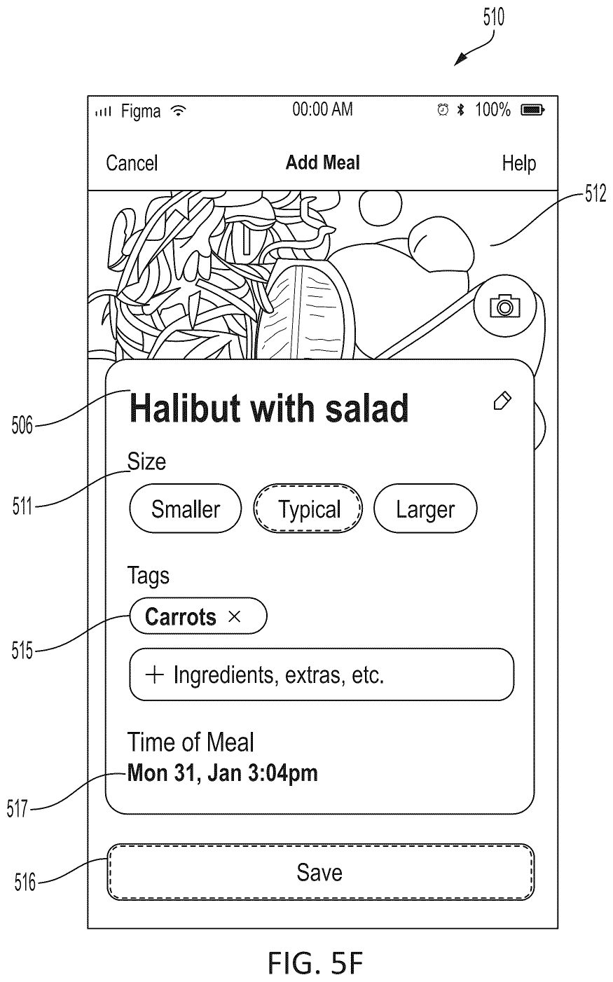

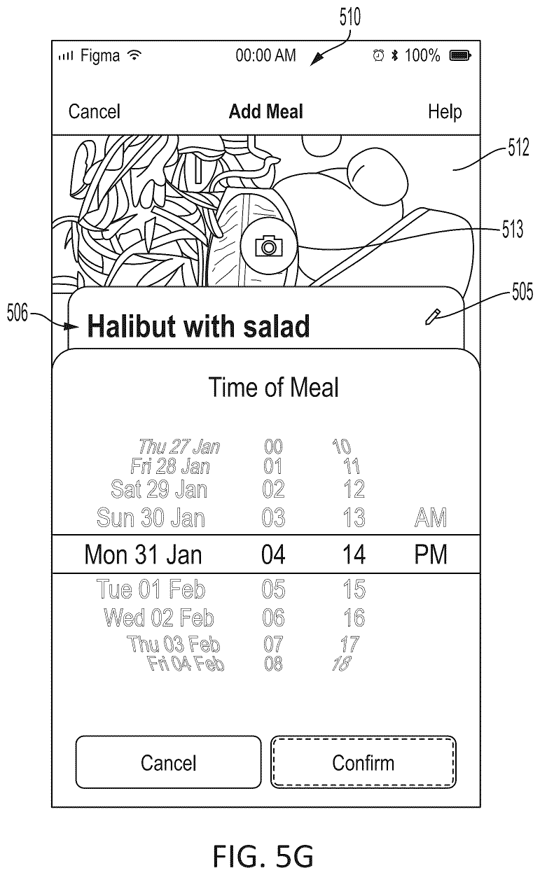

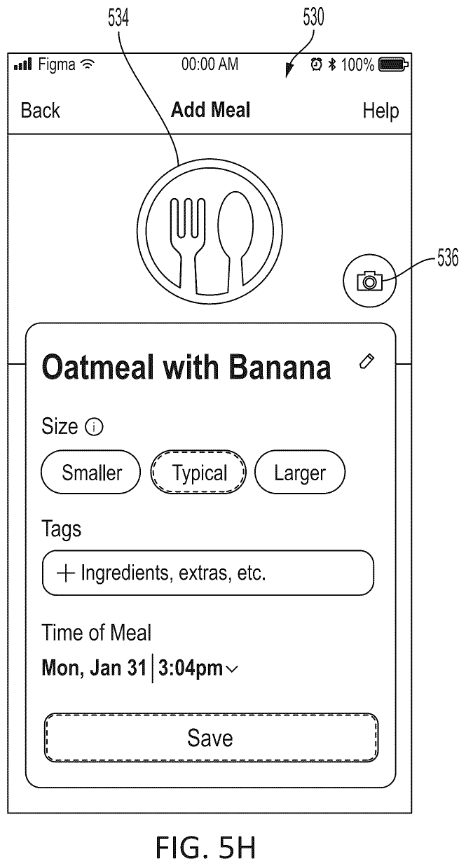

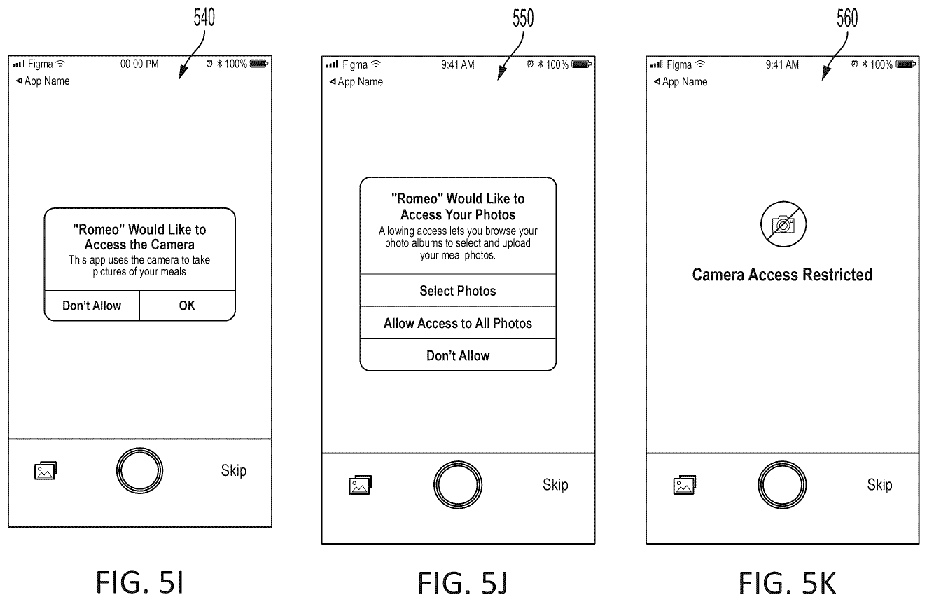

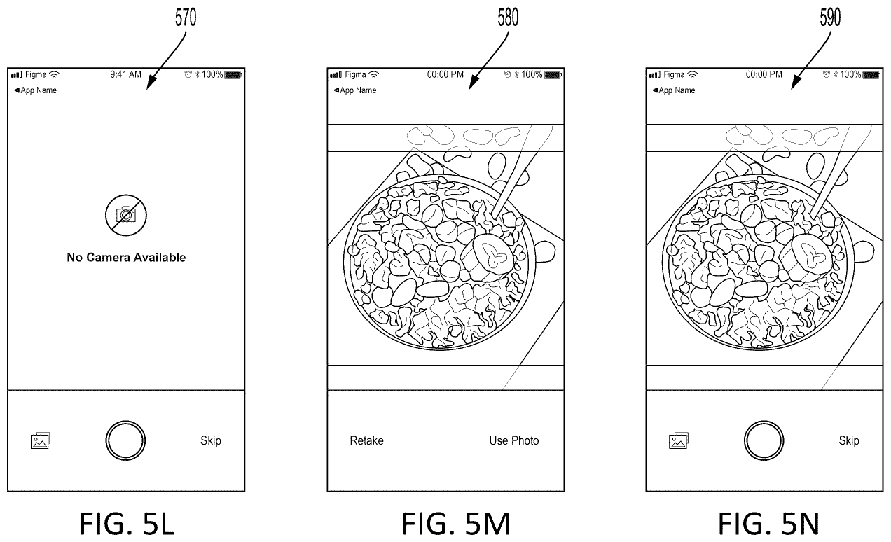

FIG. 4A-5 shows operations of a method 4300 for performing the process for an interface that displays TIR information and non-medical data from a sensor control device 102. As an initial matter, those of skill in the art will also recognize that any combination, subset, or all of the steps of method 4300 can be implemented in combination with any combination, subset, or all of the steps of methods 4000 and/or 4200 described above. For ease of illustration, the monitored analyte in this and other embodiments described below will be glucose, although other analytes can be monitored as well, as is noted herein.

Referring to FIG. 4A-5, at 4301, the method for an interface includes receiving, by at least one processor, data indicative of the analyte level (e.g., data indicative of a glucose level) from a sensor control device 102.

At 4302, the method further includes determining, by the at least one processor, an amount of time that the data indicative of the analyte level is within a range of values or, a predetermined analyte range. For example, the predetermined analyte range can be defined by a glucose range of values between 70 mg/dL and 180 mg/dL.

At 4303, the method 4300 can include providing, to a display device, an interactive graphical user interface configured for display of the data indicative of the analyte level based on the determining, wherein the display device indicates the amount of time that the data indicative of the analyte level is within the predetermined analyte range. According to some embodiments, for example, the amount of time that the data indicative of the analyte level is within the predetermined analyte range can be displayed in terms of increments or percentage values, and can be graphically represented as portions or segments of a circle or ring, with the area of each portion or segment proportional to the percentage value. In other embodiments, the graphical representation can be a pie chart, one or more bars or bar portions, a line graph, one or more harvey balls, a thermometer, a numeric gauge (e.g., speedometer), or any other geometric shape or image configured to reflect a user's progress.

Turning to FIG. 4A-6, a flow diagram depicts an example embodiment of a method 4400 for calculating a user's glycemic control over a predetermined time period. As an initial matter, those of skill in the art will also recognize that any combination, subset, or all of the steps of method 4400 can be implemented in combination with any combination, subset, or all of the steps of methods 4000, 4200, and/or 4300 described above. For ease of illustration, the monitored analyte in this and other embodiments described below will be glucose, although other analytes can be monitored as well, as is noted herein.

Specifically, method 4400 can calculate a user's glycemic control over a predetermined time period and provide customized feedback to the user by utilizing a combination of one or more metrics and/or data associated with the user's continuous glucose monitoring device.

As seen at the top of FIG. 4A-6, method 4400 begins at 4401, where one or more metrics are calculated as part of an overall score, wherein the score is indicative of the user's glycemic control over the predetermined time period. The predetermined time period can be one day, one week, two weeks, 15 days, 30 days, one month, etc. Those of skill in the art will appreciate that various other predetermined time periods can be utilized at 4401 without departing from the scope of the present disclosure.

Specifically, at least one or more of the following metrics are utilized as part of the overall score: (1) one or more metrics recommended by the American Diabetes Association (“ADA”); (2) the user's data indicative of the analyte level (e.g., the data indicative of a glucose level) received from the sensor control device; or (3) a meal scoring metric. Method 4400 can utilize one or more of the following metrics recommended by the ADA: (1) an amount of time a continuous glucose monitoring device is worn; (2) an amount of time the continuous glucose monitoring device is active (including the number of times the sensor is scanned in a period of time); (3) glycemic variability; (4) an amount of time the user's glucose level is at or below a predetermined threshold value (e.g., at or below 54 mg/dL or 70 mg/dL); and, (5) an amount of time the user's glucose level is at or above a predetermined threshold value (e.g., at or above 180 mg/dL or 250 mg/dL). Those of skill in the art will appreciate that various other metrics recommended by the ADA can be utilized with method 4400 without departing from the scope of the present disclosure.

Additionally, method 4400 and, specifically, 4401, can utilize any one of the meal scoring metrics described herein. For example, the meal scoring metrics described in methods 4200 and/or 4300 can be utilized as part of the overall score calculated in 4401. Those of skill in the art will appreciate that various other meal scoring metrics can be utilized with method 4400 without departing from the scope of the present disclosure.

At 4401, the user's continuous glucose monitoring data over a predetermined period of time is also used for the calculations. For example, 4401 can use the user's data indicative of an analyte level (e.g., data indicative of a glucose level) over, e.g., one day, one week, one month, etc., to calculate the overall score.

Subsequently, at 4402, a raw score is determined for each of the one or more metrics used to calculate the overall score at 4401. The raw score for each of the one or more metrics can be normalized such that each metric meets the ADA guidance so as to map to a predefined numerical score value (e.g., a score of 70 or 80) less than a predefined maximum score value (e.g., a score of 100). In this manner, the raw score for each of the one or more calculated metrics can be normalized so as to allow the user potential to improve past the ADA guidance.

At 4403, each normalized metric can be assigned a weight. Specifically, a weight can be assigned to the normalized raw score for each of the one or more metrics. In this regard, particular weight can be applied to emphasize one or more particular metrics. All applied weights can sum up to 1. As such, each weighted metric is provided a sub-score.

Next at 4404, the overall score for the predetermined time period (e.g., one day, one week, etc.) is calculated based on the calculated one or more metrics or the sub-scores. If corresponding weights have been applied to one or more metrics, then all weighted sub-scores are summed up in order to determine the user's overall score for the predetermined time period. The overall score, for example, can be a numerical value (e.g., a numerical value between 1 and 100). The overall score can be updated at a predetermined frequency so as to provide an updated overall score for each predetermined time period. The overall score can be one numerical daily score that is provided to the user on a daily basis. In this manner, method 4400 provides user-friendly and actionable information that allows the user to better understand their progress in glycemic control and/or areas for improvement.

At 4405, the overall score and/or information related thereto can be displayed to the user at the end of each predetermined time period, wherein the overall score and/or information related thereto is outputted through a GUI of the meal monitoring application. For example, if a daily score is provided, then an updated or new daily score can be outputted to the user on a daily basis through the meal monitoring application. For metrics which achieved an overall score above a predetermined threshold overall score value, (e.g., a score at or above 85, 90, 95, etc.), the meal monitoring application can be configured so as to output information which provides positive feedback related to glycemic control and/or progress thereof. For metrics with an overall score below a predetermined threshold overall score value, (e.g., a score at or below 65, 60, 55, etc.), the meal monitoring application can be configured so as to output information which provides tailored suggestions/recommendation on improving the user's glycemic control. The overall score, and feedback related thereto, can be outputted to the user through the various interface embodiments described herein without departing from the scope of the present disclosure.

According to another aspect of some embodiments, the GUI of the meal monitoring application can also be configured to display historical overall scores to show the user's progress over time. In addition, according to some embodiments, the GUI of the meal monitoring application can further be configured to provide insights based on certain trends in the user's historical overall scores (e.g., when the score is trending up, when the score is trending down, how the score compares to an average overall score over a predetermined period, whether the score is higher or lower than a previous predetermined period of time).

Referring to FIG. 4B, a block diagram depicting an example embodiment of a home GUI 400 for a TIR application is shown. According to one aspect of the embodiments, home GUI 400 can comprise: (1) a TIR card 401; (2) a logbook section 4008 comprising a list 402 of meal events 419; (3) a selectable home icon 403 that, when selected by the user, outputs the home GUI 400; (4) a selectable meals icon 404 that, when selected by the user, outputs a meals report GUI comprising information on one or more meal events which occurred in a previous time period (e.g., the past week); and, (5) a “+” icon 405 that, when selected by the user, outputs a meal logging GUI 500 (see, e.g., FIGS. 5A to 5G) configured to receive meal information inputted by the user. In some embodiments, the TIR card 401 can include a graphical element indicating the amount of time that the user was in the target analyte range (“time in range” or “TIR”) for a predetermined time period (e.g., a 24-hour period). In some embodiments, though not illustrated, home GUI 400 can comprise a selectable insights icon instead of the meals icon 404. Specifically, though not illustrated in FIG. 4B, when the user selects the insights icon, an insights GUI 14000 or insights GUI 150000 (e.g., FIGS. 14A-1 to 14A-5, and FIGS. 15A-1 through 15A-5, respectively) is outputted, which will be described in further detail below.

In some embodiments, the TIR card 401 can highlight hours in range using a graphical element, such as a TIR circle or ring 406. As seen in FIG. 4B, TIR card 401 shows TIR ring 406 with a daily TIR goal 407 (number of hours for TIR) inside or in an inner area of the TIR ring 406. For example, the daily TIR goal 407 can comprise a numerical value and unit of measure indicative of the predetermined TIR goal for the user (e.g., “16 hours”). In some embodiments, the current number of hours that the user has spent in the predetermined target range can be displayed in two ways: (1) a TIR value 408 can be a numerical value and a unit of measure (e.g., “9 hours”) listed inside the ring, along with the daily TIR goal 407; and (2) the number of hours can be visually displayed as a progress indicator 418 (e.g., a shaded portion) extending along the perimeter of the TIR ring 406 to X % of the total perimeter of the TIR ring 406, to indicate that the user has spent X % TIR (where X % is the amount of hours spent in range divided by 24 hours, for example). In this manner, the ratio of the perimeter of the TIR ring that comprises the progress indicator 418 (e.g., the shaded portion) and the total perimeter of the TIR ring 406 is proportional to a ratio of the amount of time that the user has spent in the predetermined target analyte range in a day. In some embodiments, the progress indicator 418 can comprise a color having a gradient configured to transition between various color shades. In this manner, the degree by which the color gradient transitions between various shades is indicative of the progress the user has made in reaching their daily TIR goal 407. In some embodiments, the TIR value 408 can be represented by a numerical value in, e.g., percentages unit or hours, without the TIR ring 406 or a progress indicator of the like.

Those of skill in the art will further recognize that other types of progress indicators (e.g., textual, numerical, or graphical) can also be utilized, and are fully within the scope of the present disclosure. Furthermore, although the graphical element shown is a circle or ring having a shaded portion comprising a gradient, those of skill in the art will understand that other geometrical shapes, colors, and numbers of portions or gradients can be utilized, and are fully within the scope of the present disclosure.

In some embodiments, the TIR card 401 can further comprise a selectable informational icon 409 that, when selected by the user, outputs a TIR informational screen 410 related to TIR card 401. For example, and as depicted in FIG. 4C, the TIR informational screen 410 provides details on how the TIR ring 406 visually displays the progress indicator 408 and daily TIR goal 407. In some embodiments, the TIR informational screen 410 also informs the user that TIR goal settings are configurable. Referring back to FIG. 4B, in some embodiments, the home GUI 400 further comprises a selectable settings icon 411 which, when selected, outputs settings GUIs wherein the user can adjust TIR goal settings, e.g., changing the daily TIR goal 407 and metrics in which the TIR data is displayed (e.g., showing the TIR value 408 or daily TIR goal 407 in hours or percentages). In some embodiments, and as illustrated in FIG. 4B, the home GUI 400 further comprises a message 412 adjacent to the TIR card, wherein the message 412 indicates when the TIR card 401 was last updated.

Referring to FIG. 4B, home GUI 400 can include the logbook section 4008 with the list 402 of meal information comprising one or more meal events 419 that occurred in a predetermined time period (e.g., one day). Each of the meal events 419 provides details of a particular meal consumed within the predetermined time period. In some embodiments, each meal event 419 can include a text description of a meal 413 (e.g., “Blueberry Oatmeal”), a portion size indicator 414 (e.g., “Typical”) describing the relative portion size of the meal (e.g., typical, larger, or smaller compared to the user's usual meal serving), and/or a timestamp 415 associated with a time the meal was consumed (e.g., “8:04 AM”). Though not illustrated, in some embodiments, a meal event 419 can include one or more meals tags associated with the meal. The user can use a meal tag (not shown) to describe the contents or a characteristic of the meal, or list types of food, e.g., vegetables, chicken, beef, pork, fish, salad, pasta, or vegetarian, to name only a few examples of tags. Additionally or alternatively, the meal event 419 can include a photograph/picture 417 associated with the meal. In some embodiments, the user can take a photograph of the meal or, alternatively, select a photograph from a library of photographs. In some embodiments, when the user does not associate a photograph 417 with the meal, a placeholder photograph/picture 417 will be displayed instead. In some exemplar embodiments, the list 402 can be ordered such that the meal event 419 corresponding to the most recently consumed meal is presented first or at the top of the list, with subsequent meal events 419 being displayed in chronological order.

As illustrated in FIG. 4B, each meal event 419 can additionally or alternatively include a rating or score 420 of a single particular meal according to the analyte response associated therewith. In some embodiments, the analyte response can be based on a change in an analyte level within a predetermined time period after the particular meal is consumed. In some embodiments, meals are given a score 420 based on the analyte level excursion value from the time the particular meal was consumed to the peak glucose within three hours of eating.

Further, the score 420 can be displayed as a numerical value. In some embodiments, the score 420 can be a number between one and five, wherein a higher assigned numerical value corresponds to a lower analyte response, and a lower assigned numerical value corresponds to a higher analyte response. For example, (1) a 0 to 20 mg/dL change in an analyte level within a two hour period post-meal can correspond to a “5” score 420; (2) a 21 to 40 mg/dL change in an analyte level within a two hour period post-meal can correspond to a “4” score 420; (3) 41 to 60 mg/dL change in an analyte level within a two hour period post-meal can correspond to a “3” score 420; (4) a 61 to 90 mg/dL change in an analyte level within a two hour period post-meal can correspond to a “2” score 420; and (5), a 91 mg/dL or higher change in an analyte level within a two hour period post-meal can correspond to a “1” score 420. As another example, (1) a 0 to 24 mg/dL change in an analyte level within a three hour period post-meal can correspond to a “5” score 420; (2) a 25 to 49 mg/dL change in an analyte level within a three hour period post-meal can correspond to a “4” score 420; (3) 50 to 74 mg/dL change in an analyte level within a three hour period post-meal can correspond to a “3” score 420; (4) a 75 to 99 mg/dL change in an analyte level within a three hour period post-meal can correspond to a “2” score 420; and (5), a 100 mg/dL or higher change in an analyte level within a three hour period post-meal can correspond to a “1” score 420. Those of skill in the art will appreciate that other changes in the analyte level can be used with different scores (including more or fewer scores) and are fully within the scope of the present disclosure.

As depicted in home GUI 400, the score 420 can be displayed in a graphical element, such as a star icon 421. In some embodiments, the star icon 421 is filled with a colored portion, such as yellow. In many embodiments, the star icon 421 is unfilled and comprises a broken outline to indicate a missing meal score 420. Those of skill in the art will appreciate that other graphical representations and symbols can be used instead of a star icon (e.g., emoticons, trophy icons, bars, triangles, squares, circles, etc.). A missing meal score 420 happens when one or both of the following occurs: (1) the user logs the next meal too soon (e.g., when the user logs the next meal before sufficient analyte data is collected to score the current meal), or (2) the TIR application does not have sufficient analyte data to score 420 the meal (e.g., the user has not scanned in a timely manner so as to ensure glucose data has been transmitted to the TIR application). In some embodiments, a score information icon 422 is displayed proximal and adjacent to the list 402 of meal information on home GUI 400. When selected by the user, score information icon 422 outputs a meal scoring informational modal comprising information on the different score options and how a meal score is determined.

Still with reference to FIG. 4B, home GUI 400 can further comprise a text description 423 indicating a current day of the week, wherein the text description 423 includes a date adjacent to a switch 424 configured to be toggled. In some embodiments, the home GUI 400 can be related to a past day, wherein the textual description includes a date associated with the past day (e.g., “Fri February 11”). In some embodiments, though not illustrated, when home GUI 400 is related to a past day, home GUI 400 can further comprise a selectable “Go To Today” link which, upon being selected, outputs home GUI 400 corresponding to the current day.

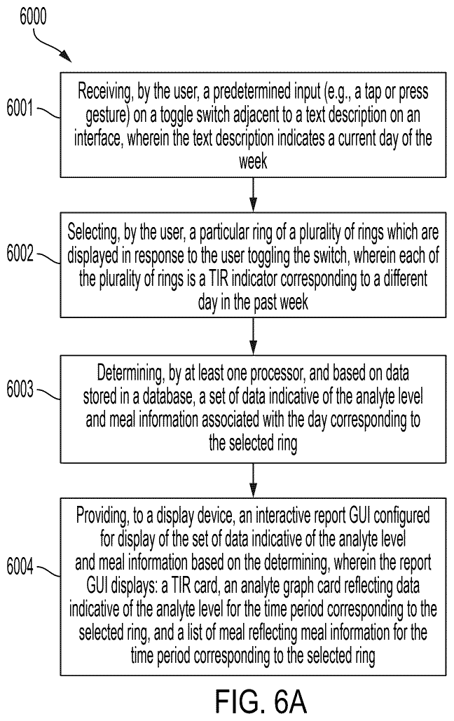

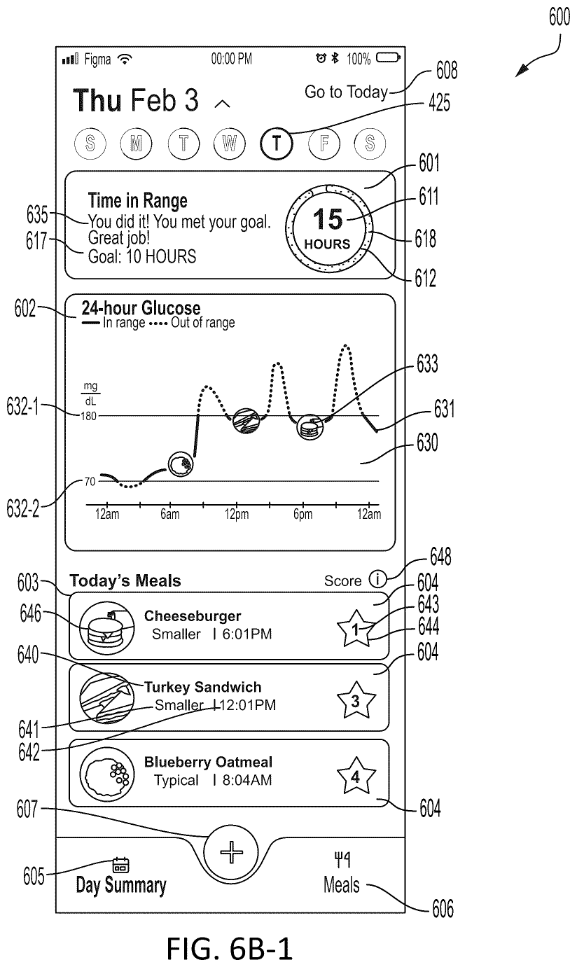

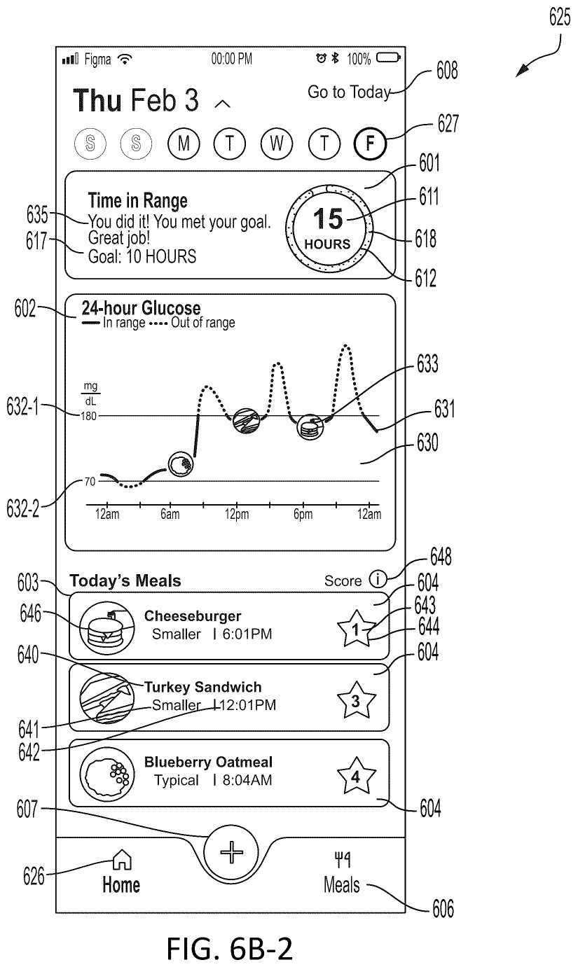

In some embodiments, when the switch 424 is toggled down by the user, the home GUI 400 is configured to display a plurality of selectable rings 425 (not shown in FIG. 4B, but see, e.g., FIG. 6B-1), each of which corresponds to a different day of the past week. For example, when the switch 424 is toggled down, the home GUI 400 can display a plurality of seven rings 425 (not shown in FIG. 4B), one corresponding to each day of the week. Similar to the TIR ring 406 displayed on the TIR card 401, each of the plurality of rings 425 represents the progress the user made towards that particular day's daily TIR goal. For example, the number of TIR hours the user spent in a particular day can be visually displayed as a progress indicator 426 (e.g., a shaded portion) extending along the perimeter of a ring 425 to X % of the total perimeter of the ring 425, to indicate that the user has spent X % TIR (where X % is the amount of hours spent in range divided by 24 hours).

In some embodiments, when a user selects a particular ring from the plurality of rings 425, a report GUI 600 will be displayed (e.g., FIG. 6B-1). In some embodiments, when the switch 424 is toggled to an up position, the plurality of rings 425 are no longer displayed. Report GUI 600 embodiments will be described in further detail below.

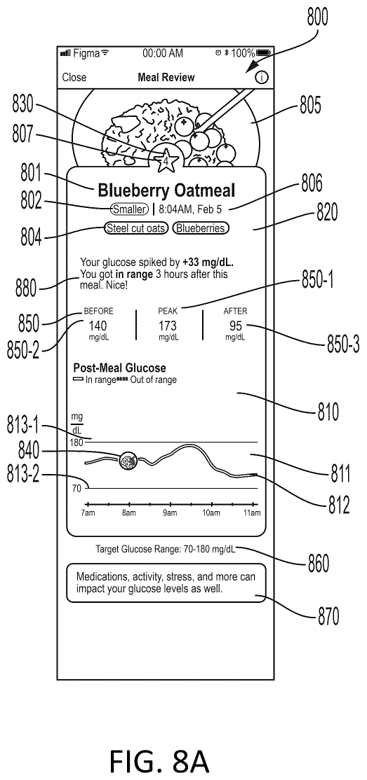

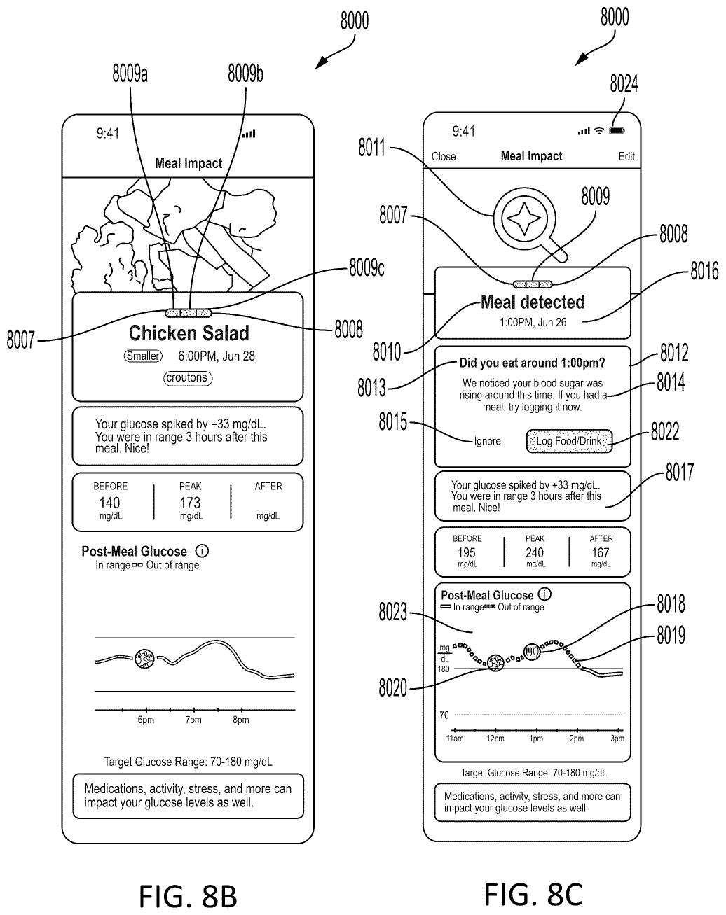

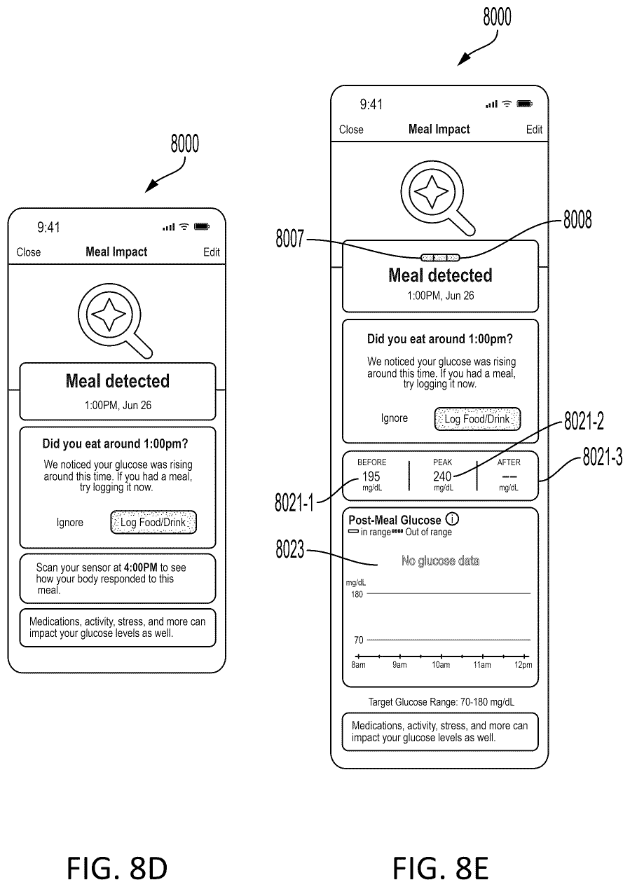

Referring to FIG. 4D, a block diagram depicting an example embodiment of home GUI 450 for a TIR application is shown. According to one aspect of the embodiments, home GUI 450 can comprise: (1) an analyte graph card 451; (2) a logbook section 4508 comprising a list 452 of meal events 469; (3) a selectable home icon 453 that, when selected by the user, outputs the home GUI 400; (4) a selectable meals icon 454 that, when selected by the user, outputs a meals impact GUI 800 or meal review GUI 8000 (FIGS. 8A and FIGS. 8B-8E, respectively) comprising information on one or more meal events 469 which occurred in a previous time period (e.g., the past week); and, (5) a “+” icon 455 that, when selected by the user, outputs a meal logging GUI 500 (not shown) configured to receive meal information inputted by the user. In some embodiments, though not illustrated, home GUI 450 can comprise a selectable insights icon instead of the meals icon 454. Specifically, when the user selects the insights icon, an insights GUI 14000 or insights GUI 150000 (e.g., FIGS. 14A-1 to 14A-5, and FIGS. 15A-1 through 15A-5, respectively) is outputted, which will be described in further detail below.

In some embodiments, home GUI 450 is displayed in response to a first predetermined input by the user on the home GUI 400, such as when the user, drags or swipes with a finger, or by some other predetermined gesture. In like manner, home GUI 400 can be displayed in response to a second predetermined input by the user on the home GUI 450, such as when the user, drags or swipes with a finger, or by some other predetermined gesture.

In some embodiments, the home GUI 450 is similar to the home GUI 400 embodiment depicted in FIG. 4B, except that the home GUI 450 displays the analyte graph card 451 rather than a TIR card. In some embodiments, the analyte graph card 451 can include an analyte graph 460 with an analyte trendline 461 reflecting the user's analyte level, based on the data indicative of the analyte level, over a predetermined time period. For example, and as shown in FIG. 4D, the x-axis of the analyte graph 460 can comprise units of time (e.g., three hour increments, etc.) covering a twelve-hour period, whereas the y-axis can comprise units of measure (e.g., mg/dL) for a measured analyte concentration of the user. Such an analyte graph 460 can show data over any desired time period, and those of skill in the art will further recognize that other predetermined periods of time (e.g., two hours, four hours, twenty-four hours, forty-eight hours, etc.) can be reflected on the x-axis, and are fully within the scope of the present disclosure. According to some aspects of the embodiments, the user's glucose concentration (in mg/dl or mmol/L) is displayed for a certain time period, e.g., one day, or part of a day. The time displayed on the x-axis can show the glucose concentrations measured starting from 12 am of the current day.

The analyte graph 460 can include solid lines 462-1, 462-2 to indicate the user's target analyte range associated with the data indicative of the analyte level. For example, the area between solid lines 462-1, 462-2 indicates a target analyte range (e.g., between 70 mg/dL and 180 mg/dL). In some embodiments, and as illustrated in FIG. 4D, the analyte trendline 461 is configured as a dashed or broken line to represent a period of time in which the user's analyte level falls out of bounds (e.g., exceeds or falls below) of the target analyte range. Further, the analyte trendline 461 is configured as a solid line to represent a period of time in which the user's analyte level is within the target analyte range. As previously mentioned, the target analyte range can be set by the user. For example, the target analyte could be set from about 80 mg/dL to about 170 mg/dL, alternatively from about 70 mg/dL to about 180 mg/dL, or from about 65 mg/dL to about 120 mg/dL. In some embodiments, and as illustrated in FIG. 4D, the home GUI 450 further comprises a message 463 adjacent to the analyte graph card 451, wherein the message 463 indicates when the analyte graph card 451 was last updated.

According to another aspect of some embodiments, one or more meal icons 464 can be displayed on the analyte trendline 461. In some embodiments, the one or more meal icons 464 can be displayed near, close to, and/or proximal to analyte trendline 461, or along the x-axis, the top of the graph window, or the bottom of the graph window. Additionally, in some embodiments, the one or more meal icons 646 can be displayed with or without lead lines. Each meal icon 464 can be a photograph or image associated with a particular meal. In some aspects of the embodiments, the meal icons 464 are positioned on the analyte trendline 461 so as to allow the user to visually associate a post-prandial analyte trace with the relevant meal. In some embodiments, the meal icons 464 are positioned on the analyte trendline 461 based on a time associated with the meal. For example, if a meal event 469 indicates that blueberry oatmeal was consumed at 8:04 AM, the meal icon 464 corresponding to the blueberry oatmeal meal event 469 will be displayed around 8:04 AM on the x-axis of on the analyte graph 460.

Still referring to FIG. 4D, and similar to home GUI 400, home GUI 450 can comprise the logbook section 4508 with the list 452 of one or more meal events 469 that occurred in a predetermined time period (e.g., one day). Each of the meal events 469 provides details of a particular meal consumed within the predetermined time period. In some embodiments, and as previously described with respect to the home GUI 400, each meal event 469 can include a text description of a meal 473 (e.g., “Blueberry Oatmeal”), a portion size indicator 474 (e.g., “Typical”) describing the relative portion size of the meal (e.g., typical, larger, or smaller compared to the user's usual meal serving), and/or a timestamp 475 associated with a time the meal was consumed (e.g., “8:04 AM”). Though not illustrated, in some embodiments, a meal event 469 can include one or more meals tags associated with the meal. Additionally or alternatively, the meal event 469 can include a photograph or picture 477 associated with the meal. In some embodiments, when the user does not associate a photograph or picture 477 with the meal, a placeholder picture 477 will be displayed instead. In some exemplar embodiments, the list 452 can be ordered such that the meal event 469 corresponding to the most recently consumed meal is presented first or at the top of the list 452, with subsequent meal events 469 being displayed in chronological order.

As depicted in FIG. 4D, and similar to the embodiments previously described herein, each meal event 469 can additionally or alternatively include a rating or score 480 of a single particular meal according to its respective analyte response. In some embodiments, the analyte response can be based on a change in an analyte level within a predetermined time period after the particular meal is consumed. In some embodiments, meals are given a score 480 based on the change in analyte level from the time the particular meal was consumed to the peak glucose within three hours of eating. Further, the score 480 can be displayed as a numerical value. In some embodiments, the score 480 can be a number between one and five, wherein a higher assigned numerical value corresponds to a lower analyte response, and a lower assigned numerical value corresponds to a higher analyte response. As depicted in home GUI 450, each score 480 can be displayed in a graphical element, such as a star icon 481. In some embodiments, each star icon 481 can be filled with a colored portion, such as yellow. In many embodiments, the star icon 481 is left unfilled and comprises a broken outline to indicate a missing meal score. In some embodiments, a score information icon 492 is displayed proximal and adjacent to the list 452 on home GUI 450. When selected by the user, score information icon 492 outputs meal scoring informational modal comprising information on the different score options and how a meal score is determined.

Similar to home GUI 400, home GUI 450 can further comprise a text description 493 indicating a current day of the week, wherein the text description 493 includes a date adjacent to a switch 494 configured to be toggled. In some embodiments, the home GUI 450 can be related to past day, wherein the textual description includes a date associated with the past day (e.g., “Fri February 11”). In some embodiments, though not illustrated, when home GUI 450 is related to a past day, home GUI 450 can further comprise a selectable “Go To Today” link which, upon being selected, outputs home GUI 450 corresponding to the current day.

In some embodiments, when the switch 494 is toggled down by the user, the home GUI 450 is configured to display a plurality of selectable rings 425 (not shown in FIG. 4D, but see, e.g., FIG. 6B-1), each of which is a TIR indicator corresponding to a different day of the past week. Each of the rings 425 for the different days can include a graphical or visual indication (progress indicator) of the amount of TIR and an indication of the goal/target percentage or TIR hours. In some embodiments, when a user selects a particular ring from the plurality of rings 425, a report GUI 600 (not shown) will be displayed for the relevant day. Those of skill in the art will recognize that other types of indicators (e.g., textual, numeric (as a percentage or a score), graphical) can also be utilized, and are fully within the scope of the present disclosure.

Further, in some embodiments, the home GUI 450 further comprises a selectable settings icon 499 which, when selected, outputs settings GUIs related to the TIR application.

Turning to FIG. 4E-1, a block diagram of an additional exemplar embodiment of a home GUI 430 for a TIR application is shown. In some embodiments, home GUI 430 can comprise: (1) a TIR card 431; (2) an analyte graph card 4311 comprising an analyte graph 4310 with an analyte trendline 4315; (3) a logbook section 4308 comprising a list 432 of meal events 4309; (4) a selectable home icon 433 that, when selected by the user, outputs the home GUI 430; (5) a selectable meals icon 434 that, when selected by the user, outputs a meals report GUI 600 (FIG. 6) comprising information on one or more meal events which occurred in a previous time period (e.g., the past week); and, (6) a “+” icon 435 that, when selected by the user, outputs a meal logging GUI 500 (see, e.g., FIGS. 5A to 5G) configured to receive meal information inputted by the user. In this regard, the TIR card 431 and analyte graph card 4311 are displayed on a single interface. In some embodiments, though not illustrated, home GUI 430 can comprise a selectable insights icon instead of the meals icon 434. Specifically, though not illustrated, when the user selects the insights icon, an insights GUI 14000 or insights GUI 150000 (e.g., FIGS. 14A-1 to 14A-5, and FIGS. 15A-1 through 15A-5, respectively) is outputted, which will be described in further detail below.

According to an aspect of the embodiments, the TIR card 431 of home GUI 430 is similar to the TIR card 401 of home GUI 400 depicted in FIG. 4B, except that the visual display of the TIR card 431 is different than the visual display of the TIR card 401. Specifically, for TIR card 431 (FIG. 4E-1), the daily TIR goal 437 is displayed outside the TIR ring 436, rather than in the inner area thereof. For example, in some embodiments, the daily TIR goal 437 is adjacent to the TIR ring 436. According to an aspect of the embodiments, the TIR ring 436 comprises a progress indicator 438 and a TIR value 4305. Similar to TIR card 401 depicted in FIG. 4B, TIR card 431 of home GUI 430 comprises a selectable informational icon 439 that, when selected by the user, outputs a TIR informational screen 440 related to TIR card 431. TIR informational screen 440 as depicted in FIG. 4F, is similar to the TIR informational screen 410 in FIG. 4C. Specifically, TIR informational screen 440 provides details on how the daily TIR ring 436 visually displays the progress indicator 438 and daily TIR goal 437. In some embodiments, the TIR informational screen 440, as shown in FIG. 4F, also informs the user that TIR goal settings are configurable.

According to another aspect of the embodiments, the analyte graph card 4311 of home GUI 430 is similar to the analyte graph card 451 of home GUI 450 depicted in FIG. 4D, except that it includes a target analyte range 4312 (e.g., “target glucose range: 70-180 mg/dL”). Further, home GUI 430, as shown in FIG. 4E-1, includes one or more selectable icons 4313 (e.g., radio button, check box, slider, switch, etc.) that allow the user to select a predefined amount of time over which the user's analyte data will be shown in the analyte graph card 4311. For example, selectable icons 4313 can be used to select a predefined amount of time of at least around four hours and/or no greater than around 24 hours, for example four hours, 12-hours, or 24-hours. Those of skill in the art will appreciate that other predefined amounts of time can be utilized and are fully within the scope of the present disclosure. In some embodiments, and as shown in FIG. 4E-1, the selectable icons 4313 can be directly adjacent to and below the analyte graph 4310 on the analyte graph card 4311.

In some embodiments, though not illustrated in FIG. 4E-1, one or more meal icons can be displayed directly adjacent to and below the analyte graph 4310, or below the x-axis. For example, the meal icons can be displayed at the bottom of the graph window 4314. According to some aspects of the embodiments, the meal icons are positioned on the analyte graph card 4311 so as to allow the user to visually associate a post-prandial analyte trace with the relevant meal. In some embodiments, the meal icons are positioned below the analyte graph 4310 and the x-axis, based on a time associated with the meal. For example, if a meal event 4309 indicates that a meal was consumed at 9 AM, the meal icon corresponding to the meal event 4309 will be displayed directly adjacent to and below 9 AM on the x-axis of on the analyte graph 4310.

In some embodiments, and as depicted in FIG. 4E-2, a notification can appear on the analyte graph card 4311, wherein the notification 4316 instructs the user that inputting a gesture (e.g., tapping or tapping and holding) on the trendline 4315 can provide a glucose reading associated with the selected area of the trendline 4315. In some embodiments, the notification 4316 can further inform the user that a swipe to scroll gesture can be utilized on the trendline 4315 so as to provide glucose readings for various selected areas of the trendline 4315. Further, FIG. 4E-3 depicts home GUI 430, wherein the analyte graph card 4311 includes a visual indicator 4317 comprising an analyte reading (e.g., a glucose reading) corresponding the selected area of the trendline 4315. In some embodiments, the visual indicator can include an analyte level value and a timestamp (for example, “117 mg/dL” and “10:00 AM”) corresponding to the selected area of the trendline 4315.

In some embodiments, and as best shown in FIG. 4E-3, the analyte graph card 4311 can comprise an analyte graph informational icon 4318 which, upon being selected by the user, outputs an analyte graph information modal 445 (FIG. 4G) which provides the user information on selecting a predefined amount of time over which the user's analyte data will be shown in the analyte graph card 4311. In some embodiments, the analyte graph information modal 445 further informs the user that tapping or holding an area of the analyte graph 4310 will allow the user to visualize the glucose reading associated with the selected area. In some embodiments, the analyte graph information modal 445 further comprises a disclaimer indicating to the user that they should consult a healthcare professional before making changes to a diet or exercise program.

In some exemplar embodiments, and as depicted in FIG. 4E-4, an announcement card 441 can be outputted to the home GUI 430. For example, an announcement card 441 can be displayed directly adjacent to and above the TIR card 431 on home GUI 430. In some embodiments, the TIR card 431, the analyte graph card 4311, and logbook section 4308 are shifted downwardly on home GUI 430 so as to allow the announcement card 441 to be positioned on the home GUI 430. In some embodiments, the announcement card 441 can inform the user that it is time to scan the sensor. In some embodiments, the announcement card 441 can further inform the user that the analyte monitoring application can be utilized to scan the sensor.

In some exemplar embodiments, and as depicted in FIG. 4E-5, an announcement card 441 can be outputted to the home GUI 430 in place of the TIR card. For example, an announcement card 441 can be displayed directly adjacent to and above the analyte graph card 4311. In some exemplar embodiments, the announcement card 441 can prompt the user to accept a challenge relating to a new TIR goal. For example, the announcement card 441 can inform the user that the user has met the TIR goal a particular number of times over a predefined period of time (e.g., five days out of the last seven days), and prompt the user to increase the target TIR goal by a particular number of hours (e.g., one hour). In some embodiments, the announcement card 441 can include an “opt out” button 442 which the user can select to deny the challenge, and an accept button 443, which the user can select to accept and initiate the challenge. According to an aspect of the embodiments, the TIR application can be configured to output the announcement card 441 comprising a challenge prompt to the user after the user has met the TIR goal a particular number of times over the predefined period of time.

The announcement cards 441 can be utilized with any of the embodiments described herein. Further, in some embodiments, announcement cards 441 can include one or more of the following: (1) information regarding meal logging (e.g., “Welcome, [User Name], Tap the “+” button below to add your first meal” or “Nicely done, [User Name]! You added your first meal! Use the Libre 2 app to scan your sensor in 3 hours and come back here to check how your body responded to the meal”); (2) information related to scanning (e.g., Ready to go? Scan your sensor with the Libre 2 app and start tracking towards your TIR goal. Aim to close you ring by midnight each day” or “Use the Libre 2 app to scan your sensor. When you are done, come back here and see how your body responded to the meal”); (3) information related to updated settings (e.g., “Your time in range goal has been set. You will see it here tomorrow” or “Your new goal is [hours], and you will see it here tomorrow. Keep it up!”); encouraging messages to the user (e.g., “Your new goal is [hours], and you will see it here tomorrow. Keep it up!” or “Great job, [User Name]! You've met your goal 5 out of the last 7 days. Challenge yourself and increase your target by one hour! Accept the challenge”); (4) prompting a user to accept a new challenge (e.g., “Great job, [User Name]! You've met your goal 5 out of the last 7 days. Challenge yourself and increase your target by one hour! Accept the challenge”); and (5) notifying the user when a score is available (e.g., “Score! Your first meal score is available. Tap the meal card below to see the details.”).

Additionally, and as best depicted in FIG. 4E-1, and according to another aspect of the embodiments, the analyte graph card 4311 can comprise a selectable arrow 4319 which, upon being selected by the user, provides a different view of the analyte graph 4310 on the analyte graph card 4311. For example, if the analyte graph card 4311 is configured to display analyte data for the predefined amount of time (e.g., a 24-hour time period), the analyte graph 4310 can be divided into a first view and a second view. In some embodiments, in the first view of the analyte graph 4310, analyte data for a first portion of the predefined amount of time is shown (e.g., analyte data is shown for a time period between 12 AM and 6 PM, or 12 AM to 9 PM). Further, though not illustrated, in the second view of the analyte graph 4310, analyte data for a second portion of the predefined amount of time is shown (e.g., analyte data is shown for a time period between 6 PM and 12 pm, or 9 PM to 12 PM).

In some example embodiments, and as depicted in, e.g., FIGS. 4E-1 to 4E-4, the TIR card 431 can be arranged directly adjacent to and above the analyte graph card 4310 on home GUI 430. Further, the logbook section 4308 can be arranged directly adjacent to and below the analyte graph card 4311 on home GUI 430.

Further, FIG. 4E-6 illustrates home GUI 430 in the first day or 24-hours that the user is utilizing the TIR application. As depicted in FIG. 4E-6, home GUI 430 comprises the analyte graph card 4311 without the analyte trendline. In some embodiments, and prior to the user inputting meals into the TIR application, home GUI 430 comprises the logbook section 4308 without the list of inputted meal events. In some embodiments, and as depicted in FIG. 4E-6, the logbook section 4308 comprises a note 446 informing the user that no meals have been added yet. According to another aspect of the embodiments, and prior to a daily TIR goal being set in the TIR application, home GUI 430 can comprise a TIR card 431 without the daily TIR goal or TIR value inside the TIR ring 436. In some embodiments, and as shown in FIG. 4E-6, and prior to a daily TIR goal being set in the TIR application, the TIR card 431 can comprise a message 447 indicating to the user that the TIR application is gathering the user's data. Further, in some embodiments, and prior to a daily TIR goal being set in the TIR application, the TIR card 431 can further comprise a selectable informational icon 448 which, upon being selected by the user, outputs an informational modal 465 (FIG. 4H). As shown in FIG. 4H, informational modal 465 can include a message 466 indicating to the user that the TIR application is in the process of calculating the first 24-hours of using the TIR application in order to set a personalized TIR goal for the user. Further, informational modal 465 can further include the message 466 with a reminder indicating that the user can scan the sensor regularly while the user waits. Informational modal 465 can be configured so as to partially obstruct or superimpose the underlying home GUI 430. In some embodiments, informational modal 465 further can include a selectable “ok” button 467 which can be pressed by the user.

FIG. 4E-7 depicts home GUI 430 after a personalized daily TIR goal 437 has been set. Specifically, FIG. 4E-7 depicts home GUI 430 with a TIR card 431 comprising the user's daily TIR goal 437. In some embodiments, the daily TIR goal 437 can be set prior to the TIR application displaying the analyte trendline or the user inputting any meal events (as shown in FIG. 4E-7). According to an aspect of the embodiments, the daily TIR goal 437 and a TIR value 4305 (not shown in FIG. 4E-7) inside the TIR ring 436 can be measured and displayed on the home GUI 430 with a numerical value and unit of measure indicative of number of hours. In some embodiments, if the daily TIR goal 437 is set but the number of TIR hours for the particular time period has not yet been detected, then a placeholder image or photograph 449 can be displayed in the TIR ring 436, as shown in FIG. 4E-7. In some embodiments, once the daily TIR goal 437 has been set, a modal 470 (FIG. 4I) can be outputted which informs the user that a new TIR goal has been set. In some embodiments, the modal 470 can further inform the user that the TIR goal can be changed through the TIR application's settings.

In some embodiments, and as best depicted in FIG. 4E-7, home GUI 430 can further comprise a text description 4333 indicating a current day of the week, wherein the text description 4333 includes a date adjacent to a switch 4334 configured to be toggled. In some embodiments, the home GUI 430 can be related to past day, wherein the textual description 4333 includes a date associated with the past day (e.g., “Fri February 11”). In some embodiments, though not illustrated, when home GUI 430 is related to a past day, home GUI 430 can further comprise a selectable “Go To Today” link which, upon being selected, outputs home GUI 430 corresponding to the current day.

Turning to FIGS. 4J-1 to 4J-3, an additional exemplar embodiment of a home GUI 485 is depicted. Home GUI 485 is similar to home GUI 430 (see, e.g., FIG. 4E-1) except that the logbook section 4808 of home GUI 485 comprises one or more Time of Day (“TOD”) cards 4801, as shown in FIGS. 4J-1 to 4J-3. Specifically, home GUI 485 comprises a TIR card 4819 comprising information related to an amount of time that the data indicative of the glucose level is within a predetermined analyte range (e.g., a predetermined glucose range), an analyte graph card 4811, a logbook section 4808, a selectable home icon 4821, and a selectable “+” icon 4823. In some embodiments, and as shown in FIGS. 4J-1 to 4J-3, home GUI 485 also comprises a selectable insights icon 4804 instead of the meals icon. Specifically, when the user selects the insights icon 4804, an insights GUI 14000 or insights GUI 150000 (e.g., FIGS. 14A-1 to 14A-5, and FIGS. 15A-1 through 15A-5, respectively) is outputted, which will be described in further detail below. Those of skill in the art will appreciate that a meals icon can be utilized with home GUI 485 in place of the insights icon 4804.

With reference to FIGS. 4J-1 to 4J-3, each TOD card 4801 in the logbook section 4808 of home GUI 485 can represent a different increment or time period of a particular day. In some embodiments, and as best shown in FIG. 4J-1, the logbook section 4808 can comprise four different TOD cards 4801, wherein each TOD card 4801 represents a different six-hour time period of a particular day. For example, and as best depicted in FIG. 4J-1, the logbook section 4808 can comprise, but is not limited to: (1) an “Overnight” TOD card 4801a (e.g., a TOD card 4801 corresponding to a 12 AM to 6 AM time period of a particular day); (2) a “Morning” Time of Day card 4801b (e.g., a TOD card 4801 corresponding to a 6 AM to 12 PM time period of a particular day); (3) an “Afternoon” TOD card 4801c (e.g., a TOD card 4801 corresponding to a 12 AM through 6 AM time period of a particular day); and (4) an “Evening” TOD card 4801d (e.g., a TOD card 4801 corresponding to a 6 PM to 12 AM time period of a particular day). Those of skill in the art will appreciate that various TOD cards 4801 and time periods associated with each TOD card 4801 can be utilized without departing from the scope of the present disclosure.

In some embodiments, and still with reference to FIGS. 4J-1 to 4J-3, each TOD card 4801 can comprise, but is not limited to: (1) a TOD indicator 4802 (e.g., “Overnight,” “Morning,” “Afternoon,” or “Evening”) configured to indicate the time of day represented by the TOD card 4801; (2) a time period indicator 4803 (e.g., “12 AM to 6 AM” for the Overnight TOD card 4801, “6 AM to 12 PM” for the Morning TOD card 4801, “12 PM to 6 PM” for the Afternoon TOD card 4801, and “6 PM to 12 AM” for the Evening TOD card 4801) configured to indicate the time period represented by the respective TOD card 4801; (3) a TOD icon 4804 configured to represent the time period associated with the TOD card 4801 (e.g., a TOD icon 4804 of a moon with “Z” letters to represent the overnight time period for the Overnight TOD card 4801, a TOD icon 4804 of a half sun or sunrise to represent the morning time period for the Morning TOD card 4801, a TOD icon 4804 of a full sun to represent the afternoon time period for the Afternoon TOD card 4801, and a TOD icon 4804 of a moon to represent the evening time period for the Evening TOD card 4801); and (4) a graphical representation 4805 indicating an average analyte score (e.g., average glucose score) associated with the time period represented by each respective TOD card 4801 (for example, an average glucose score corresponding to the time period between 12 AM-6 AM is represented by the graphical representation of the Overnight TOD card 4801). Those of skill in the art will recognize that other time ranges or time periods can be utilized to indicate various different time periods for the time period indicator 4803. According to some embodiments, the time periods represented by the time period indicator 4803 can be predetermined and/or configurable by the user. Further, according to an aspect of the embodiments, the average analyte score associated with the TOD card 4801 correlating to the current time of day is configured to continuously update. For example, if the current time of day is 9 AM, then the Morning TOD card 4801 (e.g., the TOD card 4801 representative of the time period between 6 AM-12 PM) is configured to continuously update the average analyte score for the Morning TOD card 4801.

In some embodiments, as best shown in FIG. 4J-1, the analyte graph card 4811 of home GUI 485 can comprise analyte graph TOD icons 4814 corresponding to the TOD icons 4804 displayed on each TOD card 4801 (e.g., an analyte graph TOD icon 4814a of a moon with “Z” letters to represent the overnight time period for the Overnight TOD card 4801a, a TOD icon 4814b of a half sun or sunrise to represent the morning time period for the Morning TOD card 4801b, a TOD icon 4814c of a full sun to represent the afternoon time period for the Afternoon TOD card 4801c, and a TOD icon 4814d of a moon to represent the evening time period for the Evening TOD card 4801d). Specifically, and as best shown in FIG. 4J-1, each analyte graph TOD icon 4814a, 4814b, 4814c, and 4814d, is displayed near, close to, and/or below an analyte trendline 4815 or along the x-axis, or the bottom of a graph window 484 of the analyte graph card 4811. More specifically, each analyte graph TOD icon 4814 is positioned along the x-axis so as to allow the user to visually associate the time period associated with the analyte graph TOD icon 4814 on the analyte graph card 4811.

In some exemplar embodiments, and as best shown in FIGS. 4J-1 to 4J-3, the graphical representation 4805 comprises segmented portions 4806 with each segment configured to be filled with color (or no color at all) so as to correspond with the average analyte score. In exemplar embodiments, and as best shown in FIG. 4J-1, the graphical representation 4805 can include a segmented bar with three segment portions 4806, wherein: (1) the first segment 4806a can have a red color or orange color and be indicative of an analyte score corresponding to a high glucose impact or response (e.g., an average glucose range exceeding 250 mg/dL); (2) the first segment 4806a and second segment 4806b can have a yellow color and be indicative of an analyte score corresponding to a medium glucose impact or response (e.g., an average glucose range between 181 mg/dL and 250 mg/dL, or no greater than 250 mg/dL and/or at least 181 mg/dL); or (3) the first segment 4806a, the second segment 4806b, and the third segment 4806c can have a green color and be indicative of an analyte score corresponding to a low glucose impact or response (e.g., an average glucose range between 70 mg/dL and 180 mg/dL, or no greater than 180 mg/dL). In some embodiments, though not illustrated, if the average glucose score corresponds to a low glucose impact (e.g., an average glucose range below 70 mg/dL) the graphical representation 4805 is replaced with a “LOW” icon instead of segmented portions 4806. Those of skill in the art will appreciate that various other graphical representations and colors, and color configurations can be utilized with the graphical representations without departing from the scope of the disclosure.

Further, and with particular reference to FIG. 4J-2, in some embodiments, and in response to a third predetermined input by the user, such as when the user taps on the graphical representation 4805 corresponding to a particular TOD card 4801, or by some other predetermined gesture, a visual indicator 4807 can be displayed on home GUI 485. Specifically, the visual indicator 4807 is configured to display the average analyte level value (e.g., an average glucose “117 mg/dL” associated with the selected graphical representation. Additionally, in some exemplar embodiments, an informational icon 4882 is displayed on home GUI 485, wherein the informational icon 4882, upon being selected by the user, is configured to output an analyte score information modal (which comprises information related the average analyte score, including but not limited to how the average analyte score is determined.

In some embodiments, the analyte score information modal can comprise a reference chart which provides information on average glucose scores, average glucose ranges associated with average glucose scores, and an impact level associated with the average glucose scores. In some embodiments, the analyte score information modal can further comprise details on the user's target glucose range (e.g., a target glucose range of 70-180 mg/dL) and inform the user that the target glucose range can be set or changed through the analyte monitoring application. Additionally, in some embodiments, the analyte score information modal can further comprise a disclaimer indicating to the user that they should consult a healthcare professional before making changes to a diet or exercise program. In some embodiments, the analyte score information modal can further comprise one or more informational cards, wherein each informational card provides information on the TOD cards 4801 (e.g., what is time of day, or why time of day). In some embodiments, and in response to a fourth predetermined input by the user, such as when the user taps or pulls down on a particular informational card, or by some other predetermined gesture, the selected informational card can be configured to expand so as to provide additional details related to the TOD cards 4801. However, in some embodiments, the informational cards are each in a collapsed configuration by default.

Turning again to FIGS. 4J-1 and 4J-3, each TOD card can 4801 transition between a collapsed view and an expanded view. Specifically, according to some embodiments, in the collapsed view (see, e.g., FIG. 4J-1, wherein each TOD card 4801 is configured in the collapsed view), the TOD card(s) 4801 is configured to display the TOD indicator 4802, the time period indicator 4803, the TOD icon 4804 representative of the time period associated with the TOD card 4801, and the graphical representation 4805 indicative of the average glucose score associated with the time period represented by the respective TOD card 4801. Further, according to some embodiments, in the expanded view (see, e.g., FIG. 4J-3, wherein the Evening TOD card 4801 is configured in the expanded view), the TOD card(s) in the expanded view 4801 is configured to display the TOD indicator 4802, the time period indicator 4803, the TOD icon 4804 representative of the time period associated with the TOD card 4801, the graphical representation 4805 indicative of the average glucose score associated with the time period represented by the respective TOD card 4801, and a list 4822 of meal information comprising one or more meal events 4829 that occurred in the relevant time period associated with the TOD card 4801. Further, in some embodiments, in the expanded view of a respective TOD card 4801, the TOD card 4801 is configured to display a meal logging link 4809 which, upon being selected by the user, outputs a meal logging GUI 500, as shown in FIG. 5A and which is further described below.

Specifically, in some embodiments, if the user inputs meal information through the meal logging GUI 500, then details relating to the inputted meal information will be displayed as a meal event 4829 in the list 4822 of meal information for the respective TOD card 4801 from which the meal logging link 4809 was selected. In some exemplar embodiments, and as best shown in FIG. 4J-3, the meal logging link 4809 is directly adjacent to and below the list 4822 of meal events 4829 displayed on a particular TOD card 4801 in the expanded view.

In some embodiments, and as best shown in FIG. 4J-3, each TOD card 4801 can be configured to comprise information on one or more meal events 4829 which occurred in the time period represented by the respective TOD card 4801. According to an aspect of the embodiments, each meal event 4829 can include a text description 4823 of a meal, a meal type indicator 4824 describing whether the inputted meal information describes a food or a drink, a timestamp 4825 associated with a time the meal was consumed, and/or a photograph or picture 4826 with the meal. In some embodiments, when the user does not associate a photograph or picture 4826 with the meal, a placeholder picture will be displayed instead. In some exemplar embodiments, the list 4822 can be ordered such that the meal event 4829 corresponding to the most recently consumed meal is presented first or at the top of the list 4822 for a respective TOD card 4801, with subsequent meal events 4829 being displayed in chronological order.

Further, in some exemplar embodiments, each meal event 4829 is configured to output a meal review GUI 800 or meal review GUI 8000 (as shown in FIGS. 8A and 8B-8E, respectively, and as will be described in further detail below) correlating to the selected meal event 4829 in response to a fifth predetermined input by the user, such as when the user taps on a particular meal event 4829, or by some other predetermined gesture.

According to another aspect of the embodiments, and as best shown in FIG. 4J-3, each TOD card 4801 can comprise an item counter 4810 which is configured to display the number of meal events 4829 logged for the particular time period associated with the respective TOD card 4801. For example, if the user has logged two meal events 4829 under the Evening TOD card 4801, then the item counter 4810 associated with the evening TOD card 4801 will indicate “two items.” Further, according to some embodiments, each TOD card 4801 can be configured to display the item counter 4810 in both the collapsed view and the expanded view.

According to yet another aspect of the embodiments, each TOD card 4801 is configured to transition between the collapsed view and the expanded view in response to a sixth predetermined input by the user on the respective TOD card 4801, such as when the user taps, pulls the TOD card 4801 down with a finger, pulls the TOD card 4801 up with a finger, or by some other predetermined gesture. For example, in exemplar embodiments, each TOD card 4801 can transition from displaying the collapsed view to the expanded view in response to a received input by the user (e.g., by a tap gesture, a pull down gesture, or by selecting or pressing a corresponding area of the TOD card 4801). Moreover, in some embodiments, the TOD card 4801 can transition from displaying the expanded view to the collapsed view in response to a received input by the user (e.g., by a tap gesture, a pull up gesture, or by selecting or pressing a corresponding area of the TOD card 4801).

In some embodiments, and with reference to FIGS. 4J-1 to 4J-3, home GUI 485 is configured to display by default the TOD card 4801 corresponding to the current time of day in the expanded view. For example, if the current time of day is 9 AM, then the Morning TOD card 4801 is configured to be displayed in the expanded view on home GUI 485. Additionally, if a new time of day has occurred, for example, if the day transitions form morning time of day to afternoon time of day, then the TOD card 4801 corresponding to the new time of day (e.g., the afternoon TOD card 4801) will populate in the logbook section 4808 of home GUI 485. Specifically, the TOD card 4801 corresponding to the new time of day will be outputted to the home GUI 485 in the expanded view by default. In some embodiments, the TOD card 4801 corresponding to the current time of day is displayed by default in the expanded view (e.g., the Afternoon TOD card), while the TOD cards 4801 corresponding to past TOD cards 4801 for that particular day are displayed by default in the collapsed view (e.g., Overnight TOD card 4801 and Morning TOD card 4801).

In some embodiments, the TOD card 4801 corresponding to the current time of day is displayed by default in the collapsed view. In some embodiments, the logbook section 4808 is configured to display by default each TOD card 4801 in the collapsed view.

Further, in some embodiments, though not illustrated, the TOD card 4801 corresponding to the current time of day is configured to be highlighted as active by comprising a colored portion. For example, if the current time of day is 7 PM, then the Afternoon TOD card 4801 can comprise a colored portion to indicate it is active. Specifically, the TOD icon 4804 and/or the TOD indicator 4802 of the TOD card 4801 corresponding to the current time of day can comprise a colored portion (e.g., be highlighted or colored, for example, in blue, whereas all other TOD indicators 4802 and TOD icons 4804 corresponding to other TOD cards 4801 are not highlights or colored).

According to another aspect of the embodiments, and as best shown in FIG. 4J-1, the logbook section 4808 is further configured to display TOD cards 4801 correlating to the elapsed or historical time periods for a particular day represented on the interface and the current time period. For example, in some embodiments, and as shown in FIG. 4J-1, if the current time of day is 7 PM, then the logbook section 4808 is configured to display the Overnight TOD card 4801aa, the Morning TOD card 4801b, the Afternoon TOD card 4801c, and the Evening TOD card 4801d. In some embodiments, the time period indicator for the TOD card 4801 corresponding to the current time of day (for example, the Afternoon TOD card 4801 if the current time of day is 3 PM) is configured to display a progress stamp 4812 (e.g., “Now”) to indicate the particular time period is current or in progress. In some embodiments, the progress stamp 4812 is displayed as part of the TOD indicator 4802. For example, if the current time of day is 3 PM, then the afternoon TOD card 4801 can display the time period indicator 4803 “12 PM-now” to indicate that the current time of day is within the time period associated with the TOD card 4801 in the active state (e.g., 12 PM-6 PM for the Afternoon TOD card 4801).

In some embodiments, and as best shown in FIG. 4J-1, home GUI 485 can further comprise a text description 4833 indicating a current day of the week, wherein the text description 4833 includes a date adjacent to a switch 4834 configured to be toggled. In some embodiments, the home GUI 485 can be related to past day, wherein the textual description 4833 includes a date associated with the past day (e.g., “Fri February 11”). In some embodiments, though not illustrated, when home GUI 485 is related to a past day, home GUI 485 can further comprise a selectable “Go To Today” link which, upon being selected, outputs home GUI 485 corresponding to the current day.

Turning to FIGS. 4K-1 to 4K-3, a block diagram of an additional exemplar embodiment of a home GUI 495 for a TIR application is shown. In some embodiments, home GUI 495 can comprise: (1) a daily score card 4951; (2) an analyte graph card 4911; (3) a logbook section 4958; (4) a selectable home icon 4953 that, when selected by the user, outputs the home GUI 495; (5) a selectable insights icon 4954 that, when selected by the user, outputs an insights GUI 14000 or insights GUI 150000 (e.g., FIGS. 14A-1 to 14A-5, and FIGS. 15A-1 through 15A-5, respectively) is outputted, which will be described in further detail below; and, (6) a “+” icon 4957 that, when selected by the user, outputs a meal logging GUI 500 (see, e.g., FIGS. 5A to 5G) configured to receive meal information inputted by the user.

According to an aspect of the embodiments, home GUI 495 is similar to home GUI 485 (see, e.g., FIGS. 4J-1 to 4J-3) except that it comprises the daily score card 4951 instead of the TIR card. In some embodiments, the daily score card 4951 can highlight the user's “daily score” based on the meals the user logs and/or the user's analyte (e.g., glucose) control using a graphical element, such as a Daily Score circle or ring 4903. As seen in, e.g., FIG. 4K-1, daily score card 4951 shows daily score ring 4903 with a daily score value 4904 (points earned daily) inside or in an inner area of the daily score ring 4903. For example, the daily score value 4904 can comprise a numerical value and unit of measure indicative of the user's earned daily score (e.g., “80 points”). In some embodiments, the current daily score that the user has earned relative to the predetermined target daily score can be displayed in two ways: (1) the daily score value 4904 can be a numerical value and a unit of measure (e.g., “80 points”) listed inside the daily score ring 4904; and (2) the daily score value 4904 can be visually displayed as a progress indicator 4905 (e.g., a shaded portion) extending along the perimeter of the daily score ring 4903 to X % of the total perimeter of the daily score ring 4903, to indicate that the user has earned X % of the predetermined target daily score (where X % is the daily score value 4904 divided by the predetermined target daily score, for example). In this manner, the ratio of the perimeter of the daily score ring 4903 that comprises the progress indicator 4905 (e.g., the shaded portion) and the total perimeter of the daily score ring 4903 is proportional to a ratio of the daily score value 4904 that the user has earned in the predetermined daily score range in a day. In some embodiments, the progress indicator 4905 can comprise a color having a gradient configured to transition between various color shades. In this manner, the degree by which the color gradient transitions between various shades is indicative of the progress the user has made in reaching their predetermined target daily score goal.

Those of skill in the art will further recognize that other types of progress indicators 4905 (e.g., textual, numerical, or graphical) can also be utilized, and are fully within the scope of the present disclosure. Furthermore, although the graphical element shown is a circle or ring having a shaded portion comprising a gradient, those of skill in the art will understand that other geometrical shapes, colors, and numbers of portions or gradients can be utilized, and are fully within the scope of the present disclosure.

In some embodiments, and as best depicted in FIG. 4K-1, the daily score card 4951 can further comprise a selectable informational icon 4907 that, when selected by the user, outputs a daily score informational screen 4950 (FIG. 4L) related to daily score card 4951. For example, and as depicted in FIG. 4L, the daily score informational screen 4950 provides details on how the daily score ring 4903 visually displays the progress indicator 4905 and daily score value 4904. In some embodiments, the daily score informational screen 4950 also informs the user that the daily score is calculated daily and resets at a predetermined hour (e.g., at midnight).

In some embodiments, as shown in FIG. 4K-2, the daily score card 4951 further can comprise a message 4906 indicating to the user the user's daily score at the end of a particular day (e.g., “You reached 87 points!”). Further, and as best shown in FIG. 4K-2, in some embodiments, the logbook section 4958 can comprise a list 4952 of meal events 4959 instead of one or more TOD cards.

Further, FIG. 4K-3 illustrates home GUI 495 after the user has first utilized TIR application and prior to meals being inputted. As depicted in FIG. 4K-3, home GUI 495 comprises the analyte graph card 4911 without the analyte trendline. In some embodiments, and prior to the user inputting meals into the TIR application, home GUI 495 comprises the logbook section 4958 without the list of inputted meal events. According to another aspect of the embodiments, and as best depicted in FIG. 4K-3, an announcement card 4908 is displayed in place of the daily score card on home GUI 495. Specifically, the announcement card 4908 can inform the user that the user can use a predetermined gesture (e.g., a tap) on the “+” icon 4957 to input meals into the TIR application.

In some embodiments, and as best shown in FIG. 4K-3, home GUI 495 can further comprise a text description 4933 indicating a current day of the week, wherein the text description 4933 includes a date adjacent to a switch 4934 configured to be toggled. In some embodiments, the home GUI 495 can be related to past day, wherein the textual description 4933 includes a date associated with the past day (e.g., “Fri February 11”). In some embodiments, though not illustrated, when home GUI 495 is related to a past day, home GUI 495 can further comprise a selectable “Go To Today” link which, upon being selected, outputs home GUI 495 corresponding to the current day.

Turning to FIGS. 4M-1 and 4M-2, a block diagram of an additional exemplar embodiment of a home GUI 499 for a TIR application is shown. In some embodiments, home GUI 499 can comprise: (1) an average meal rating card 4991; (2) an analyte graph card 4910; (3) a logbook section 4998 comprising a list 4992 of meal events 4999; (4) a selectable home icon 4995 that, when selected by the user, outputs the home GUI 499; (5) a selectable insights icon 4994; and, (6) a “+” icon 4997 that, when selected by the user, outputs a meal logging GUI 500 (see, e.g., FIGS. 5A to 5G) configured to receive meal information inputted by the user.

In some embodiments, and as best shown in FIG. 4M-1, home GUI 499 can further comprise a text description 4973 indicating a current day of the week, wherein the text description 4973 includes a date adjacent to a switch 4974 configured to be toggled. In some embodiments, the home GUI 499 can be related to past day, wherein the textual description 4973 includes a date associated with the past day (e.g., “Fri February 11”). In some embodiments, though not illustrated, when home GUI 499 is related to a past day, home GUI 499 can further comprise a selectable “Go To Today” link which, upon being selected, outputs home GUI 499 corresponding to the current day.

As illustrated in FIGS. 4M-1 to 4M-2, each meal event 4999 can additionally or alternatively include a rating or score 4922 of a single particular meal according to the analyte response (e.g., glucose response) associated therewith. In some embodiments, the analyte response can be based on a change in an analyte level (e.g. glucose level) within a predetermined time period after the particular meal is consumed. In some embodiments, meals are given a score 4922 based on the analyte level excursion value from the time the particular meal was consumed to the peak glucose within three hours of eating.

Further, the score 4922 can be displayed as a numerical value. In some embodiments, the score 4922 can be a number between one and five, wherein a higher assigned numerical value corresponds to a lower analyte response, and a lower assigned numerical value corresponds to a higher analyte response.

For example, (1) a 0 to 20 mg/dL change in an analyte level within a two hour period post-meal can correspond to a “5” score 4922; (2) a 21 to 40 mg/dL change in an analyte level within a two hour period post-meal can correspond to a “4” score 4922; (3) 41 to 60 mg/dL change in an analyte level within a two hour period post-meal can correspond to a “3” score 4922; (4) a 61 to 90 mg/dL change in an analyte level within a two hour period post-meal can correspond to a “2” score 4922; and (5), a 91 mg/dL or higher change in an analyte level within a two hour period post-meal can correspond to a “1” score 4922. As another example, (1) a 0 to 24 mg/dL change in an analyte level within a three hour period post-meal can correspond to a “5” score 4922; (2) a 25 to 49 mg/dL change in an analyte level within a three hour period post-meal can correspond to a “4” score 4922; (3) 50 to 74 mg/dL change in an analyte level within a three hour period post-meal can correspond to a “3” score 4922; (4) a 75 to 99 mg/dL change in an analyte level within a three hour period post-meal can correspond to a “2” score 4922; and (5), a 100 mg/dL or higher change in an analyte level within a three hour period post-meal can correspond to a “1” score 4922. Those of skill in the art will appreciate that other changes in the analyte level can be used with different scores and are fully within the scope of the present disclosure.

Further, and according to another aspect of the embodiments, home GUI 499 is similar to home GUI 430 depicted in, e.g., FIG. 4E-1, except that instead of a TIR card, the average meal rating card 4991 is displayed. Specifically, according to an aspect of the embodiments, and as shown in FIG. 4M-1 through 4M-3, the average meal rating card 4991 comprises an average meal rating 4920 that can be displayed as a numerical value, and a graphical indication 4921 corresponding to the average meal rating 4920. More specifically, the average meal rating 4920 is an average of the scores 4922 of all scored meal events 4999 for a predetermined time period (e.g., for a particular one-day period), or can be based on an average of the scores 4922 of a plurality of the scored meal events 4999 for a predetermined period of time (e.g. the scores for all scored meal events of a particular type of meal, such as the scores 4922 for all scored breakfasts). In some embodiments, and as best depicted in FIGS. 4M-1 through 4M-3, the average meal rating 4920 can be a number between one and five, wherein a higher assigned numerical value corresponds to a lower average analyte response, and a lower assigned numerical value corresponds to a higher average analyte response. In some embodiments, the average meal rating 4920 is displayed as a numerical value comprising one or more decimal values (e.g., an average meal rating of “5.0”).

For example, if one meal event 4999 is logged for a particular day, wherein the one meal event 4999 was given a “5” score 4922, then the average meal rating card 4991 would display an average meal rating of “5” or “5.0.” Further, if two meal events 4999 are logged for a particular day, wherein one meal event 4999 is given a “5” score 4922, and the second meal event 4999 is given a “1” score, then the average meal rating 4920 will be “3” or “3.0,” an average of the two meal events' 4999 scores 4922 (as shown in FIG. 4M-1). In some embodiments, if the user has logged two meal events 4999 but only one has been scored, then the average meal rating 4920 will only take into account the scored meal event 4999, as shown in FIG. 4M-2. In some embodiments, the average meal rating 4920 is configured to update continuously and automatically so as to account for all meal events 4999 that are being scored for a predetermined time period.

As best illustrated in FIGS. 4M-1 through 4M-3, and according to another aspect of the embodiments, the average meal rating card 4991 further comprises the graphical indication 4921 of the average meal rating 4920. Specifically, the graphical indication 4921 can comprise an average meal rating indicator 4923 that visually illustrates the average meal rating provided for the predetermined period of time (e.g., for a particular one day period). In some embodiments, the graphical indication 4921 is a plurality of stars that comprise a colored portion, wherein a ratio of each star that comprises a colored portion and a total plurality of stars is proportional to a ratio of the average meal rating 4920 to a maximum available meal rating. Those of skill in the art will appreciate that other graphical indications 4921, indicators 4923, and symbols can be used instead of a star icon (e.g., emoticons, trophy icons, bars, triangles, squares, circles, etc.) without departing from the scope of the present disclosure.

In some embodiments, and as best depicted in FIG. 4M-3, when the average meal rating 4920 is not a whole number (e.g., an average meal rating 4920 of “3.4”), then the graphical indication 4921 can comprise at least one star with a half-colored portion. For example, if the average meal rating 4920 is a “3.4,” then the graphical indication 4921 can display three of five stars with fully colored portions, and one of five stars with a half-colored portion, as depicted in FIG. 4M-3.

According to an aspect of the embodiments, the average meal rating 4920 is only provided after the user has received a first score for a meal event 4999 logged in the predetermined period of time. In some embodiments, and as depicted in FIG. 4M-4, an announcement card 4924 will be displayed directly adjacent to and proximal relative to the analyte graph card 4910, wherein the announcement “+” icon to add a first meal. In some embodiments, though not illustrated, an announcement card will be displayed directly adjacent to and proximal relative to the average meal rating card, wherein the announcement card comprises a message indicating to the user that the first meal rating or score is available and that the user can tap the particular meal event which was scored to see further details. Further, in some embodiments, and as depicted in FIG. 4M-4, a banner can be displayed on the analyte graph card 4910, wherein the banner 49001 indicates that the user can tap on the analyte trendline 49015 to view analyte readings (e.g., glucose readings) and swipe horizontally to scroll through the analyte graph 49011.

In some embodiments, and with reference to FIG. 4M-5, if the user deletes all the scored meals, if the user does not receive an average meal rating for the predetermined time period (e.g., for the day), or if no meal events have been scored yet for the predetermined time period, then the average meal rating card 4991 does not display an average meal rating, and the graphical indication 4921 corresponding to the average meal rating comprises a plurality of stars, wherein none of the stars comprise a colored portion and are unfilled. In some embodiments, if the user deletes all the scored meals or if no meal events have been scored yet for the predetermined time period, a tip 4925 (see, e.g., FIG. 4M-5) will appear on the average meal rating card 4991. Specifically, and as shown in FIG. 4M-5, the tip 4925 can inform the user that the user will get an average meal rating 4920 once the user logs a meal and gets the first meal rating for the predetermined time period (e.g., for the day). In some embodiments, one the user logs their first meal and/or dismisses the tip 4925, e.g., by tapping on an exit button, then the tip 4925 is removed from the average meal rating card 4991 and will not appear again.

Further, in some embodiments, and with reference to FIG. 4M-6, if the user does not receive an average meal rating for the predetermined time period (e.g., for the day), then the average meal rating card 4991 does not display an average meal rating, and the graphical indication 4921 comprises a plurality of stars, wherein none of the stars comprise a colored portion and the plurality of stars are unfilled. Additionally, in some embodiments, if the user does not receive an average meal rating for the predetermined time period (e.g., for the day), a message 4926 will appear on the average meal rating card 4991 indicating to the user that no meal ratings were available during the predetermined time period (e.g., “No meal ratings available on this day.”).

In some embodiments, and as depicted in FIGS. 4M-1 to 4M-4, and 4M-6, the average meal rating card 4991 comprises an informational icon 4927 which, upon being selected, outputs an average meal rating modal 4990 (FIG. 4N). Specifically, and as shown in FIG. 4N, the average meal rating modal 4990 can inform the user that the average meal rating is based on all the user's meal logs that have a rating for a predetermined time period (e.g., between midnight and 11:59 P.M.). Further, the average meal rating modal 4990 can also inform the user that the user's average meal rating may change as new meal ratings or scores become available throughout the predetermined time period (e.g., throughout the day).

According to another aspect of some embodiments, and with particular reference to FIGS. 4M-1 to 4M-3, one or more meal icons 49014 can be displayed on the analyte trendline 49015. In some embodiments, the one or more meal icons 49014 can be displayed near, close to, and/or below analyte trendline 49015, or along the x-axis, the top of the graph window 49904, or the bottom of the graph window 49904. Additionally, in some embodiments, the one or more meal icons 49014 can be displayed with or without lead lines. Each meal icon 49014 can be a photograph or image associated with a particular meal. In some aspects of the embodiments, the meal icons 49014 are positioned on the analyte trendline 49015 so as to allow the user to visually associate a post-prandial analyte trace with the relevant meal. In some embodiments, the meal icons 49014 are positioned on the analyte trendline 49015 based on a time associated with the meal. For example, if a meal event 4992 indicates that blueberry oatmeal was consumed at 8:04 AM, the meal icon 49014 corresponding to the blueberry oatmeal meal event 4992 will be displayed around 8:04 AM on the x-axis of on the analyte graph 49011.

It will be understood by those of skill in the art that any of the GUIs (or portions thereof) described herein, are meant to be illustrative only, and that the individual elements, or any combination of elements, depicted and/or described for a particular embodiment or figure are freely combinable with any other element, or any combination of other elements, depicted and/or described with respect to any of the other embodiments.

### Example Embodiments of Meal Logging GUIs

Turning to FIGS. 5A through 5N, example embodiments of meal loggings interfaces, and features related thereto, will be described. Referring first to FIG. 5A, meal logging GUI 500 is displayed when the user selects the “+” icon on any of the GUI embodiments comprising the “+” icon that are described herein, or a meal logging button on a notification corresponding to a detected missed meal on any of the GUI embodiments described herein (as will be described in further detail below). In some embodiments, the TIR application can prompt users to log a meal, e.g., if a meal event is detected or a predetermined period of time has lapsed. Users of the TIR application can also voluntarily log a meal without any prompting. As seen in FIG. 5B, if the user wants to voluntarily add a meal, the user can select an option to add a meal, e.g., by tapping the “+” icon on any GUI embodiments described herein comprising the “+” icon, after which the meal logging GUI 500 will populate or be displayed. As shown in FIGS. 5A and 5B, meal logging GUI 500 can include a meal entry field 501, which allows the user to enter the name or description of the food for the meal entry. As the user enters the characters of the name of the meal or food into meal entry field 501 (FIG. 5B), the TIR application can auto-suggest entries based on the user's past meal entries. For example, in some embodiments, the TIR application can retrieve information relating to past meal entries with the same (or similar) food name and display it in results section 502, for example, as a list of past meal events 503. If the desired food name appears in results section 502, the user can conveniently select the meal entry without the need to type the full name of the food or meal.

Furthermore, although FIG. 5B shows results section 502 in the form of a list, those of skill in the art will appreciate that other interfaces can be implemented, such as by the manual entry of text, by selection of the meal name from a list (e.g., a picklist or drop-down list), by selection of the meal picture from a group of pictures, by selection of recognizable indicia (e.g., a tag) of the meal, or any combination thereof. In some embodiments, a score information icon 522 (e.g., FIG. 5D) is displayed with the results section, wherein the score information icon 522, when selected, outputs meal scoring informational modal comprising information on the different score options and how a meal score is determined.

According to another aspect of the embodiments, and as shown in FIG. 5D, if meal information is entered that is not recognized as a past meal, the results section 502 will indicate no past meals were found. Once new meal information is entered, the user can select continue button 504 which can cause the display of a modal 510 configured to request additional information, as seen, for example, in FIG. 5E. The user can edit the meal entry as needed. For example, the user can click on a pencil icon 505 that is adjacent to the name of the meal or food as it appears in a meal entry description 506.

The modal 510 can contain portion size indicator 511 options that indicate the relative size of the meal consumed, e.g., selectable buttons indicating different sizes of meals such as “Smaller,” “Typical,” and “Larger.” The user can also add a photo/picture 512 of the meal to be associated with the meal. For example, the modal 510 can include a camera icon 513 that, when selected or tapped, opens the camera of the device on which the time-in-range application is installed, and the user can take a picture of the food or alternatively select a picture from a library of photos. The modal 510 can further a meal tag entry 514, wherein the user can type one or more meal tags 515 to associate with the meal consumed. The meal tags 515 (e.g., FIG. 5F) can be keywords that specify or describe one or more of the ingredients and/or a characteristic of the meal, or additional content consumed with the meal. For instance, the meal tags 515 can relate to the specific content of the meal or sides consumed with the meal, and list types of food, e.g., carrots, lettuce, blueberries, cheese, banana, fries. As illustrated in FIG. 5F, for example, a “carrots” meal tag is selected. After the meal information has been entered, the user can click “save” 516 to log the information.

In some embodiments, the modal 510 further includes a time field 517 that includes a time and date associated with the meal entry. In some embodiments, the time field 517 can be automatically associated with the meal entry or be auto-populated based on the time that the user selected the continue button 504 (e.g., FIG. 5D). According to one aspect of some embodiments, and as illustrated in FIG. 5G, the auto-populated time field 517 can still be edited by the user.

According to another aspect of the embodiments, and as shown in FIG. 5C, if the user selects a meal that was already entered, the meal entry can prepopulate with the past meal's information and assign the current date and time. Moreover, as shown in FIG. 5C, a meal entry modal 520 (also referred to as a meal entry modal window) will appear comprising a time field 527 with the current date and time, and information associated with the past meal event 503. The user can edit the meal entry as needed. The user can edit the name of the meal by clicking on the pencil icon 525 adjacent thereto. The user can edit the meal entry to add a name of the meal. The user can also select a camera icon 531 and edit the picture 532 associated with the meal entry. The user can also edit the meal tags 533 associated with the past meal event 503. The user can click “add meal” 535 to log the information.

Referring now to FIG. 5H, a block diagram depicting an exemplar embodiment of modal 530 is shown. Modal 530 is similar to modal 510 (FIG.E), except that a photo/picture associated with a meal is not displayed. In some embodiments, and as depicted in FIG. 5H, when the user does not associate a photograph or picture with the meal, a placeholder picture 534 will be displayed instead. Similar to modal 510, modal 530 can include a camera icon 536 that, when selected or tapped, opens the camera of the device on which the TIR application is installed, and the user can take a picture of the food or alternatively select a picture from a library of photos.

FIGS. 5I to 5N are various block diagrams depicting example embodiments of camera interfaces, or features related thereto, any of which can be utilized with the embodiments described herein. Referring to FIG. 5I, a camera GUI 540 is depicted which can be displayed after the user selects camera icon 513 (FIG. 5E) or 531 (FIG. 5H) on previous interface embodiments described herein. In some embodiments, the camera of the device cannot be utilized until the user grants the TIR application permission to access the camera. Camera GUI 540 is directed to enabling camera access. In some embodiments, camera GUI 540 can include a modal that prompts the user to enable camera access.

In some embodiments, the TIR application cannot utilize photos stored on the device until access has been granted by the user. FIG. 5J illustrates a block diagram depicting an example embodiment of a camera GUI 550 directed to enabling photo access. In some embodiments, camera GUI 540 can include a modal that prompts the user to enable photo access. In one aspect of the embodiments, the user can allow access to select photos. In another aspect of the embodiments, the user can allow access to all photos. In yet another aspect of the embodiments, the user can restrict access to any photos.

FIG. 5K illustrates a block diagram depicting an example embodiment of a camera GUI 560, wherein camera GUI 560 comprises an indicator specifying that camera access has been restricted. FIG. 5L illustrates a block diagram depicting an example embodiment of camera GUI 570, wherein camera GUI 570 comprises an indicator specifying the device's camera is not available.

FIGS. 5M and 5N illustrate block diagrams depicting example embodiments of camera GUIs 580 and 590, respectively, which are directed to photo capturing and/or selection. In some embodiments, and with reference to FIG. 5M, camera GUI 580 can comprise a retake button which, upon being selected by the user, removes the previously taken photo and allows the user to take another photo to associate with the particular meal. In some embodiments, camera GUI 580 comprises a “Use Photo” button which, upon being selected by the user, allows the user to use the captured photo and associate it with the particular meal. Further, FIG. 5N depicts an example embodiment of camera GUI 590, wherein a photo is displayed thereon. In some embodiments, camera GUI 590 comprises a library button which, upon being selected by the user, allows the use to browse photos stored on the device. In some embodiments, camera GUI 590 further comprises a “skip” button which, upon being selected by the user, allows the user to skip utilizing the photo displayed on camera GUI 590.

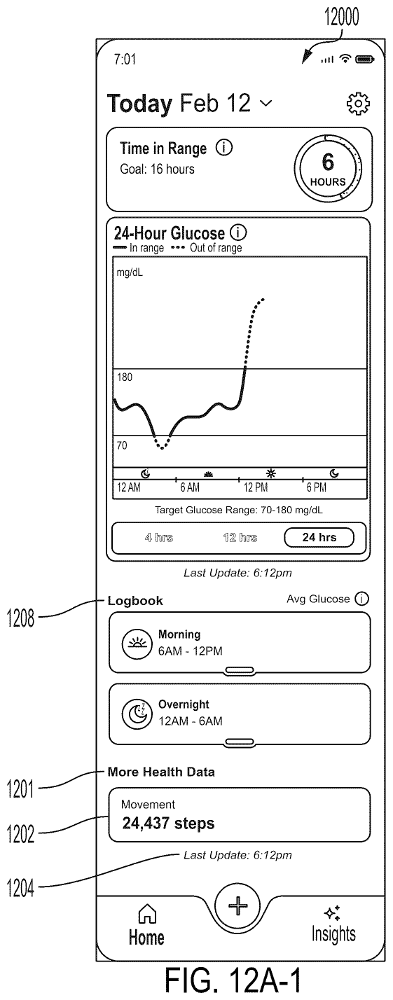

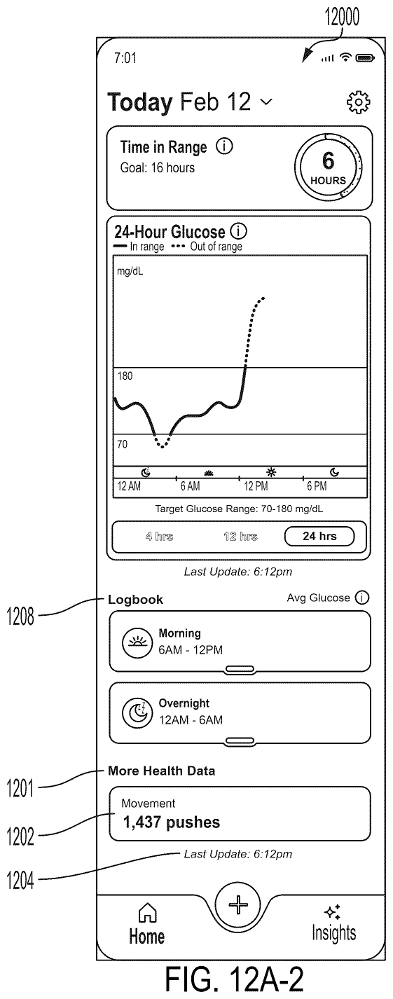

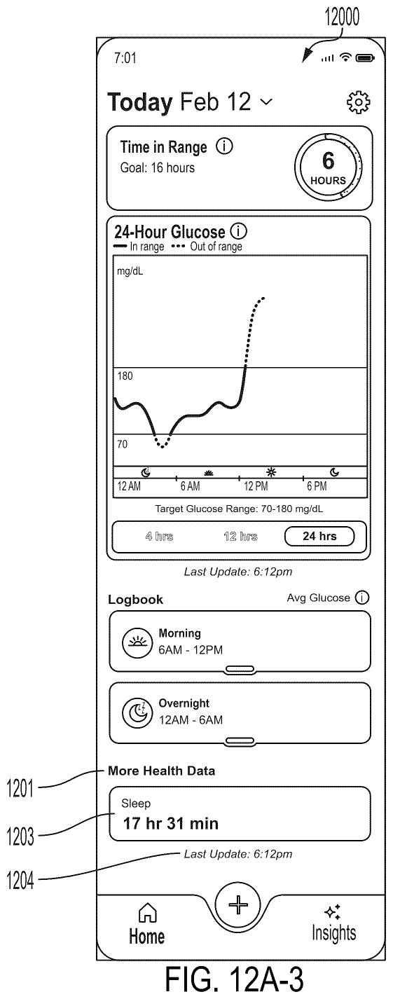

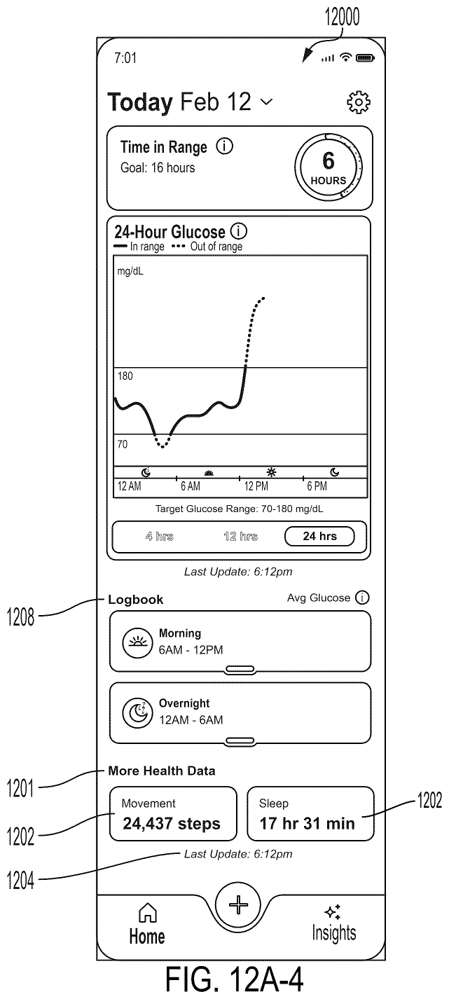

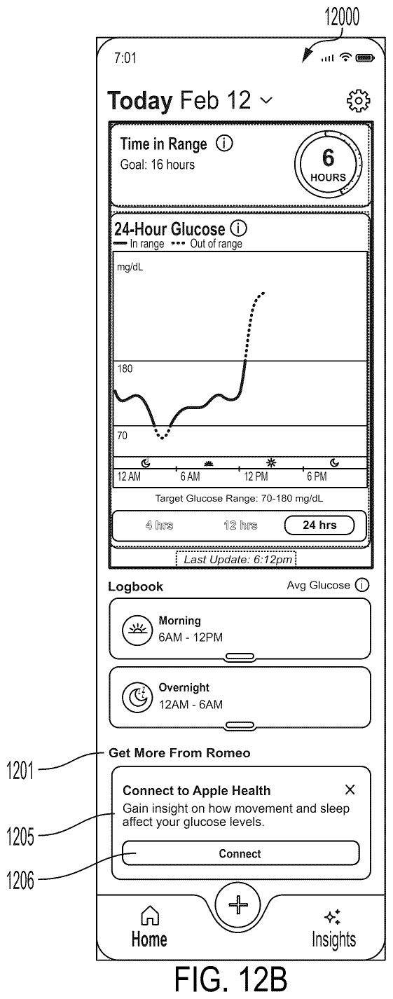

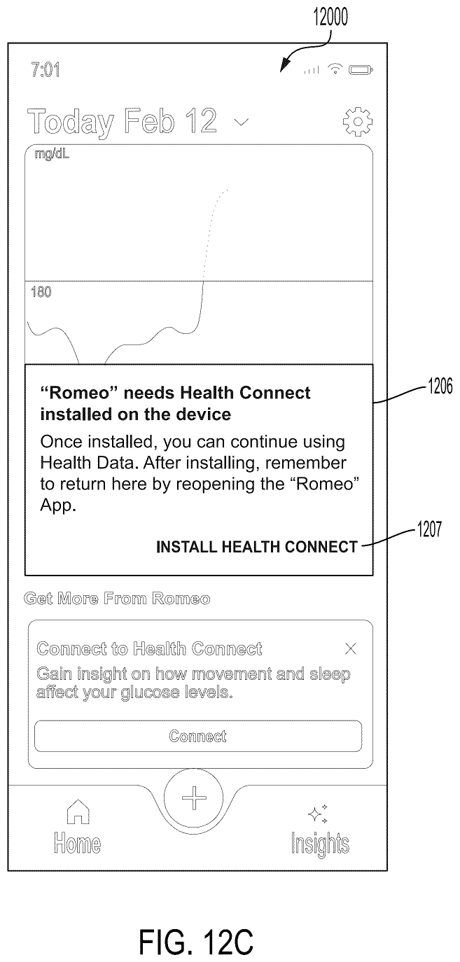

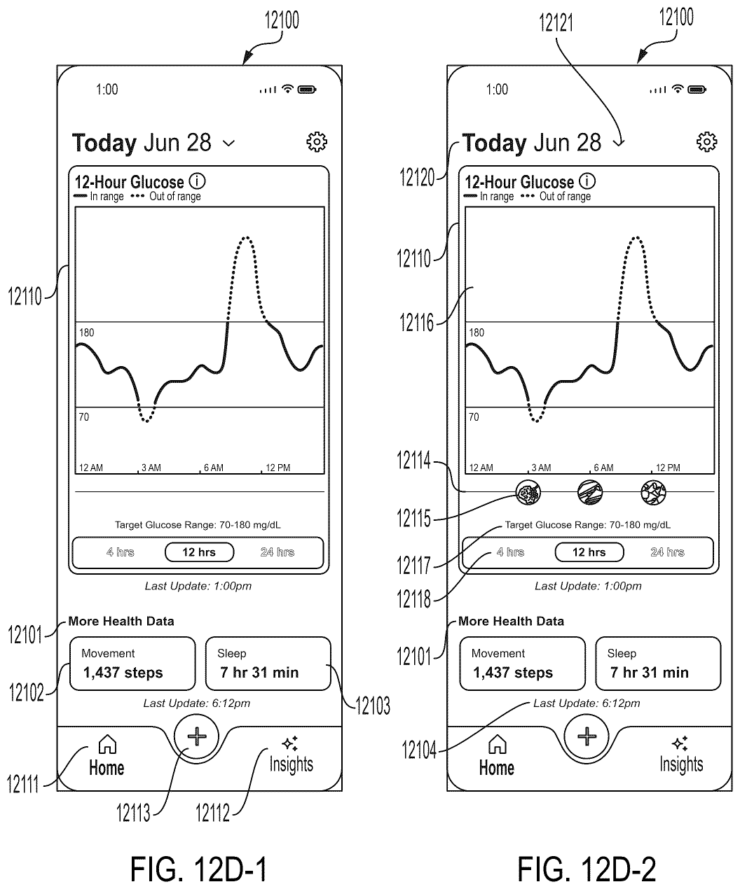

According to some embodiments, once the user has successfully logged and saved a meal, the user will be redirected to the GUI from which the user was directed to the meal logging GUI 500 from. For example, if the user selected the “+” icon on GUI 12100 (FIGS. 12D-1 and 12D-2), then the user will be redirected to GUI 12100 upon successfully logging a meal. In this manner, a corresponding meal icon 12115 to the newly logged meal can also appear on the meal timeline 12114 of GUI 12100 (FIG. 12D-2). As such, if the user associated a photograph or picture with the meal, then the meal icon 12115 (FIG. 12D-2) will comprise the photograph or picture. Additionally, if the user selected the “+” icon 15007 or the meal logging button 15023 on a notification 15004 corresponding to one or more missed meals on insights GUI 15000 (FIGS. 15A-1 to 15A-5) as described herein, the user will be redirected to insights GUI 15000 upon successfully logging a meal. In some embodiments, though not illustrated when a new meal is logged, and as will be described in further detail below, the insights GUI 15000 (shown in FIG. 15A-1 to 15A-5) can be updated so as to display a meal event corresponding to the newly logged meal. Further, according to some embodiments, and as will be described in further detail below, when a new meal is logged, a total average meal score for a predetermined period of time can be updated so as to account for the newly logged meal.

### Example Embodiments of Report GUIs and Related Methods

An example embodiment of a method for outputting a report GUI will be described. In particular, FIG. 6A shows operations of a method 6000 for performing the process for a report GUI that displays data indicative of an analyte level for a particular day, and non-medical statistics related thereto. As an initial matter, those of skill in the art will recognize that the method steps described herein can comprise software instructions stored in a memory of a computing device of system 100 (e.g., a reader 120, a local computer system 170, a trusted computer system 180), such that the instructions, when executed by one or more processors of the computing device, cause the one or more processors to perform any or all of the method steps described herein. Referring again to FIG. 6A, at 6001, the method for a report GUI can include receiving, by the user, a seventh predetermined input (e.g., a tap or press gesture) on a toggle switch adjacent to a text description on an interface, wherein the text description indicates a current day of the week. In one aspect of the embodiments, when the switch has been toggled down by the user, the displayed interface is configured to display a plurality of selectable rings, each of which is a TIR indicator corresponding to a different day in the past week. For example, in some embodiments, a plurality of seven selectable rings are displayed on the displayed interface.

At 6002, the method for a report GUI further includes the user selecting a particular ring of the plurality of rings which are displayed in response to the user toggling the switch, wherein each of the plurality of selectable rings is a TIR indicator corresponding to a different day in the past week.

At 6003, determining, by at least one processor, and based on data stored in a database, a set of data indicative of the analyte level and meal information which is associated with the day corresponding to the selected ring.

At 6004, providing, to a display device, an interactive report GUI configured for display of the set of data indicative of the analyte level and meal information based on the determining, wherein the report GUI displays: a TIR card, an analyte graph card reflecting data indicative of the analyte level for the time period corresponding to the selected ring, and/or a list of meal reflecting meal information for the time period corresponding to the selected ring. Those of skill in the art will recognize that method 4000 (FIG. 4A-1) either in its entirety or any one or more of the individual steps, can be combined with, or implemented as part of, method 6000 of FIG. 6A. In like manner, those of skill in the art will recognize that method 4100 (FIG. 4A-2) either in its entirety or any one or more of the individual steps, can be combined with, or implemented as part of, method 6000 of FIG. 6A.

As stated earlier, those of skill in the art will recognize that the method steps described herein can comprise instructions (e.g., software, firmware, etc.) stored in non-transitory memory of sensor control device 102, reader device 120, or any other computing device or system that is part of, or in communication with, analyte monitoring system 100. Further, the method steps described herein can be performed by a single centralized device or by multiple devices.

Turning to FIG. 6B-1, a block diagram depicting an example embodiment of a report GUI 600 for a TIR application is shown. As previously described, a report GUI 600 can be displayed in response to the user selecting a particular ring from the plurality of rings 425 displayed on the home GUI 400 or home GUI 450. Specifically, and as illustrated in FIG. 6B-1, a report GUI 600 corresponding to the relevant day associated with the selected ring 425 will be displayed. The report GUI 600 can comprise a (1) TIR card 601; (2) an analyte graph card 602 reflecting data indicative of the analyte level and meal information corresponding to the particular day associated with the selected ring 425; (3) a list 603 of meal information comprising one or more meal events 604; (4) a selectable “Day Summary” icon 605 that, when selected by the user, outputs the report GUI 600 for the relevant day; (5) a selectable meals icon 606 that, when selected by the user, outputs a meals impact GUI 650 (not shown in FIG. 6B-1) comprising information on one or more meal events 604 which occurred in a previous time period (e.g., the past week); and/or, (6) a “+” icon 607 that, when selected by the user, outputs the meal logging GUI 500 (FIG. 5A) configured to receive meal information inputted by the user.

According to some aspects of the embodiment, the plurality of rings 425, each of which corresponds to a different day in the week, are displayed above and adjacent to the TIR card 601. In some embodiments, only the particular ring 425 corresponding to the displayed report GUI 600 will be highlighted, bolded, or colored so as to indicate the report GUI 600 is associated with that particular ring 425. According to another aspect of the embodiments, the user can transition between report GUIs 600 corresponding to a particular day by selecting the ring from the plurality of rings 425 associated with the particular day. Further, in some embodiments, the report GUI 600 can comprise a “Go to Today” link 608 which, when selected, outputs the home GUI 400 comprising data corresponding to the current day or a report GUI 600 corresponding to the current day.

The TIR card 601 displayed on the report GUI 600 comprises a TIR ring 612 with a TIR value 611 inside the TIR ring 612. In some embodiments, and as illustrated in FIG. 6B-1, the TIR value 611 can comprise a numerical value and unit of measure indicative of number of hours the user spent in the predetermined target/goal TIR range for the relevant day (e.g., “15 hours”). Further, the number of hours spent in the TIR range can be visually displayed as a progress indicator 618 (e.g., a shaded portion) extending along the perimeter of the TIR ring 612 to X % of the total perimeter of the TIR ring 612. In this manner, the ratio of the perimeter of the TIR ring 612 comprising the progress indicator 618 and the total perimeter of the TIR ring 612 is proportional to a ratio of the amount of time that the user spent in the target TIR range and a predetermined daily TIR goal 617 for the relevant day. In some embodiments, the TIR card further comprises a message 635 related to the user's daily TIR goal 617. In some embodiments, the TIR card 601 further comprises the daily TIR goal 617 indicative of the predetermined TIR goal (e.g., “10 hours”). The daily TIR goal 617 can be displayed below the message 635 related to the user's daily TIR goal 617.

Still referring to FIG. 6B-1, and similar to home GUI 450 previously described, the analyte graph card 602 in report GUI 600 can include an analyte graph 630 comprising an analyte trendline 631 reflecting the user's analyte level, based on the data indicative of the analyte level, over a predetermined time period. For example, and as shown in FIG. 6B-1, the x-axis of the analyte graph 630 can comprise units of time (e.g., three-hour increments, etc.) covering a twenty-four-hour period, whereas the y-axis can comprise units of measure (e.g., mg/dL) for a measured analyte concentration of the user. For example, and as illustrated in FIG. 6B-1, the time displayed on the x-axis can show the glucose concentrations measured starting from 12 am of the current day.

The analyte graph 630 can include solid lines 632-1, 632-2 to indicate the user's analyte goal range associated with the data indicative of the analyte level. For example, the area between solid lines 632-1, 632-2 indicates a target or goal analyte range (e.g., between 70 mg/dL and 180 mg/dL). In some embodiments, and as illustrated in FIG. 6B-1, the analyte trendline 631 is configured as a dashed or broken line to represent a period of time in which the user's analyte level exceeds the analyte TIR goal range. Further, the analyte trendline 631 is configured as a solid line to represent a period of time in which the user's analyte level is within the analyte goal range. As previously described, the TIR goal range can be configured by the user.

According to another aspect of the embodiments, one or more meal icons 633 can be displayed on the analyte trendline 631. Each meal icon 633 can be a photograph or image associated with a particular meal. In some aspects of the embodiments, the meal icons 633 are positioned on the analyte trendline 631 so as to indicate a post-prandial analyte trace associated with the relevant meal. In some embodiments, the meal icons 633 are positioned on the analyte trendline 631 based on a time associated with the meal.

Still referring to FIG. 6B-1, and similar to home GUI 400 and home GUI 450, report GUI 600 can comprise a list 603 of meal information comprising one or more meal events 604 that occurred in the relevant day described in the report GUI 600. Each of the meal events 604 provides details of a particular meal consumed within the relevant day. In some embodiments, and similar to the previously described GUI embodiments, each meal event 604 can include a text description of a meal 640, a portion size indicator 641 describing the relative portion size of the meal, and/or a timestamp 642 associated with a time the meal was consumed, and/or a score 643. In some embodiments, the score 643 corresponding to a particular meal event 604 can be displayed as a numerical value inside a star icon 644. Though not illustrated, in some embodiments, a meal event 604 can include one or more meals tags associated with the meal. Additionally or alternatively, the meal event 604 can include a photograph or picture 646 associated with the meal. In some embodiments, when the user does not associate a photograph or picture 646 with the meal, a placeholder picture 646 will be displayed instead. In some exemplar embodiments, the list 603 can be ordered such that the meal event 604 corresponding to the most recently consumed meal is presented first or at the top of the list 603, with subsequent meal events 604 being displayed in chronological order. In some embodiments, a score information icon 648 is displayed proximal and adjacent to the meal card on report GUI 600. When selected by the user, score information icon 648 outputs meal scoring informational modal comprising information on the different score options and how a meal score is determined.

Turning to FIG. 6B-2, a block diagram of another example embodiment of a report GUI 625 for a TIR application is depicted. The report GUI 625 depicted in FIG. 6B-2 is similar to report GUI 600 in FIG. 6B-1, except that it comprises a selectable home icon 626 instead of a selectable “Day Summary” icon 605 (FIG. 6B-1). Further, report GUI 625 comprises a plurality of rings 627, each of which corresponds to a different day in the week. Similar to the plurality of rings 425 depicted in report GUI 600, each of the plurality of rings 627 depicted in report GUI 625 represents the progress the user made towards that particular day's daily TIR goal. In report GUI 625, however, the number of TIR hours the user spent in a particular day can be visually displayed by a color gradient of particular ring of the plurality of rings 627. For example, in some embodiments, and as depicted in FIG. 6B-2, the darkness or shade of the color gradient can indicate the amount of time the user spent in range for a particular day associated with a particular ring of the plurality of rings 627.

### Example Embodiments of Meals Impact GUIs

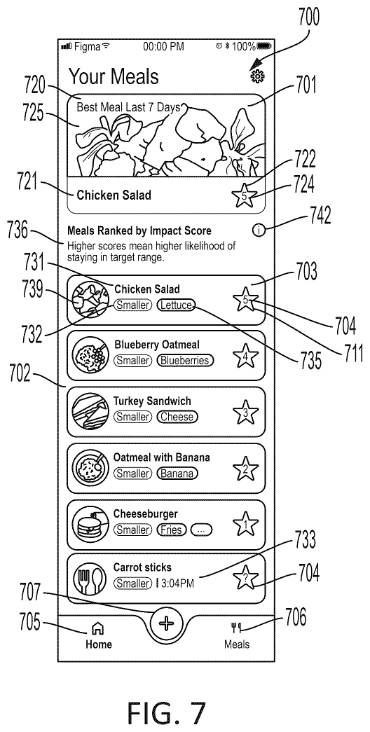

Example embodiments of a meals impact GUI for analyte monitoring systems will now be described. FIG. 7 is a block diagram depicting an example embodiment of meals impact GUI 700 for use with an analyte monitoring system. As seen in FIG. 7, meals impact GUI 700 can provide additional insights to the user as to the impact of consuming particular foods. In some embodiments, the meals impact GUI 700 can provide information related to the meals and data indicative of the analyte level for a particular time period, e.g., the last seven days.

According to an aspect of the embodiments, the meals impact GUI 700 comprises: (1) a meal highlight card 701; (2) a list 702 of meal information comprising meal events 703 ranked according to a score 704 assigned based on the corresponding analyte response recorded; (3) a selectable home icon 705; (4) a selectable meals icon 706; and (5) a selectable “+” icon 707. A meals impact GUI 700 can be accessed from the meals icon 706 or the meals icon described with respect to other GUI embodiments described herein. The meals impact GUI 700 can list all of the meal events 703 logged in a predetermined time period (e.g., one week). In the meal list 702, the meals can be ordered from highest score 704 to the lowest score 704, or, alternatively, from lowest score 704 to the highest score 704, where the score 704 is an assigned numerical value that corresponds to an analyte response based on the data indicative of the analyte level for the user. In some embodiments, when one or more meals are given a same score 704, the meals can be ordered by analyte level variance in the meal list 702. For example, if two meals were provided a score 704 of “3,” then the meals could be ordered from the lowest PeakDelta or analyte level variance value to the highest PeakDelta or analyte level variance value. Further, in some embodiments, if one or more meals are providing the same score 704 and a same analyte level variance, then the meals can be ordered chronologically.

In some embodiments, the analyte response can be based on a change in an analyte level within a predetermined time period after the particular meal is consumed. In some embodiments, meal events 703 are given a score 704 based on the change in analyte level from the time the particular meal was consumed to the peak glucose within three hours of eating.

In some embodiments, the score 704 can be a number between one and five, wherein a higher assigned numerical value corresponds to a lower analyte response, and a lower assigned numerical value corresponds to a higher analyte response. For example, and as previously detailed, (1) a 0 to 24 mg/dL change in an analyte level within a three hour period post-meal can correspond to a “5” score 704; (2) a 25 to 49 mg/dL change in an analyte level within a three hour period post-meal can correspond to a “4” score 704; (3) 50 to 74 mg/dL change in an analyte level within a three hour period post-meal can correspond to a “3” score 704; (4) a 75 to 99 mg/dL change in an analyte level within a three hour period post-meal can correspond to a “2” score 704; and (5), a 100 mg/dL or higher change in an analyte level within a three hour period post-meal can correspond to a “1” score 704. As depicted in meals impact GUI 700, the score 704 can be displayed in a graphical element, such as a star icon 711. In some embodiments, the star icon 711 is filled with a colored portion, such as yellow. In many embodiments (not illustrated), the star icon 711 is unfilled and comprises a broken outline to indicate a missing meal score 704. In some embodiments, and as depicted in FIG. 7, a missing meal score 704 is indicated by a “?” character. In some embodiments, the “?” character is displayed in the star icon 711 instead of a numerical value so as to represent that a particular meal was not scored. A missing meal score 704 happens when one or both of the following occurs: (1) the user logs the next meal too soon, or (2) the time-in-range application does not have sufficient analyte data to score the meal (e.g., the user has not scanned in a timely manner so as to ensure glucose data has been transmitted to the time-in-range application).

According to one aspect of the embodiments, the meal highlight card 701 is configured to display information related to a meal event 703 which occurred in a predetermined time period (e.g., the past week). In particular, the meal highlight card 701 displays information related to the meal event 703 that comprises the “best meal” and includes a title, “Best Meal Last 7 Days” 720. In some embodiments, the best meal is defined by the highest scoring meal event 703 within the predetermined time period. In some embodiments, the best meal is defined by the meal event 703 within the last seven-day period which provided the most desired analyte response, based on the data indicative of the analyte level. In some embodiments, the meal highlight card 701 comprises a text description 721 of the best meal and a star icon 722 comprising the assigned score 724 corresponding to the best meal. The meal highlight card 701 can further comprise a photograph or picture 725 associated with the best meal, wherein the text description 721 and star icon 722 are overlayed thereon so as to obstruct a portion of the photograph or picture 725.

According to another aspect of the embodiments, each of the meal events 703 listed on the meals impact GUI 700 provides details on a particular meal consumed within the predetermined time period (e.g., one week period). In some embodiments, each meal event 703 can include a text description of a meal 731, photograph or picture 739 associated with the meal, a portion size indicator 732 describing the relative portion size of the meal, a timestamp 733 associated with a time the meal was consumed, and/or the score 704 associated with the meal. In some embodiments, a meal event 703 can additionally or alternatively include one or more meals tags 735 associated with the meal (e.g., “lettuce,” “blueberries,” “cheese,” “banana,” fries,”). In some embodiments, an informational message 736 is displayed proximal to the list 702, wherein the informational message 736 informs the user that a higher score 704 means a higher likelihood of staying in target TIR range.

Still with reference to FIG. 7, meals impact GUI 700 can further include a score information icon 742. In some aspects of the embodiments, each of the meal events 703 are selectable and, upon being selected by the user, output a meal review GUI 800 or meal review GUI 8000 (FIGS. 8A and 8B-8E, respectively) providing details related to the selected meal event 803, as will be described further below.

In some embodiments, though not illustrated, meals impact GUI 700 can include a graphical representation, such as segmented bar, instead of a star icon 711, to indicate the score 704 to the user. Specifically, though not illustrated, meals impact GUI 700 can include a segmented bar with three segment portions, wherein: (1) the first segment can have a red color or orange color and be indicative of an analyte score corresponding to a high glucose impact or response (e.g., an average glucose range exceeding 250 mg/dL); (2) the first segment and second segment can have a yellow color and be indicative of an analyte score corresponding to a medium glucose impact or response (e.g., an average glucose range between 181 mg/dL and 250 mg/dL, or no greater than 250 mg/dL and/or at least 181 mg/dL); or (3) the first segment, the second segment, and the third segment can have a green color and be indicative of an analyte score corresponding to a low glucose impact or response (e.g., an average glucose range between 70 mg/dL and 180 mg/dL, or no greater than 180 mg/dL). Those of skill in the art will appreciate that various other graphical representations and colors, and color configurations can be utilized with the graphical representations without departing from the scope of the disclosure.

### Example Embodiments of Meal Review GUIs

Example embodiments of meal review GUIs for analyte monitoring systems, and other related features will now be described. FIG. 8A is a block diagram depicting an example embodiment of a meal review GUI 800 for use with an analyte monitoring system. As seen in FIG. 8A, the meal review GUI 800 can provide additional insights to the user as to the impact of consuming particular foods. As seen in FIG. 8A, the user can select a meal event from a list on the report GUI 600, meals impact GUI 700, home GUI 400, home GUI 450, home GUI 430, home GUI 485, home GUI 495, home GUI 499, or any other interface described herein which can be configured as a home interface. Once a meal event is selected by the user, meal review GUI 800 can open to show details related to a selected meal event 803, including a photograph/picture 805, description 801, portion size indicator 802, meal tags 804, timestamp 806 comprising a date and time associated with the meal, score 807, and/or an analyte graph card 810 comprising an analyte graph 811 with an analyte trend line 812 containing data indicative of an analyte level for at least the relevant time period in which the meal was eaten. In some embodiments, though not illustrated in FIG. 8A, the analyte graph card 810 can comprise a selectable informational icon which, upon being selected, outputs analyte graph information modal 445, as shown in FIG. 4G.

In particular, the meal review GUI 800 includes a meal description card 820 which includes the description 801, portion size indicator 802, meal tags 804, and timestamp 806 associated with the meal. Further, the score 807 is displayed in a star icon 830, and is positioned above the description 801 of the meal. In some embodiments, and as illustrated in FIG. 8A, the meal description card 820 partially obstructs the photograph or picture 805 associated with the meal.

Still referring to FIG. 8A, and similar to analyte graph card described in previous GUI embodiments, the analyte graph card 810 in meal review GUI 800 can include the analyte graph 811 with the analyte trendline 812 reflecting the user's analyte level, based on the data indicative of the analyte level, over a predetermined time period. For example, and as shown in FIG. 8A, the x-axis of the analyte graph 811 can comprise units of time (e.g., one-hour increments, etc.) covering a predetermined time period, whereas the y-axis can comprise units of measure (e.g., mg/dL) for a measured analyte concentration of the user. The analyte graph card 810 can include solid lines 813-1, 813-2 to indicate the user's analyte goal range associated with the data indicative of the analyte level. For example, the area between solid lines 813-1, 813-2 indicates a target analyte range (e.g., between 70 mg/dL and 180 mg/dL). In some embodiments, and as illustrated in FIG. 8A, the analyte trendline 811 is configured as a dashed or broken line to represent a period of time in which the user's analyte level exceeds the analyte TIR goal range. Further, the analyte trendline 812 is configured as a solid line to represent a period of time in which the user's analyte level is within the analyte goal range.

According to another aspect of the embodiments, a meal icon 840 corresponding to the meal event 803 can be displayed on the analyte trendline 812. The meal icon 840 can be a photograph or image associated with the meal of the meal event. In some aspects of the embodiments, the meal icon 840 is positioned on the analyte trendline 812 so as to indicate a post-prandial analyte trace associated with the relevant meal. In some embodiments, the meal icon 840 is positioned on the analyte trendline 812 based on a time associated with the meal. In other embodiments, the meal icon 840 can be positioned above or below the analyte trendline 812. In some embodiments, the meal icon 840 can be positioned above or below the analyte graph 811. In some embodiments, though not illustrated in FIG. 8A, the meal icon 840 can be positioned on a meal timeline separate and distinct from the analyte graph 811 and/or analyte trendline 812, as will be described in further detail below with reference to other interface embodiments (see, e.g., FIG. 12D-2).

In some embodiments, the meal review GUI 800 includes analyte level statistics 850 related to the meal event 803. For example, and as illustrated in FIG. 8A, the meal review GUI 800 can comprise analyte level statistics related to a peak analyte level associated with the meal 850-1 (e.g., “173 mg/dL”), an analyte level before the meal was eaten 850-2 (e.g., “140 mg/dL”), and an analyte level after the meal was eaten 850-3 (e.g., “95 mg/dL”). In some embodiments, the analyte level statistics 850 are displayed proximal and adjacent to the analyte graph card 810. In this manner, the user can refer to the analyte graph card 810 for a visual illustration of the analyte level statistics 850 associated with the meal. Further, in some embodiments, a message 860 indicating the target glucose range (e.g., “Target Glucose Range: 70-80 mg/dL”) is displayed below the analyte graph card 810. In some aspects of the embodiments, an informational message 870 is also displayed on the meals review GUI 800, wherein the informational message 870 indicates that medications, activity, stress, and other facts can impact glucose levels. In yet another aspect of the embodiments, the meals review GUI 800 can further include a meal summary message 880, wherein the meal summary message 880 describes a glucose spike associated with the meal events, statistics related to the user's TIR, and can comprise a motivating message (e.g., “Your glucose spiked by +33 mg/dL. You got in range 3 hours after this meal. Nice!”).

FIG. 8B-8E are block diagrams depicting an additional example embodiment of a meal review GUI 8000 for use with an analyte monitoring system. Meal review GUI 8000 can be similar to meal review GUI 800, except that the score 8007 is not displayed in a star icon and instead includes a different graphical representation. Specifically, the score 8007 can be represented by a segmented bar 8008 with three segment portions 8009, wherein: (1) the first segment 8009a can have a red color or orange color and be indicative of an analyte score corresponding to a high glucose impact or response (e.g., an average glucose range exceeding 250 mg/dL); (2) the first segment 8009a and second segment 8009b can have a yellow color and be indicative of an analyte score corresponding to a medium glucose impact or response (e.g., an average glucose range between 181 mg/dL and 250 mg/dL, or no greater than 250 mg/dL and/or at least 181 mg/dL); or (3) the first segment 8009a, the second segment 8009b, and the third segment 8009c can have a green color and be indicative of an analyte score corresponding to a low glucose impact or response (e.g., an average glucose range between 70 mg/dL and 180 mg/dL, or no greater than 180 mg/dL). Those of skill in the art will appreciate that various other graphical representations and colors, and color configurations can be utilized with the graphical representations without departing from the scope of the disclosure. For example, FIG. 8B depicts a segmented bar 8008 with the first segment 8009a and second segment 8009b comprising a yellow color, indicating an analyte score corresponding to a medium glucose impact or score.

In like manner to meal review GUI 800, the meal review GUI 8000 provides additional insights to the user as to the impact of consuming particular foods. For example, the user can select a meal event from a list on the report GUI 600, meals impact GUI 700, GUI 12100, any of the home interfaces described herein, or any other interfaces described herein which comprise a selectable meal event or meal icon. Once a meal event or meal icon is selected by the user, meal review GUI 8000 can open to show details related to the selected meal event or meal icon.

In some embodiments, and as shown in FIG. 8C, if meal review GUI 8000 corresponds to a detected missed meal which has not yet been logged by the user (as will be described in further detail below), then meal review GUI 8000 can include a description 8010 indicating that a meal has been detected (“Meal Detected”), and a placeholder image 8011 instead of a photograph/picture. Further, and as shown in FIG. 8C, when a missed meal is detected, then meal review GUI 8000 can also include an in-app notification 8012 relating to the detected missed meal. The in-app notification 8012 can be positioned between a meal description card 8016 and a meal summary message 8017.

In some embodiments, and with reference to FIG. 8C, the in-app notification 8012 comprises a query 8013 asking whether the user has eaten (e.g., “Did you eat around 1:00 pm?”), and a message 8014 indicating to the user that the user's glucose was rising around a particular time and that the user should log a meal (e.g., “We noticed your glucose was rising around this time. If you had a meal, try logging it now.”). According to an aspect of the embodiments, the in-app notification 8012 can include a selectable “ignore” button 8015 and a selectable meal logging button 8022. Specifically, upon the user selecting the ignore button 8015, a modal (not shown) will appear which indicates to the user that ignoring the missed meal will delete the meal detection and return the user to a home interface. In some embodiments, though not illustrated, the modal will include a confirmation button.

Further, in some embodiments, if the user selects the meal logging button 8022, meal logging GUI 500 (FIG. 5A) is outputted. Moreover, in some embodiments, meal entry modal 520 (as shown in FIG. 5C) can appear, wherein the time field 527 is prepopulated with a date and time associated with the detected missed meal associated with the in-app notification 8012 from which the meal logging button 8022 was selected. In other embodiments, meal entry modal 520 (as shown in FIG. 5C) can appear, wherein the user sets the date and time associated with the detected missed meal in the time field 527. In some embodiments, when meal logging GUI 500 is outputted through the user selecting the meal logging button 8022 of meal review GUI 8000, a camera interface (e.g., FIGS. 5I to 5N) is not shown to the user. In some embodiments, one the user has logged a meal for the particular time associated with the missed meal detection (e.g., by selecting the meal logging button 8022 in the in-app notification 8012), the respective in-app notification 1301 will be removed from GUI 8000 (see, e.g., FIG. 8B).

According to another aspect of the embodiments, and with particular reference to FIG. 8C, a missed meal episode marker 8018, such as a missed meal icon 8018 can be displayed near, close to, and/or proximal to analyte trendline 8019 on the analyte graph 8023 shown on meal review GUI 8000. In some embodiments, the missed meal icon 8018 comprises a placeholder image to indicate that the missed meal icon 8018 corresponds to an unlogged meal. In some embodiments, the analyte trendline 8019 can include meal icons 8020 corresponding to other meal events that occurred during a predetermined time period (e.g., the last 24-hours) represented by the analyte graph 8023. Further, in some embodiments, meal review GUI 8000 can include a selectable edit link 8024 which, upon being selected, allows the user to edit the information provided in the meal review GUI 8000.

In some embodiments, and as best depicted in FIG. 8D, if a missed meal is detected yet not scored (e.g., if a predetermined time period after the particular meal is consumed has not yet elapsed, such as a three-hour time period post-meal, then a score is not yet available for the detected missed meal), then the meal review GUI 8000 does not display the graphical representation (e.g., the segmented bar 8008) corresponding to a score of a meal.

In some embodiments, and as depicted in FIG. 8E, if a missed meal is detected yet only some of the analyte data (e.g., glucose data) is available, then the meal review GUI 8000 can display the graphical representation for the missed meal if the score is available. For example, and as shown in FIG. 8E, the segmented bar 8008 corresponding to the score 8007 of the missed meal is shown. Further, and as shown in FIG. 8E, if only some of the analyte data is available for a detected missed meal, then meal review GUI 8000 can display the analyte graph 8023 without an analyte trendline. In some embodiments, the analyte graph 8023 will include a message indicating to the user that there is no glucose data. Further, in some embodiments, if only some of the analyte data is available for a detected missed meal, then meal review GUI 8000 can display only a portion of the analyte level statistics. For example, and as shown in FIG. 8E, meal review GUI 8000 can display analyte level statistics related to a peak analyte level associated with the detected meal 8021-1 (e.g., “195 mg/dL”), an analyte level before the detected missed meal was eaten 8021-2 (e.g., “195 mg/dL”), but not an analyte level after the meal was eaten 8021-3 (e.g., “-- mg/dL”).

In summary, in some exemplar embodiments, if it is determined that an episode has been detected and at least a predetermined period of time (e.g., three hours) has elapsed since a respective meal associated with the episode was consumed, meal review GUI 8000 can display the score 8007, the in-app notification 8012, and the missed meal episode marker 8018, as best depicted shown in FIG. 8C. In this regard, a meal is detected and scored. Further, in some embodiments, if it is determined that sufficient time has elapsed to identify a peak analyte value in the analyte data but not enough time has elapsed to accurately score a meal (e.g., between 30-minutes and three hours subsequent to a meal being consumed), then meal review GUI 8000 is displayed without a graphical representation corresponding to a score of the meal, as best depicted in FIG. 8D. In this regard, the meal is detected but not yet scored. Additionally, according to some embodiments, if is determined that sufficient time has elapsed to identify a peak analyte value in the analyte data and to accurately score the meal associated with a detected episode, but there is missing analyte data or another meal is logged subsequent to and in close time-proximity to a first meal associated with the detected episode (e.g., within a one-hour window of the first meal associated with the detected episode), then meal review GUI 8000 can display the in-app notification 8012 relating to a detected missed meal but may not display a score for the detected missed meal (e.g., similar to meal review 8000 embodiment shown in FIG. 8F, except that FIG. 8F depicts a score for the detected missed meal). In this regard, a meal is detected but un-scoreable.

Further, in some embodiments, once a detected meal event has been successfully logged, the TIR application will output meal review GUI 8000, as depicted in FIG. 8B. Further, in some embodiments, if a missed meal is detected yet not yet logged, the user has a predetermined amount of time to log the missed meal prior to it automatically being deleted. For example, in some embodiments, the user has seven days to retroactively log a detected missed meal in the TIR application.

### Example Embodiments of Onboarding GUIs

The TIR application can also include onboarding interfaces, any of which can be utilized with the embodiments described herein. According to one aspect of the embodiments, an onboarding GUI can be displayed when the user launches the TIR application. In some embodiments, onboarding GUIs can provide the user with a brief introduction to the TIR application. For example, in some embodiments, an onboarding GUI can inform the user that the TIR application can help the user understand how meals impact a user's glucose levels and overall glucose management by giving the user a daily score, an average analyte rating, or by tracking the user's time-in-range. In some embodiments, an onboarding GUI explains to the user that (1) the TIR application provides a daily score to the user based on the meals the user logs and the user's glucose control, and (2) that the daily score is calculated daily and resets at a predetermined time each day (e.g., at midnight). Further, in some exemplar embodiments, an onboarding GUI can explain to the user that TIR is the amount of time spent within a target glucose range (e.g., between 70 mg/dL and 180 mg/dL or 3.9 mmol/L to 10 mmol/L) and that positive health outcomes are more likely to be experienced with longer TIR. In some exemplar embodiments, an onboarding GUI explains to the user that (1) the TIR application helps the user track how different foods impact the user's glucose levels and provides the user with an average meal rating; (2) the rating is an average of the ratings the user receives from the meals logged for a particular day; and, (3) over time, the user will be able to see which foods work best to improve the user's average meal rating. In one aspect of the embodiments, onboarding GUIS can be provided to explain the requirements of the TIR application to the user. For example, an onboarding GUI can inform the user that the following are needed: (1) the TIR application; (2) another analyte monitoring application; and (3) a sensor.

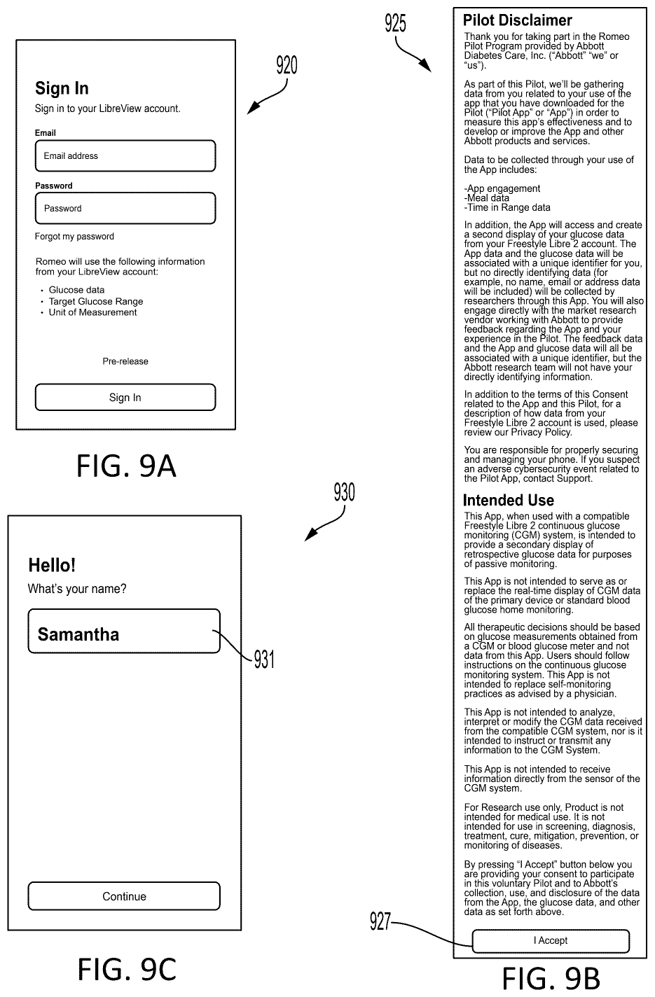

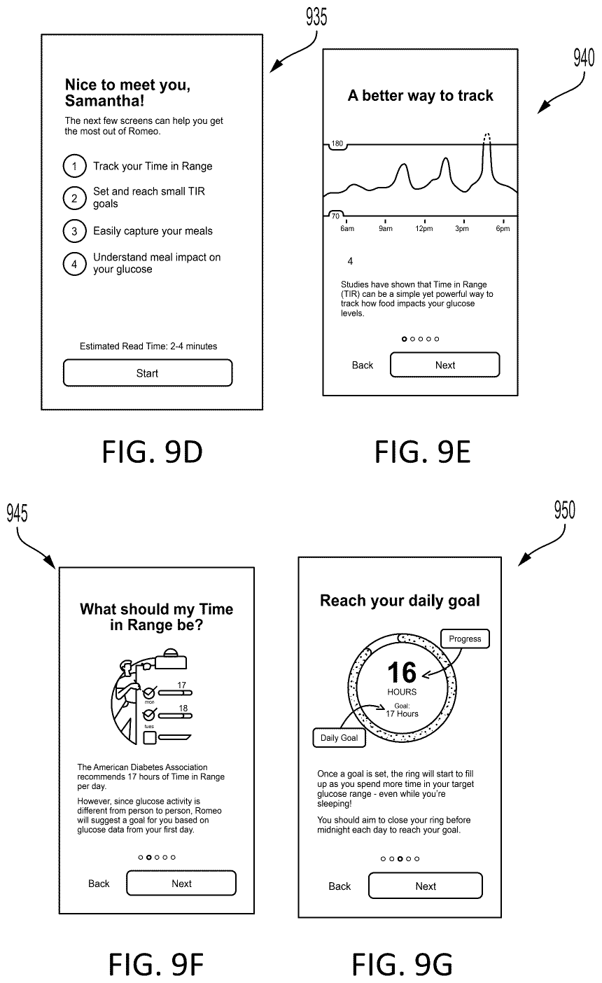

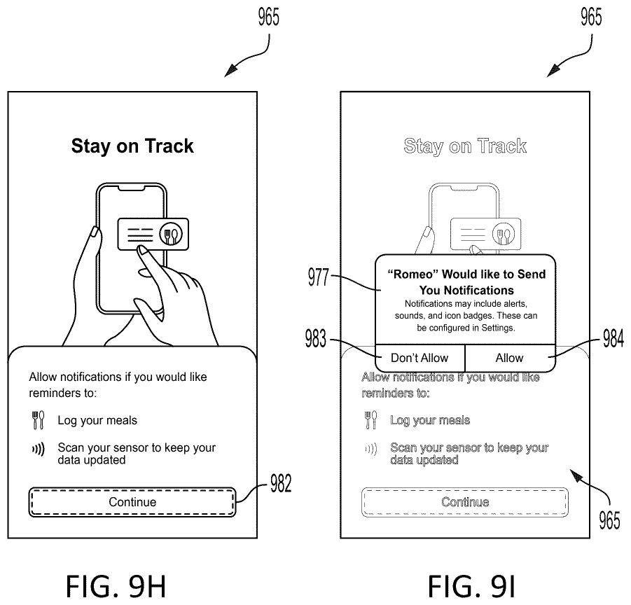

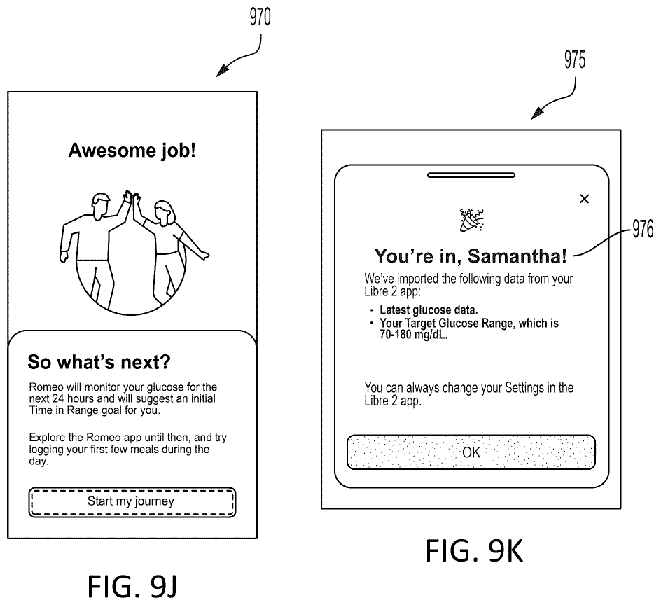

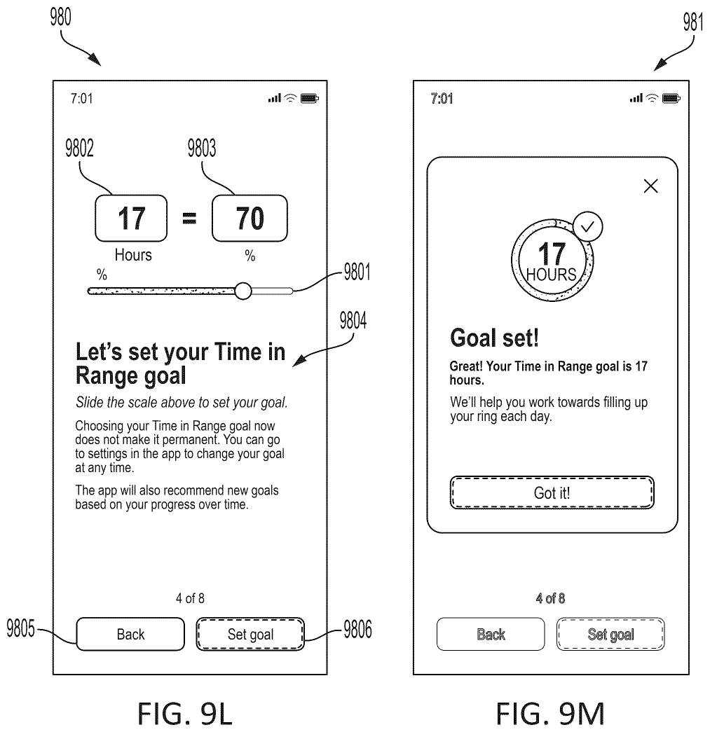

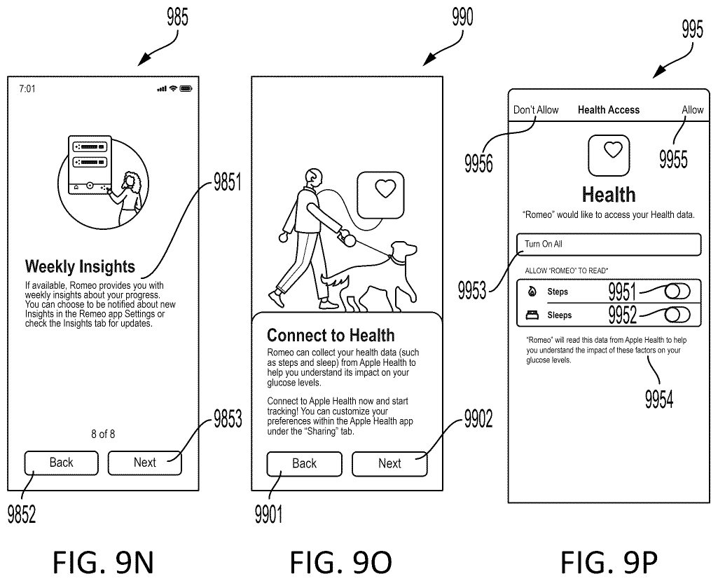

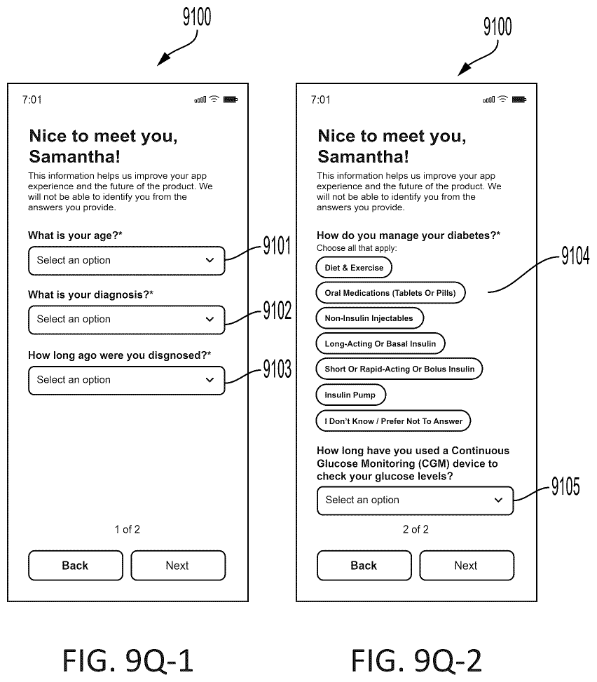

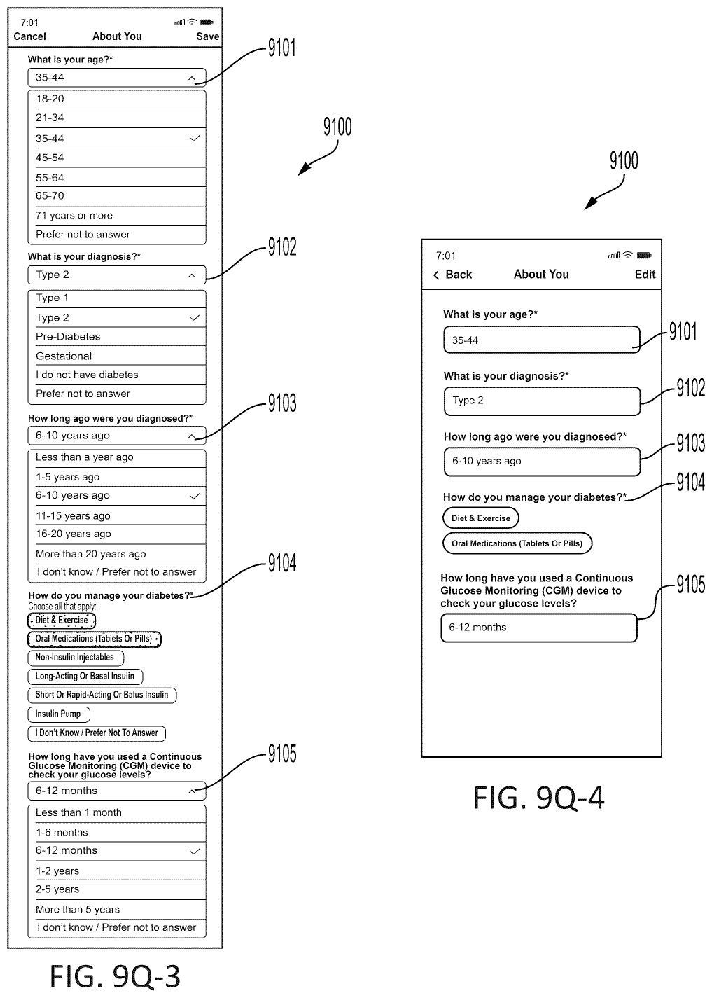

Further, FIG. 9A through FIG. 9Q-4 are block diagrams depicting example embodiments on onboarding GUIs and features related thereto. As depicted in FIG. 9A, onboarding GUI 920 can be utilized to login the user to an account. Next, FIG. 9B depicts onboarding GUI 925, which can provide the user with a pilot disclaimer. In some embodiments, onboarding GUI 925 includes a confirmation button 927 (e.g., ““I Accept” button) that can be pressed by the user to confirm agreement with the terms of the TIR application. Next, onboarding GUI 930 (FIG. 9C) is displayed to the user. As depicted in FIG. 9C, onboarding GUI 930 comprises a name entry field 931, wherein the user can input a name to be associated with user's account credentials.

Next, in some embodiments, and as depicted in FIG. 9D, an onboarding GUI 935 can provide the user an introductory greeting comprising the name inputted in preceding onboarding GUI 930. In some embodiments, onboarding GUI 935 can provide the user with an introductory greeting and indicate to the user the benefits of utilizing the TIR application (e.g., (1) the user can make the most of the TIR application by capturing what the user eats, (2) seeing impact on the user's analyte level's, such as glucose levels, and/or (3) tracking the user's average meal scores or total average meal score). Further, onboarding GUI 935 can provide a brief explanation of the content which appears in the subsequent onboarding GUIs along with an estimated read time for the content which appears in the subsequent onboarding GUIs. Following this, onboarding GUIs 940 (FIG. 9E), 945 (FIG. 9F), and 950 (FIG. 9G) can be utilized to provide the user with a further introduction to the TIR application.

In some embodiments, onboarding GUI 940 (FIG. 9E) can inform the user about studies which have shown that TIR can be a simple yet powerful way to track how food impacts glucose levels. Further, and with reference to FIG. 9F, onboarding GUI 945 can inform the user that the American Diabetes Association recommends 17 hours of TIR per day. In some embodiments, onboarding GUI 945 can also inform the user that since glucose activity is different from person to person, the TIR can vary as well. In some embodiments, onboarding GUI 945 can also inform the user that since glucose activity depends on the user, the TIR application will suggest a goal based on glucose data from the user's first day. With reference to FIG. 9G, a block diagram depicting onboarding GUI 950 is shown, wherein onboarding GUI 950 can inform the user that once a goal is set, the TIR ring will fill as the user spends more time in their TIR, even while the user sleeps. Onboarding GUI 950 can also include a message encouraging the user to close the TIR ring before midnight each day in order to reach their goal.

Further, in some embodiments, an onboarding GUI can be displayed which provides the user with brief instructions on how to log a meal in the TIR application. Further, in some embodiments, an onboarding GUI can be displayed which comprises a link which, upon being selected by the user, outputs a meal logging informational modal comprising tips for adding meals through the TIR application. In some embodiments, an onboarding GUI is displayed which provides the user with information related to meal rating or score. For example, in some embodiments, though not illustrated, an onboarding GUI can explain to the user that the TIR application rates meals based on glucose changes after eating. Further, in some embodiments, the onboarding GUI can also explain that a 5-star meal will cause the smallest amount of rise in glucose, while a 1-star meal will cause the highest. In other exemplar embodiments, the onboarding GUI can further explain that a meal that receives a filled green bar will cause the smallest amount of rise in the user's glucose, while an unfilled orange bar will cause the highest, and that overtime, the user will see which foods work best to improve the user's total average meal score. In some embodiments, though not illustrated, an onboarding GUI is displayed which provides the user with information related to the functional aspects of meal logging (e.g., including tags and portion sizes when logging meals).

In some embodiments, the onboarding GUI providing information related to meal ratings can comprise a link which, upon being selected, outputs a meal scoring informational modal comprising information on how a meal is ranked or scored in the TIR application, as described above. In some embodiments, the meal scoring information modal comprising information on how a meal is ranked can further comprise emoticons to indicate the impact on glucose. In some embodiments, the meal scoring information modal comprising information on how a meal is ranked can further comprise how a score bar is displayed when there is a missing score and a selectable link which, upon being selected, outputs an informational modal which comprises information related to missing scores. Further, according to some aspects of the embodiments, the meal scoring informational modal can comprise a link which, when selected by the user, outputs another meal scoring informational modal comprising details on why the score is measured three hours after a meal is consumed. In some embodiments, the meal scoring informational modal comprising details on why the score is measured three hours after a meal is consumed can also include a tip which provide the user with suggestions on how to avoid being out of range for longer periods of time. In some embodiments, the meal scoring information modal further comprises details on why the user should allow time between meals. Further, the meal scoring information modal further comprises details on logging all foods eaten in a 30-minute window under the same meal so as to minimize the number of meals that receive a missing score or rating.

Additionally, in some embodiments, an informational GUI can be provided which comprises details on why a meal is missing a score or rating. For example, the informational GUI can comprise one or more informational cards, wherein each of the informational cards describe different scenarios which could lead to an unscored meal (e.g., the user logged the next meal too soon, or the TIR application does not have enough glucose data to score the meal).

In some embodiments, an informational GUI can be provided which comprises detail on how the average meal score is calculated based on a predetermined duration of time (e.g., the average meal score is calculated based on the last 24 hours).

In another aspect of the embodiments, an onboarding GUI 965 (FIG. 9H) can be displayed, which informs the user that notifications must be allowed in order to receive reminders from the TIR application. Further, onboarding GUI 965 can further inform the user that notifications must be allowed in order to receive reminders that the user should scan the sensor to keep the user's data updated or to receive reminders that the user should log meals. Additionally, in some embodiments, onboarding GUI 965 informs the user that notifications must be allowed in order to receive notifications when new “insights” are available.

In some embodiments, though not illustrated in FIG. 9H, onboarding GUI 965 can comprise a “back” button which, upon being selected by the user, returns the user to the onboarding GUI previously displayed before onboarding GUI 965. Further, in some embodiments, and as depicted in FIG. 9H, onboarding GUI 965 comprises a “Continue” button 982 which, upon being selected by the user, outputs a notification modal window 977. Specifically, notification modal window 977 can include a “Don't Allow” button 983 which, when selected by the user, does not allow the TIR application to send notifications to the user. Notification modal window 977 can further include an “Allow” button 984 which, when selected by the user, allows the TIR application to send notifications to the user. In some embodiments, and as depicted in FIG. 9I, notification modal window 977 can be configured so as to partially obstruct or superimpose the underlying onboarding GUI 965 from view. Further, in some embodiments, when the “Allow” button 984 is selected by the user, onboarding GUI 970 is outputted (FIG. 9J) to confirm the onboarding process is complete.

Turning to FIG. 9K, a pop-up modal 975 is depicted. Pop-up modal 975 comprises a message indicating to the user that a particular set of data (e.g., the latest glucose data and the user's target glucose range) has been imported from another analyte monitoring application to the TIR application. Pop-up modal 975 further comprises a message header 976 including the user's inputted name (e.g., “You're in, [User's name]!”). Pop-up modal 975 can further comprise a confirmation “Ok” button that can be pressed by the user. Pop-up modal 975 can further comprise celebratory emoticons.

In some embodiments, the user can set their TIR goal through the onboarding process. Specifically, and as depicted in FIG. 9L, onboarding GUI 980 can be utilized to set the user's TIR goal. Onboarding GUI 980 can include a time entry field 9802, by which the user can manually input a desired TIR goal in time units (e.g., “17” hours). In another aspect of the embodiments, and as best depicted in FIG. 9L, a percentage entry field 9803 is provided by which the user can manually input a desired TIR goal in percentage units (e.g., “70” percent). In some embodiments, and as depicted in FIG. 9L, onboarding GUI 980 can further comprise a selectable bar 9801 configured to update the TIR goal in response to an eight predetermined input by the user, such as when the user, drags the selectable bar with a finger, or by some other predetermined gesture.

In some embodiments, if the user updates the TIR goal through one manner (e.g., through the selectable bar 9801, time entry field 9802, or percentage entry field 9803), onboarding GUI 980 will be updated so as to reflect the updated TIR goal in the time entry field 9802 and percentage entry field 9803 (e.g., 17 hours equals 70 percent). In this manner, the onboarding GUI 980 allows the user to visualize the new TIR goal in terms of both hours and percentages. In some embodiments, onboarding GUI 980 can further include an informational section 9804 that informs the user that choosing a TIR goal does not make it permanent and that the user can change the TIR goal at any time by going in the settings. Further, informational section 9804 can also inform the user that the TIR application will also recommend new TIR goals based on the user's progress over time.

As shown in FIG. 9L, the user can select a “back” button 9805 to return to the previous onboarding GUI. Further, in some embodiments, and as shown in FIG. 9L, after the new TIR goal settings have been inputted, the user can click “set goal” 9806 to save the TIR goal. In some embodiments, once “set goal” 9806 has been clicked by the user, a confirmation in-app modal 981 (as best depicted in FIG. 9M) is displayed on onboarding GUI 980, confirming the user's TIR goal has been set. In some embodiments, and as depicted in FIG. 9L, the confirmation in-app modal 981 comprises a “confirmation” button 9807 which the user can select to confirm the message provided in the in-app modal 981.

Turning to FIG. 9N, onboarding GUI 985 is depicted. Onboarding GUI 985 comprises information related to “insights,” which will be described in further detail below, provided by the TIR application. In particular, onboarding GUI 985 can comprise an informational section 9851 which informs that user that the TIR application can provide the user with weekly insights about their progress. Further, in some embodiments, informational section 9851 further informs the user that the user can choose to be notified about new insights in the TIR application's settings or check the “insights” tab or icon for updates. Onboarding GUI 985 can include a “back” button 9852 which, when selected by the user, returns to the user to the previously displayed onboarding GUI. Further, onboarding GUI 985 can further include a “next” button 9853 which, when selected by the user, displays a subsequent onboarding GUI.

In some embodiments, onboarding GUI 990 (depicted in FIG. 9O) is provided during the onboarding process of the TIR application. Specifically, onboarding GUI 990 can inform the user that the TIR application can collect health data (e.g., steps and sleep data) from a third-party health monitoring application (e.g., Apple Health, or Samsung Health or Google Fit through Health Connect) to help the user understand its impact on the user's glucose levels. Onboarding GUI 990 can further inform the user that the user can connect to the third-party health monitoring application (e.g., Apple Health or Health Connect) during the onboarding process and further customize preferences within the third-party health monitoring application under the “sharing” tab therein. In some embodiments, the onboarding GUI 990 comprises a “back” button 9901 which, when selected by the user, returns to the user to the previously displayed onboarding GUI.

In some embodiments, onboarding GUI 990 comprises a “next” button or “continue” button 9902 which, if selected for the first time by the user, displays onboarding GUI 995, which allows the user to connect to the third-party health monitoring application. Specifically, and as shown in FIG. 9P, onboarding GUI 995 is depicted, wherein onboarding GUI 995 can comprise a movements setting 9951, which can be configured by the user such that, when enabled, allows the third-party health monitoring application to share data related to the user's movements (e.g., steps or pushes) with the TIR application. In this manner, the TIR application is configured to read data from the third-party health monitoring application relating to the user's movement to better understand the impact thereof on the user's glucose levels. Onboarding GUI 995 can further comprise and a sleep setting 9952, which can be configured by the user such that, when enabled, allows the third-party health monitoring application to share data related to the user's sleep (e.g., hours of sleep) with the TIR application. In this regard, the TIR application is configured to receive data from the third-party health monitoring application relating to the user's sleep to better understand the impact thereof on the user's glucose levels. Further, onboarding GUI 995 can include a selectable “Turn on All” button 9953 which, when selected by the user, allows the TIR application to access health data from third-party health monitoring application relating to both the user's movement and sleep. Onboarding GUI 995 can also include an informational message 9954 which informs the user that the TIR application will read data from third-party health monitoring application to help the user understand the impact of the movement and sleep factors on the user's glucose levels. According to another aspect of the embodiments, onboarding GUI 995 can also include an “allow” button 9955 which, when selected by the user, outputs onboarding GUI 965 depicted in FIG. 9H. In some embodiments, onboarding GUI 995 can also include a “don't allow” button 9956.

Furthermore, and according to another aspect of the embodiments, if the user selects onboarding GUI's 995 “next” button or “continue” button 9902 for a second time or a subsequent time after the first selection, a notification modal will be displayed which informs the user that in order to allow the TIR application access to third-party health monitoring application, the user must go to the third-party health monitoring application's settings (e.g., Apple Health or Health Connect settings), under the “sharing” tab. In some embodiments, the notification modal comprises an “ok” button which the user can select to confirm acknowledgement of the notification. In some embodiments, an onboarding modal, though not illustrated, can be displayed which informs the user that the TIR application requires access to third-party health monitoring application (e.g., Health Connect or Apple Health). Specifically, the onboarding modal can include a (1) selectable installation link and/or (2) a continue link which, upon being selected, allows the user to continue utilizing the TIR application without connecting to a third-party health monitoring application.

In some exemplar embodiments, the user can set up profile settings through the onboarding process. For example, in some embodiments, and with reference to FIGS. 9Q-1 to 9Q-4, an onboarding GUI 9100 can be outputted which can include a plurality of selectable options or fields to prompt the user for profile information or preferences. For example, and as depicted in FIGS. 9Q-1 to 9Q-4, the plurality of selectable fields can include an age field 9101, wherein the user can input an age range; a medical condition field 9102, wherein the user can input whether they are diagnosed with a medical condition (e.g., Type 1 diabetes, Type 2 diabetes, or other condition); a diagnosis field 9103, wherein the user can input how long ago they were diagnosed with said medical condition; a medication field 9104, wherein the user can confirm how they manage their medical condition (e.g., whether they take diabetes-related medication(s), such as, long-acting/basal insulin or oral medication, or whether they diet, exercise, or use an insulin pump, or choose not to answer); and a medication history field 9105, wherein the user can input how long, if it all, they have been utilizing a continuous glucose monitoring device to check glucose levels. In some embodiments, though not illustrated, the medication history field 9105 can be utilized to input how many diabetes-related medications the user takes.

According to some embodiments, the onboarding GUI 9100 includes a collapsed view (best shown in FIGS. 9Q-1 and 9Q-2) and an expanded view (best shown in FIGS. 9Q-3). For example, in the expanded view, and as depicted in FIG. 9Q-3, the age field 9101 comprises plurality of selectable age range options (e.g., “18-20,” “21-34,” “35-44,” “45-54,” “55-64,” “65-70,” “71 years or more,” and in some embodiments, a “prefer not to answer” option). In the collapsed view, and as shown in FIG. 9Q-1, the selected age range option is displayed on the age field 9101 (e.g., “35-44”). Additionally, in the expanded view, and as shown in FIG. 9Q-3, the medical condition field 9102 comprises a plurality of selectable medical condition options (e.g., “Type 1,” “Type 2,” “Pre-Diabetes,” “Gestational,” “I do not have diabetes,” or “Prefer not to answer”). In the collapsed view, and as shown in FIG. 9Q-1, the selected medical condition option is displayed on the medical condition field 9102 (e.g., “Type 2”). In some embodiments, in the expanded view, and as shown in FIG. 9Q-3, the diagnosis field 9103 comprises a plurality of selectable time range options corresponding to a time period in which the user was diagnosed with a medical condition (e.g., “Less than a year ago,” “1-5 years ago,” “6-10 years ago,” “11-15 years ago,” “16-20 years ago,” “More than 20 years ago,” and “I don't know/Prefer not to answer”). In the collapsed view, and as depicted in FIG. 9Q-1, the selected time range option is displayed on the diagnosis field 9103.

According to some embodiments, in the collapsed view of onboarding GUI 9100, onboarding GUI 9100 is divided between a first page and a second page (FIGS. 9Q-1 and 9Q-2). For example, and as depicted in FIG. 9Q-1, on the first page of onboarding GUI 9100 in the collapsed view, only a first section of the plurality of selectable fields are displayed (e.g., the age field 9101, medical condition field 9102, and the diagnosis field 9103). The first page of onboarding GUI 9100 can include a back button 9106, and a next button 9107 which, upon being selected by the user, outputs the second page of onboarding GUI 9100 in the collapsed view. Further, and as depicted in FIG. 9Q-2, in the second page of onboarding GUI 9100 in the collapsed view, only a second section of the plurality of selectable fields are displayed (e.g., the medication field 9104 and the medication history field 9105). In some embodiments, the second page of onboarding GUI 9100 in the expanded view comprises a next button 9108, and a back button 9109 which, upon being selected, returns the user to the first page of onboarding GUI 9100 in the collapsed view.

According to an aspect of the embodiments, in the expanded view of onboarding GUI 9100, and as depicted in FIG. 9Q-3, the medication field 9104 comprises a plurality of selectable medication options, wherein the user can select one or more of the medication options that apply to the user (e.g., “Diet & Exercise,” “Oral Medications (Tables Or Pills),” “Non-Insulin Injectables,” “Long-Acting Or Basal Insulin,” “Short Or Rapid-Acting Or Bolus Insulin,” “Insulin Pump,” and/or “I don't know/Prefer Not To Answer”). In some embodiments, in the expanded view of onboarding GUI 9100, and as depicted in FIG. 9Q-3, each of the medication options selected by the user comprises a shadowed or colored portion to indicate that the medication option has been selected. In the collapsed view of onboarding GUI 9100, though not illustrated, the one or more selected medication options are displayed in the medication field 9104, wherein the one or more selected medication options comprise the shadowed or colored portion when displayed in the collapsed view of onboarding GUI 9100.

Further, and with reference to FIG. 9Q-3, in the expanded view of onboarding GUI 9100, the medication history field 9105 comprises a plurality of selectable time range options (e.g., “Less than one month ago,” “1-6 months,” “6-12 months,” “1-2 years,” “2-5 years,” “More than 5 years,” and “I don't know/Prefer not to answer”) corresponding to a time period in which the has been utilizing a medical device (e.g., continuous glucose monitoring device) to check glucose levels. In the collapsed view of onboarding GUI 9100, and as depicted in FIG. 9Q-2, the selected time range option corresponding to the time period in which the has been utilizing a medical device is displayed in the medication history field 9105.

According to an aspect of the embodiments, if the user has not yet created a profile, onboarding GUI 9100 is displayed in the collapsed view, wherein the onboarding GUI 9100 can be divided between the first page and the second page of the collapsed view (FIGS. 9Q-1 to 9Q-2). In some embodiments, however, the medication field 9104 can display a plurality of selectable medication options when in the collapsed view (FIG. 9Q-2). Further, in some embodiments, if the user is in the process of creating a profile or is inputting a selection to one or more of the plurality of selectable fields, the onboarding GUI 9100 can be displayed in the expanded view (FIG. 9Q-3). Further, in some embodiments, when the user has successfully made a selection for each of the plurality of selectable fields, then the onboarding GUI 9100 can be displayed in the collapsed view, wherein a corresponding selection chosen by the user is displayed for each of the plurality of selectable fields. Additionally, in some embodiments (though not illustrated) when the user has not yet made a selection for a particular one of the plurality of selectable fields, then a message is included on each of the particular plurality of selectable fields indicating to the user that they can select an option.

In some embodiments, when an “I Don't Know/Prefer Not to Answer” selectable option is selected for one of the plurality of selectable fields, then other options for the particular selectable fields will consequently be unselected in response thereto such that the “I Don't Know/Prefer Not to Answer” option is the only selected option for the selectable field. According to another aspect of the embodiments, when the user is editing their user profile and has selected a particular option for one of the plurality of selectable fields, then a checkmark will appear adjacent to the selected option (e.g., if the user selects “6-10 years ago” as the time range option corresponding to the time period in which the user was diagnosed with a medical condition, then a checkmark will appear by the “6-10 years ago” option). Those of skill in the art will appreciate that other indicators can be utilized to indicate a selected option for a selectable field without departing from the scope of the present disclosure.

Additionally, in some embodiments (though not illustrated), a disclaimer modal can be provided on onboarding GUI 9100, wherein the disclaimer modal comprises a message informing the user that treatment decisions based on data displayed in the TIR application should not be made, and that the user should always consult a healthcare provider before making changes to the user's management program.

FIG. 9Q-4 illustrates a block diagram of an exemplar embodiment of onboarding GUI 9100, wherein the user has made selections related to the user profile setup. Specifically, onboarding GUI 9100 is displayed in the collapsed view, wherein the age field 9101, medication condition field 9102, diagnosis field 9103, medication field 9104, and medication history field 9105 are all displayed on one page of onboarding GUI 9100. More specifically, when the user has made selections related to the user profile setup, onboarding GUI 9100 is displayed, wherein the age field 9101, medication condition field 9102, diagnosis field 9103, medication field 9104, and medication history field 9105 are all displayed with the respective selections shown in each respective field. In some embodiments, and as depicted in FIG. 9Q-4, the selections for the medication field 9104 are individually shown with a shadowed or colored portion to indicate that the medication option has been selected.

### Example Embodiments of Account and Settings GUIs

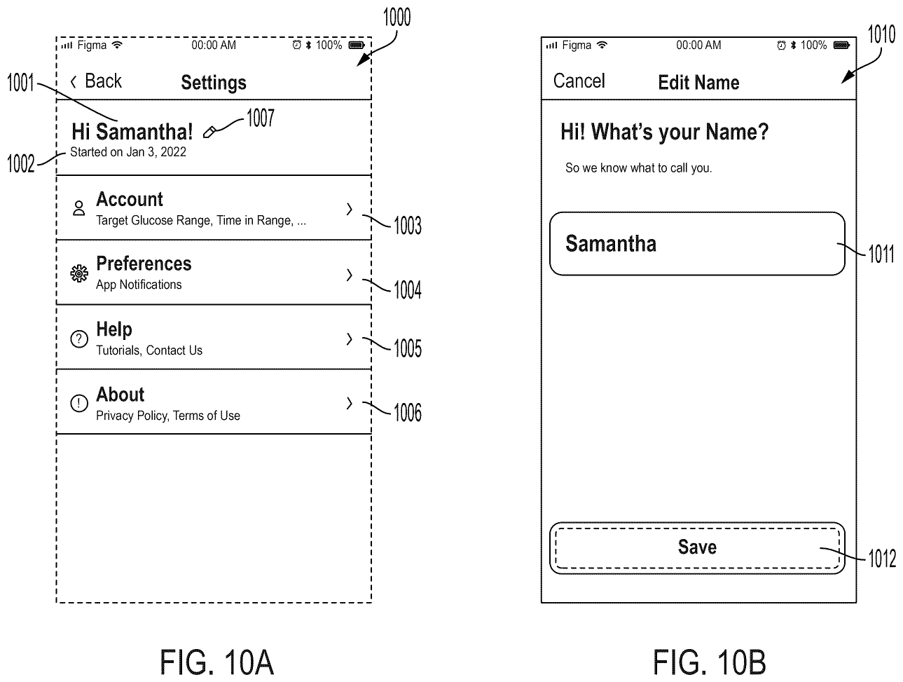

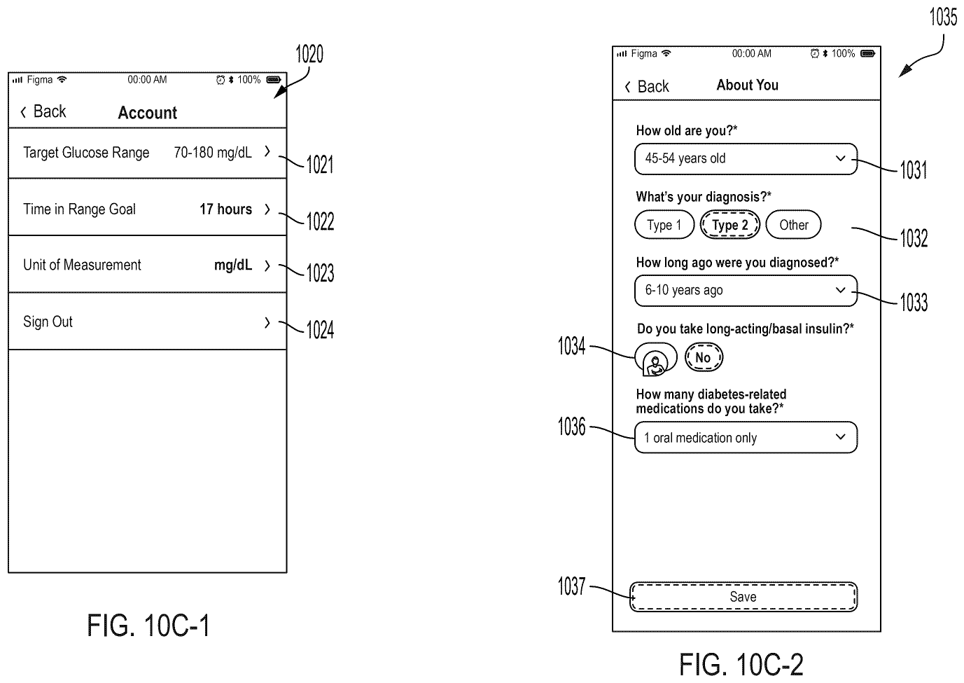

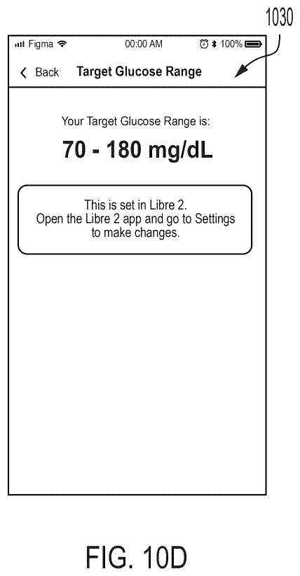

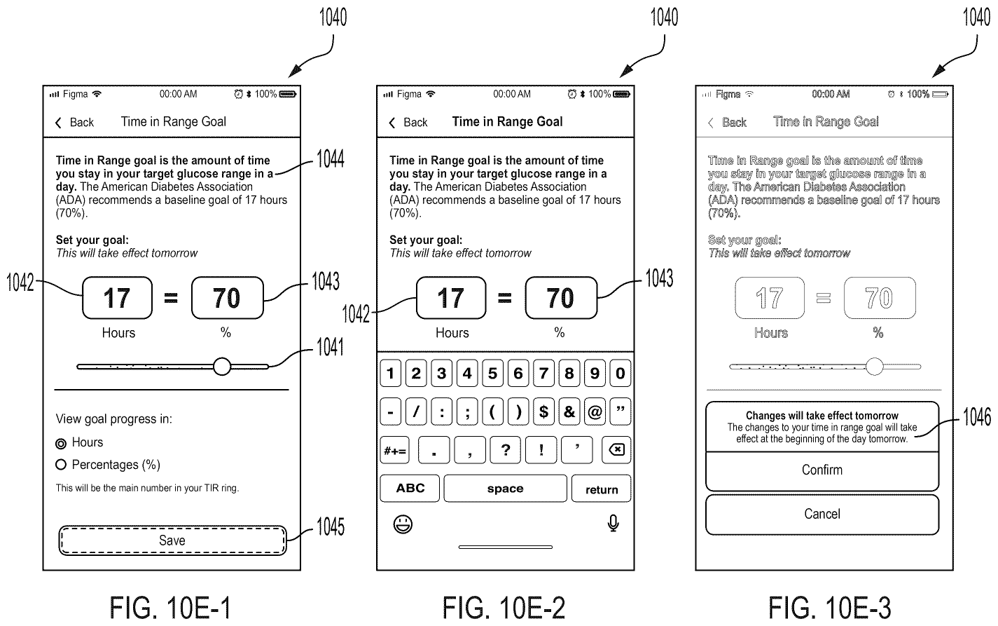

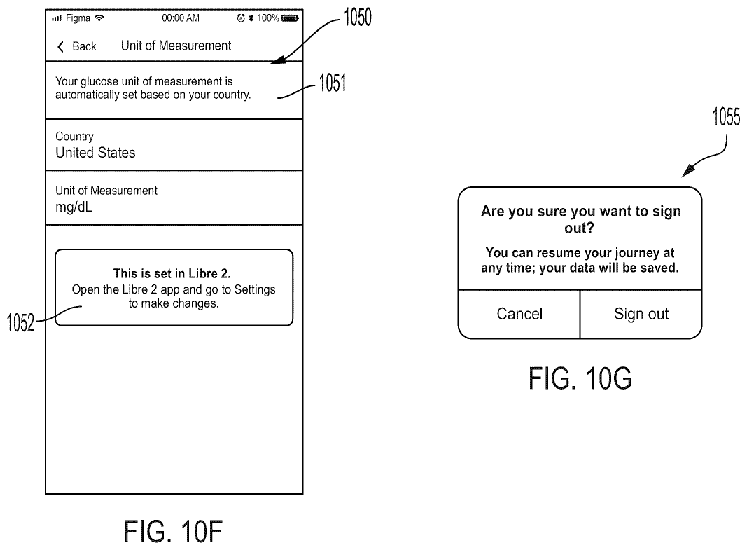

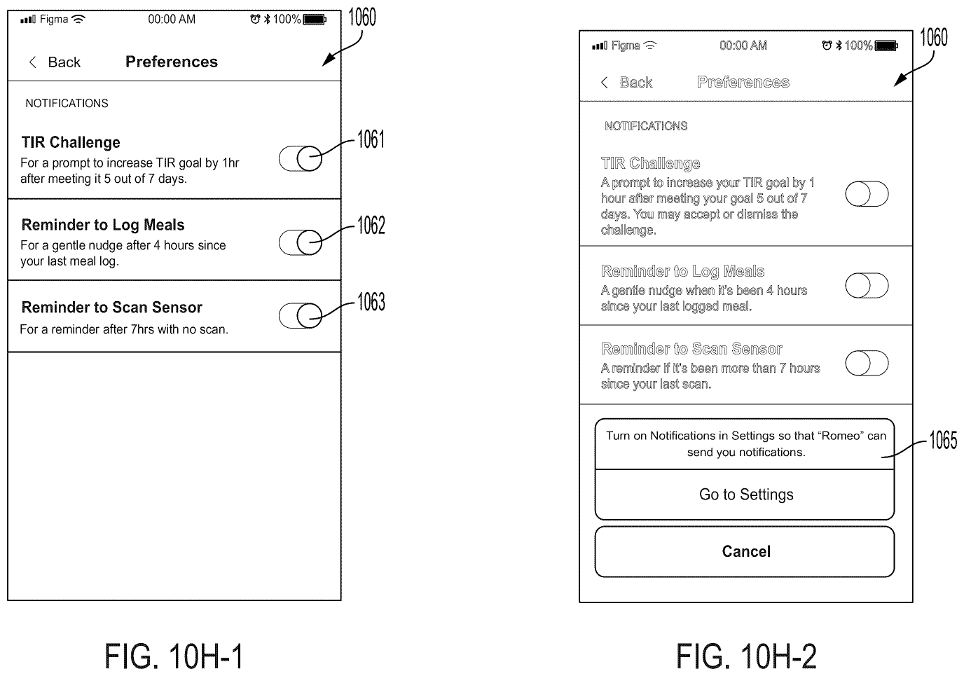

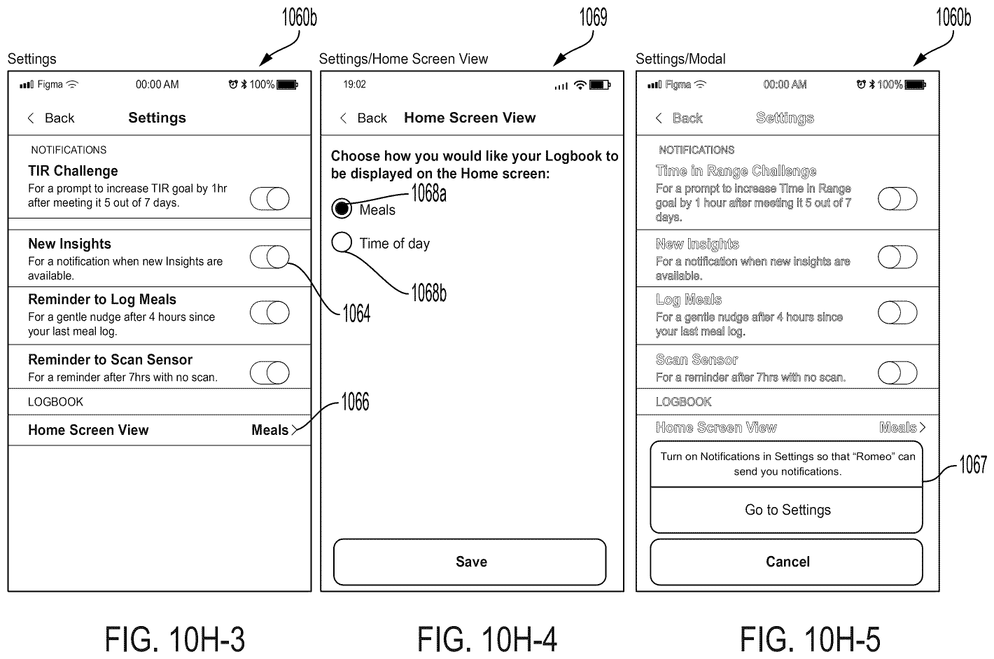

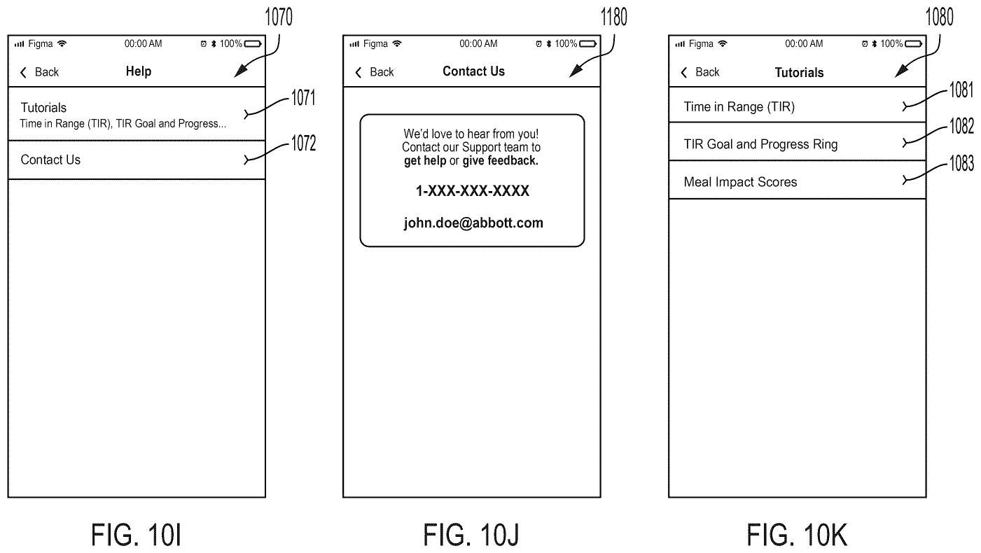

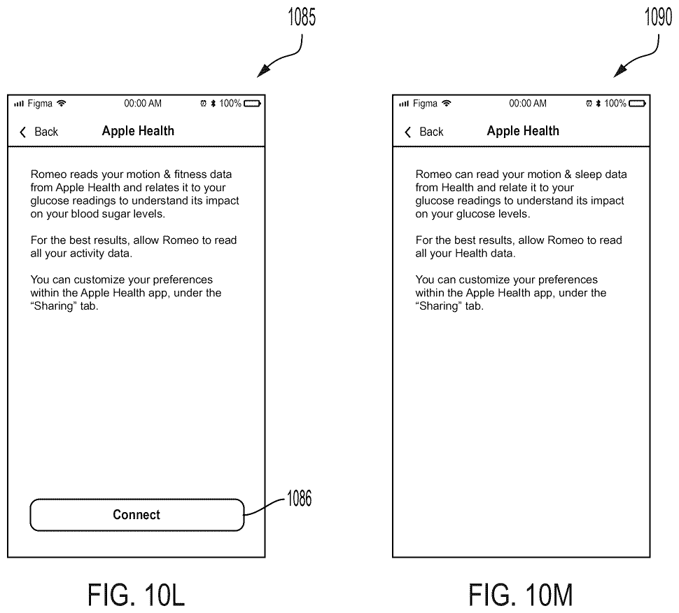

FIGS. 10A through 10M depict block diagrams of example embodiments of account and settings interfaces, or features related thereto, any of which can be utilized with the embodiments described herein. Referring to FIG. 10A, a settings GUI 1000 is depicted. Settings GUI 1000a can comprise (but is not limited to): a textual greeting 1001 comprising the user's inputted name (e.g., “Hi Samantha!”); a text description 1002 indicating a commencement period associated with the user (e.g., “Started on Jan. 3, 2022”), wherein the text description 1002 is displayed directly adjacent and distal to (e.g. below) the textual greeting 1001; an “Account” section 1003; a “Preferences” or “Settings” section 1004; a “Help” section 1005; and an “About” section 1006 which when selected, outputs information related to the TIR application.

In some embodiments, and as illustrated in FIG. 10A, a selectable pencil icon 1007 is displayed adjacent to the textual greeting 1001, which, upon being selected by the user, outputs settings GUI 1010 (FIG. 10B) comprising a name entry field 1011, wherein the user can edit a name previously inputted and associated with the user's account credentials.

In some embodiments, though not illustrated in FIG. 10A, settings GUI 1000 can further comprise an “activities” section (e.g., an “Apple Health” section or “Health Connect” section) which, when selected, outputs interfaces related to activity settings (see, e.g., FIGS. 10L and 10M).

Further, in some embodiments, though not illustrated in FIG. 10A, settings GUI 1000 can further comprise a notifications section which, upon being selected, outputs interfaces related to notifications and reminders.

Settings GUI 1010 (FIG. 10B) further comprises a “Save” button 1012 that the user can press to save the name inputted in the name entry field 1011.

FIG. 10C-1 is an example embodiment of an account settings interface. Account settings GUI 1020 is depicted in FIG. 10C-1. According to one aspect of the embodiments, account settings GUI 1020 can be displayed when the user selects the “Account” section 1003 in, e.g., previous settings GUI 1000 (FIG. 10A). According to another aspect of the embodiments, and as depicted in FIG. 10C-1, settings GUI 1020 can comprise a target glucose range setting 1021 and a unit of measurement setting 1023. In some embodiments, though not illustrated, the target glucose range setting 1021 can be utilized so that the user can configure a target glucose range. In certain embodiments, and as depicted in FIG. 10C-1, the target glucose range setting 1021 can be non-configurable and is displayed for informational purposes only (e.g., informing the user that the target glucose range is between 70 mg/dL and 180 mg/dL). For example, upon the user selecting the target glucose range setting 1021 in settings GUI 1020, settings GUI 1030 (FIG. 10D) is outputted, which comprises information related to the target glucose range for the user. Other combinations of configurable and non-configurable settings are possible, and those of skill in the art will recognize that these combinations are fully within the scope of the present disclosure.

As shown in FIG. 10C-1, in some embodiments, account settings GUI 1020 further comprises a configurable TIR goal setting 1022. Further, in some embodiments, though not illustrated, account settings GUI 1020 can further comprise a selectable “send research data” option which, upon being selected by the user, can output a “send research data” GUI. The send research data GUI comprises an informational section which informs the user that information from their account will be sent via email to the research team in order to help improve their TIR application experience. The informational section of the send research data GUI further provides details on what information or data will be sent to the research team and ensures the user that no personal identifiable information (“PII”) will be shared. Further, the send research data GUI can further comprise an export information section which indicates the date the user last sent an export of research data to the research team. According to an aspect of the embodiments, the send research data GUI can include a “send” button 126 which, when selected by the user, outputs a research confirmation modal. Specifically, the research confirmation modal is configured to partially obstruct or overlay the underlying interface (e.g., the send research data GUI). In some embodiments, research confirmation modal requests confirmation that the user wishes to send their data (e.g., the user's meals data and glucose data) to the research team. In some embodiments, research confirmation modal can include a “send” button which, when selected by the user, sends the user's data (e.g., the user's meals data and glucose data) to the research team. The research confirmation modal can also include a “cancel” button which, when selected by the user, cancels the request to send the user's data to the research team.

Though not illustrated, in some exemplar embodiments, account settings GUI 1020 (FIG. 10C-1 GUI) can further comprise a selectable “About You” option which, when selected by the user, outputs an About You settings GUI 1035 (see FIG. 10C-2). About You settings GUI 1035 (FIG. 10C-2) can include a plurality of selectable options to prompt the user for profile information or preferences. For example, the plurality of selectable options can include an age field 1031, wherein the user can input an age range; a medical condition field 1032, wherein the user can input whether they are diagnoses with a medical condition (e.g., Type 1 diabetes, Type 2 diabetes, or other condition); a diagnosis field 1033, wherein the user can input how long ago they were diagnosed with said medical condition; a medication field 1034, wherein the user can confirm whether they take diabetes-related medication(s) (e.g., long-acting/basal insulin); and a medication history field 1036, wherein the user can input how many diabetes-related medications the user takes. About You settings GUI 1035 further comprises a “save” button 1037 configured to be selectable by only upon the user making changes to the profile information and preferences detailed in the About You settings GUI 1035.

Further, FIGS. 10E-1 to 10E-3 depict an example embodiment of a settings GUI 1040, which is an interface related to the TIR goal. Settings GUI 1040 can be displayed when the user selects the TIR goal setting 1022 in previous settings GUI 1000 (FIG. 10A). According to one aspect of the embodiments, and as depicted in FIGS. 10E-1 to 10E-3, the TIR goal can be configured by the user such that the user can select a TIR goal, where a TIR goal will be reached when the user is within the predetermined target glucose range for a selected period of time (e.g., 17 hours). In some embodiments, and as depicted in FIG. 10E-1, settings GUI 1040 comprises a selectable bar 1041 configured to update the TIR goal in response to a ninth predetermined input by the user, such as when the user, drags the selectable bar with a finger, or by some other predetermined gesture.

In one aspect of the embodiments, and as best depicted in FIG. 10E-2, a time entry field 1042 is also provided by which the user can manually input a desired TIR goal in time units (e.g., “17” hours). In another aspect of the embodiments, and as best depicted in FIG. 10E-2, a percentage entry field 1043 is provided by which the user can manually input a desired TIR goal in percentage units (e.g., “70” percent). In some embodiments, if the user updates the TIR goal through one manner (e.g., through the selectable bar 1041, time entry field 1042, or percentage entry field 1043), settings GUI 1040 will be updated so as to reflect the updated TIR goal in the time entry field 1042 and percentage entry field 1043 (e.g., 17 hours equals 70 percent). In this manner, the settings GUI 1040 allows the user to visualize the new TIR goal in terms of both hours and percentages. In some embodiments, settings GUI 1040 includes a message indicating to the user that the TIR goal changes will take effect the following day.

Still referring to FIGS. 10E-1 to 10E-3, in some embodiments, settings GUI 1040 further comprises an informational section 1044 that informs the user of the TIR goal recommended by the American Diabetes Association. Further, in some embodiments, the settings GUI 1040 allows the user to select a specific progress indicator for the TIR goal. For example, and as best depicted in FIG. 10E-1, the user can choose to view TIR goal progress in units of hours or percentages. In other embodiments (not illustrated) the goal progress view is non-configurable. For example, in some embodiments, the user can only view the TIR goal progress in terms of hours.

As shown in FIG. 10E-1, after the new TIR goal settings have been inputted, the user can click “save” 1045 to update the information. In some embodiments, once “save” 1045 has been clicked by the user, and as best depicted in FIG. 10E-3, a confirmation in-app modal 1046 is displayed on settings GUI 1040, allowing the user to confirm the TIR goal changes. In some embodiments, and as depicted in FIG. 10E-3, the confirmation in-app modal 1046 comprises a message indicating to the user that the TIR goal changes will take effect the following day.

Further, FIG. 10F is a settings GUI 1050 depicting an interface related to the unit of measurement. According to one aspect of the embodiments, settings GUI 1050 can be displayed when the user selects the unit of measurement section 1023 in previous settings GUI 1020 (FIG. 10C). In some embodiments, though not illustrated, the unit of measurement is configurable (e.g., mg/dL or mmol/L). In some embodiments, and as depicted in FIG. 10F, the unit of measurement is non-configurable and is displayed in a unit of measurement determined by the third party's region or country (e.g., mg/dL for the United States). As shown in FIG. 10F, settings GUI 1050 comprises an informational message 1051 indicating the glucose unit of measurement is automatically set based on the user's country. In some embodiments, settings GUI 1050 further comprises an instructional message 1052 indicating that the user can make changes to the units of measurement through another analyte monitoring application (e.g., “Libre 2”).

FIG. 10G is an in-app modal notification 1055 providing information regarding the logging off of the TIR application. According to one aspect of the embodiments, in-app modal notification 1055 can be displayed when the user selects a “Sign Out” option 1024 in previous settings GUI 1020 (FIG. 10C). According to another aspect of the embodiments, in-app modal notification GUI 1020 can be configured so as to partially obstruct or superimpose the underlying interface (e.g., settings GUI 1000) from view.

Further, FIG. 10H-1 is a settings GUI 1060a depicting a settings preferences interface comprising a plurality of notifications options for the TIR application, wherein the user can turn on and/or off one or more notifications. According to one aspect of the embodiments, settings GUI 1060a can be displayed when, e.g., the user selects the “Preferences” section 1004 in previous account settings GUI 1000 (FIG. 10A). According to another aspect of the embodiments, and as best illustrated in FIG. 10H-1, settings GUI 1060a can comprise one or more of the following (but is not limited to): a textual label and description for a “TIR challenge” notification adjacent to a switch 1061 configured to be toggled between an on position and an off position; a textual label and description for a “Reminder to Log Meals” notification adjacent to a switch 1062 configured to be toggled between an on position and an off position; and, a textual label and description for a “Reminder to Scan Sensor” notification adjacent to a switch 1063 configured to be toggled between an on position and an off position. In some embodiments, a notification reminding the user to scan their sensor can be provided after seven hours with no scan. Those of skill in the art will understand that, instead of a toggle, settings GUI 1060a can include any one or more of: an on-off checkbox, on-off slider switch, on-off radio buttons, on-off buttons, and the like.

According to an aspect of the embodiments, if the “Reminder to Log Meals” notification has been turned on, after a first reminder and if the user has not logged a meal, the TIR application is configured to nudge the user after a predetermined period of time has passed (e.g., every six hours). In some embodiments, if the “Reminder to Log Meals” notification has been turned on, after a first reminder and if the user has logged a meal, the TIR application is configured to nudge the user after a predetermined period of time has passed (e.g., every four hours).

FIG. 10H-2 depicts settings GUI 1060a with all notifications turned off. Further settings GUI 1060a, as depicted in FIG. 10H-2, comprises an in-app modal 1065 providing information regarding disabled notifications condition along with a “Go to Settings” button that opens up the corresponding settings interface to allow the user to enable notifications.

FIG. 10H-3 depicts an additional exemplar embodiment of a settings GUI. Specifically. FIG. 10H-3 depicts settings GUI 1060b, which is similar to settings GUI 1060a, except that it further comprises a textual label and description for a “New Insights” notification adjacent to a switch 1064 configured to be toggled between an on position. In some embodiments, settings GUI 1060b further includes a configurable Home Screen view setting 1066. According to an aspect of the embodiments, and as depicted in FIG. 10H-4, settings GUI 1069 can be outputted in response to the user selecting the home screen view setting 1066 in settings GUI 1060B. Specifically, through settings GUI 1069, Home Screen view setting 1066 can be configurable by the user such that the user can select between one or more home screen views 1068, e.g., a meals view 1068a (e.g., the list of meal events) of the logbook section on the home GUI 400 (FIG. 4B), home GUI 450 (FIG. 4D), home GUI 430 (FIGS. 4E-1 to 4E-7), home GUI 485 (FIGS. 4J-1 to 4J-3), home GUI 495 (FIGS. 4K-1 to 4K-3), or home GUI 499 (FIGS. 4M-1 to 4M-6), or a Time of Day view 1068b (e.g., the Time of Day cards) of the logbook section on the home GUI 400 (FIG. 4B), home GUI 450 (FIG. 4D), home GUI 430 (FIGS. 4E-1 to 4E-7), home GUI 485 (FIGS. 4J-1 to 4J-3), home GUI 495 (FIGS. 4K-1 to 4K-3), home GUI 499 (FIGS. 4M-1 to 4M-6), or any of the interfaces described herein that can be configured as a home interface (e.g., GUI 12100, as shown in FIGS. 12D-1 to 12D-2, or GUI 13100, as shown in FIGS. 13E-1 to 13E-5). For example, in FIG. 10H-4, the meals view 1068a has been selected.

FIG. 10H-5 depicts settings GUI 1060b with all notifications turned off. Further settings GUI 1060b, as depicted in FIG. 10H-5, comprises an in-app modal 1067 providing information regarding disabled notifications condition along with a “Go to Settings” button that opens up the corresponding settings interface to allow the user to enable notifications.

FIG. 101 depicts an example embodiment of an account interface. Account GUI 1070 is depicted in FIG. 10I, which can be displayed when the user selects the “Help” section 1005 in previous account settings GUI 1000 (FIG. 10A). According to some embodiments, account GUI 1070 comprises (but is not limited to): a tutorials section 1071 and a “Contact Us” section 1072.

FIG. 10J depicts an account GUI 1180 comprising contact information provided for TIR application help and feedback. Account GUI 1180 can be displayed when the user selects the “Contact Us” section 1072 in previous account GUI 1070 (FIG. 10I).

In some embodiments, and in response to the user selecting the TIR tutorial section 1071 in account GUI 1070 (FIG. 10I), a settings interface is outputted which comprises a: (1) TIR tutorial option which, upon being selected, can output tutorial interfaces relating to TIR; (2) small daily goals tutorial option which, upon being selected, can output tutorial interfaces relating to small daily goals; (3) meal logging tutorial option which, upon being selected, can output tutorial interfaces relating to meal logging; (4) impact scores tutorial option which, upon being selected, can output tutorial interfaces relating to impact scores; and (5) weekly insights tutorial option which, upon being selected, can output tutorial interfaces relating to weekly insights.

In some embodiments, and in response to the user selecting the TIR tutorial section 1071 in account GUI 1070 (FIG. 10I), tutorial interfaces relating to the TIR application can be displayed. For example, in some embodiments, a tutorial interface can be displayed which informs the user about studies which have shown that TIR can be a simple yet powerful way to track how food impacts an individual's glucose levels. According to another aspect of the embodiments, a tutorial interface can be displayed which provides (1) the user information on the TIR ring and, in particular, explains the TIR ring represents the user's progress towards their goal and will fill up as the user spends more time within target range, and (2) a message encouraging the user to close the TIR ring before midnight each day in order to reach their goal. In another aspect of the embodiments, a tutorial interface can be displayed which informs the user that TIR can vary from person to person and that the American Diabetes Association recommends around 17 hours of TIR per day.

In yet another aspect of the embodiments, a tutorial interface can be displayed which (1) provides the user with brief instructions on how to log a meal in the TIR application, and (2) comprises a link which, upon being selected by the user, outputs a meal logging informational modal comprising tips for adding meals through the TIR application. Further, in some embodiments, a tutorial interface is displayed which provides the user with information related to meal rating or score. For example, in some embodiments, a tutorial interface can be displayed which (1) explains to the user that the TIR application rates meals based on glucose changes after eating, and that a 5-star meal will cause the smallest amount of rise in glucose, while a 1-star meal will cause the highest, and (2) comprises a link which, upon being selected, outputs a meal scoring informational modal comprising information on how a meal is ranked or scored in the TIR application, as described above. In some embodiments, the meal scoring information modal comprising information on how a meal is ranked can further comprise emoticons to indicate the impact on glucose. According to some aspects of the embodiments, the meal scoring informational modal can comprise a link which, when selected by the user, outputs another meal scoring informational modal comprising details on why the score is measured three hours after a meal is consumed. In some embodiments, the meal scoring informational modal comprising details on why the score is measured three hours after a meal is consumed can also include a tip which provide the user with suggestions on how to avoid being out of range for longer periods of time. In some embodiments, the meal scoring information modal further comprises details on why the user should allow time between meals. Further, the meal scoring information modal further comprises details on logging all foods eaten in a 30-minute window under the same meal so as to minimize the number of meals that receive a missing score or rating. Further, in some embodiments, the meal scoring information modal further comprises details on scanning at least every eight hours to ensure that the TIR application has the glucose data needed to provide a meal rating or score. Additionally, in some embodiments, the meal scoring information modal informs the user that for accurate meal ratings or scores, the TIR application needs the user's glucose readings at the beginning of and two hours following each meal, and that if the user missed a scan or had a sensor issue, the TIR application might not be able to rate the user's meal.

In yet another aspect of the embodiments, a tutorial interface can be displayed comprising (1) details on why it is suggested to wait around three hours between meals to get an accurate glucose reading, and (2) details on logging all foods eaten in a 30-minute window under the same meal so as to minimize the number of meals that receive a missing score or rating.

Further, in some embodiments, a tutorial interface can be displayed which informs the user that the TIR application can provide weekly insights about the user's progress and that the user can choose to be notified about new insights in the TIR application's settings or by checking the “insights” tab for updates.

FIG. 10K is a block diagram depicting an account tutorials GUI 1080, which can be displayed when the user selects the tutorials section 1071 in previous account GUI 1070 (FIG. 10I). As depicted in FIG. 10K, account tutorials GUI 1080 can comprise a TIR tutorial section 1081, TIR Goal and Progress Ring tutorial section 1082, and a Meal Impact Score tutorial section 1083.

In response to the user selecting the TIR tutorial section 1081 in account tutorials GUI 1080 (FIG. 10K), tutorial interfaces relating to TIR can be displayed. For example, in some embodiments, a tutorial interface can be displayed which explains that TIR represents how long the user has stayed in target glucose range. According to another aspect of the embodiments, a tutorial interface can be displayed which informs the user that users who maintain their daily TIR are more likely to experience positive health outcomes. In another aspect of the embodiments, a tutorial interface can be displayed which informs the user that the standard target glucose range for most people is between 70 mg/dL and 180 mg/dL, but that the user's doctor may have a different recommendation.

In another aspect of the embodiments, and in response to the user selecting the TIR Goal and Progress Ring tutorial section 1082 in account tutorials GUI 1080 (FIG. 10K), tutorial interfaces relating to TIR goal and the progress ring can be displayed. For example, in some embodiments, a tutorial interface can be displayed which informs the user that the American Diabetes Association says a person's TIR should be around 17 hours per day. In another example embodiment, a tutorial interface can be displayed which informs the user that the TIR application calculates the user's current TIR and suggests an ideal first goal. In yet another exemplar embodiment, a tutorial interface can be displayed which provides the user information on the TIR ring and, in particular, explains the TIR ring represents the user's progress towards their goal and will fill up with each hour spent within target range.

In yet another aspect of the embodiments, and in response to the user selecting the Meal Impact Score tutorial section 1083 in account tutorials GUI 1080 (FIG. 10K), tutorial interfaces relating to meal impact scores can be displayed. In some embodiments, for example, a tutorial interface can be displayed which explains to the user one of the purposes behind scoring (e.g., the TIR application scores the user's meals so that the user can understand which foods work best to keep the user in range). In some exemplar embodiments, a tutorial interface can be displayed which explains to the user that scores are based on the changes to the user's glucose level within three hours of eating. In other exemplar embodiments, a tutorial interface can be displayed which provides the user with a key to the different score options and what each represents with respect to glucose change.

According to another aspect of the embodiments, the TIR application can also include account interfaces relating to general account and TIR application information. For example, an “about” interface can be provided, which provides general information relating to the TIR application. Further, in some embodiments, the about interface can comprise a selectable pilot disclaimer section which, upon being selected by the user, outputs an account interface comprising the pilot disclaimer and information related to intended use of the TIR application. Specifically, the account interface with the pilot disclaimer can further comprise an informational section indicating the date the user signed the TIR application agreement (in previous onboarding GUI 925, as seen in FIG. 9B).

Further, in some embodiments, the about interface can further comprises a selectable licenses section which, upon being selected by the user, outputs an interface which comprises information related to licenses and notices for open source software used in the TIR application.

In another aspect of the embodiments, an interface comprising the Terms of Use for the TIR application can be outputted from the about interface. In some embodiments, the interface comprising the Terms of Use is displayed upon the user first installing and/or opening the TIR application. Further, in some embodiments, an interface comprising the Privacy Policy for the TIR application can be outputted from the about interface. In some embodiments, the interface comprising the Privacy Policy is displayed upon the user first installing and/or opening the TIR application.

Turning to FIG. 10L, an example embodiment of an activity settings GUI 1085 is depicted. According to one aspect of the embodiments, activity settings GUI 1085 can be displayed when the user selects the “activities” section as described with reference to settings GUI 1000 (FIG. 10A). Specifically, activity settings GUI 1085, as depicted in FIG. 10L, is displayed if the user has not previously connected to a third-party health monitoring application, e.g., Apple Health or Health Connect, during the onboarding process or in their settings. More specifically, in some embodiments, activity settings GUI 1085 comprises an informational section which informs the user that the TIR application reads the user's motion and fitness data from the third-party health monitoring application and relates it to the user's glucose readings to understand its impact on the user's blood sugar levels. In some embodiments, the informational section further informs the user that preferences within the third-party health monitoring application can be customized by going to the “Sharing” tab or from within the third-party health monitoring application itself. Further, in some embodiments, activity settings GUI 1085 comprises a “connect” button 1086 which, upon being selected, allows the user to connect the TIR application to the third-party health monitoring application. In some embodiments, upon the user selecting the connect button 1086, a confirmatory modal is outputted on the activity settings GUI 1085 which confirms to the user that the TIR application would like access to the third-party health monitoring application. In some embodiments, the confirmatory modal (though not illustrated), comprises a selectable confirmation “ok” button.

FIG. 10M depicts an additional exemplar embodiment of an activity settings GUI. Specifically, FIG. 10M depicts activity settings GUI 1090, which is displayed when the user selects the “activities” section as described with reference to settings GUI 1000 (FIG. 10A). More specifically, activity settings GUI 1090 is displayed if the user has previously connected to the third-party health monitoring application (e.g., Apple Health, or Samsung Health or Google Fit through Health Connect) during the onboarding process or in their settings. Even more specifically, activity settings GUI 1090 is similar to activity settings GUI 1085 (as shown in FIG. 10L), except that it does not comprise the “connect” button.

### Example Embodiments of Data Loss GUIs and Related Features

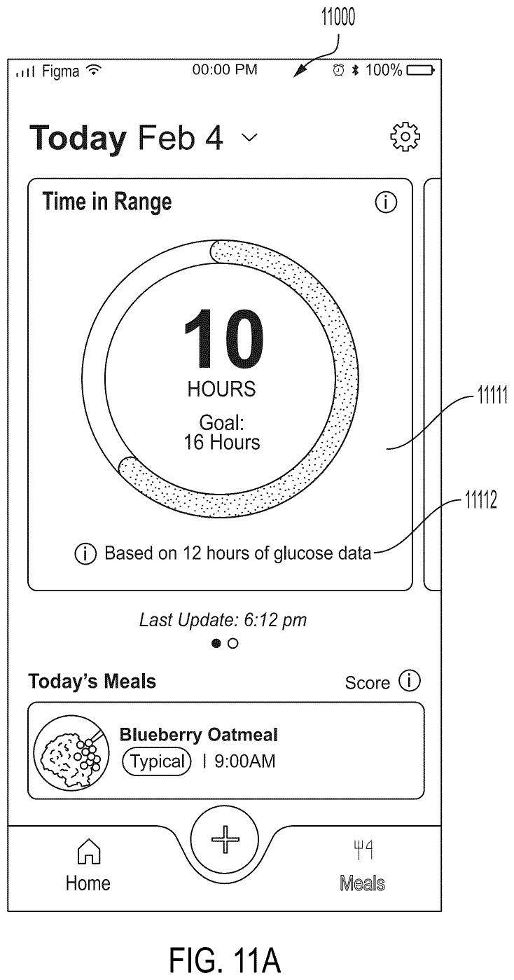

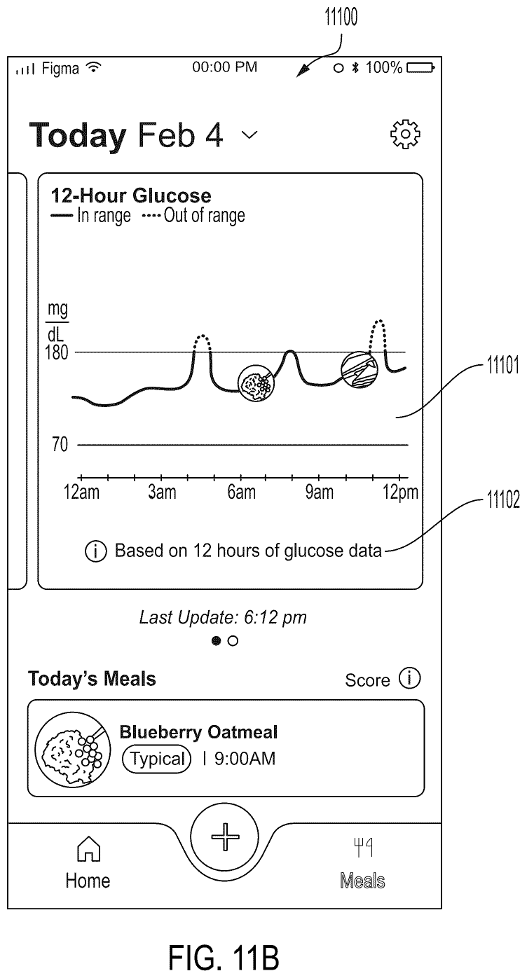

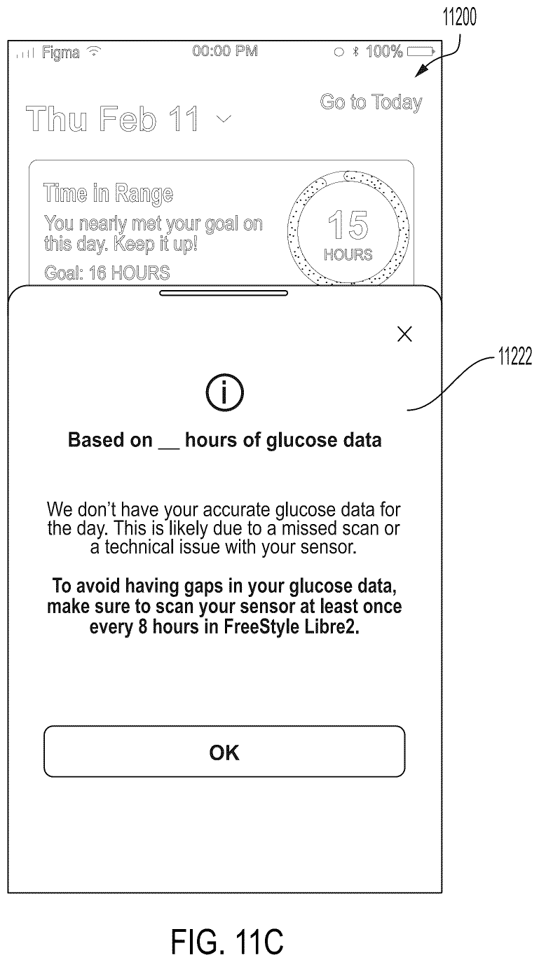

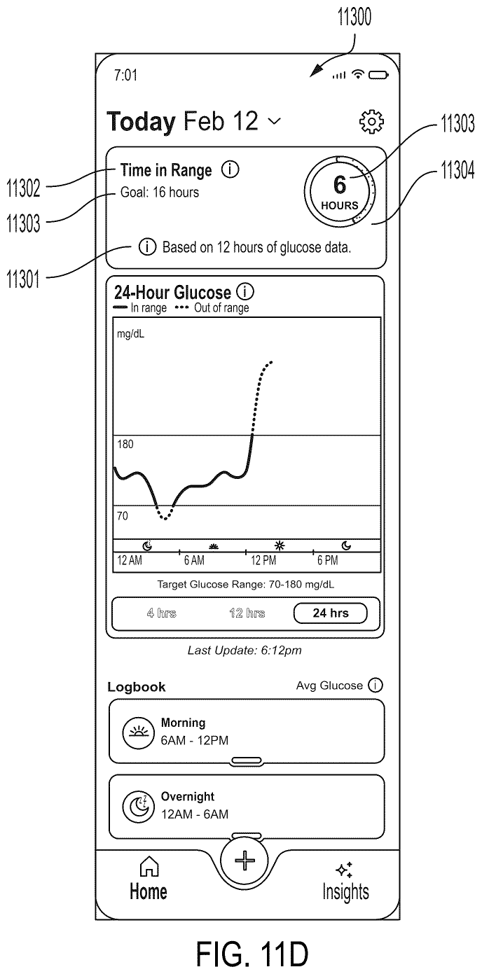

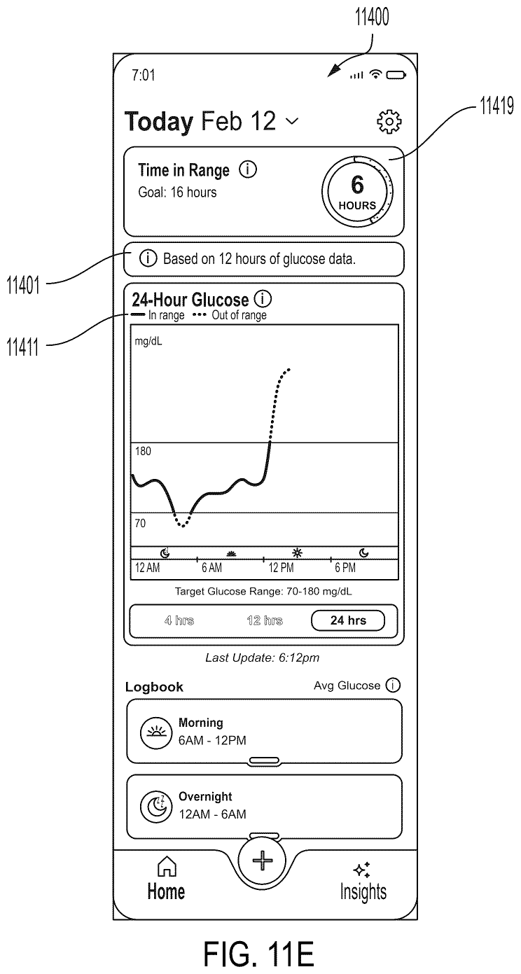

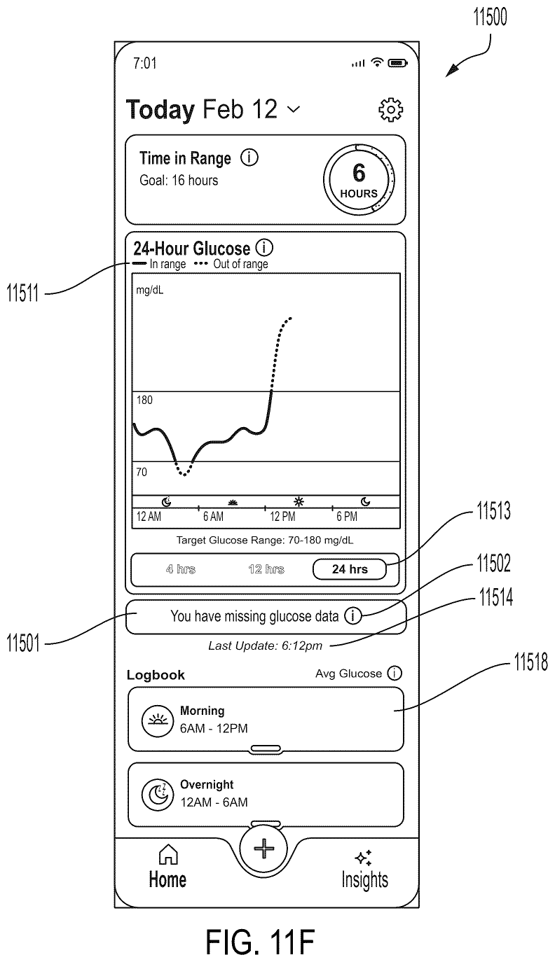

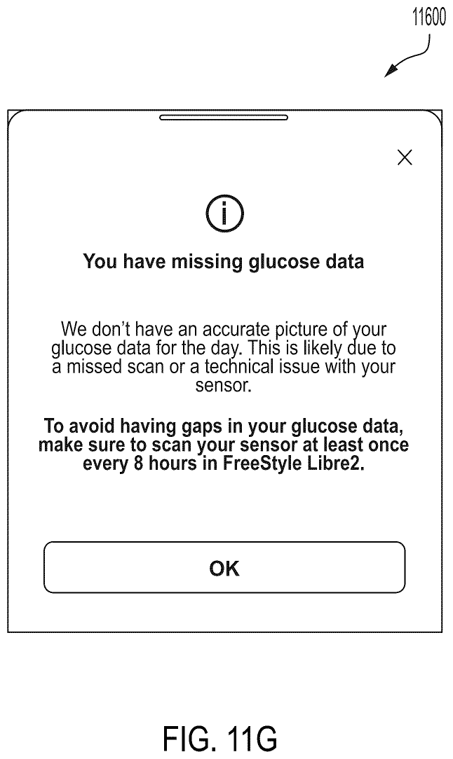

FIGS. 11A through 11G are block diagrams depicting example embodiments of data loss interfaces, or features related thereto, any of which can be utilized with any of the embodiments described herein.

Referring to FIG. 11A, an example embodiment of a block diagram of a GUI 11000 for a TIR application is depicted. GUI 11000 is similar to home GUI 400 (FIG. 4B), except that it further comprises a notification associated with one or more data loss conditions (e.g., “Based on 12 hours of glucose data”). In some embodiments, and as depicted in FIG. 11A, the notification associated with the one or more data loss conditions can be presented as an in-app notification 11111 positioned on a TIR card 11112.

FIG. 11B depicts a block diagram of another example embodiment of a GUI 11100 for the TIR application. GUI 11100 is similar to home GUI 450 (FIG. 4D), except that it further comprises a notification associated with one or more data loss conditions (e.g., “Based on 12 hours of glucose data”). In some embodiments, and as depicted in FIG. 11B, the notification associated with the one or more data loss condition can be presented as an in-app notification 11101 positioned on an analyte graph card 11102.

Further, in the exemplar embodiment depicted in FIG. 11C, GUI 11200 comprises an in-app modal notification 11222 associated with a data loss condition. In some embodiments, the in-app modal notification 11222 can include an informational tip to resolve one or more data loss conditions. Moreover, in-app modal notification 11222 can be configured so as to partially obstruct or superimpose the underlying interface from view.

FIG. 11D depicts a block diagram of another example embodiment of a GUI 11300 for the TIR application. GUI 11300 is similar to home GUI 485 (FIGS. 4J-1 to 4J-3), except that it further comprises a notification 11301 associated with one or more data loss conditions (e.g., “Based on 12 hours of glucose data”). In some embodiments, and as depicted in FIG. 11D, the notification 11301 associated with the one or more data loss conditions can be presented as an in-app notification 11301 positioned on a TIR card 11302 on GUI 11300. In some embodiments, the in-app notification 11301 can be positioned directly adjacent to and below the daily TIR goal 11302. In some embodiments, and as best depicted in FIG. 11D, the in-app notification 11301 can be positioned below the daily TIR goal 11302 and the TIR ring 11304 comprising the TIR value 11303 therein.

FIG. 11E depicts a block diagram of another example embodiment of a GUI 11400 for the TIR application. GUI 11400 is similar to home GUI 485 (FIGS. 4J-1 to 4J-3), except that it further comprises a notification 11401 associated with one or more data loss conditions (e.g., “Based on 12 hours of glucose data”). In some embodiments, and as depicted in FIG. 11E, the notification 11401 associated with the one or more data loss conditions can be presented as an in-app banner positioned between a TIR card 11419 and an analyte graph card 11411 of GUI 11400.

FIG. 11F depicts a block diagram of another example embodiment of a GUI 11500 for the TIR application. GUI 11500 is similar to home GUI 485 (FIGS. 4J-1 to 4J-3), except that it further comprises a notification 11501 associated with one or more data loss conditions (e.g., “Based on 12 hours of glucose data,” “Incomplete glucose data,” or “You have missing glucose data”). In some embodiments, and as depicted in FIG. 11F, the notification 11501 associated with the one or more data loss conditions can be presented as an in-app banner positioned between an analyte graph card 11511 and a logbook section 11518 of GUI 11500. Specifically, the notification or in-app banner 11501 can be positioned directly adjacent to and below one or more selectable icons 11513 and directly adjacent to and above a message 11514 indicating when GUI 11500 was last updated. In some embodiments, the notification or in-app banner 11501 can comprise an informational icon 11502 which, upon being selected by the user, outputs an informational modal 11600 (as depicted in FIG. 11G) which provides further information on the data loss condition.

Specifically, as shown in FIG. 11G, the informational modal 11600 can inform the user that likely due to a missed sensor scan or a technical issue with the user's sensor, the TIR application does not have an accurate picture of the user's analyte data (e.g., glucose data) for the day. Further, in some embodiments, the informational modal 11600 can provide the user a reminder message reminding the user that to avoid having gaps in the analyte data (e.g., glucose data), the user should scan the sensor at least once every eight hours in the analyte monitoring application. The informational modal 11600 can also include a selectable “ok” button.

Those of skill in the art will appreciate that the notifications and data loss condition features described herein are meant to be illustrative only, and that the individual elements, or any combination of elements, depicted and/or described for a particular embodiment or figure are freely combinable with any other element, or any combination of other elements, depicted and/or described with respect to any of the other embodiments,

### Example Embodiments of In-App Banner Notifications

The TIR application can also include in-app banner notifications, any of which can be utilized with the embodiments described herein. In some embodiments, in-app banner notifications can include information related to one or more of the following: (1) meal logging (e.g., “meal added” or “meal deleted”); (2) updated settings (e.g., “time in Range updated”); (3) scanning (e.g., “successful scan,” “ready to scan,” “6 hours since last scan,” or “scan unsuccessful”); a detected error (e.g., prompting the user to try again later, or sign out and sign in to continue); and (3) no internet connection (e.g., notifying the user internet connection is needed to get the most up to date TIR and glucose data).

### Example Embodiments of Activity Cards and Features Related Thereto

FIGS. 12A-1 through 12D-2 depict block diagrams of example embodiments of interfaces comprising activity cards, or features related thereto, any of which can be utilized with the embodiments described herein.

FIGS. 12A-1-12A-4 depicts a block diagram of another example embodiment of a GUI 12000 for the TIR application. GUI 12000 is similar to home GUI 485 (as depicted in FIGS. 4J-1 to 4J-3), except that it further comprises a health section 1201 which can include one or more activity cards 1202, 1203 (e.g., a movement card 1202 and a sleep card 1203). Specifically, health section 1201 can be displayed on GUI 12000 when the user, through, e.g., onboarding GUI 995 (shown in FIG. 9P), enables a third-party health monitoring application, e.g., Apple Health, or Samsung Health or Google Fit through Health Connect, to share data related to the user's activity, such as, movement, sleep, or both. For example, if through, e.g., the movement setting 9951 of onboarding GUI 995, the user allows the third-party health monitoring application to share only data related to the user's movement, then GUI 12000 will display a health section 1201 comprising a movement card 1202, as depicted in FIGS. 12A-1 and 12A-2. Specifically, FIG. 12A-1 depicts a movement card 1202 which displays the number of steps (e.g., “24,437 steps”) the user has taken in a predetermined period of time (e.g., for a particular one-day period). In some embodiments, and as depicted in FIG. 12A-2, the movement card 1202 displays the number of wheelchair pushes (e.g., “1,437 pushes”) the user has taken in a predetermined period of time (e.g., for a particular one-day period). As depicted in FIGS. 12A-2 and 12-3, the health section 1201 comprising the movement card 1202 can be directly adjacent to and distal relative to (e.g., below) a logbook section 1208 of GUI 12000.

In some embodiments, and as best shown in FIG. 12A-3, if through, e.g., the sleep setting 9952 of onboarding GUI 995, the user allows the third-party health monitoring application to share only data related to the user's sleep, then GUI 12000 will display the health section 1201 comprising a sleep card 1203 which displays the time (e.g., “17 hours and 31 minutes”) the user has spent sleeping for a past predetermined period of time (e.g., the previous night). Further, in some embodiments, and as best depicted in FIG. 12A-4, if through, e.g., the movement setting 9951 and sleep setting 9952 of onboarding GUI 995, the user allows third-party health monitoring application (e.g., Apple Health, or Samsung Health or Google Fit through Health Connect) to share data related to both the user's movement and sleep, then GUI 12000 will display the health section 1201 comprising the movement card 1202 and the sleep card 1203. In some embodiments, the movement card 1202 can be directly adjacent to the sleep card 1203. Further, in some embodiments, the health section 1201 can be directly adjacent to and distal relative to (e.g., below) a logbook section 1208 of GUI 12000. Additionally, in some embodiments, and as shown in FIGS. 12A-1 to 12A-4, the health section 1201 further comprises a message 1204 adjacent to and distal relative to (e.g., below) the movement card 1202 and/or sleep card 1203, wherein the message 1204 indicates when the data displayed in the health section 1201 was last updated. In some embodiments, the sleep card 1203 and movement card 1202 are configured to update each time the TIR application is opened.

According to yet another aspect of the embodiments, and as best shown in FIG. 12B, if in the initial setup or through the onboarding process (e.g., onboarding GUI 995, as shown in FIG. 9P) the user declines the TIR application to access the third-party health monitoring application, e.g., Apple Health, or Samsung Health or Google Fit through Health Connect, and connect thereto, then GUI 12000 can display the health section 1201 comprising a connection card 1205. Specifically, in some embodiments, the connection card 1205 can comprise a message indicating that the user can gain insight on how movement and sleep affect the user's glucose levels by connecting to the third-party health monitoring application. Further, in some embodiments, the connection card 1205 can include a selectable “connect” button 1206 which, upon being selected, outputs a connection modal 1206 (FIG. 12C) on an interface (e.g., GUI 12000), which comprises a message indicating to the user that the TIR application requires installation of a third-party health monitoring application and that once the third-party health monitoring application is installed, the user can continue using health data. In some embodiments, and as shown in FIG. 12C, connection modal 1206 comprises a selectable installation link 1207.

In some embodiments, though not illustrated, the user can install/download a third-party health monitoring application through a third-party application store on a reader device, such as a mobile communication device or a smartphone. Specifically, the user can select an install link corresponding to the third-party health monitoring application on the third-party application store. Following this, and once installed, the user can select an open link which allows the user to open the downloaded third-party health monitoring application. The user can then navigate through onboarding and setting interfaces on the third-party health monitoring application which allow the user to grant the TIR application access to choose data the user wants the TIR application to read and/or write to the third-party health monitoring application. In some embodiments, the user can customize the type of data the third-party health monitoring application can read, such as, data related to basal metabolic rate, blood glucose, blood pressure, body fat, distance, exercise, heart rate, and/or height, to name a few. The user can also choose to allow the third-party health monitoring application to read all collected health data. Further, in some embodiments, the user can customize the type of data the TIR application can have access to, such as, sleep, steps, and/or push data. Specifically, the user can customize whether the TIR application can have access to read sleep, steps, and/or push data. Further, the user can also customize whether the TIR application can have access to write sleep, steps, and/or push data. In some embodiments, the user can select an “allow all” button which allows the user to grant the TIR application to read and write all sleep, stapes, and/or push data provided by the underlying third-party health monitoring application. In some embodiments, the third-party health monitoring application will output a connection interface which confirms to the user that the third-party health monitoring application has successfully granted access to the TIR application.

Those of skill in the art will appreciate that the health section 1201, activity cards 1202, 1203, and features related thereto, are meant to be illustrative only, and that the individual elements, or any combination of elements, depicted and/or described for a particular embodiment or figure are freely combinable with any other element, or any combination of other elements, depicted and/or described with respect to any of the other embodiment.

Specifically, an additional exemplar embodiment of a block diagram of GUI 12100 is shown in FIGS. 12D-1 and 12D-2, wherein GUI 12100 comprises a health section 12101 which can include one or more activity cards 12102, 12103 (e.g., a movement card 12102 and/or a sleep card 12103). More specifically, GUI 12100 can be configured as a home interface for the TIR application, and can comprise: (1) an analyte graph card 12110; (2) the health section 12101; (3) a selectable home icon 12111 that, when selected by the user, outputs the GUI 12100; (4) a selectable insights icon 12112 that, when selected by the user, outputs an insights GUI 14000 or insights GUI 150000 (e.g., FIGS. 14A-1 to 14A-4, and FIGS. 15A-1 through 15A-4, respectively), which will be described in further detail below; and/or (5) a “+” icon 12113 that, when selected by the user, outputs a meal logging GUI 500 (FIG. 5A) configured to receive meal information inputted by the user. In some embodiments, and as depicted in FIG. 12D-2, GUI 12100 can further comprise a meal timeline 12114 which comprises one or more meal icons 12115, wherein each of the one or more meal icons 12115 corresponds to a meal event. In some embodiments, the meal timeline 12114 is only displayed on GUI 12100 when one or more meal events are detected. Further, according to some embodiments (though not illustrated) when there is no analyte data or meal events detected, GUI 12100 will display the analyte graph card 12110 without an analyte trendline and without the meal timeline 12114.

Specifically, in some embodiments, and with reference to FIG. 12D-2, each of the one or more meal icons 12115 is positioned along the meal timeline 12114 so as to correspond to a time associated with the meal event (e.g., a detected time of the meal event or an inputted time associated with when the meal event occurred). In some embodiments, the meal timeline 12114 can be displayed near, close to, and/or distal to (e.g., below) an analyte graph 12116 of the analyte graph card 12110, below the x-axis, and/or at the bottom of the graph window. In some embodiments, the GUI 12100 includes a target analyte range 12117 (e.g., “target glucose range: 70-180 mg/dL”), and one or more selectable icons 12118 (e.g., radio button, check box, slider, switch, etc.) that allow the user to select a predefined amount of time over which the user's analyte data will be shown in the analyte graph card 12110. Specifically, in some embodiments, the meal timeline 12114 can be positioned distal relative to (e.g., below) the analyte graph 12116, and between the x-axis and one or more selectable icons 12118. More specifically, the x-axis can comprise units of time (e.g., three hour increments, etc.) covering a twenty-four hour period. In this manner, the meal timeline 12114 can be positioned such that the user can visually associate a post-prandial analyte trace with the relevant meal event associated with each meal icons 12115 displayed on the meal timeline 12114.

Further, in some embodiments, each meal icon 12115 can be a photograph or image associated with a particular meal. In some embodiments, a meal icon 12115 can include placeholder image instead of a photograph or image selected by the user. According to an aspect of the embodiments, each meal icon 12115 along the meal timeline 12114 is selectable. Specifically, upon the user selecting particular a particular meal icon 12115, a meal review GUI corresponding to the selected meal icon 12115 is outputted (e.g., meal review GUI 800 or meal review GUI 8000, as shown in FIGS. 8A and 8B-8E, respectively).

In some embodiments, and as illustrated in FIGS. 12D-1 and 12D-2, the GUI 12100 further comprises a message 12119 adjacent to the analyte graph card 12110, wherein the message 12119 indicates when the analyte graph card 12110 was last updated. Additionally, in some embodiments, and as shown in FIGS. 12D-1 and 12D-2, the health section 12101 further comprises a message 12104 adjacent to and distal relative to (e.g., below) the movement card 12102 and/or sleep card 12103, wherein the message 12104 indicates when the data displayed in the health section 12101 was last updated. In some embodiments, the sleep card 12103 and movement card 12102 are configured to update each time the TIR application is opened.

In some embodiments, and as best shown in FIGS. 12D-1 and 12D-2, GUI 12100 can further comprise a text description 12120 indicating a current day of the week (e.g., “Today June 28”), wherein the text description 12120 can include a date adjacent to a switch 12121 configured to be toggled. In some embodiments, though not illustrated, the GUI 12100 can be related to past day, wherein the textual description 12120 includes a date associated with the past day (e.g., “Fri February 11”). In some embodiments, though not illustrated, when the switch 12121 is toggled down by the user, the GUI 12100 is configured to display a plurality of selectable rings, each of which corresponds to a different day of the past week (similar to the plurality of rings 425 depicted in FIG. 6B-1). For example, when the switch 12121 is toggled down, the GUI 12100 can display a plurality of seven rings, one corresponding to each day of the week. In some embodiments, though not illustrated, when a user selects a particular ring from the plurality of rings, GUI 12100 will display data pertaining to the day corresponding to the selected ring. In some embodiments, when the switch 12121 is toggled to an up position, the plurality of rings are no longer displayed. Further, in some embodiments, though not illustrated, when GUI 12100 is related to a past day, GUI 12100 can further comprise a selectable “Go To Today” link which, upon being selected, outputs GUI 12100 comprising data corresponding to the current day.

### Example Embodiments of Meal Detection GUIs, Methods, and Features Related Thereto

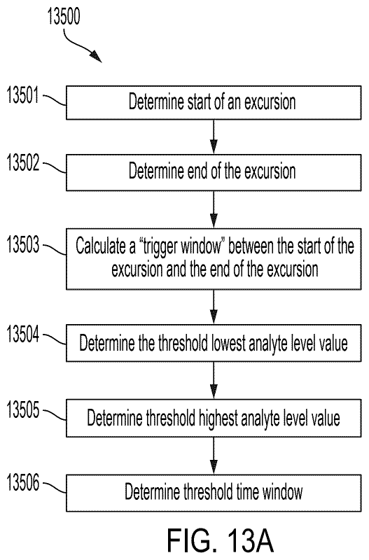

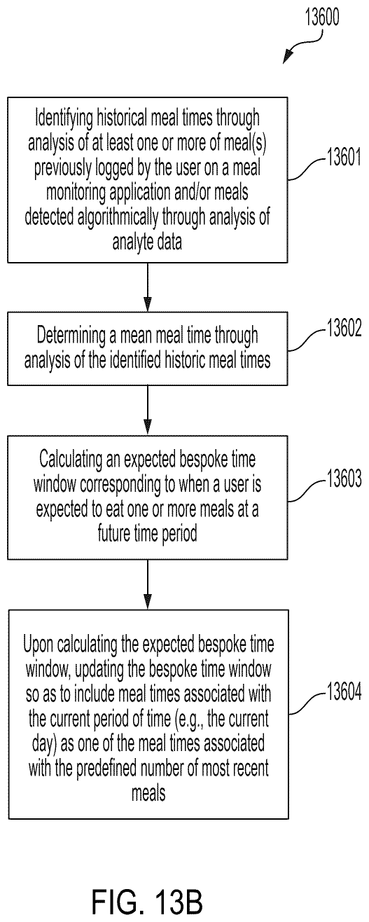

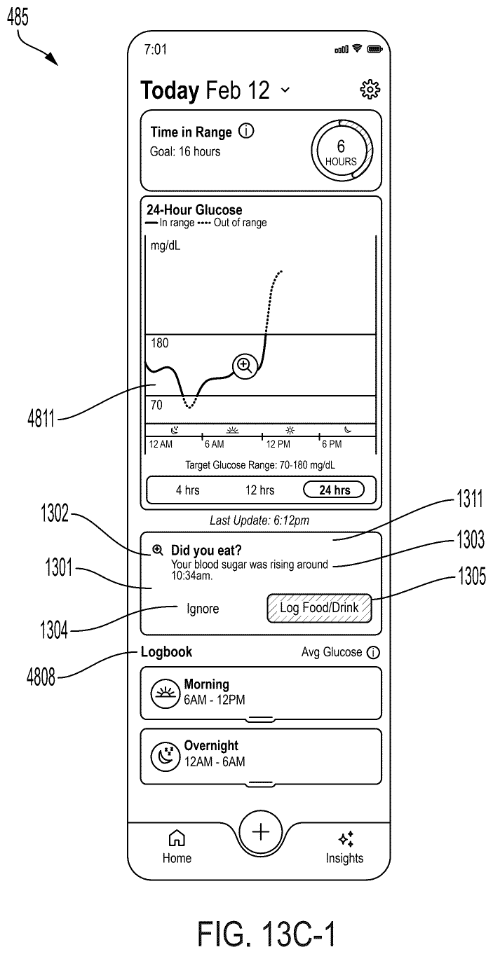

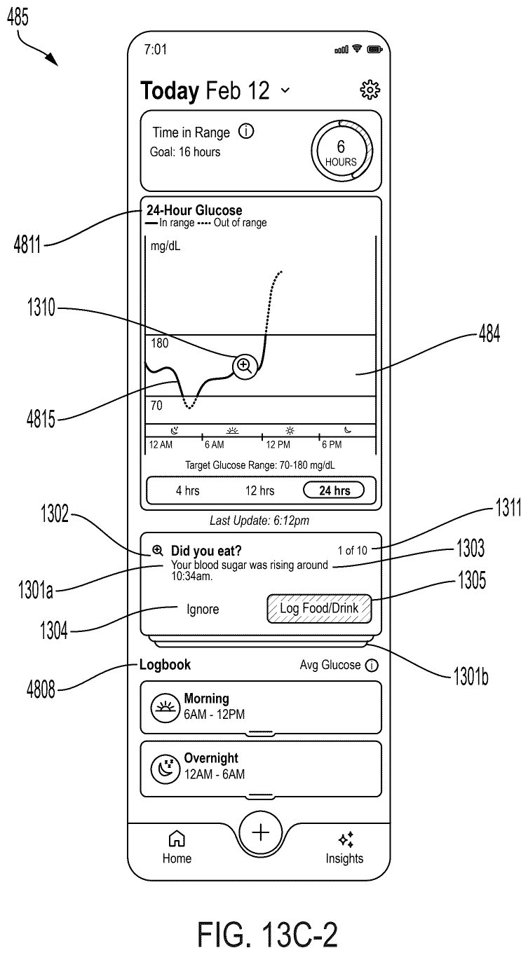

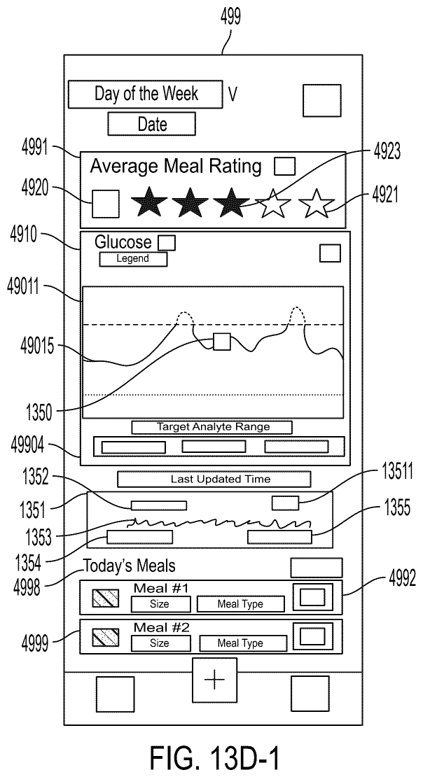

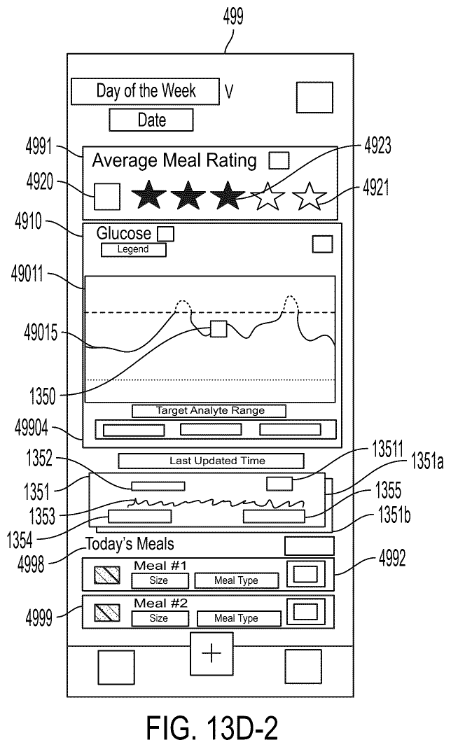

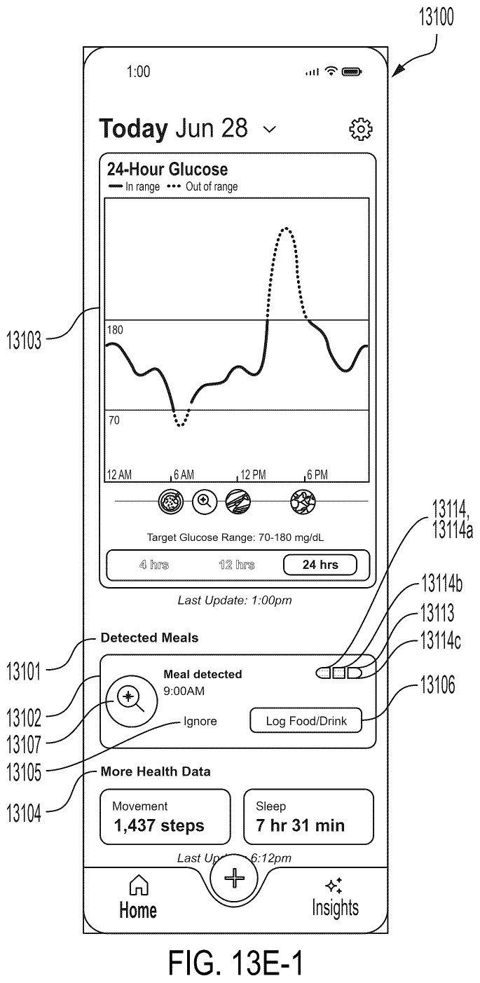

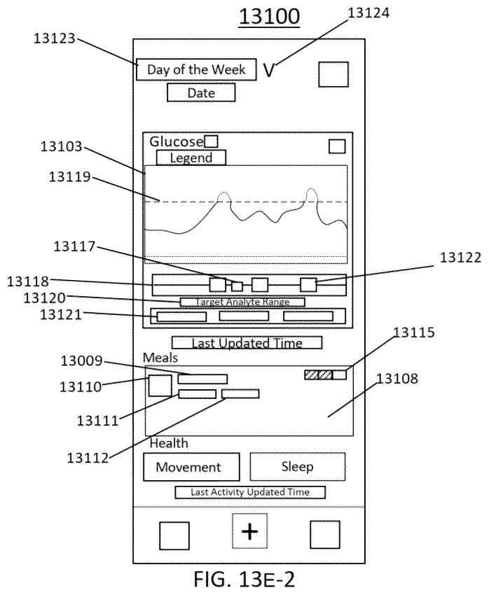

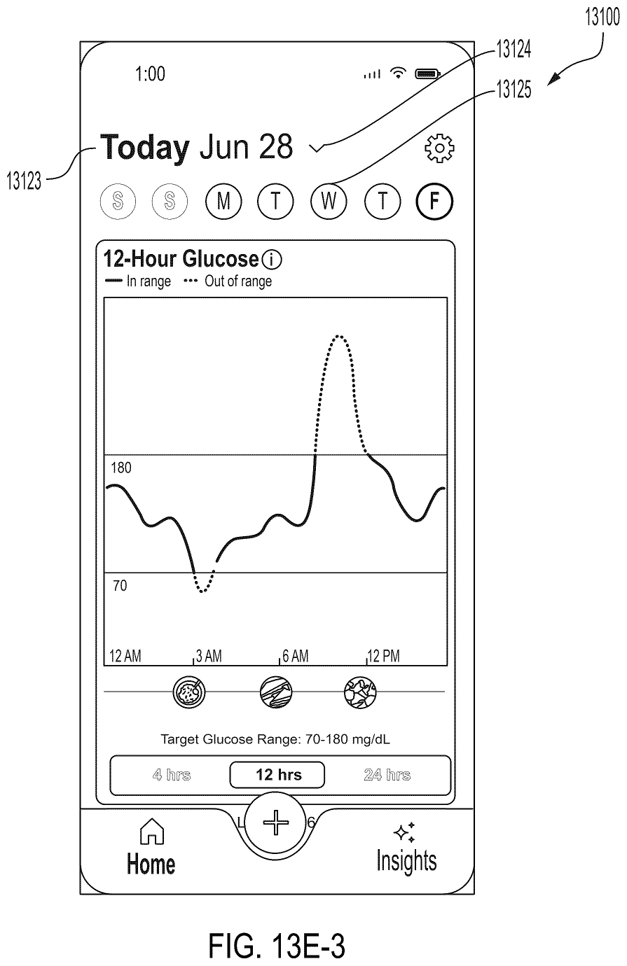

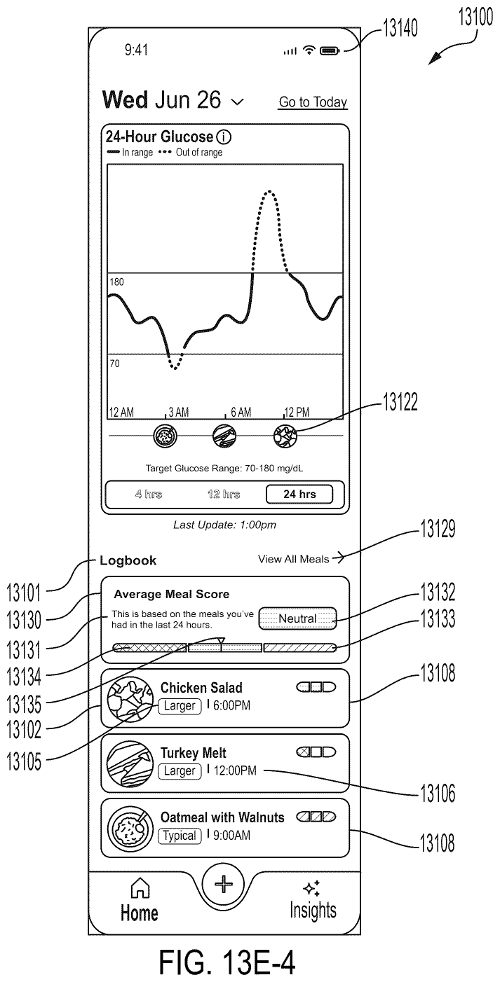

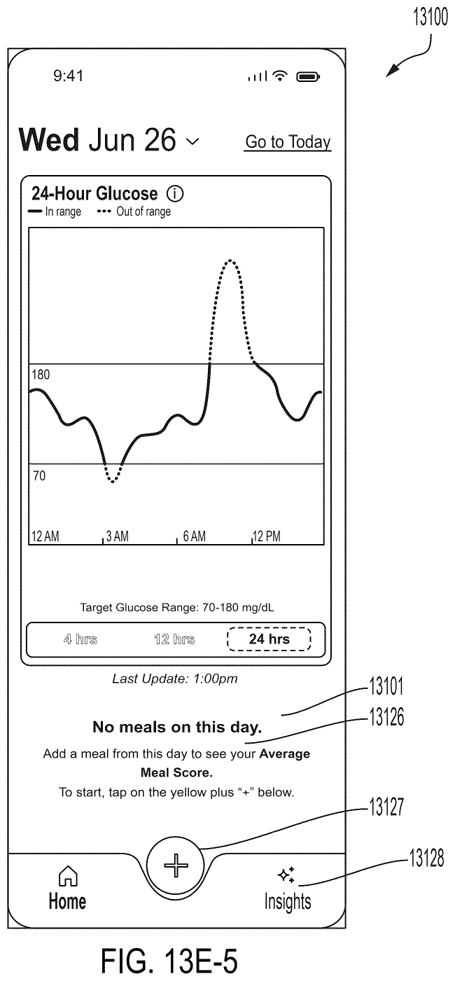

Example embodiments of methods for detecting whether a user has consumed a meal will now be described. As an initial matter, those of skill in the art will recognize that the method steps described herein can comprise software instructions stored in a memory of a computing device of system 100 (e.g., a reader 120, a local computer system 170, a trusted computer system 180), such that the instructions, when executed by one or more processors of the computing device, cause the one or more processors to perform any or all of the method steps described herein. Turning to FIG. 13A, a flow diagram depicts an example embodiment of a method 13500 for detecting whether the user has potentially consumed a meal (an “episode” or “excursion”) based on received data indicative of an analyte level (e.g., glucose level).

According to an aspect of the embodiments, method 13500 can utilize one or more parameters related to analyte level excursions (e.g., glucose level excursions) in determining meal detection. For example, a first parameter can include a minimum rate of analyte level change (e.g., glucose level change) over time needed to start the excursion (“Trigger Excursion Change”). In some embodiments, other parameters can be utilized to validly detect a meal episode or excursion, which include but are not limited to the following: (1) a threshold analyte level excursion duration (e.g., a minimum analyte level excursion duration or maximum analyte level excursion duration); (2) a threshold change in analyte level throughout an episode (e.g., a maximum change or minimum change in glucose level throughout an episode); (3) a threshold highest analyte level value and/or a threshold lowest analyte level value being met (e.g., a maximum lowest glucose value, a minimum lowest glucose value, a minimum highest glucose value, or a maximum highest glucose value); and (4) a threshold time window allowed between analyte level readings (e.g., a maximum amount of time allowed between glucose level readings).

Those of skill in the art will appreciate that the parameters related to excursions and validly detecting an episode can utilize various different threshold values and numbers.

As seen at the top of FIG. 13A, method 13500 begins at 13501, wherein a slope associated with incoming analyte level measurements (e.g., glucose level measurements) is calculated to determine the start of the excursion. In some embodiments, if the slope exceeds the Trigger Excursion Change parameter, a previous timestamp is determined to be the start of the excursion.

At 13502, once the start of the excursion has been determined, the slope continues to be calculated to determine the end of the excursion. In some exemplar embodiments, when the slope is less than the Trigger Excursion Change, the previous timestamp is determined to be the end of the excursion.

At 13503, and upon the end of the excursion being determined, a “trigger window” is calculated as the time (e.g., in minutes) between the start of the excursion and the end of the excursion. According to an aspect of the embodiments, the total analyte level change (e.g., the total glucose level change) is calculated as the difference in analyte level values (e.g., the difference in glucose level values) between the start of the excursion and the end of the excursion.

At 13504, the threshold lowest analyte level value needed for a valid episode detection is determined, wherein the threshold lowest analyte level value is calculated as the minimum analyte value (e.g., glucose value) during the excursion.

At 13505, the threshold highest analyte level value needed for a valid episode detection is determined, wherein the threshold highest analyte level value is calculated as the maximum analyte value (e.g., glucose value) during the excursion.

At 13506, the threshold time window allowed between analyte level readings (e.g., glucose level readings) is determined, wherein the threshold time window is calculated as the different in time between analyte level readings (e.g., glucose level readings) within the episode.

In some exemplar embodiments, the episode is determined to be valid if certain thresholds conditions are met, which includes but is not limited to the following conditions: (1) the threshold time window allowed between analyte level readings is determined to be between the minimum analyte level excursion duration and the maximum analyte level excursion duration; (2) the total analyte level change (e.g., the total glucose level change) is determined to be between the maximum change in analyte level and the minimum change in analyte level calculated throughout an episode; (3) the threshold highest analyte level value for the excursion is between a minimum highest analyte level value and a maximum highest analyte level value; and (4) the threshold time window are all less than the maximum amount of time allowed between analyte level readings for valid episode. In this regard, if the episode is valid, a meal has been detected for the episode.

In some exemplar embodiments, when one or more meals are inputted within a short time window, the one or more inputted meal can be configured such that they are considered as part of a single meal entry. In some embodiments, when one or more inputted meals occur within a short time window, the one or more inputted meal are considered as a single meal event and will be provided a singular score.

In some embodiments, when multiple meals are inputted within a predetermined time period of a consumed meal (e.g., within one hour of a meal), the multiple meals will be logged as a single meal. In some embodiments, the multiple meals that are logged as a single meal can comprise a timestamp of the earliest meal of the multiple inputted meals for the purposes of scoring the meal event. According to some embodiments, concatenation of meals does not extend to meals past a predetermined time within the initial meal (e.g., one hour from the initial meal).

Turning to FIG. 13B, a flow diagram depicts an example embodiment of a method 13600 for determining bespoke time windows for when a user is expected to eat one or more meals, based on historical data. For example, the bespoke time windows can be determined by utilizing at least one or more of the following: historical analyte data and/or data related to historically consumed and/or logged meals. In this regard, the meal monitoring application is configured to output notifications or reminders to the user associated with the bespoke time windows so as to prompt meal logging.

As seen at the top of FIG. 13B, method 13600 begins at 13601, wherein historic meal times are identified. Historical meal times, or meal times of a past time period, can be identified through analysis of meals previously logged by the user on the meal monitoring application and/or meals detected algorithmically through analysis of analyte data (e.g., glucose data). The logged meals can be meal information received through an input of a reader device. Historical meal times can be determined through any of the meal detection methods described herein (e.g. method 13500, shown in FIG. 13A).

At 13602, a mean meal time is determined through analysis of the identified historical meal times. 13602 requires a predefined minimum number of historical meal time instances and a predefined maximum number of historical meal time instances in order to determine the mean meal time. The predefined maximum number of historical meal time instances can be the meal times associated with a predefined number of the most recent meals. Additionally, the mean meal time can be calculated by using data corresponding to the meal times associated with the most recent meals, or a predetermined elapsed period of time, wherein data related to the most recent periods of time (e.g., the most recent days) are given more weight.

At 13603, an expected bespoke time window corresponding to when a user is expected to eat one or more meals at a future time period (e.g., an expected time window for when a user is expected to eat one or more meals for the subsequent day) is calculated. The expected bespoke time window can be calculated as: (1) a time window calculated as a multiple hour range, centered on the calculated mean meal time; (2) a time window determined by using meal times associated with a predefined number of the most recent meals that fall within a predetermined multiple of z-scores from the mean meal time; and (3) a time window determined through a machine learning-based model. According to some embodiments, for example, a machine learning-based model can be trained with certain data sets including, but not limited to, data indicative of the user's glucose levels obtained by the on-body unit of the continuous glucose monitoring system, meals logged by the user on the meal monitoring application, meals detected algorithmically through analysis of the analyte data, population and demographic data relevant to the user, and other sources of information. According to another aspect of some embodiments, the time window may be a predetermined period (e.g., 2-4 hours), or can be determined based on statistical analysis (e.g., one hour if the user is consistent regarding meal times, 4 hours if the user is inconsistent regarding meal times). In another aspect, the time window may be the same or different for the same meal on different days of the week. For example, the time window for lunch may be the same for Monday through Friday, but may be different for Saturday and/or Sunday. In still other embodiments, the time windows may be associated with geographical locations.

At 13604, and upon calculating the expected bespoke time window for a future time period, the bespoke time window can be updated so as to include meal times associated with the current period of time (e.g., the current day) as one of the meal times associated with the predefined number of the most recent meals. 13604 can be repeated with each new instance of a logged meal time associated with a consumed meal so as to continuously update the bespoke time window. In this manner, method 13600 is dynamic such that it can change the estimated bespoke time window based on the user's most recent eating trends.

Further, the bespoke time window can be utilized as a meal reminder for the user. Specifically, once the entirety of the bespoke time window has expired, the meal monitoring application can output a reminder or notification on a GUI on a meal monitoring application, wherein the reminder or notification is configured to indicate to the user to eat a meal (e.g., when current CGM data does not suggest a meal was consumed and/or current CGM level is below a threshold) and/or log a meal through the input on a reader device (e.g., when the CGM data suggests a meal was consumed). According to an aspect of many embodiments, the meal monitoring application can be further configured to output the reminder or notification as soon as a meal is detected, which can occur before the expiration of the time window. Notifications associated with meals are described herein, e.g., with reference to FIGS. 13C-1 to 13E-1, and 13E-4, as described below, however, those of skill in the art will appreciate that various other notifications can be utilized with any of the interface embodiments described herein, without departing from the scope of the present disclosure.

FIGS. 13C-1 and 13C-2 are block diagrams depicting example embodiments of meal detection interfaces, or features related thereto, any of which can be utilized with the embodiments described herein.

Referring to FIGS. 13C-1 and 13C-2, an example embodiment of a block diagram of a home GUI 485, wherein home GUI 485 further comprises one or more notifications 1301 (also herein referred to as in-app notifications 1301) associated with a missed meal detection or episode. In some embodiments, and as depicted in FIGS. 13C-1 and 13C-2, the one or more notifications 1301 associated with the missed meal detection can be presented as an in-app notification 1301 positioned between the analyte graph card 4811 and the logbook section 4808. Those of skill in the art will appreciate that the in-app notification 1301 can be displayed in various other positions on home GUI 485 without departing from the scope of the present disclosure.

According to an aspect of the embodiments, the in-app notifications 1301 can prompt users to log a meal. In some embodiments, and with reference to FIGS. 13C-1 and 13C-2, each of the in-app notifications 1301 comprises a query 1302 asking whether the user has eaten (e.g., “Did you eat?”), and a message 1303 indicating to the user that the user's blood sugar was rising around a particular time (e.g., “Your blood sugar was rising around 10:34 am.”) associated with the missed meal detection. According to an aspect of the embodiments, each of the in-app notifications 1301 can include a selectable “ignore” button 1304 and a selectable meal logging button 1305. Specifically, upon the user selecting the ignore button 1304 on a particular in-app notification 1301, the in-app notification 1301 is removed from the display of home GUI 485.

More specifically, in some embodiments, if the user selects the meal logging button 1305, meal logging GUI 500 (FIG. 5A) is outputted. Moreover, in some embodiments, meal entry modal 520 (as shown in FIG. 5C) can appear, wherein the time field 527 is prepopulated with a date and time associated with the detected missed meal associated with the in-app notification 1301 from which the meal logging button 1305 was selected. In other embodiments, meal entry modal 520 (as shown in FIG. 5C) can appear, wherein the user sets the date and time associated with the detected missed meal in the time field 527. In some embodiments, one the user has logged a meal for the particular time associated with the missed meal detection (e.g., by selecting the meal logging button 1305 in the in-app notification 1301), the respective in-app notification 1301 will be removed from home GUI 485. Further, the logged meal will be displayed in the logbook section 4808 of home GUI 485, e.g., in the appropriate TOD card 4801 under the list 4822 of meal events 4829.

According to yet another aspect of the embodiments, and as best shown in FIG. 13C-2, one or more missed episode markers 1310, such as one or more missed meal icons 1310 can be displayed near, close to, and/or proximal to analyte trendline 4815, or along the x-axis, the top of the graph window 484, or the bottom of the graph window 484. In some aspects of the embodiments, each missed meal icon 1310 is positioned on the analyte graph card 4811 so as to allow the user to visually associate a post-prandial analyte trace with the relevant missed meal and the particular time in which the missed meal was detected. In some embodiments, when the user selects the ignore button 1304 on a particular in-app notification 1301 associated with a detected missed meal, the corresponding missed meal icon 1310 will be removed from the analyte graph card 4811. In some embodiments, though not illustrated, when the user logs a meal associated with a particular previously detected missed meal, the missed meal icon 1310 is replaced with a meal icon.

In some embodiments, if the user ignores or fails to act on the in-app notification 1301, the in-app notification 1301 along with the corresponding missed meal icon 1310 are removed from home GUI 485 after a predetermined time period (e.g., after 24 hours).

According to another aspect of the embodiments, and as best depicted in FIG. 13C-2, if two in-app notifications 1301 associated with a detected missed meals are displayed on home GUI 485, the in-app notifications 1301 are oriented such that a first in-app notification 1301a is stacked or is partially overlayed on a second in-app notification 1301b. In some embodiments, the in-app notifications 1301 are stacked chronologically. For example, the most recent in-app notification 1301a is positioned such that it is stacked or partially overlayed over a past or preceding in-app notification 1301b. In this manner, the most recent in-app notification 1301 is more predominantly displayed on home GUI 485 when compared to the past in-app notification 1301 which is positioned underneath or partially underneath the most recent in-app notification 1301 in the stack of in-app notifications 1301. Further, in some embodiments, if two in-app notifications 1301 are displayed on home GUI 485, then each in-app notification 1301 comprises a pagination 1311, indicating the in-app notification 1301 is a particular in-app notification 1301 from a total number of in-app notifications 1301 displayed on home GUI 485 (e.g., “1 of 2” if there is a total of two in-app notifications 1301 and the most recent in-app notification 1301 is predominantly displayed on home GUI 485).

According to yet another aspect of the embodiments, and as best shown in FIG. 13C-2, if more than two in-app notifications 1301 associated with a detected missed meals are displayed on home GUI 485, the in-app notifications 1301 are configured such that they are stacked or partially overlayed on one another. In some embodiments, the in-app notifications 1301 are stacked chronologically. Further, in some embodiments, if more than two in-app notifications are displayed on home GUI 485, then each in-app notification 1301 comprises a pagination 1311, indicating the in-app notification 1301 is a particular in-app notification 1301 from a total number of in-app notifications 1301 displayed on home GUI 485 (e.g., “1 of 10” if there is a total of ten in-app notifications 1301 and the most recent in-app notification 1301 is predominantly displayed on home GUI 485). Additionally, according to some embodiments, once an in-app notification is removed from GUI 485 (e.g., through the user ignoring the in-app notification or logging a meal corresponding to the particular time associated with the missed meal detection, the pagination is configured to update so as to reflect the new total number of in-app notifications (e.g., if the user selects the ignore button on one of ten in-app notifications, then the pagination on the in-app notifications will update to reflect “1 of 9” instead of “1 of 10”).

Further, according to another aspect of the embodiments, and in response to a tenth predetermined input by the user, such as when the user taps or swipes on a particular in-app notification 1301, selects the ignore button 1304, or by some other predetermined gesture, the underlying in-app notification 1301 will surface to the top of the stack of in-app notifications 1301. For example, if the user selects the ignore button 1304 on one of three in-app notifications 1301, then the second in-app notification 1301 will be predominantly displayed on home GUI 485. In some embodiments, the second in-app notification 1301 of the three in-app notifications 1301 will further display a pagination 1311 indicating it is “2 of 3” in-app notifications 1301. Further, if the user selects the ignore button 1304 on the second in-app notification 1301, then the third in-app notification 1301 will be predominantly displayed on home GUI 485. In some embodiments, the third in-app notification 1301 of the three in-app notifications 1301 will further display a pagination 1311 indicating it is “3 of 3” in-app notifications 1301.

In some embodiments, the analyte graph card 4811 of home GUI 485 is configured to update such that the missed meal icon 1310 corresponding to the predominantly displayed in-app notification 1301 is displayed on the analyte graph card 4811. For example, if the user swipes from the first in-app notification 1301 such that the second in-app notification 1301 is predominantly displayed on home GUI 485, then the analyte graph card 4811 is configured to update such that the missed meal icon 1310 associated with the first in-app notification 1301 is removed from the analyte graph card 4811 and the missed meal icon 1310 associated with the second in-app notification 1301 is displayed on the analyte graph card 4811.

Though the notifications 1301 associated with the missed meal detection have been described herein with reference to home GUI 485, those of skill in the art will appreciate that the notification 1301 and features related thereto can be utilized with home GUI 400 (FIG. 4B), home GUI 450 (FIG. 4D), home GUI 430 (FIGS. 4E-1 to 4E-7), home GUI 485 (FIGS. 4J-1 to 4J-3), home GUI 495 (FIGS. 4K-1 to 4K-3), and home GUI 499 (FIGS. 4M-1 to 4M-2), without departing from the scope of the disclosure.

For example, FIGS. 13D-1 and 13D-2 are block diagrams depicting home GUI 499 comprising meal detection features. Specifically, and with reference to FIGS. 13D-1 and 13D-2, one or more notifications 1351 associated with a missed meal can be presented as an in-app notification 1351 on home GUI 499, wherein the home GUI 499 comprises: the average meal rating card 4991 which includes the average meal rating 4920, the graphical indication 4921 corresponding to the average meal rating 4920, and the average meal rating indicator 4923 that visually illustrates the average meal rating provided for the predetermined period of time; the analyte graph card 4910 comprising the analyte graph 49011 which includes the analyte trendline 49015; and the logbook section 4998 comprising the list 4992 of meal events 4999. In some exemplar embodiments, an in-app notification can be positioned between the analyte graph card 4910 and the logbook section 4998 of home GUI 499. Those of skill in the art will appreciate that the in-app notification 1351 can be displayed in various other positions on home GUI 499 without departing from the scope of the present disclosure.

In some embodiments, and still with reference to FIGS. 13D-1 and 13D-2, each of the in-app notifications 1351 comprises a query 1352 asking whether the user has eaten (e.g., “Did you eat?”), and a message 1353 indicating to the user that the user's blood sugar was rising around a particular time (e.g., “Your blood sugar was rising around 10:34 am.”) associated with the missed meal detection. According to an aspect of the embodiments, each of the in-app notifications 1351 can include a selectable “ignore” button 1354 and a selectable meal logging button 1355. Specifically, upon the user selecting the ignore button 1354 on a particular in-app notification 1351, the in-app notification 1351 is removed from the display of home GUI 499.

According to an aspect of the embodiments, if the user selects the meal logging button 1355, meal logging GUI 500 (FIG. 5A) is outputted. Moreover, in some embodiments, meal entry modal 520 (as shown in FIG. 5C) can appear, wherein the time field 527 is prepopulated with a date and time associated with the detected missed meal associated with the in-app notification 1351 from which the meal logging button 1355 was selected. In other embodiments, meal entry modal 520 (as shown in FIG. 5C) can appear, wherein the user sets the date and time associated with the detected missed meal in the time field 527. In some embodiments, one the user has logged a meal for the particular time associated with the missed meal detection (e.g., by selecting the meal logging button 1355 in the in-app notification 1351), the respective in-app notification 1351 will be removed from home GUI 499. Further, the logged meal will be displayed in the logbook section 4998 of home GUI 499, e.g., under the list 4992 of meal events 4999.

According to yet another aspect of the embodiments, and as best shown in FIG. 13D-2, one or more missed episode markers 1350, such as one or more missed meal icons 1350 can be displayed near, close to, and/or proximal to analyte trendline 49015, or along the x-axis, the top of the graph window 49904, or the bottom of the graph window 49904. In some aspects of the embodiments, each missed meal icon 1350 is positioned on the analyte graph card 49011 so as to allow the user to visually associate a post-prandial analyte trace with the relevant missed meal and the particular time in which the missed meal was detected. In some embodiments, when the user selects the ignore button 1354 on a particular in-app notification 1351 associated with a detected missed meal, the corresponding missed meal icon 1350 will be removed from the analyte graph card 49011. In some embodiments, though not illustrated, when the user logs a meal associated with a particular previously detected missed meal, the missed meal icon 1350 is replaced with a meal icon.

In some embodiments, if the user ignores or fails to act on the in-app notification 1351, the in-app notification 1351 along with the corresponding missed meal icon 1350 are removed from home GUI 499 after a predetermined time period (e.g., after 24 hours).

According to another aspect of the embodiments, and as best depicted in FIG. 13D-2, if two in-app notifications 1351 associated with a detected missed meals are displayed on home GUI 499, the in-app notifications 1351 are oriented such that a first in-app notification 1351a is stacked or is partially overlayed on a second in-app notification 1351b. In some embodiments, the in-app notifications 1351 are stacked chronologically. For example, the most recent in-app notification 1351a is positioned such that it is stacked or partially overlayed over a past or preceding in-app notification 1351b. In this manner, the most recent in-app notification 1351 is more predominantly displayed on home GUI 499 when compared to the past in-app notification 1351 which is positioned underneath or partially underneath the most recent in-app notification 1351 in the stack of in-app notifications 1351. Further, in some embodiments, if two in-app notifications 1351 are displayed on home GUI 499, then each in-app notification 1351 comprises a pagination 13511, indicating the in-app notification 1351 is a particular in-app notification 1351 from a total number of in-app notifications 1351 displayed on home GUI 499 (e.g., “1 of 2” if there is a total of two in-app notifications 1351 and the most recent in-app notification 1351 is predominantly displayed on home GUI 499).

According to yet another aspect of the embodiments, and as best shown in FIG. 13D-2, if more than two in-app notifications 1351 associated with a detected missed meals are displayed on home GUI 499, the in-app notifications 1351 are configured such that they are stacked or partially overlayed on one another. In some embodiments, the in-app notifications 1351 are stacked chronologically. Further, in some embodiments, if more than two in-app notifications are displayed on home GUI 499, then each in-app notification 1351 comprises a pagination 13511, indicating the in-app notification 1351 is a particular in-app notification 1351 from a total number of in-app notifications 1351 displayed on home GUI 499 (e.g., “1 of 10” if there is a total of ten in-app notifications 1351 and the most recent in-app notification 1351 is predominantly displayed on home GUI 499). Additionally, according to some embodiments, once an in-app notification is removed from GUI 499 (e.g., through the user ignoring the in-app notification or logging a meal corresponding to the particular time associated with the missed meal detection, the pagination is configured to update so as to reflect the new total number of in-app notifications (e.g., if the user selects the ignore button on one of ten in-app notifications, then the pagination on the in-app notifications will update to reflect “1 of 9” instead of “1 of 10”).

Further, according to another aspect of the embodiments, and in response to an eleventh predetermined input by the user, such as when the user taps or swipes on a particular in-app notification 1351, selects the ignore button 1354, or by some other predetermined gesture, the underlying in-app notification 1351 will surface to the top of the stack of in-app notifications 1351. For example, if the user selects the ignore button 1354 on one of three in-app notifications 1351, then the second in-app notification 1351 will be predominantly displayed on home GUI 499. In some embodiments, the second in-app notification 1351 of the three in-app notifications 1351 will further display a pagination 13511 indicating it is “2 of 3” in-app notifications 1351. Further, if the user selects the ignore button 1354 on the second in-app notification 1351, then the third in-app notification 1351 will be predominantly displayed on home GUI 499. In some embodiments, the third in-app notification 1351 of the three in-app notifications 1351 will further display a pagination 13511 indicating it is “3 of 3” in-app notifications 1351.

In some embodiments, the analyte graph card 4910 of home GUI 499 is configured to update such that the missed meal icon 1350 corresponding to the predominantly displayed in-app notification 1351 is displayed on the analyte graph card 4910. For example, if the user swipes from the first in-app notification 1351 such that the second in-app notification 1351 is predominantly displayed on home GUI 499, then the analyte graph card 4910 is configured to update such that the missed meal icon 1350 associated with the first in-app notification 1351 is removed from the analyte graph card 4910 and the missed meal icon 1350 associated with the second in-app notification 1351 is displayed on the analyte graph card 4910.

FIGS. 13E-1 to 13E-5 are block diagrams depicting additional exemplar embodiment of interfaces comprising meal detection features, any of which can be utilized with the embodiments described herein. Specifically, FIG. 13E-1 depicts GUI 13100 for the TIR application. Specifically, GUI 13100 can be configured as a home interface for the TIR application. More specifically, GUI 13100 is similar to GUI 12100 depicted in FIG. 12D-2, except that it further comprises a meals section 13101 which can include one or more notifications 13102 associated with a missed meal detection or episode (only one notification 13102 depicted in FIG. 13E-1). In some embodiments, and as depicted in FIGS. 13E-1, the one or more notifications 13102 associated with the missed meal detection can be presented as an in-app notification 13102 positioned between the analyte graph card 13103 and the health section 13104. Those of skill in the art will appreciate that the in-app notification 13102 can be displayed in various other positions on GUI 13100 without departing from the scope of the present disclosure. In some embodiments (though not illustrated), wherein GUI 13100 does not display a health section 13104, the one or more notifications 13102 are displayed directly adjacent to and distal relative to (e.g., below) the analyte graph card 13103.

According to an aspect of the embodiments, the in-app notifications 13102 can prompt users to log a meal. In some embodiments, and with reference to FIG. 13E-1, each of the in-app notifications 13102 comprises a message indicating that a meal was detected. Further, in some embodiments, each of the in-app notifications 13102 can include a timestamp 13107 indicating a time associated with the detected meal (e.g., “9:00 AM”). Additionally or alternatively, in some exemplar embodiments, each of the in-app notifications 13102 can include a graphical representation 13113, such as a segmented bar 13113, representing a score associated with the detected meal. Specifically, the score can be represented by a segmented bar 13113 with three segment portions 13114, wherein: (1) the first segment 13114a can have a red color or orange color and be indicative of an analyte score corresponding to a high glucose impact or response (e.g., an average glucose range exceeding 250 mg/dL); (2) the first segment 13114a and second segment 13114b can have a yellow color and be indicative of an analyte score corresponding to a medium glucose impact or response (e.g., an average glucose range between 181 mg/dL and 250 mg/dL, or no greater than 250 mg/dL and/or at least 181 mg/dL); or (3) the first segment 13114a, the second segment 13114b, and the third segment 13114c can have a green color and be indicative of an analyte score corresponding to a low glucose impact or response (e.g., an average glucose range between 70 mg/dL and 180 mg/dL, or no greater than 180 mg/dL). Those of skill in the art will appreciate that various other graphical representations and colors, and color configurations can be utilized with the graphical representations without departing from the scope of the disclosure.

According to an aspect of the embodiments, each of the in-app notifications 13102 can include a selectable “ignore” button 13105 and a selectable meal logging button 13106. Specifically, upon the user selecting the ignore button 13105 on a particular in-app notification 13102, the in-app notification 13102 is removed from the display of GUI 13100. More specifically, in some embodiments, if the user selects the meal logging button 13106, meal logging GUI 500 (FIG. 5A) is outputted. Moreover, in some embodiments, meal entry modal 520 (as shown in FIG. 5C) can appear, wherein the time field 527 is prepopulated with a date and time associated with the detected missed meal associated with the in-app notification 13102 from which the meal logging button 13106 was selected. In other embodiments, meal entry modal 520 (as shown in FIG. 5C) can appear, wherein the user sets the date and time associated with the detected missed meal in the time field 527. In some embodiments, when meal logging GUI 500 is outputted through the user selecting the meal logging button 13106 of GUI 13100, a camera interface (e.g., FIGS. 5I to 5N) is not shown to the user. Further, in some embodiments, one the user has logged a meal for the particular time associated with the meal detection, the respective in-app notification 13102 will be replaced with a meal event 13108 corresponding to the logged meal (see, e.g., FIG. 13E-2).

In some embodiments, though not illustrated, once a user has successfully logged a meal, an in-app banner can be displayed on GUI 13100, indicating that a meal was added. Additionally, the in-app banner can comprise a selectable “View Meal” link which, upon being selected, outputs a meal review GUI 800 or meal review GUI 8000 (FIGS. 8A and 8B-8E, respectively) corresponding to the newly logged meal. Further, in some embodiments (though not illustrated), and upon the user editing a logged meal entry, the in-app banner can be displayed on GUI 13100, wherein the in-app banner indicates to the user that a meal was updated. In some embodiments, when a meal is updated, the in-app banner (not illustrated) can further include a “View Meal” link which, upon being selected, outputs a meal review GUI 800 or meal review GUI 8000 (FIGS. 8A and 8B-8E, respectively) corresponding to the updated logged meal.

In some embodiments, and as best shown in FIG. 13E-2, each meal event 13108 can include a text description 13109 of a meal, photograph or picture 13110 associated with the meal, a portion size indicator 13111 describing the relative portion size of the meal, a timestamp 13112 indicating the time associated with the logged meal, and/or a graphical representation 13115, such as a segmented bar 13115, representing a score associated with the logged meal.

According to yet another aspect of the embodiments, and as best shown in FIG. 13E-2, one or more missed episode markers 13117, such as one or more missed meal icons 13117 can be displayed on a meal timeline 13118. In some embodiments, the meal timeline 13118 is only displayed on GUI 13110 when one or more missed meals are detected or one or more meal events have occurred. Specifically, in some embodiments, and with reference to FIG. 12D-1, each of the one or more missed meal icons 13117 is positioned along the meal timeline 13118 so as to correspond to a time associated with the detected missed meal. In some embodiments, the meal timeline 13118 can be displayed near, close to, and/or distal to (e.g., below) an analyte graph 13119 of the analyte graph card 13103, below the x-axis, and/or at the bottom of the graph window. In some embodiments, the GUI 13100 includes a target analyte range 13120 (e.g., “target glucose range: 70-180 mg/dL”), and/or one or more selectable icons 13121 (e.g., radio button, check box, slider, switch, etc.) that allow the user to select a predefined amount of time over which the user's analyte data will be shown in the analyte graph card 13103. Specifically, in some embodiments, the meal timeline 13118 can be positioned distal relative to (e.g., below) the analyte graph 13119, and between the x-axis and one or more selectable icons 13121. More specifically, the x-axis can comprise units of time (e.g., three hour increments, etc.) covering a twenty-four hour period. In this manner, the meal timeline 13118 can be positioned such that the user can visually associate a post-prandial analyte trace with the relevant detected missed meal associated with each missed meal icons 13117 displayed on the meal timeline 13118.

In some embodiments, when the user selects the ignore button 13105 on a particular in-app notification 13102 associated with a detected missed meal, the corresponding missed meal icon 13117 will be removed from the meal timeline 13118. In some embodiments, though not illustrated, when the user logs a meal associated with a particular previously detected missed meal, the missed meal icon 13117 is replaced with a logged meal icon 13122. In some embodiments, though not illustrated, if the user ignores or fails to act on the in-app notification 13102, the in-app notification 13102 along with the corresponding missed meal icon 13117 are removed from GUI 13100 after a predetermined time period (e.g., after 24 hours).

Further, in some embodiments, a missed meal icon 13117 can include placeholder image instead of a photograph or image selected by the user. In some embodiments, once the user has logged a meal associated with the detected missed meal, the missed meal icon 13117 is replaced with the logged meal icon 13122, wherein the logged meal icon 13122 can include a photograph or image associated with a particular meal. According to an aspect of the embodiments, each missed meal icon 13117 and/or logged meal icon 13122 is selectable. Specifically, upon the user selecting particular a particular each missed meal icon 13117, a meal review GUI 8000 corresponding to the selected missed meal icon 13117 is outputted (e.g., meal review GUI 8000 as shown in FIGS. 8C to 8E). More specifically, upon the user selecting particular a particular logged meal icon 13122, a meal review GUI 800 or meal review GUI 8000 corresponding to the selected logged meal icon 13122 is outputted (e.g., meal review GUI 800 as shown in FIG. 8A, or meal review GUI 8000 as shown in FIG. 8B-8E).

In some embodiments, and as shown in FIGS. 13E-1 and 13E-2, GUI 13100 can further comprise a text description 13123 indicating a current day of the week (e.g., “Today June 28”), wherein the text description 13123 can include a date adjacent to a switch 13124 configured to be toggled. In some embodiments, and as shown in FIG. 13E-3, when the switch 13124 is toggled down by the user, the GUI 13100 is configured to display a plurality of selectable rings 13125, each of which corresponds to a different day of the past week (similar to the plurality of rings 425 depicted in FIG. 6B-1). For example, when the switch 13124 is toggled down, the GUI 13100 can display a plurality of seven rings 13125, one corresponding to each day of the week. In some embodiments, when a user selects a particular ring from the plurality of rings 13125 (e.g., “W” ring 13125, which corresponds to Wednesday), GUI 13100 will display data pertaining to the past day corresponding to the selected ring 13125, as shown in FIG. 13E-4. As shown in FIG. 13E-4, when GUI 13100 comprises data pertaining to a past day, the text description 13123 will update so as to include a date associated with the past day (e.g., “Wed June 26”). In some embodiments, when the switch 13124 is toggled to an up position, the plurality of rings is no longer displayed (see, e.g., FIGS. 13E-1 and 13E-2). Further, in some embodiments, and as shown in FIG. 13E-4, when GUI 13100 is related to a past day, GUI 13100 can further comprise a selectable “Go To Today” link 13140 which, upon being selected, outputs GUI 13100 comprising data corresponding to the current day (FIGS. 13E-1 and 13E-2).

According to an aspect of the embodiments, and as depicted in FIG. 13E-4, GUI 13100 corresponding to a past day can include a selectable “All Meals” link 13129 and meals section 13101 comprising one or more notifications 13102 associated with a missed meal detection from the past day and/or one or more meal events 13108 corresponding to logged meals for the past day. In this manner, the user can select the meal logging button 13106 on one of the one or more notifications 13101 to log a meal through meal logging GUI 500, or select one of the one or more meal events 13108 so as to output meal review GUI 800 or meal review GUI 8000 corresponding to the selected meal event 13108 (e.g., meal review GUI 800 as shown in FIG. 8A, or meal review GUI 8000 as shown in FIG. 8B-8E).

According to another aspect of the embodiments, and as best depicted in FIG. 13E-4, GUI 13100 can further comprise an average meal score card 13130 configured to display data related to a total average meal score, wherein the total average meal score is a total score for all scored meal events 13108 and all scored detected missed meals. Specifically, the average meal score card 13130 can comprise: (1) a message 13131, wherein the message 13131 indicates to the user that the total average meal score is based on the meals the user has had in a predetermined period of time (e.g., the last 24 hours); (2) a status indicator 13132, wherein the status indicator 13132 summarizes the total average meal score (e.g., “Poor,” “Fair,” “Neutral,” “Medium,” and “Excellent”); and/or (3) a graphical representation 13133 corresponding to a total average meal score of the meal events 13108 and scored detected missed meals, collectively, for a predetermined time period (e.g., for a 24-hour time period of one day). According to some embodiments, the total average meal score is calculated after a predetermined period of time has elapsed (e.g., after full day or a full 24-hours has elapsed). In some embodiments, the total average meal score is determined for a predetermined frequency of time (e.g., the preceding or trailing 24-hours period). In some embodiments, the total average meal score is continuously being calculated at a predetermined frequency to reflect a new total average meal score for a new predetermined period of time.

Further, and according to some embodiments, the graphical representation 13133 can be a gradient scale 13133. The gradient scale 13133 can include a gradient 13134, wherein each portion of the gradient 13134 corresponds to a different status of the total average meal score. Specifically, in some embodiments, the gradient scale 13133 can include a color gradient 13134, wherein each color of the color gradient 13134 corresponds to a different status of the total average meal score (e.g., red/red-orange color represents poor status, orange color represents fair status, yellow color represents neutral status, dark-yellow/light-green color represents medium status, and green color represents excellent status). According to some embodiments, the total average meal score can be a number between one and 100. In this manner, the gradient scale 13133 can represent a total average meal score range between one and 100. In some embodiments, the gradient scale 13133 comprises a picker 13135 configured to align with a portion of the color gradient 13134 corresponding to the total average meal score so as to visually identify for the user the total average meal score (e.g., the picker 13135 can be aligned with the yellow color of the color gradient 13134 if the total average meal score is in a neutral status). In this manner, the picker 13135 is configured to align with a color of the gradient scale 13133 corresponding to a status of the total average meal score. In some embodiments, the status indicator 13132 comprises a colored or shaded portion, wherein the color or shaded portion corresponds to the color the picker 13135 is aligned with on the color gradient 13134 so as to visually illustrate to the user the status of the total average meal score. Those of skill in the art will appreciate that various other scoring ranges, graphical representations, gradients, color gradients, colors, and configurations can be utilized to represent the total average meal score without departing from the scope of the disclosure.

According to yet another aspect of the embodiments, and as best shown in FIG. 13E-5, if there are no missed meal detections or meal events for a past day, then GUI 13100 corresponding to the past day will include meals section 13101 with no notifications associated with a missed meal detection or meal events. Specifically, and as depicted in FIG. 13E-5, meals section 13101 can include a message 13126 indicating to the user that there are no meals on this particular day, and notifying the user that they can add a meal from this day to see a total average meal score. In some embodiments, the message 13126 can further inform the user that to start adding meals, the user can tap on the “+” icon 13127 on GUI 13100.

In some embodiments, the total average meal score corresponding to a particular time period (e.g., for a 24-hour period or day), can continuously change as meals are logged or detected for the particular time period. Additionally, if the user deletes a meal event 13108 or selects the “ignore” button 1305 for a particular in-app notification 1301 corresponding to a detected missed meal, then the total average meal score will update so as to reflect the deletion of the meal event 13108 or detected missed meal. In this manner, if the total average meal score had initially accounted for the deleted meal event 13108 or detected missed meal, then the total average meal score will be recalculated so as to remove the deleted meal event 13108 or detected missed meal from the total average meal score.

Further, in some embodiments, the TIR application is configured to automatically score detected missed meals even if the user has not yet logged the meal, wherein the total average meal score will be calculated in light of the automatically scored detected missed meal. In other embodiments, the TIR application is configured to automatically score detected missed meals even if the user has not yet logged the meal, wherein the total average meal score will be calculated without the automatically scored detected missed meal. In yet other embodiments, the TIR application is configured to not score the detected missed meal(s) until the user has successfully logged the meal, wherein the total average meal score will be calculated in light of the newly logged meal only after it is successfully logged by the user.

In some embodiments, once a detected meal event has been successfully logged, the TIR application will output meal review GUI 8000, as depicted in FIG. 8B. Further, in some embodiments, if a missed meal is detected yet not yet logged, the user has a predetermined amount of time to log the missed meal prior to it automatically being deleted. For example, in some embodiments, the user has seven days to retroactively log a detected missed meal in the TIR application.

Further, in some embodiments, and as bests shown in FIG. 13E-5, GUI 13100 can comprise an insights icon 13128 which, upon being selected by the user, outputs insights GUI 14000 or insights GUI 150000 (e.g., FIGS. 14A-1 to 14A-5, and FIGS. 15A-1 through 15A-5, respectively), as will be further detailed below.

### Example Embodiments of Insights GUIs, Methods, and Features Related Thereto

Example embodiments of methods for identifying high analyte level impact and low analyte level impact periods of time based on meal excursions will now be described. As an initial matter, those of skill in the art will recognize that the method steps described herein can comprise software instructions stored in a memory of a computing device of system 100 (e.g., a reader 120, a local computer system 170, a trusted computer system 180), such that the instructions, when executed by one or more processors of the computing device, cause the one or more processors to perform any or all of the method steps described herein. For ease of illustration, the high analyte level impact and low analyte level impact in this and other embodiments described below will be high glucose level impact and low glucose level impact, respectively, although other high analyte level impact and low analyte level impact can be identified as well, as is noted herein.

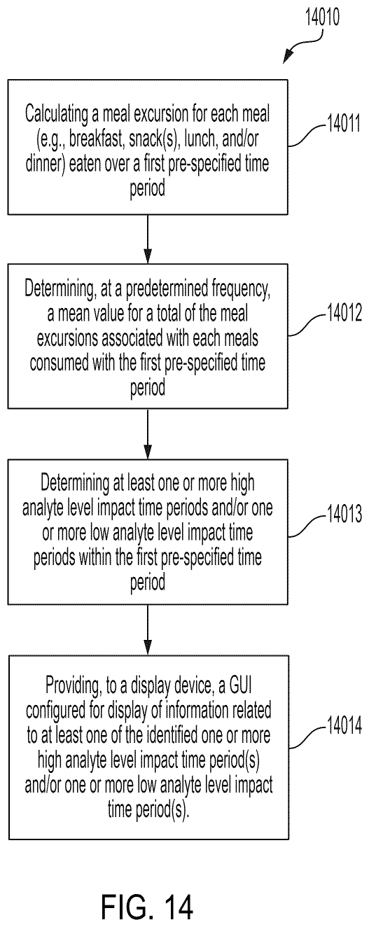

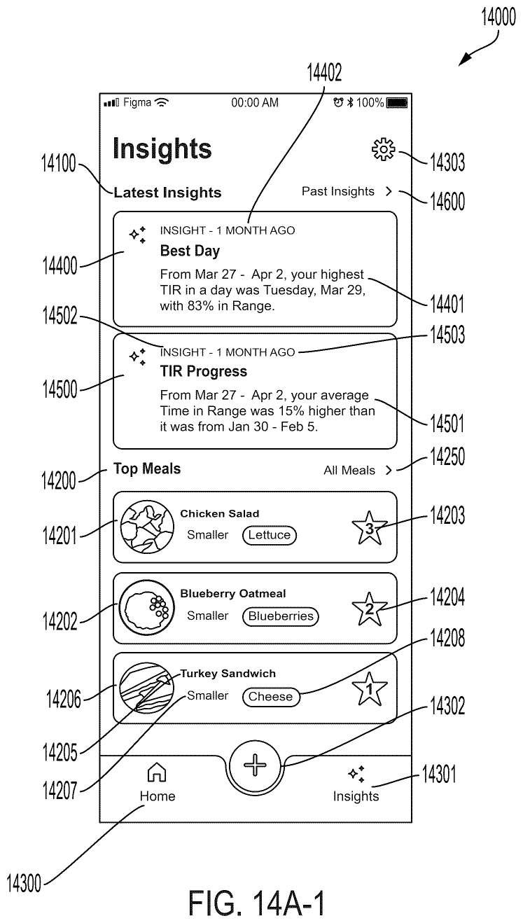

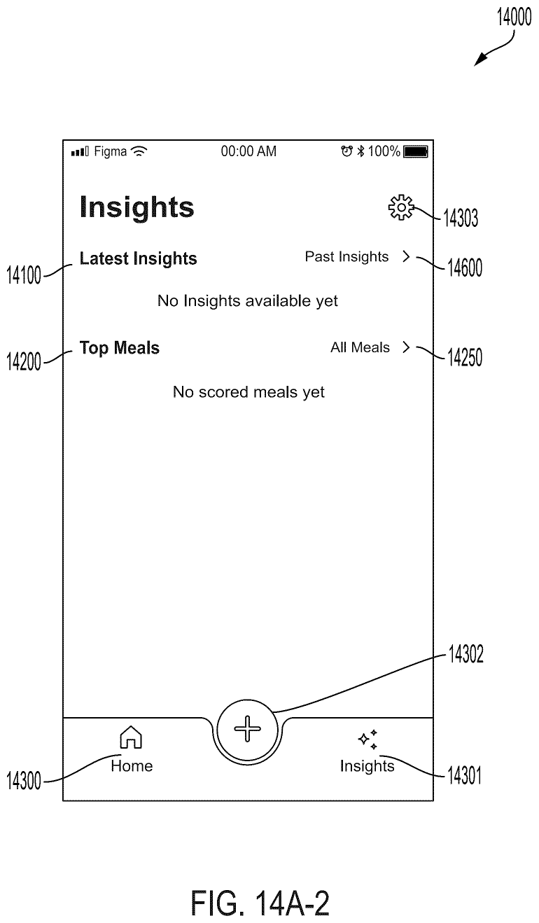

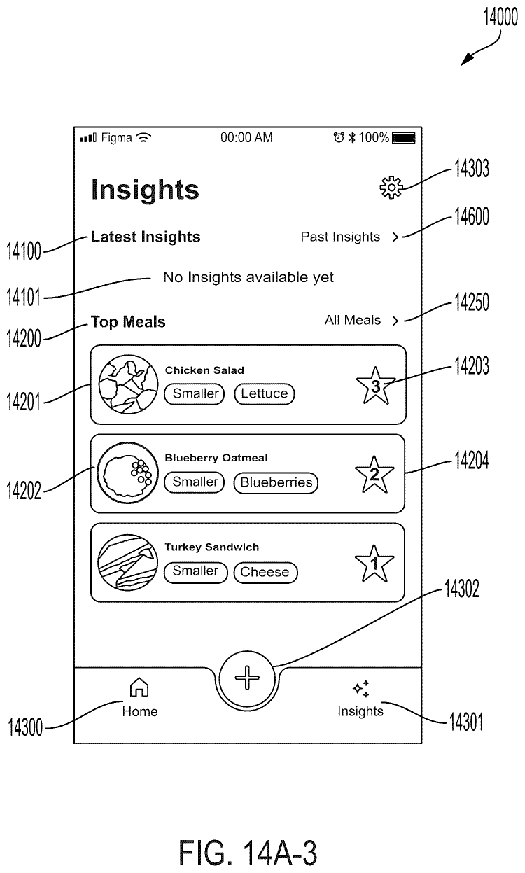

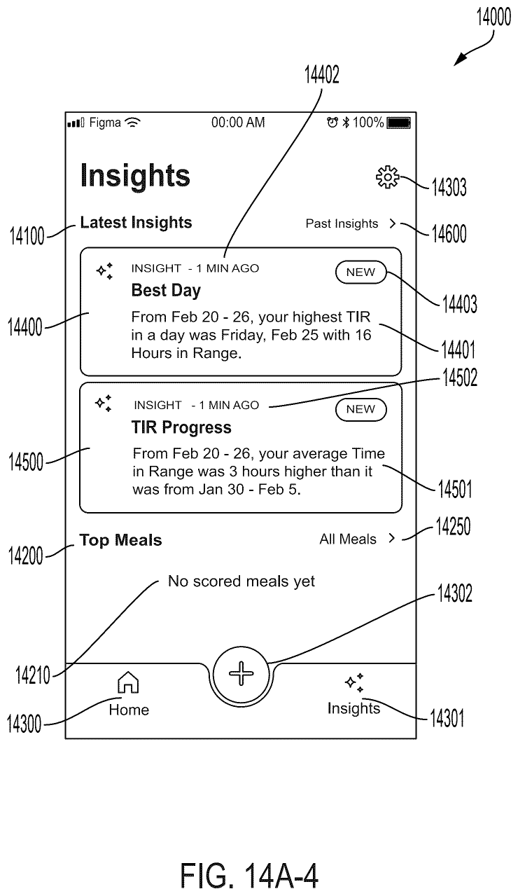

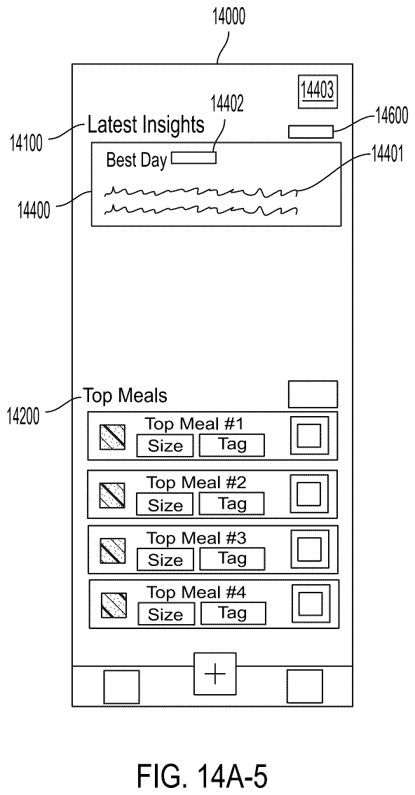

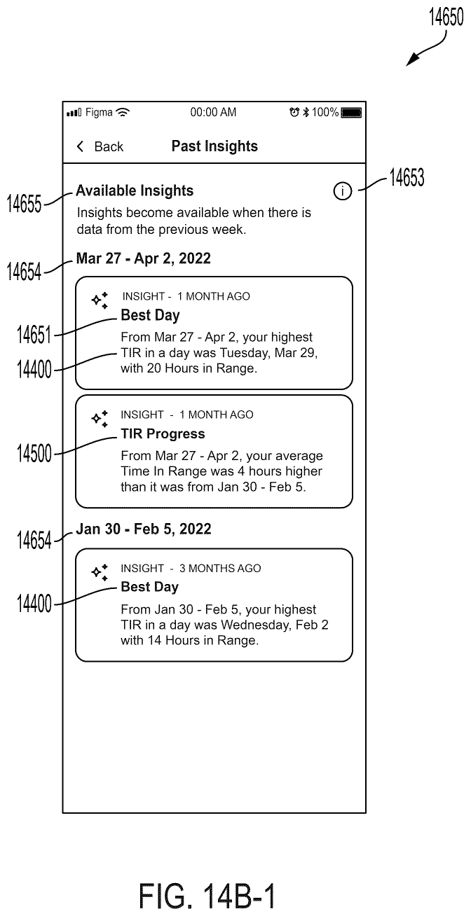

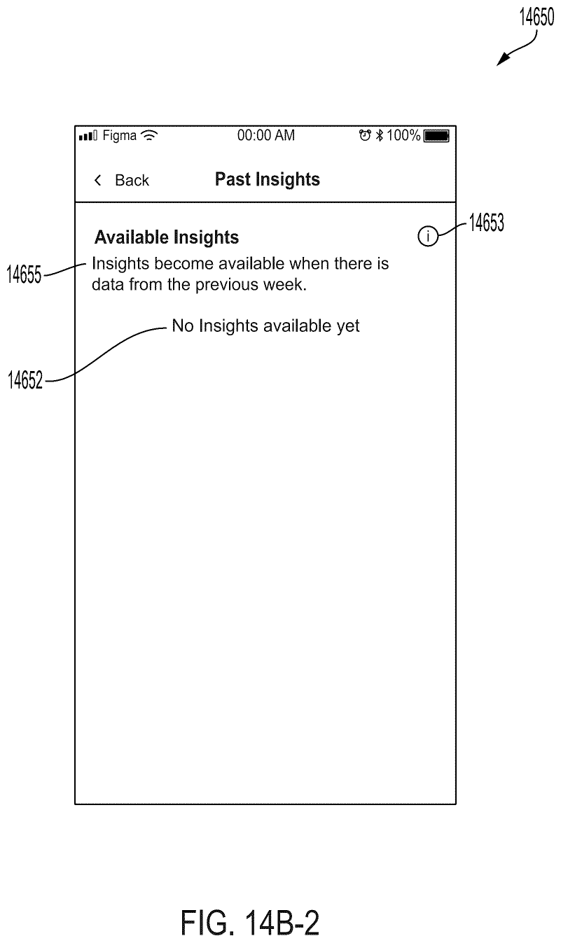

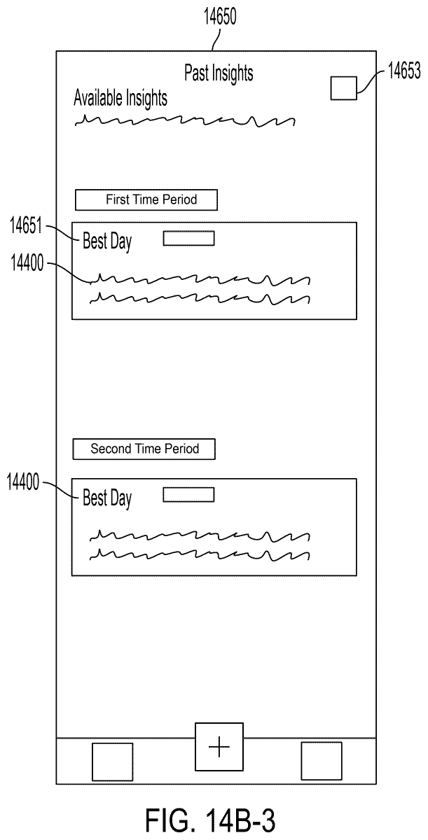

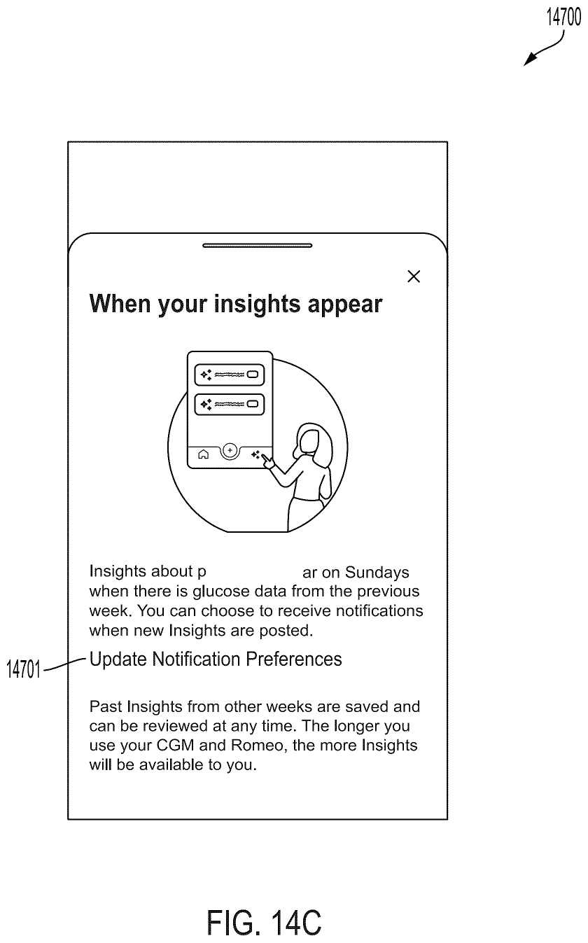

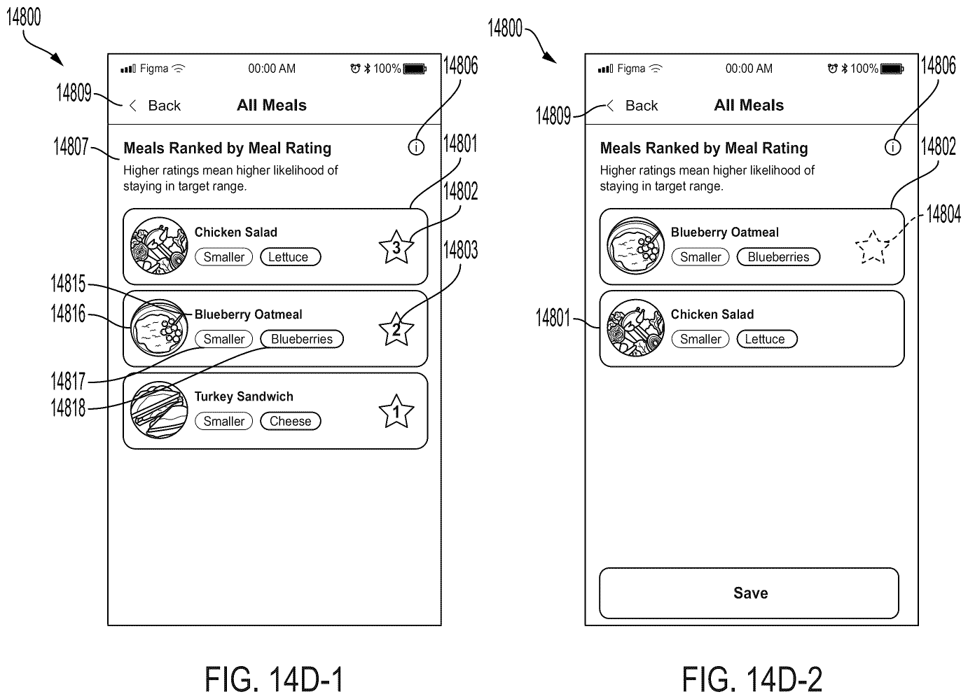

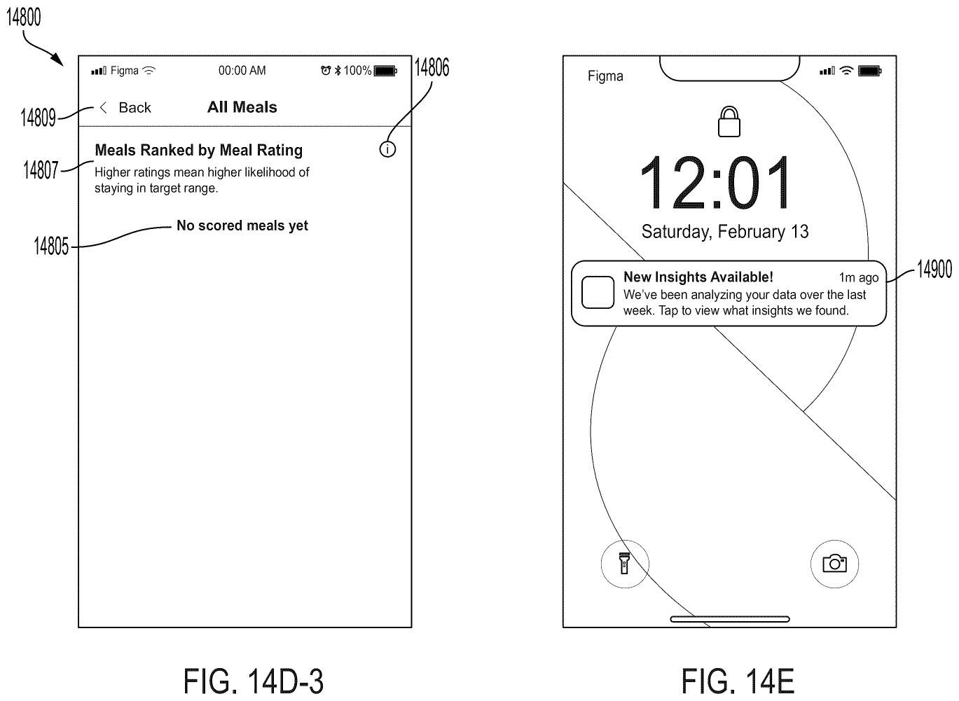

FIG. 14 is a flow diagram depicting an example embodiment of a method 14010 for identifying high analyte level impact and low analyte level impact time periods based on meal excursions of the user. As seen at the top of FIG. 14, method 14010 begins at 14011, a meal excursion for each meal (e.g., breakfast, snack(s), lunch, and/or dinner) eaten over a first pre-specified time period (e.g., per day) is calculated. The first pre-specified time period can exist as a moving time window or one or more serial blocks of time. Additionally, the data corresponding to each first pre-specified time period can be weighted so as to preference particular times over others.

Specifically, the meal excursion for each meal can be calculated by one or more of the following: (1) the delta between the highest glucose level value experienced within a predetermined period of time post-meal (e.g., two hours, three hours, etc.) and an initial glucose level value at or close to the time of the meal; (2) the delta between the initial glucose level value and the highest glucose level value when a glucose rate of change (e.g., increase) exceeds a set threshold; (3) the delta between the initial glucose level value and the highest glucose level value when a glucose rate of change shifts (e.g., from positive to negative); and (4) the delta between the initial glucose level value and the highest glucose level value determined by a machine-learning based model.

At 14012, a mean value for a total of the meal excursions associated with each meal consumed within the first pre-specified time period (e.g., over a day) is determined at a predetermined frequency (e.g., each day, every week, at the end of a sensor wear period, or any other predetermined frequency). The mean value is a value of the total of the meal excursions associated with each consumed over the first-pre-specified time period, divided by the number of meal excursions occurring over the first pre-specified time period. The mean value can be determined from a predetermined number of most recent one or more meal excursions consumed during the day.

At 14013, one or more high analyte level impact time periods and/or one or more low analyte level impact time periods within the first pre-specified time period are determined. Specifically, the mean value for each first pre-specified time period is compared to a mean value for a second pre-specified time period in order to identify whether the mean value for each first pre-specified time period is a high analyte level impact time period or low analyte level impact time period. More specifically, the second pre-specified time period is a longer time period than the first pre-specified time period. For example, the first pre-specified time period can be a one day time period, and the second pre-specified time period can be a one week time period. Those of skill in the art will appreciate that various other first pre-specified time periods and second pre-specified time periods can be utilized without departing from the scope of the present disclosure. The second pre-specified time period can include some or all of the first pre-specified time period. In some examples, the first pre-specified time period can be a period of time before or after the pre-specified time period.

At 14013, the mean value for each first pre-specified time period is compared with the mean value for the second pre-specified time period to determine whether there is a threshold different in value therebetween. Specifically, the threshold difference in value is determined by utilizing one or more of the following: (1) a calculated statistical “Z-score” for the first pre-specified time period; (2) a statistical “T-test”; (3) and a machine learning based-model. More specifically, if the threshold difference in value is such that the mean value for a first pre-specified time period is greater than the mean value for the second pre-specified time period, then the first-pre-specified time period is identified as a high analyte level impact time period (e.g., a high impact day). Additionally, if the threshold difference in value is such that the mean value for a first pre-specified time period is less than the mean value for the second pre-specified time period, then the first-pre-specified time period is identified as a low analyte level impact time period (e.g., a low impact day).

At 14014, the method can include providing, to a display device, a GUI configured for display of information related to the identified one or more high analyte level impact time period(s) and/or one or more low analyte level impact time period(s). According to some embodiments, the information related to the identified high analyte level impact time period(s) and/or low analyte level impact time period(s) can be displayed to the user as part of review, report, calendar, or insights interface. For example, the information related to the identified high analyte level impact time period(s) and/or low analyte level impact time period(s) can be presented to the user at the end of a predetermined period of time as part of a review interface. For example, the information related to the identified high analyte level impact time period(s) and/or low analyte level impact time period(s) can be presented to the user at the end of each week as part of a weekly review. In this regard, the user is encouraged to be mindful of meals that contributed to identified high analyte level impact time period(s) and commended on their choices on low analyte level impact time period(s). In some embodiments, the information related to the identified high analyte level impact time period(s) and/or low analyte level impact time period(s) can be displayed through insights interfaces, as will be described in further detail below. In some embodiments, the information can be presented as part of a “Best Day” insights card or a “Progress” insights card, as will be further described below. Though information related to the identified high analyte level impact time period(s) and/or low analyte level impact time period(s) has been described herein with reference to insights interfaces, those of skill in the art will appreciate that these examples are meant to be illustrative only and that the information related to the identified high analyte level impact time period(s) and/or low analyte level impact time period(s) can be implemented and/or displayed to the user through various other interfaces described herein, without departing from the scope of the present disclosure.

FIGS. 14A-1 through 14E are block diagrams depicting example embodiments of insights interfaces, or features related thereto, any of which can be utilized with the embodiments described herein. Referring to FIGS. 14A-1 to 14A-5, an example embodiment of a block diagram of an insights GUI 14000 for a TIR application is depicted. Insights GUI 14000 can be displayed when the user selects the insights icon home GUI 400 (FIG. 4B), home GUI 450 (FIG. 4D), home GUI 430 (FIGS. 4E-1 to 4E-7), home GUI 485 (FIGS. 4J-1 to 4J-3), home GUI 495 (FIGS. 4K-1 to 4K-3), home GUI 499 (FIGS. 4M-1 to 4M-6), or any of the interfaces described herein that can be configured as a home interface (e.g., GUI 12100, as shown in FIGS. 12D-1 to 12D-2, or GUI 13100, as shown in FIGS. 13C-1 to 13C-5). According to some embodiments, and as depicted in FIGS. 14A-1 to 14A-4, the insights GUI 14000 comprises: (1) an insights section 14100, which can include one or more insights cards 14400, 14500; (2) a top meals section 14200, which can include a list 14201 of meal information comprising the highest scored meal events 14202 ranked according to a score 14203 assigned based on the corresponding analyte response recorded; (3) a selectable home icon 14300; (4) a selectable insights icon 14301; (5) a selectable “+” icon 14302; and (6) a selectable settings icon 14303.

According to an aspect of the embodiments, and as best depicted in FIG. 14A-1, the insights section 14100 can comprise one or more insights cards 14400, 14500, including but not limited to, a “Best Day” insights card 14400 and a “Progress” insights card 14500, which can be based on the meal information and the data indicative of the analyte level. Specifically, the meal information and the data indicative of the analyte level (e.g., the glucose level) can be utilized to determine the user's progress for a predefined period of time, including but not limited to the user's “best” day (e.g., the day in which the user stayed in TIR the longest, had the highest daily score, or the highest average meal rating for a predefined period of time, for example, a one-week period). The user's progress is measured with respect to a particular criterion related to the user's meal information and data indicative of the analyte level (e.g., criterion related to TIR, daily score, average meal rating, etc.). The insights GUI is configured to insights cards 14400 and 14500 with statistics related to the user's progress.

Specifically, in some exemplar embodiments, the Best Day insights card 14400 can include a summary message 14401 with statistics detailing the user's “best” TIR day in a predefined period of time (e.g., one week), or what the user's highest TIR in a particular day was for the predefined period of time (e.g., “From February 6-12, your highest TIR in a day was Friday, February 11 with 16 Hours in Range.”). According to some embodiments, the message 14401 can indicate the user's highest TIR in terms of hours or percentage values.

In some embodiments, if the user did not go above zero hours in range, then the Best Day insights card 14400 will display the message 14401, wherein the message 14401 indicates that the user had zero hours in range (e.g., “From February 20-26, your highest TIR in a day was Friday, February 25 with 0 hours in Range”).

In some embodiments, the Best Day insights card 14400 can include the summary message 14401 detailing the user's best day within a predefined period of time, wherein the user received the highest daily score (e.g., “From February 6-February 12, the day with the highest score was Wednesday, February 9 with 87 points”).

In some embodiments, the Best Day insights card 14400 can include the summary message 14401 detailing the user's highest average meal rating day within the predefined period of time (e.g., “From January 30-February 5, the day with the highest rating was Wednesday February 2 with an average rating of 4.8”).

In some embodiments, the Best Day insights card 14400 is displayed on insights GUI 14000 after the predefined period of time has elapsed (e.g., a one-week or seven-day time period). For example, after a seven-day time period has elapsed (for example, on every Sunday of every week), a Best Day insights card 14400 is displayed on insights GUI 14000, wherein the Best Day insights card 14400 comprises the message 14401. In some embodiments, the insights GUI 14000 is configured to update such that the displayed Best Day insights card 14400 is replaced with a new Best Day insights card 14400 at a predefined frequency or after every subsequent predefined period of time has elapsed (e.g., every Sunday or every seven days a new Best Day insights card 14400 is displayed on insights GUI 14000). In this regard, the insights GUI 14000 is configured to update so as to display to the user the latest Best Day insights card 14400 corresponding to the latest predefined period of time.

In some embodiments, if there is no recent Best Day insights card 14400 (e.g., if the user intermittently monitors analyte data, or if a predefined period of time has elapsed without the user monitoring analyte data for that respective time or without the TIR application determining the user's progress, then the insights GUI 14000 is configured to display the most current Best Day insights card 14400 (e.g., a Best Day insights card 14400 from one month ago if the user last monitored analyte data one month ago).

In some embodiments, though not illustrated, when a new Best Day insights card 14400 is available, the “insights” icon displayed on any interface outputted by the TIR application is configured to provide an animation to indicate to the user that a new insights card 14400 is available. For example, in some embodiments, the insights icon is configured to change color (e.g., comprise a colored portion, such as a blue color), to indicate that the new insights card is available.

In some exemplar embodiments, and as best shown in FIGS. 14A-1 and 14A-4, the Best Day insights card 14400 can further include a time-of-insight indicator 14402 which is configured to indicate the amount of time elapsed since the Best Day insights card 14400 was displayed on insights GUI 14000 (e.g., “1 MIN AGO,” “1 WEEK AGO,” etc.). As shown in FIG. 14A-4, in some embodiments, the Best Day insights card 14400 can further include a “New” tag 14403 which indicates to the user that the Best Day insights card 14400 has been newly added to the insights GUI 14000 or has not yet been viewed by the user. In some embodiments, the “New” tag 14403 is removed from the Best Day insights card 14400 in response to a seventh predetermined gesture by the user, such as a tap gesture, or some other predetermined gesture. In some embodiments, the “New” tag 14403 is automatically removed from the Best Day insights card 14400 after a predetermined period of time has elapsed (e.g., after 24-hours).

In some embodiments, the insights GUI 14000 is configured to display the insights section 14100 comprising only the Best Day insights card 14400 (see, e.g., FIG. 14A-5). In other embodiments, and as depicted in FIGS. 14A-1 and 14A-4, the insights GUI 14400 can display the insights section 14100 comprising both the Best Day insights card 14400 and the progress insights card 14500 (see, e.g., FIGS. 14A-1 and 14A-4).

According to some embodiments, and with reference to FIGS. 14A-1 and 14A-4, the Progress insights card 14500 can include a summary message 14501 detailing the user's TIR or daily score progress over the predefined period of time (e.g., “From February 6-12, your average Time In Range was 2 hours higher than it was from January 30-February 5,” or “From February 6-12, your average daily score was 4 points higher than it was from January 30-February 5.”). In some embodiments, the TIR application is configured to compare the user's average TIR or daily score for a recent predefined period of time with the user's average TIR or daily score for a previous predefined period of time (e.g., the user's average TIR or daily score for the last seven days is compared with the user's average TIR or daily score the preceding seven days). According to some embodiments, the message 14501 can comprise indicate the user's TIR Progress in terms of hours or percentage values.

In some embodiments, the TIR application is configured to compare the user's average TIR or daily score for a recent predefined period of time with the user's average TIR or daily score for the first week of using the TIR application. In some embodiments, the Progress insights card 14500 is displayed only after the TIR application has the user's average TIR or daily score for two predefined periods of time so as to make a progress comparison. In some embodiments, the insights GUI 14000 is configured to update such that the displayed progress insights card 14500 is replaced with a new progress insights card 14500 at a predefined frequency following the display of the first progress insights card 14500 (e.g., every Sunday or every seven days a new progress insights card 14500 is displayed on insights GUI 14000). In this regard, the insights GUI 14000 is configured to update so as to display the user's latest progress (e.g., the user's latest TIR progress).

In some embodiments, if there is no recent Progress insights card 14500 (e.g., if the user intermittently monitors analyte data, or if a predefined period of time has elapsed without the user monitoring analyte data for that respective time, then the insights GUI is configured to display the most current Progress insights card 14500 (e.g., a Progress insights card 14500 from one month ago).

In some embodiments, when a new progress insights card 14500 is available, the “insights” icon on the interface outputted by the TIR application is configured to provide an animation to indicate to the user that a new insights card 14500 is available. For example, in some embodiments, the insights icon is configured to change color (e.g., comprise a colored portion, such as a blue color), to indicate that the new insights card 14500 is available.

In some aspects of the embodiments, if the user's average TIR is equal to or lower than the user's average TIR for the first week of using the TIR application, the Progress insights card 14500 comprising the user's TIR progress is not displayed on the insights GUI 14000.

In some exemplar embodiments, and as shown in FIGS. 14A-1 and 14A-4, the Progress insights card 14500 can further include a time-of-insight indicator 14502 which is configured to indicate the amount of time elapsed since the Progress insights card 14500 was displayed on insights GUI 14000 (e.g., “1 MIN AGO,” “1 WEEK AGO,” etc.). Further, in some embodiments, and as best depicted in FIG. 14A-1, the Progress insights card 14500 can further include a “New” tag 14503 which indicates to the user that the Progress insights card 14500 has been newly added to the insights GUI 14000 or has not yet been viewed by the user. In some embodiments, and as best depicted in FIG. 14A-4, the “New” tag 14503 is removed from the Progress insights card 14500 in response to an eighth predetermined gesture by the user, such as a tap gesture, or some other predetermined gesture. In some embodiments, the “New” tag 14503 is automatically removed from the Progress insights card 14500 after a predetermined period of time has elapsed (e.g., after 24-hours).

With reference to FIGS. 14A-1 to 14A-4, In some embodiments, the insights GUI 14000 comprises a “Past Insights” link 14600 which, upon being selected by the user, outputs a past insights GUI 14650, as shown in FIGS. 14B-1 to 14B-3. As best shown in FIG. 14B-1, the past insights GUI 14650 is configured to display an insights log 14651, wherein the insights log 14651 can include historical Best Day insights cards 14400 and historical Progress insights cards 14500, along with the most recent Best Day insights card 14400 and most recent Progress insights card 14500. Specifically, in some embodiments, the insights log 14651 is configured to display the Best Day insights cards 14400 and/or Progress insights card 14500 in chronological order, wherein the most recent Best Day insights card 14400 and/or most recent Progress insights card 14500 is displayed at a top portion of the insights log 14651. As shown in FIG. 14B-3, the past insights GUI 14650 can be configured to display insights log 14651 comprising only Best Day insights cards 14400 (e.g., historical Best Day insights cards 14400 and the most recent Best Day insights card 14400).

In some embodiments, if the user can select a particular Best Day insights card 14400 or Progress insights card 14500 from the past insights GUI 14650, then either a report GUI 600 (e.g., FIGS. 6B-1 and 6B-2) associated with the particular day corresponding to the Best Day insights card 14400 or Progress insights card 14500 is outputted, or a home interface corresponding to the past day is outputted.

According to an aspect of the embodiments, the past insights GUI 14650 is configured to display Best Day insights cards 14400 and Progress insights cards 14500 for periods of relevant time for which the TIR application has data. For example, if data required to output the Best Day insights card 14400 and/or Progress insights card 14500 is missing for a time period Feb. 6, 2022 and Mar. 26, 2022, then the past insights GUI 14650 can display historical Best Day insights cards 14400 and Progress insights cards 14500 for Mar. 27, 2022 through Apr. 2, 2022, and historical Best Day insights cards 14400 and Progress insights cards 14500 for Jan. 30, 2022 through Feb. 5, 2022.

In some embodiments, and as shown in FIG. 14B-2, if no insights are available yet (e.g., a first predefined period of time has not yet elapsed for a Best Day insights card 14400 to be displayed, or a first recent predefined period of time has not yet elapsed which can be compared to a previous predefined period of tip for a Progress insights card 14500), the past insights GUI 14650 will display a message 14652 indicating to the user that no insights are available yet. Further in some embodiments, the past insights GUI 14650 can further inform the user that insights become available when there is data from the previous week. In some embodiments, and as depicted in FIGS. 14B-1 and 14B-2, the past insights GUI 14650 can include an informational icon 14653 which, upon being selected by the user, outputs an informational modal 14700 (FIG. 14C) which informs the user that insights can appear at a predetermined frequency (e.g., every Sunday of each week, or every seven days) when there is analyte (e.g., glucose) data from a previous predefined period of time (e.g., the previous week), and that the user can choose to receive notifications when new insights cards are posted. Further, in some embodiments, the informational modal 14700 can inform the user that the user can choose to receive notifications when new insights are posted, and that past insights from other predetermined periods of time (e.g., past weeks) are saved and can be reviewed at any time. In some embodiments, the informational modal 14700 notes that the longer the user uses continuous glucose monitoring and the TIR application, the more insights will be available. In some embodiments, the informational modal 14700 comprises a selectable notification preferences link 14701.

As best depicted in FIG. 14B-1, in some embodiments, the insights log 14651 is divided into subsections 14654, wherein each subsection 14654 represents a different predetermined period of time. For example, the insights log 14651 can be divided into subsections 14654 of seven-day or one-week periods. In this regard, the Best Day insights card 14400 and Progress insights card 14500 for each seven-day or one-week period is displayed in the corresponding subsection 14654 of the insights log 14651. For example, the Best Day insights card 14400 and Progress insights card 14500 corresponding to Feb. 6, 2022 through Feb. 12, 2022 is displayed in the subsection 14654 pertaining to the Feb. 6, 2022 through Feb. 12, 2022 time period. The insights log 14651 can be configured to be scrollable and/or dynamic. As such, in some embodiments, the content displayed on the insights log 14651 can vary in response to an twelfth predetermined input by the user, such as when the user scrolls, drags, pulls the screen, or by some other predetermined gesture. In this manner, the past insights GUI 14650 can comprise a plurality of views, wherein each view is configured to display a portion of past insights GUI 14650 to the user, wherein different Best Day insights cards 14400 and Progress insights cards 14500 can be displayed depending on the view.

According to an aspect of the embodiments, the past insights GUI 14650 can include an insights availability message 14655 (FIGS. 14B-1 and 14B-2) indicating to the user that insights become available when there is data from the previous week.

Turning back to FIGS. 14A-1 to 14A-3, and according to another aspect of the embodiments, the top meals section 14200 of the insights GUI 14000 can list the highest scored meal events 14202 logged in a predetermined time period (e.g., one or more weeks, two weeks, multiple consecutive weeks, etc.). In the meal list 14201, and with reference to FIGS. 14A-1 and 14A-3, the meals can be ordered from highest score 14203 to the lowest score 14203, in descending order of an assigned numerical value, wherein the score 14203 is the assigned numerical value that corresponds to an analyte response based on the data indicative of the analyte level for the user. In some embodiments, the analyte response can be based on a change in an analyte level within a predetermined time period after the particular meal is consumed. In some embodiments, meal events 14202 are given a score 14203 based on the change in analyte level from the time the particular meal was consumed to the peak glucose within three hours of eating. In some embodiments, the top meals section 14200 is configured to list a predefined number of the highest scored meal events 14202 logged in a predetermined time period (e.g., the top meals section 14200 is configured to list up to three of the highest scored meal events 14202 or the top three highest scored meal events 14202 logged in a predetermined time period).

In some embodiments, the score 14203 can be a number between one and five, wherein a higher assigned numerical value corresponds to a lower analyte response, and a lower assigned numerical value corresponds to a higher analyte response. For example, and as previously detailed, (1) a 0 to 24 mg/dL change in an analyte level within a three hour period post-meal can correspond to a “5” score 14203; (2) a 25 to 49 mg/dL change in an analyte level within a three hour period post-meal can correspond to a “4” score 14203; (3) 50 to 74 mg/dL change in an analyte level within a three hour period post-meal can correspond to a “3” score 14203; (4) a 75 to 99 mg/dL change in an analyte level within a three hour period post-meal can correspond to a “2” score 14203; and (5), a 100 mg/dL or higher change in an analyte level within a three hour period post-meal can correspond to a “1” score 14203.

As depicted in insights GUI 14000 of FIGS. 14A-1 and 14A-3, the score 14203 can be displayed in a graphical element, such as a star icon 14204. In some embodiments, the star icon 14204 is filled with a colored portion, such as yellow. In many embodiments (not illustrated), the star icon 14204 is unfilled and comprises a broken outline to indicate a missing meal score 14203. In some embodiments, though not illustrated, a missing meal score 14203 is indicated by a “?” character. In some embodiments, the “?” character is displayed in the star icon 14204 instead of a numerical value so as to represent that a particular meal was not scored. A missing meal score 14203 happens when one or both of the following occurs: (1) the user logs the next meal too soon, or (2) the time-in-range application does not have sufficient analyte data to score the meal (e.g., the user has not scanned in a timely manner so as to ensure glucose data has been transmitted to the time-in-range application).

With reference to FIGS. 14A-1 and 14A-3, and according to another aspect of the embodiments, each of the meal events 14202 listed on the insights GUI 14000 provides details on a particular meal consumed within the predetermined time period (e.g., one or more weeks, two weeks, multiple consecutive weeks, etc.). In some embodiments, and as best shown in FIG. 14A-3, each meal event 14202 can include a text description 14205 of a meal, photograph or picture 14206 associated with the meal, a portion size indicator 14207 describing the relative portion size of the meal, and/or the score 14203 associated with the meal. In some embodiments, and as best shown in FIG. 14A-3, a meal event 14202 can additionally or alternatively include one or more meals tags 14208 associated with the meal (e.g., “lettuce,” “blueberries,” “cheese”).

According to an aspect of the embodiments, and with reference to FIG. 14A-3, when meals have been inputted and scored but no insights are yet available, the insights GUI 1400 is configured to display the insights section 14100 comprising a message 14101 indicating the no insights are available yet, and the top meals section 14200 comprising the list 14201 of meal information.

According to another aspect of the embodiments, and as shown in FIG. 14A-4, when meals have not yet been inputted or scored, but insight are available, the insights GUI 14000 is configured to display the insights section 14100 comprising the Best Day insights card 14400 and/or the Progress insights card 14500, and the top meals section 14200 comprising a message 14210 indicating that no meals have been scored yet.

In some embodiments, and as shown in FIG. 14A-4, when meals have not yet been inputted or logged by the user, the insights GUI 14000 can comprise an insights section 14100 comprising the Best day insights card 14400 and/or the Progress insights card 14500, and the top meals section 14200 comprising the message 14210 indicating that no meals have been scored yet.

Further, according to an aspect of the embodiments, each of the meal events 14202 listed in the top meals section 14200 of insights GUI 14000 is selectable and, upon being selected by the user, output a meal review GUI 800 or meal review GUI 8000 (FIG. 8A and FIGS. 8B-8E, respectively) providing details related to the selected meal event 14202. In some embodiments (though not illustrated in FIG. 8A or 8B-8E), meal review GUI 800 can include a “back” button which, upon being selected by the user, outputs the previous interface from which the particular meal event corresponding to the meal review GUI 800 or meal review GUI 8000 that was selected.

With reference to FIGS. 14A-1 to 14A-4, insights GUI 14000 can further include an “All Meals” link 14250 which, upon being selected by the user, outputs a meals GUI 14800 (as shown in FIGS. 14D-1 through 14D-3) which provides additional insights to the user as to the impact of consuming particular foods. According to an aspect of the embodiments, and with reference to FIGS. 14D-1 to 14D-2, the meals GUI 14800 comprises a list 14801 of meal information comprising all meal events 14802 logged in the same predetermined time period (e.g., one or more weeks, two weeks, multiple consecutive weeks, etc.) as the meal events 14202 logged in the insights GUI 14000. For example, if the top meals section 14200 of insights GUI 14000 displayed the highest scored meal events 14202 logged in a two-week time period, then meals GUI 14800 is configured to display a list 14801 of all meal events 14802 which were logged in the two-week time period. Specifically, and similar to the meal events 14202 listed in the top meals section 14200 of insights GUI 14000 (FIGS. 14A-1 and 14A-3), the meal events 14802 on meal GUI 14800 are ranked according to a score 14803 assigned based on the corresponding analyte response recorded. In some embodiments, in the meal list 14801, the meals can be ordered from highest score 14803 to the lowest score 14803, wherein the score 14803 is an assigned numerical value that corresponds to an analyte response based on the data indicative of the analyte level for the user. In some embodiments, when one or more meals are given a same score 14802, the meals can be ordered by analyte level variance in the meal list 14801. For example, if two meals were provided a score 13802 of “3,” then the meals could be ordered from the lowest PeakDelta or analyte level variance value to the highest PeakDelta or analyte level variance value. Further, in some embodiments, if one or more meals are provided the same score 14802 and a same analyte level variance, then the meals can be ordered chronologically.

In some embodiments, and as best shown in FIG. 14D-2, when meals have been inputted by not yet scored, each unscored meal event 14802 displayed on the list 14801 of meal information either comprises (1) an unfilled star icon 14804 comprising a broken outline to indicate the missing meal score or, in some instances, or (2) no star icon to indicate the missing meal score.

In some embodiments, and as best shown in FIG. 14D-3, when meals have yet to be inputted or logged by the user, the meals GUI 14800 displays a message 14805 indicating to the user that no meals have been scored yet. Further, in some embodiments, the meals GUI 14800 can further inform the user that higher ratings mean higher likelihood of staying in target range.

In some embodiments, and as shown in FIGS. 14D-1 through 14D-3, meals GUI 14800 further comprises an informational icon 14806 which, upon being selected, outputs a meal scoring informational modal comprising information on how a meal is ranked or scored in the TIR application. In some embodiments, the meal scoring information modal comprising information on how a meal is ranked can further comprise emoticons to indicate the impact on glucose.

According to another aspect of the embodiments, and similar to the meal events 14202 listed on the insights GUI 14000 (as best shown in FIGS. 14A-1 and 14A-3), each of the meal events 14802 listed on the meals GUI 14800 provides details on a particular meal consumed within the predetermined time period (e.g., one or more weeks, two weeks, multiple consecutive weeks, etc.). In some embodiments, and as best shown in FIG. 14D-1, each meal event 14802 on meal GUI 14800 can include a text description 14815 of a meal, a photograph or picture 14816 associated with the meal, a portion size indicator 14817 describing the relative portion size of the meal, and/or the score 14803 associated with the meal. In some embodiments, a meal event 14802 can additionally or alternatively include one or more meals tags 14818 associated with the meal (e.g., “lettuce,” “blueberries,” “cheese”).

According to an aspect of the embodiments, and in response to a ninth predetermined gesture by the user, such as when the user pulls down or scrolls on the meals GUI 14800, or by some other predetermined gesture, the user can scroll through the meals GUI 14800 such that they can scroll through different meals events 14802 listed thereon.

In some embodiments, and still with reference to FIG. 14D-1 to 14D-3, the meals GUI 14800 can comprise an informational message 14807 indicating to the user that a higher score means a higher likelihood of staying in target range. Additionally, meals GUI 14800 can further include a back button 14809 which, upon being selected by the user, outputs insights GUI 14000 (see, e.g., FIG. 14A-1 to 14A-4).

Further, according to an aspect of the embodiments, each of the meals events 14802 listed in the list of meals GUI 14800 are selectable and, upon being selected by the user, output a meal review GUI 800 or meal review GUI 8000 (FIGS. 8A and FIGS. 8B-8E, respectively) providing details related to the selected meal event 14802. In some embodiments, though not illustrated in FIG. 8A or FIG. 8B-8E, meal review GUI 800 and meal review GUI 8000 can include a “back” button which, upon being selected by the user, outputs the previous interface from which the particular meal event corresponding to the meal review GUI 800 was selected.

According to an aspect of the embodiments, a notification 14900 related to insights can be presented as a pop-up window or a banner notification 14900 displayed to the user outside of the TIR application (e.g., on a lock screen), as seen in FIG. 14E. In some exemplar embodiments, when a new insight card is available, the notification 14900 will be displayed comprising a message notifying the user that the user's data has been analyzed over a predefined time period (e.g., the last week). Further, in some embodiments, the notification 14900 can prompt to user to tap the notification to view the newly available insights card.

FIGS. 15A-1 through 15A-6 are block diagrams depicting additional example embodiments of insights interfaces, or features related thereto, any of which can be utilized with the embodiments described herein. Referring to FIGS. 15A-1 to 15A-6, an example embodiment of a block diagram of an insights GUI 15000 for a TIR application is depicted. Insights GUI 15000 can be displayed when the user selects the insights icon home GUI 400 (FIG. 4B), home GUI 450 (FIG. 4D), home GUI 430 (FIGS. 4E-1 to 4E-7), home GUI 485 (FIGS. 4J-1 to 4J-3), home GUI 495 (FIGS. 4K-1 to 4K-3), home GUI 499 (FIGS. 4M-1 to 4M-6), or any of the interfaces described herein that can be configured as a home interface (e.g., GUI 12100, as shown in FIGS. 12D-1 to 12D-2, or GUI 13100, as shown in FIGS. 13C-1 to 13C-5). According to some embodiments, the insights GUI 15000 comprises: (1) an average meal score card 15001; (2) a meals section 15002, which can include a list of meal events 15003 and/or notifications 15004 corresponding to one or more missed meals for a predetermined period of time (e.g., the last 24-hours); (3) a selectable home icon 15005; (4) a selectable insights icon 15006; (5) a selectable “+” icon 15007 which, upon being selected, outputs meal logging GUI 500 (FIG. 5A); and/or (6) a selectable settings icon 15008.

According to another aspect of the embodiments, and as best depicted in FIGS. 15A-1, 15A-2, and 15A-4, the average meal score card 15001 can comprise: (1) a message 15010 (e.g., in some embodiments, the message 15010 indicates to the user that the total average meal score is based on the meals the user has had in a predetermined period of time, such as the last 24 hours); (2) a status indicator 15011, wherein the status indicator 15011 summarizes the total average meal score (e.g., “Poor,” “Fair,” “Neutral,” “Medium,” and “Excellent”); and/or (3) a graphical representation 15012 corresponding to a total average meal score of the meal events 15003 and scored detected missed meals, collectively, for a predetermined period of time (e.g., for the preceding or trailing 24-hours). In some embodiments, the total average meal score is determined for a predetermined frequency of time. In this manner, the total average meal score can be determined in light of the meal events 15003 and scored detected missed meals for a trailing 24-hour period. As such, in some instances, the total average meal score can be calculated in light of the meal events 15003 and scored detected missed meals from a previous day and a current day (e.g., if it is 5 P.M., then the total average meal score will consider all meal events 15003 and scored detected missed meals within the last preceding 24-hour period, up until 5 P.M. the previous day). Additionally, in this regard, the total average meal score can continuously update so as to account for the meal events 15003 and scored detected missed meals within a new cycle of a predetermined frequency of time. For example, if the time changes from 4:59 PM to 5 P.M., then the total average meal score can update so as to remove all meal events 15003 and scored detected missed meals that were accounted for at 4:59 PM the previous day and account for new meal events 15003 and scored detected missed meals that occurred at 5 P.M. today.

In some embodiments, the TIR application is configured to automatically score detected missed meals even if the user has not yet logged the meal, wherein the total average meal score will be calculated in light of the automatically scored detected missed meal. In other embodiments, the TIR application is configured to automatically score detected missed meals even if the user has not yet logged the meal, wherein the total average meal score will be calculated without the automatically scored detected missed meal. In yet other embodiments, the TIR application is configured to not score the detected missed meal(s) until the user has successfully logged the meal, wherein the total average meal score will be calculated in light of the newly logged meal only after it is successfully logged by the user.

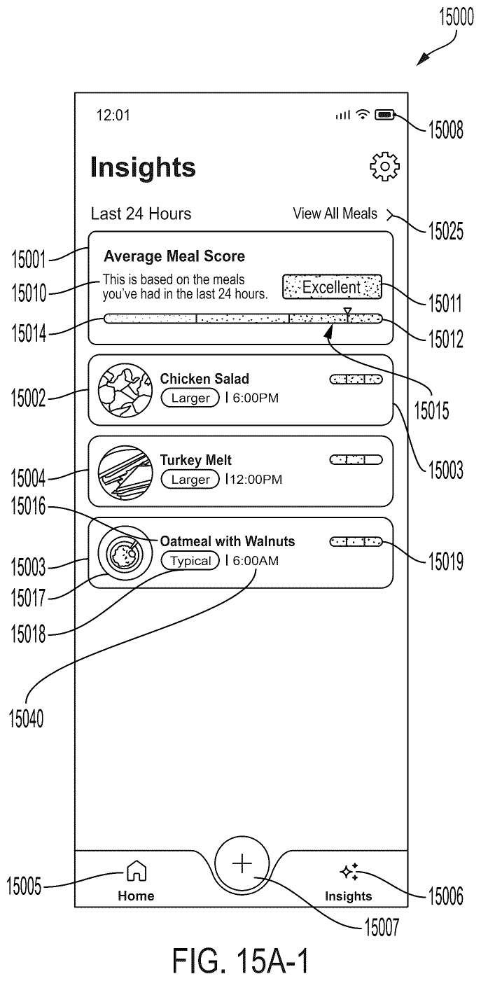

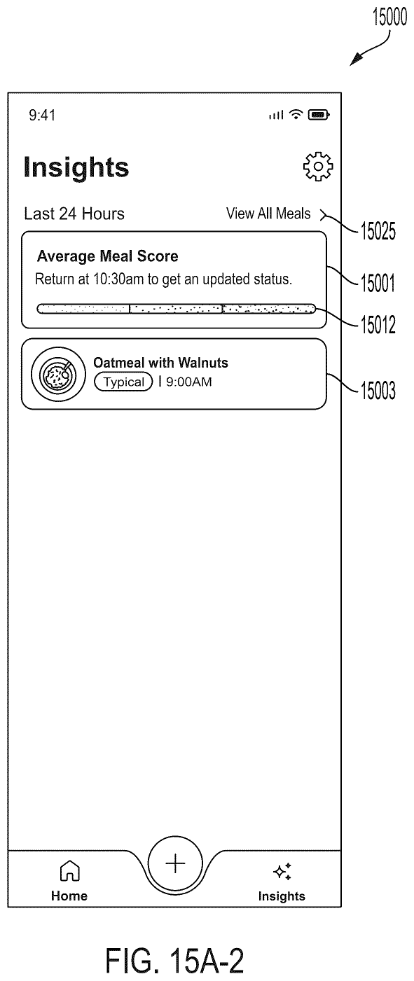

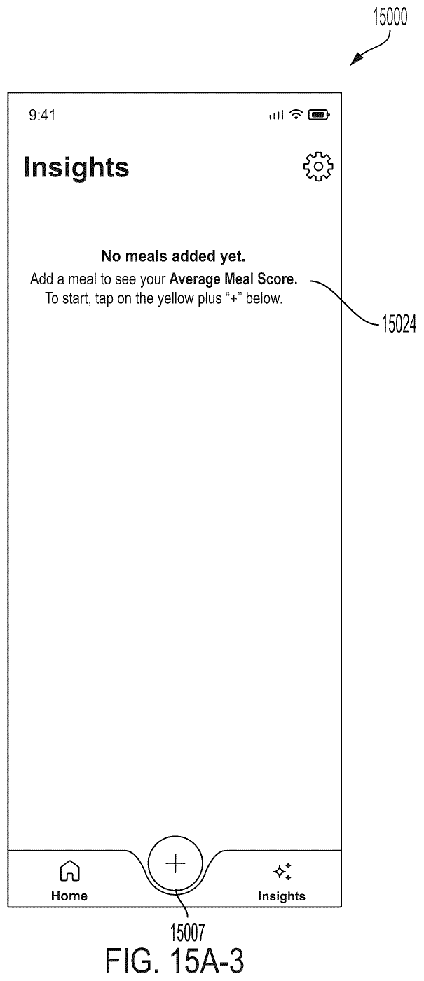

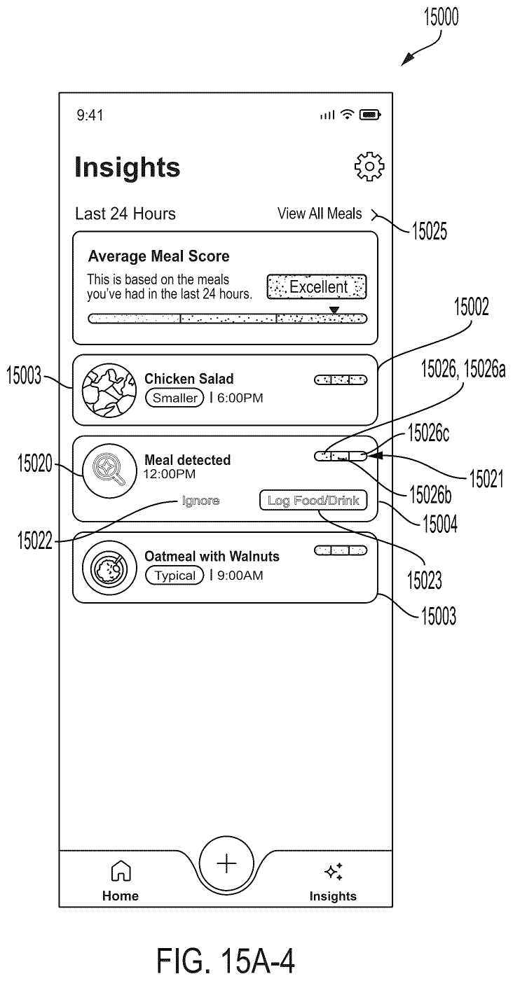

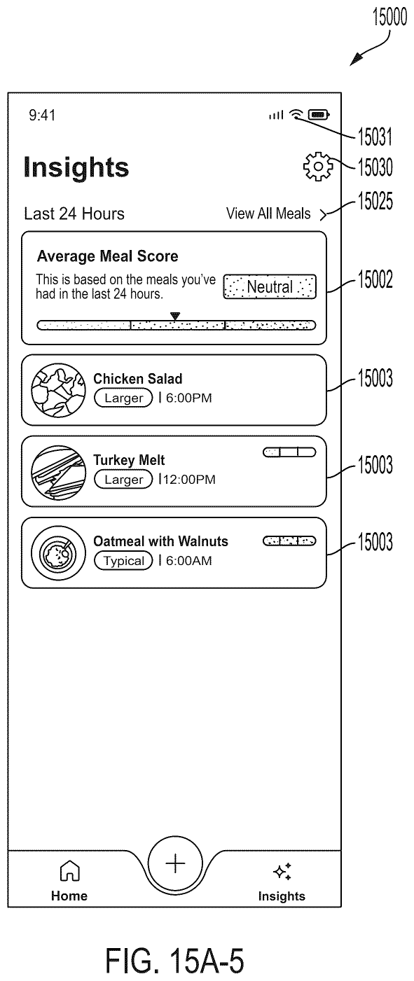

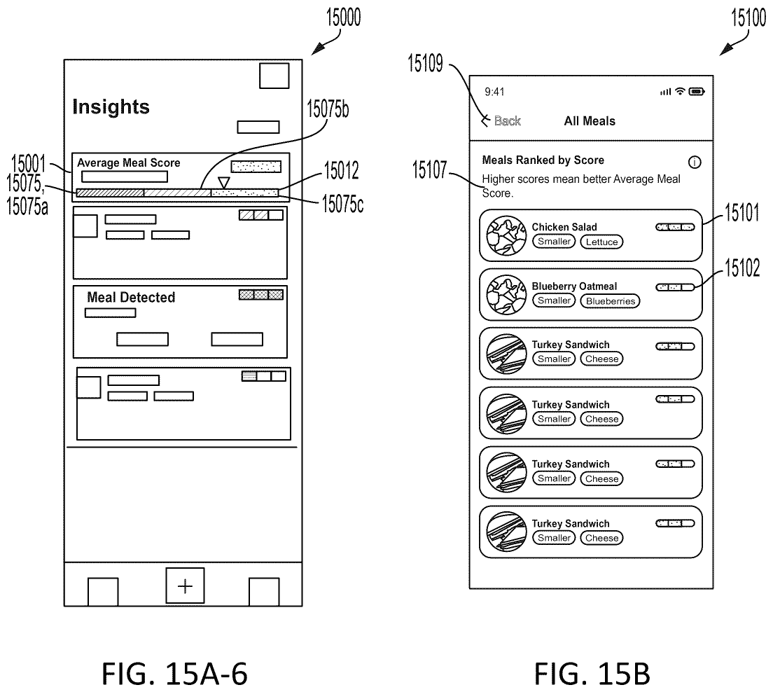

Further, and according to some embodiments, and with reference to FIG. 15A-1, the graphical representation 15012 can be a gradient scale 15012. The gradient scale 15012 can include a color gradient 15014, wherein each color of the color gradient 15014 corresponds to a different status of the total average meal score (e.g., red/red-orange color represents poor status, orange color represents fair status, yellow color represents neutral status, dark-yellow/light-green color represents medium status, and green color represents excellent status). According to some embodiments, the total average meal score can be a number between one and 100. In this manner, the gradient scale 15012 can represent a total average meal score range between one and 100. In some embodiments, the gradient scale 15012 comprises a picker 15015 configured to align with a portion of the color gradient 15014 corresponding to the total average meal score so as to visually identify for the user the total average meal score (e.g., the picker 15015 can be aligned with the yellow color of the color gradient 15014 if the total average meal score is in a neutral status). In some embodiments, the status indicator 15011 comprises a colored or shaded portion, wherein the color or shaded portion corresponds to the color the picker 15011 is aligned with on the color gradient 15014 so as to visually illustrate to the user the status of the total average meal score. In other embodiments, and as best depicted in FIG. 15A-6, the average meal score card 15001 includes a graphical representation 15012 with a segmented or tiered bar with a plurality of tier portions 15075. Specifically, in some embodiments, the plurality of tier portions 15075 can comprise three tier portions 15075, wherein: (1) the first tier 15075a can have a red color or orange color and be indicative of a poor status or fair status; (2) the second tier 15075b can have a yellow color and be indicative of a neutral status or medium; and (3) the third tier 15075c can have a green color and be indicative of a good or excellent status. In other embodiments, though not illustrated, the plurality of tier portions 15075 can comprise five tier portions, wherein each of the tier portions is a different shade of color and indicative of a different status. Those of skill in the art will appreciate that various other scoring ranges, graphical representations, gradients, color gradients, colors, and configurations can be utilized to represent the total average meal score without departing from the scope of the disclosure.

According to another aspect of the embodiments, and with reference to FIGS. 15A-1, 15A-2, and 15A-4, the meals section 15002 can include the list of meal events 15003, wherein each meal event can comprise a text description 15016 of a meal, a photograph or picture 15017 associated with the meal, a portion size indicator 15018 describing the relative portion size of the meal, a timestamp 15040 indicating the time associated with the logged meal, and/or a graphical representation 15019, such as a segmented bar 15019, representing a score associated with the logged meal.

In some embodiments, and as shown in FIG. 15A-2, if a meal has been logged but has not yet been provided an individual score, the corresponding meal event 15003 for the logged meal will not display a graphical representation 15019, such as the segmented bar 15019. Specifically, and as shown in FIG. 15A-2, if no scores are provided for logged meals or detected missed meals provided on the meals section 15002, then the average meal score card 15002 can display the message 15010, wherein the message 15010 indicates when the user can return for an updated status on the total average meal score (e.g., three hours from the time of the meal logging). For example, in some embodiments, the message 15010 can state that the user should “Return at 10:30 am to get an updated status.”) Further, if no scores are provided for logged meals or detected missed meals, then the respective meal event(s) 15003 and/or notification(s) 15004 can be displayed without a graphical representation (e.g., segmented bar 15019 or segmented bar 15021) shown on the corresponding meal event(s) 15003 and/or notification(s) 15004. Additionally, in some embodiments, and as depicted in FIG. 15A-2, if no scores are provided for logged meals or detected missed meals, then the average meal score card 15002 can be displayed without a picker or status indicator. In some embodiments, if a total average meal score is not yet available, then the average meal score card 15002 can display a graphical representation 15012, wherein the graphical representation comprises a faded color portion so as to indicate that the total average meal score is not available.

In some embodiments, and with reference to FIG. 15A-3, when no meals have been logged and no missed meals have been detected for a predetermined period of time reflected by the insights GUI, then the insights GUI 15000 will not display the average mal score card or the meals section 15002. Rather, in some embodiments, when no meals have been logged or missed meals have been detected, the insights GUI 15000 comprises a message 15024 indicating to the user that no meals have been added yet. In some embodiments, the message 15024 can further inform the user that they can add a meal to see the total average meal score. Further, in some embodiments, the message 15024 can inform the user that they can start adding meals by selecting the “+” icon 15007. In some embodiments, when no meals have been logged or missed meals have been detected, the all meals link will not appear on insights GUI 15000.

Further, in some embodiments, and with particular reference to FIG. 15A-4, each of the notifications 15004 can include a timestamp 15020 indicating a time associated with the detected meal (e.g., “9:00 AM”). Further, in some exemplar embodiments, each of the notifications 15004 can include a graphical representation 15021, such as a segmented bar 15021, representing a score associated with the detected meal. Specifically, the score can be represented by a segmented bar 15021 with three segment portions 15026, wherein: (1) the first segment 15026a can have a red color or orange color and be indicative of an analyte score corresponding to a high glucose impact or response (e.g., an average glucose range exceeding 250 mg/dL); (2) the first segment 15026a and second segment 15026b can have a yellow color and be indicative of an analyte score corresponding to a medium glucose impact or response (e.g., an average glucose range between 181 mg/dL and 250 mg/dL, or no greater than 250 mg/dL and/or at least 181 mg/dL); or (3) the first segment 15026a, the second segment 15026b, and the third segment 15026c can have a green color and be indicative of an analyte score corresponding to a low glucose impact or response (e.g., an average glucose range between 70 mg/dL and 180 mg/dL, or no greater than 180 mg/dL). Those of skill in the art will appreciate that various other graphical representations and colors, and color configurations can be utilized with the graphical representations without departing from the scope of the disclosure.

According to another aspect of the embodiments, and still with particular reference to FIG. 15A-4, each of the notifications 15004 can include a selectable “ignore” button 15022 and a selectable meal logging button 15023. Specifically, upon the user selecting the ignore button 15022 on a particular notification 15004, the notification 15004 is removed from the display of Insights GUI 15000. In some embodiments, removing the notification 15004 removes the detected missed meal corresponding thereto and, as such, removes the deleted missed meal from the total average meal score. In this manner, the total average meal score can update to account for the removal of the deleted detected missed meal. Specifically, the total average meal score will update so as to reflect the deletion of the detected missed meal. More specifically, if the total average meal score had initially accounted for the detected missed meal, then the total average meal score will be recalculated so as to remove the deleted detected missed meal from the total average meal score. Further, in some embodiments, if the user selects the meal logging button 15023, meal logging GUI 500 (FIG. 5A) is outputted. Moreover, in some embodiments, meal entry modal 520 (as shown in FIG. 5C) can appear, wherein the time field 527 is prepopulated with a date and time associated with the detected missed meal associated with the notification 15004 from which the meal logging button 15023 was selected. In other embodiments, meal entry modal 520 (as shown in FIG. 5C) can appear, wherein the user sets the date and time associated with the detected missed meal in the time field 527. In some embodiments, when meal logging GUI 500 is outputted through the user selecting the meal logging button 15023 of insights GUI 15000, a camera interface (e.g., FIGS. 5I to 5N) is not shown to the user. Further, in some embodiments, one the user has logged a meal for the particular time associated with the meal detection, the respective notification 15004 will be replaced with a meal event 15003 corresponding to the logged meal (see, e.g., FIG. 15A-5).

In some embodiments, and with reference to FIG. 15A-5, once a user has successfully logged a meal, an in-app banner 15030 can be displayed on insights GUI 15000, indicating that a meal was added. Additionally, the in-app banner 15030 can comprise a selectable “View Meal” link 15031 which, upon being selected, outputs a meal review GUI 800 or meal review GUI 8000 (FIGS. 8A and 8B, respectively) corresponding to the newly logged meal. Further, in some embodiments, and upon the user editing a logged meal entry, the in-app banner 15030 can be displayed on insights GUI 15000, wherein the in-app banner 15030 indicates to the user that a meal was updated. In some embodiments, when a meal is updated, the in-app banner 15030 can further include a “View Meal” link 15031 which, upon being selected, outputs a meal review GUI 800 or meal review GUI 8000 (FIGS. 8A and 8B, respectively) corresponding to the updated logged meal.

According to another aspect of the embodiments, and as best shown in FIGS. 15A-1, 15A-4, and 15A-5, the meals section 15002 can include the list of meal events 15003 and/or notifications 15004 corresponding to one or more missed meals for a predetermined period of time (e.g., the last 24-hours), wherein the meal events 15003 and/or notifications 15004 are listed in chronological order. In some embodiments, the meals section 15002 is configured to update continuously so as to account for meal events 15003 and/or notifications 15004 for the predetermined period of time. Specifically, the meals section 15002 can continuously update at a predetermined frequency (e.g., every minute) to reflect new meal events 15003 and new notifications 15004 for a subsequent predetermined period of time. For example, the meals section 15002 can be configured to account for meal events 15003 and/or notifications 15004 of the preceding 24-hour time period. In this regard, the meals section 15002 can be updated every minute. As such, if a displayed meal event 15003 includes a timestamp from yesterday at 3:55 P.M., then at 3:56 P.M. on the current day, the meal event 15003 will be removed from the meals section 15002. Additionally, in this example, any new meal events 15003 and/or notifications 15004 that occurred at 3:56 P.M. will also be displayed on the meals section 15002.

With reference to FIGS. 15A-1, 15A-2, 15A-4, and 15A-5, insights GUI 15000 can further include an “All Meals” link 15025 which, upon being selected by the user, outputs a meals GUI 15100 (as shown in FIG. 15B) which provides additional insights to the user as to the impact of consuming particular foods. Meals GUI 15100 is similar to meals GUI 14800 (FIGS. 14D-1 to 14D-3), except that a segmented bar 15102 representing a score associated with a logged meal, and a segmented bar representing a score for a detected missed meal is displayed for each meal event 15101 and/or notification (not shown), respectively, instead of a numerical score. According to an aspect of the embodiments, the logged meals and detected missed meals displayed on meals GUI 15100 ranked based on their respective score. Further, in some embodiments, when a meal event 15101 is not scored yet, a segmented bar will not appear on the respective meal event 15101 displayed on meals GUI 15100.

Further, and with reference to FIG. 15B, a list 15101 of meal information is displayed on meals GUI 15100, wherein the list 15101 can include one or more notifications (not shown) corresponding to one or more missed meals or episodes for a predetermined period of time (e.g., the last 24-hours). According to an aspect of the embodiments, each of the notifications can be similar to the notifications 15004. Specifically, each notification can include a timestamp indicating a time associated with the detected meal (e.g., “9:00 AM”). Further, in some exemplar embodiments (though not illustrated), each of the notifications can include a graphical representation, such as a segmented bar, representing a score associated with the detected meal detected meal has been scored. Specifically, the score can be represented by a segmented bar with three segment portions, wherein: (1) the first segment can have a red color or orange color and be indicative of an analyte score corresponding to a high glucose impact (e.g., an average glucose range exceeding 250 mg/dL); (2) the first segment and second segment can have a yellow color and be indicative of an analyte score corresponding to a medium glucose impact (e.g., an average glucose range between 181 mg/dL and 250 mg/dL, or no greater than 250 mg/dL and/or at least 181 mg/dL); or (3) the first segment, the second segment, and the third segment can have a green color and be indicative of an analyte score corresponding to a low glucose impact (e.g., an average glucose range between 70 mg/dL and 180 mg/dL, or no greater than 180 mg/dL). Those of skill in the art will appreciate that various other graphical representations and colors, and color configurations can be utilized with the graphical representations without departing from the scope of the disclosure.

Still with reference to FIG. 15B, (though not illustrated) each of the notifications can include a selectable “ignore” button and a selectable meal logging button. Specifically, in some embodiments, upon the user selecting the ignore button on a particular notification, the notification is removed from the display of meals GUI 15100. In some embodiments, removing the notification removes the detected missed meal corresponding thereto and, as such, removes the deleted missed meal from the total average meal score determined by the TIR application. In this manner, the total average meal score can update to account for the removal of the deleted detected missed meal. Specifically, the total average meal score will update so as to reflect the deletion of the detected missed meal. More specifically, if the total average meal score had initially accounted for the detected missed meal, then the total average meal score will be recalculated so as to remove the deleted detected missed meal from the total average meal score.

Further, in some embodiments, and still with reference to FIG. 15B (though not illustrated), if the user selects the meal logging button on a notification, meal logging GUI 500 (FIG. 5A) is outputted. Moreover, in some embodiments, meal entry modal 520 (as shown in FIG. 5C) can appear, wherein the time field 527 is prepopulated with a date and time associated with the detected missed meal associated with the notification (not illustrated) from which the meal logging button was selected. In other embodiments, meal entry modal 520 (as shown in FIG. 5C) can appear, wherein the user sets the date and time associated with the detected missed meal in the time field 527. In some embodiments, when meal logging GUI 500 is outputted through the user selecting the meal logging button on a notification of meal GUI 15100, a camera interface (e.g., FIGS. 5I to 5N) is not shown to the user. Further, in some embodiments, once the user has logged a meal for the particular time associated with the meal, the respective notification (not shown in FIG. 15B) on the list 15101 will be replaced with a meal event 15102 corresponding to the logged meal.

Further, in some embodiments, and as shown in FIG. 15B, meal GUI 15100 further comprises a message 15107, wherein the message 15107 indicates to the user that higher scores mean better total average meal score (“e.g., Higher scores mean better Average Meal Score”).

In some embodiments, meal events 15101 and notifications (not illustrated) are listed chronologically on meals GUI 15100. In other embodiments (though not illustrated), meal events 15101 and notifications are listed in order of score rank on meals GUI 15100.

According to an aspect of the embodiments, meal GUI 15100 (FIG. 15B) comprises all meal events 15101 and notifications, collectively, for a predetermined period of time (e.g., for the preceding or trailing 24-hours). As such, in some instances, the list 15101 can include meal events 15102 and notifications (not illustrated) from a previous day and a current day (e.g., if it is 5 P.M., then the list 15101 can include all meal events 15102 and notifications within the last preceding 24-hour period, up until 5 P.M. the previous day). Additionally, in this regard, the list 15101 can continuously update so as to display meal events 15102 and notifications within a new cycle of the predetermined period of time. For example, if the time changes from 4:59 PM to 5 P.M., then the list 15101 can update so as to remove all meal events 15102 and notifications that were accounted for at 4:59 PM the previous day and account for new meal events 15101 and notifications that occurred at 5 P.M. today.

Further, in some embodiments, and as shown in FIG. 15B, meal GUI 15100 can further comprise a selectable back button 15109 which, upon being selected by the user, outputs insights GUI 15000 (FIGS. 15A-1 to 15A-5).

Various aspects of the present subject matter are set forth above, in review of, and/or in supplementation to, the embodiments described thus far, with the emphasis here being on the interrelation and interchangeability of the following embodiments. In other words, an emphasis is on the fact that each feature of the embodiments can be combined with each and every other feature unless explicitly stated otherwise or logically implausible. The embodiments described herein are restated and expanded upon in the following paragraphs without explicit reference to the figures.

Systems, devices, and methods for detecting, measuring and classifying meals for an individual based on analyte measurements. These results and related information can be presented to the individual to show the individual which meals are causing the most severe analyte response. These results can be organized and categorized based on preselected criteria or previous meals and results so as to organize and present the results in a format with reference to glucose as the monitored analyte. Various embodiments disclosed herein relate to methods, systems, and software applications intended to engage an individual by providing direct and timely feedback regarding the individual's meal-related glycemic response.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

To the extent the embodiments disclosed herein include or operate in association with memory, storage, and/or computer readable media, then that memory, storage, and/or computer readable media are non-transitory. Accordingly, to the extent that memory, storage, and/or computer readable media are covered by one or more claims, then that memory, storage, and/or computer readable media is only non-transitory.

In many instances, entities are described herein as being coupled to other entities. It should be understood that the terms “coupled” and “connected” (or any of their forms) are used interchangeably herein and, in both cases, are generic to the direct coupling of two entities (without any non-negligible (e.g., parasitic) intervening entities) and the indirect coupling of two entities (with one or more non-negligible intervening entities). Where entities are shown as being directly coupled together, or described as coupled together without description of any intervening entity, it should be understood that those entities can be indirectly coupled together as well unless the context clearly dictates otherwise.

As used herein and in the appended claims, the singular forms “a”, “an”, and “the” include plural referents unless the context clearly dictates otherwise.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

While the embodiments are susceptible to various modifications and alternative forms, specific examples thereof have been shown in the drawings and are herein described in detail. These embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the scope of the claims by features, functions, steps, or elements that are not within that scope.

The disclosure of this application also contains systems, devices, and methods for detecting and measuring an amount of time an individual is within a predetermined analyte range based on analyte measurements. These results and related information are presented to the individual to show the individual an analyte response associated with consumed meals, or change in an analyte level within a predetermined time period after meals are consumed. These results can be organized based on a ranking system so as to allow the individual to visualize analyte responses and range impact associated with the meals. Various embodiments disclosed herein relate to methods, systems, and software applications intended to engage an individual by providing direct and timely feedback regarding the individual's meal-related analyte response.

The disclosure of this application also contains the following numbered clauses:

1. A glucose monitoring system, comprising:
a reader device, comprising:
   wireless communication circuitry configured to receive data indicative of a glucose level from a sensor control device; and
   one or more processors coupled with a memory, the memory storing a meal monitoring application that, when executed by the one or more processors, causes the one or more processors to:
      detect one or more episodes in the data indicative of the glucose level, wherein the one or more episodes are associated with one or more meals consumed by the user, wherein meal information related to the consumed one or more meals associated with the one or more detected episodes has not been received by the meal monitoring application; and
      output a home graphical user interface (GUI) comprising at least one of:
         a glucose graph card comprising a glucose graph with a glucose trendline reflecting the data indicative of the glucose level;
         a meals section configured to display one or more notifications, wherein each of the one or more notifications corresponds to one of the one or more episodes; and
         a health section comprising one or more activity cards, wherein each of the one or more activity cards is configured to display data related to the user's activity.

2. The glucose monitoring system of clause 1, wherein each of the one or more notifications is positioned between the glucose graph card and the health section on the home GUI.

3. The glucose monitoring system of clause 1 or 2, wherein each of the one or more notifications comprises a message indicating that the episode was detected.

4. The glucose monitoring system of any preceding clause, wherein each of the one or more notifications comprises a timestamp configured to indicate a time associated with the one of the one or more episodes.

5. The glucose monitoring system of any preceding clause, wherein each of the one or more notifications comprises a graphical representation configured to represent a score associated with the one of the one or more episodes.

6. The glucose monitoring system of clause 5, wherein the graphical representation includes a segmented bar, wherein the segmented bar comprises a plurality of segment portions, wherein each of the plurality of segment portions is configured to correspond to a glucose impact based on the data indicative of the glucose level.

7. The glucose monitoring system of clause 6, wherein the plurality of segment portions includes three segment portions, wherein a first segment of the three segment portions comprises a first color and is indicative of a score corresponding to a high glucose impact based on the data indicative of the glucose level, wherein a second segment of the three segment portions comprises a second color and is indicative of a score corresponding to a medium glucose impact based on the data indicative of the glucose level, and wherein a third segment of the three segment portions comprises a third color and is indicative of a score corresponding to a low glucose impact based on the data indicative of the glucose level.

8. The glucose monitoring system of any preceding clause, wherein each of the one or more notifications comprises a selectable ignore button and a selectable meal logging button.

9. The glucose monitoring system of clause 8, wherein the reader device further comprises a touchscreen, and wherein the meal monitoring application, when executed by the one or more processors, further causes the one or more processors to:
in response to a selection of the ignore button on one of the one or more notifications, remove the one of the one or more notifications from the home GUI.

10. The glucose monitoring system of clause 8 or 9, wherein the reader device further comprises a touchscreen, and wherein the meal monitoring application, when executed by the one or more processors, further causes the one or more processors to:
in response to a selection of the meal logging button, output a meal logging GUI configured to receive inputted meal information related to the consumed one of the one or more meals associated with the one of the one more episodes associated with the one of the one or more notifications.

11. The glucose monitoring system of clause 10, wherein the meal monitoring application, when executed by the one or more processors, further causes the one or more processors to:
in response to the meal logging GUI receiving inputted meal information, remove the one of the one or more notifications from the home GUI and add a meal event corresponding to the inputted meal information to the home GUI, wherein the meal event is displayed in the meals section.

12. The glucose monitoring system of clause 10 or 11, wherein the meal monitoring application, when executed by the one or more processors, further causes the one or more processors to:
in response to the meal logging GUI receiving inputted meal information, output an in-app banner on the home GUI, wherein the in-app banner comprises a message indicating that meal information was received.

13. The glucose monitoring system of clause 12, wherein the in-app banner further comprises a selectable link which, upon being selected, outputs a meal review GUI comprising information related to the meal event.

14. The glucose monitoring system of any preceding clause, wherein the one or more activity cards includes a first activity card comprising data related to the user's movement for a predetermined period of time and a second activity card comprising data related to the user's sleep for a predetermined period of time.

15. The glucose monitoring system of clause 14, wherein the data related to the user's movement includes data related to the user's steps and/or wheelchair pushes for the predetermined period of time.

16. The glucose monitoring system of clause 14 or 15, wherein the data related to the user's sleep includes data related to the user's hours of sleep for the predetermined period of time.

17. The glucose monitoring system of any preceding clause, wherein the meal monitoring application, when executed by the one or more processors, further causes the one or more processors to:
in response to detecting one or more episodes, display an episode marker associated with each of the detected one or more episodes on a meal timeline.

18. The glucose monitoring system of clause 17, wherein the glucose graph card further comprises an x-axis comprising units of time, wherein the meal timeline is positioned below the x-axis.

19. The glucose monitoring system of clause 18, wherein the episode marker associated with each of the detected one or more episodes is positioned along the meal timeline for the user to visually associate the episode marker associated with each of the detected one or more episodes with a corresponding data indicative of the glucose level reflected on the glucose trendline.

20. The glucose monitoring system of any of clauses 17 to 19, wherein the episode marker associated with each of the detected one or more episodes includes a missed meal icon.

21. The glucose monitoring system of any of clauses 17 to 20, wherein each of the one or more notifications comprises a selectable ignore button and a selectable meal logging button, wherein the reader device further comprises a touchscreen, and wherein the meal monitoring application, when executed by the one or more processors, further causes the one or more processors to:
in response to a selection of the meal logging button, output a meal logging GUI configured to receive inputted meal information related to the consumed one of the one or more meals associated with each of the detected one or more episodes.

22. The glucose monitoring system of clause 21, wherein the meal monitoring application, when executed by the one or more processors, further causes the one or more processors to:
in response to the meal logging GUI receiving inputted meal information, remove the episode marker associated with each of the detected one or more episodes from the meal timeline and replace the removed episode marker with a logged meal icon.

23. The glucose monitoring system of clause 22, wherein the logged meal icon comprises a photograph or image associated with the inputted meal information.

24. The glucose monitoring system of any of clauses 17 to 23, wherein each of the one or more notifications comprises a selectable ignore button and a selectable meal logging button, wherein the reader device further comprises a touchscreen, and wherein the meal monitoring application, when executed by the one or more processors, further causes the one or more processors to:
in response to a selection of the ignore button, remove the episode marker from the meal timeline.

25. The glucose monitoring system of any preceding clause, wherein the home GUI further comprises a text description indicating a current day of a week, wherein the text description comprises a date adjacent to a switch configured to be toggled between a first position and a second position.

26. The glucose monitoring system of clause 25, wherein the home GUI displays a plurality of selectable rings in response to the switch being toggled to the first position, wherein each of the selectable rings corresponds to a different day of a past week.

27. The glucose monitoring system of clause 26, wherein the reader device further comprises a touchscreen, and wherein the meal monitoring application, when executed by the one or more processors, further causes the one or more processors to:
in response to the user selecting one of the plurality of selectable rings, display data associated with the different day of the past week corresponding to the selected one of the plurality of selectable rings on the home GUI.

28. The glucose monitoring system of any preceding clause, wherein the meals section is further configured to display one or more meal events, wherein each of the one or more meal events corresponds to a meal logged by the user, and wherein the meal monitoring application, when executed by the one or more processors, further causes the one or more processors to:
determine a score for each of one or more meal events and the one or more episodes, wherein each determined score corresponds to a glucose response based on the data indicative of the glucose level associated with each of the one or more meal events and the one or more episodes.

29. The glucose monitoring system of clause 28, wherein the home GUI further comprises an average meal score card configured to reflect data related to a total average meal score, wherein the total average meal score is a total score for all determined scores for each of the one or more meal events and the one or more episodes for a predetermined period of time.

30. The glucose monitoring system of clause 29, wherein the predetermined period of time is 24 hours.

31. The glucose monitoring system of clause 29 or 30, wherein the total average meal score is configured to continuously update at a predetermined frequency to reflect a new total average meal score.

32. The glucose monitoring system of any of clauses 29 to 31, wherein the average meal score comprises a message, a status indicator configured to provide a summary related to the total average meal score, and/or a graphical representation corresponding to the total average meal score.

33. The glucose monitoring system of clause 32, wherein the graphical representation includes a gradient scale comprising a gradient, wherein each portion of the gradient corresponds to a different status of the total average meal score.

34. The glucose monitoring system of clause 33, wherein the gradient includes a color gradient, wherein each color of the color gradient corresponds to the different status of the total average meal score.

35. The glucose monitoring system of clause 33 or 34, wherein the total average meal score can be a numerical value between one and 100, wherein the gradient scale is configured to represent a total average meal score range between one and 100.

36. The glucose monitoring system of clause 34 or clause 35 when dependent on clause 34, wherein the gradient scale comprises a picker configured to align with a color of the gradient scale corresponding to a status of the total average meal score.

37. The glucose monitoring system of any of clauses 26 to 36, wherein the home GUI further comprises a selectable Go To Today link, and wherein the reader device further comprises a touchscreen, and wherein the meal monitoring application, when executed by the one or more processors, further causes the one or more processors to:
in response to the user selecting the Go To Today link, display data associated with the different day of the past week corresponding to the current day of the week on home GUI.

38. A glucose monitoring system, comprising:
a reader device, comprising:
   wireless communication circuitry configured to receive data indicative of a glucose level from a sensor control device;
   an input configured to receive meal information indicative of one or more meals consumed by a user; and
   one or more processors coupled with a memory, the memory storing meal monitoring application that, when executed by the one or more processors, causes the one or more processors to:
      detect one or more episodes in the data indicative of the glucose level, wherein each of the one or more episodes is associated with meal information of one or more meals consumed by the user but not been received by the input;
      determine a score for one or more meal events and each of the one or more detected episodes, wherein each of the one or more meal events is configured to reflect the meal information indicative of one or more meals consumed by a user that is received by the input, and wherein the score corresponds to a glucose response based on the data indicative of the glucose level associated with each of the one or more meal events and the one or more detected episodes; and
      output an insights graphical user interface (GUI) comprising at least one of:
         an average meal score card configured to reflect data related to a total average meal score, wherein the total average meal score is a total score for all determined scores for each of the one or more meal events and the one or more episodes for a predetermined period of time; and
         a meals section comprising a list configured to display the one or more meal events and one or more notifications corresponding to the one or more episodes.

39. The glucose monitoring system of clause 38, wherein the predetermined period of time is 24 hours.

40. The glucose monitoring system of clause 38 or 39, wherein the total average meal score is configured to continuously update at a predetermined frequency to reflect a new total average meal score.

41. The glucose monitoring system of any of clauses 38 to 40, wherein the average meal score comprises a message, a status indicator configured to provide a summary related to the total average meal score, and a graphical representation corresponding to the total average meal score.

42. The glucose monitoring system of clause 41, wherein the graphical representation includes a gradient scale comprising a gradient, wherein each portion of the gradient corresponds to a different status of the total average meal score.

43. The glucose monitoring system of clause 42, wherein the gradient includes a color gradient, wherein each color of the color gradient corresponds to the different status of the total average meal score.

44. The glucose monitoring system of clause 42 or 43, wherein the total average meal score can be a numerical value between one and 100, wherein the gradient scale is configured to represent a total average meal score range between one and 100.

45. The glucose monitoring system of clause 43 or clause 44 when dependent on clause 43, wherein the gradient scale comprises a picker configured to align with a color of the gradient scale corresponding to a status of the total average meal score.

46. The glucose monitoring system of any of clauses 38 to 45, wherein each of the one or more meal events comprise a text description of a meal, a photograph associated with the meal, a timestamp associated with the meal, a portion size indicator associated with the meal, and/or a graphical representation configured to represent the determined score associated with one of the one or more meal events.

47. The glucose monitoring system of clause 46, wherein the graphical representation includes a segmented bar, wherein the segmented bar comprises a plurality of segment portions, wherein each of the plurality of segment portions is configured to correspond to the glucose response based on the data indicative of the glucose level.

48. The glucose monitoring system of clause 47, wherein the plurality of segment portions includes three segment portions, wherein a first segment of the three segment portions comprises a first color and is indicative of a score corresponding to a high glucose response based on the data indicative of the glucose level, wherein a second segment of the three segment portions comprises a second color and is indicative of a score corresponding to a medium glucose response based on the data indicative of the glucose level, and wherein a third segment of the three segment portions comprises a third color and is indicative of a score corresponding to a low glucose response based on the data indicative of the glucose level.

49. The glucose monitoring system of any of clauses 38 to 48, wherein each of the one or more notifications comprises a message indicating that an episode was detected.

50. The glucose monitoring system of any of clauses 38 to 49, wherein each of the one or more notifications comprises a timestamp configured to indicate a time associated with the one corresponding one of the one or more episodes.

51. The glucose monitoring system of any of clauses 38 to 50, wherein each of the one or more notifications comprises a graphical representation configured to represent the determined score associated with corresponding one of the one or more episodes.

52. The glucose monitoring system of clause 51, wherein the graphical representation includes a segmented bar, wherein the segmented bar comprises a plurality of segment portions, wherein each of the plurality of segment portions is configured to correspond to the glucose impact based on the data indicative of the glucose level.

53. The glucose monitoring system of clause 52, wherein the plurality of segment portions includes three segment portions, wherein a first segment of the three segment portions comprises a first color and is indicative of a score corresponding to a high glucose response based on the data indicative of the glucose level, wherein a second segment of the three segment portions comprises a second color and is indicative of a score corresponding to a medium glucose response based on the data indicative of the glucose level, and wherein a third segment of the three segment portions comprises a third color and is indicative of a score corresponding to a low glucose response based on the data indicative of the glucose level.

54. The glucose monitoring system of any of clauses 38 to 53, wherein each of the one or more notifications comprises a selectable ignore button and a selectable meal logging button.

55. The glucose monitoring system of clause 54, wherein the reader device further comprises a touchscreen, and wherein the meal monitoring application, when executed by the one or more processors, further causes the one or more processors to:
in response to a selection of the ignore button on one of the one or more notifications, remove the one of the one or more notifications from the meals section and the corresponding detected one of the one or more episodes.

56. The glucose monitoring system of clause 55, wherein the total average meal score is configured to update in response to the removal of the detected one of the one or more episodes.

57. The glucose monitoring system of any of clauses 54 to 56, wherein the reader device further comprises a touchscreen, and wherein the meal monitoring application, when executed by the one or more processors, further causes the one or more processors to:
in response to a selection of the meal logging button, output a meal logging GUI configured to receive inputted meal information related to a meal associated with the one of the one more episodes corresponding to the one of the one or more notifications.

58. The glucose monitoring system of clause 57, wherein the meal monitoring application, when executed by the one or more processors, further causes the one or more processors to:
in response to the meal logging GUI receiving inputted meal information, remove the one of the one or more notifications from the home GUI and replace the removed one of the one or more notifications with a new meal event, wherein the new meal event is displayed in the meals section.

59. The glucose monitoring system of clause 57 or 58, wherein the meal monitoring application, when executed by the one or more processors, further causes the one or more processors to:
in response to the meal logging GUI receiving inputted meal information, output an in-app banner on the insights GUI, wherein the in-app banner comprises a message indicating that meal information was received.

60. The glucose monitoring system of any of clauses 38 to 59, wherein the meals section is configured to display the one or more meal events and the one or more notifications in chronological order.

61. The glucose monitoring system of any of clauses 38 to 60, wherein the one or more detected episodes are scored automatically.

62. The glucose monitoring system of any of clauses 38 to 61, wherein the one or more detected episodes are scored after the input has received the meal information corresponding to the one of the one or more detected episodes.

63. The glucose monitoring system of any of clauses 38 to 62, wherein the predetermined period of time is 24 hours.

64. The glucose monitoring system of any of clauses 38 to 63, wherein the meals section is configured to continuously update at a predetermined frequency to reflect one or more new meal events and one or more new notifications for a subsequent predetermined period of time.

65. The glucose monitoring system of any of clauses 38 to 64, wherein the insights GUI further comprises a selectable All Meals link, wherein the reader device further comprises a touchscreen, and wherein the meal monitoring application, when executed by the one or more processors, further causes the one or more processors to:
in response to a selection of the All Meals link, output a meals GUI comprising information related to all of the one or more meal events and one or more episodes corresponding to the predetermined period of time.

65. Apparatus comprising:
means for receiving data indicative of a glucose level from a sensor control device;
means for detecting one or more episodes in the data indicative of the glucose level, wherein the one or more episodes are associated with one or more meals consumed by the user, wherein meal information related to the consumed one or more meals associated with the one or more detected episodes has not been received by the meal monitoring application; and
means for outputting a home graphical user interface (GUI) comprising at least one of:
   a glucose graph card comprising a glucose graph with a glucose trendline reflecting the data indicative of the glucose level;
   a meals section configured to display one or more notifications, wherein each of the one or more notifications corresponds to one of the one or more episodes; and
   a health section comprising one or more activity cards, wherein each of the one or more activity cards is configured to display data related to the user's activity.

66. Apparatus comprising:
means for receiving data indicative of a glucose level from a sensor control device;
means for receiving meal information indicative of one or more meals consumed by a user;
means for detecting one or more episodes in the data indicative of the glucose level, wherein each of the one or more episodes is associated with meal information of one or more meals consumed by the user but not been received by the input;
means for determining a score for one or more meal events and each of the one or more detected episodes, wherein each of the one or more meal events is configured to reflect the meal information indicative of one or more meals consumed by a user that is received by the input, and wherein the score corresponds to a glucose response based on the data indicative of the glucose level associated with each of the one or more meal events and the one or more detected episodes; and
means for outputting an insights graphical user interface (GUI) comprising at least one of:
   an average meal score card configured to reflect data related to a total average meal score, wherein the total average meal score is a total score for all determined scores for each of the one or more meal events and the one or more episodes for a predetermined period of time; and
   a meals section comprising a list configured to display the one or more meal events and one or more notifications corresponding to the one or more episodes.

67. A method comprising the steps of:
receiving data indicative of a glucose level from a sensor control device;
detecting one or more episodes in the data indicative of the glucose level, wherein the one or more episodes are associated with one or more meals consumed by the user, wherein meal information related to the consumed one or more meals associated with the one or more detected episodes has not been received by the meal monitoring application; and
outputting a home graphical user interface (GUI) comprising at least one of:
   a glucose graph card comprising a glucose graph with a glucose trendline reflecting the data indicative of the glucose level;
   a meals section configured to display one or more notifications, wherein each of the one or more notifications corresponds to one of the one or more episodes; and
   a health section comprising one or more activity cards, wherein each of the one or more activity cards is configured to display data related to the user's activity.

68. A method comprising the steps of:
receiving data indicative of a glucose level from a sensor control device;
receiving meal information indicative of one or more meals consumed by a user;
detecting one or more episodes in the data indicative of the glucose level, wherein each of the one or more episodes is associated with meal information of one or more meals consumed by the user but not been received by the input;
determining a score for one or more meal events and each of the one or more detected episodes, wherein each of the one or more meal events is configured to reflect the meal information indicative of one or more meals consumed by a user that is received by the input, and wherein the score corresponds to a glucose response based on the data indicative of the glucose level associated with each of the one or more meal events and the one or more detected episodes; and
outputting an insights graphical user interface (GUI) comprising at least one of:
   an average meal score card configured to reflect data related to a total average meal score, wherein the total average meal score is a total score for all determined scores for each of the one or more meal events and the one or more episodes for a predetermined period of time; and
   a meals section comprising a list configured to display the one or more meal events and one or more notifications corresponding to the one or more episodes.

69. A computer program, computer program product or computer readable medium comprising software code adapted, when executed by a computer system, to perform a method as set out in clause 67 or 68.

1-109. (canceled)

110. An analyte monitoring system, comprising:
a sensor control device comprising a glucose sensor, wherein at least a portion of the glucose sensor is configured to be positioned under a skin surface and in fluid contact with a bodily fluid of a subject; and
a reader device, comprising:
wireless communication circuitry configured to receive data indicative of a glucose level of the subject from the sensor control device; and
one or more processors coupled to a memory, the memory storing an analyte monitoring software application that, when executed by the one or more processors, cause the one or more processors to:
calculate one or more metrics using the received data indicative of the glucose level of the subject for a predetermined time period, wherein the calculated one or more metrics comprises a corresponding one or more raw values;
normalize the corresponding one or more raw values for each of the one or more calculated metrics;
weigh each of the normalized corresponding one or more raw values, wherein the each of the weighted normalized corresponding raw value is provided a sub-score;
calculate an overall score for the predetermined time period based on a function of the sub-scores; and
output, to a graphical user interface, at the end of the predetermined time, information related to the overall score.

111. The analyte monitoring system of claim 110, wherein the predetermined time period is one day, one week, or one month.

112. The analyte monitoring system of claim 110, wherein the one or more metrics comprise a meal scoring metric.

113. The analyte monitoring system of claim 110, wherein the one or more metrics comprise an amount of time the sensor control device is worn by the user.

114. The analyte monitoring system of claim 110, wherein the one or more metrics comprise an amount of time the sensor control device is active.

115. The analyte monitoring system of claim 110, wherein the one or more metrics comprise a glycemic variability.

116. The analyte monitoring system of claim 110, wherein the one or more metrics comprise an amount of time the glucose level of the user is below a predetermined low glucose threshold value.

117. The analyte monitoring system of claim 116, wherein the predetermined low glucose threshold value is 54 mg/dL or 70 mg/dL.

118. The analyte monitoring system of claim 110, wherein the one or more metrics comprise an amount of time the glucose level of the user is above a predetermined high glucose threshold value.

119. The analyte monitoring system of claim 118, wherein the predetermined high glucose threshold value is 180 mg/dL or 250 mg/dL.

120. The analyte monitoring system of claim 110, wherein weighing the normalized raw value for each of the one or more calculated metrics comprises applying a differing weight to at least one or more calculated metrics.

121. The analyte monitoring system of claim 110, wherein the overall score is a numerical value between 1 and 100.

122. The analyte monitoring system of claim 110, wherein calculating the overall score for the predetermined time period comprises summing up a total of the sub-scores.

123. The analyte monitoring system of claim 110, wherein the overall score is a numerical daily score.

124. The analyte monitoring system of claim 110, wherein the one or more processors are further caused to:
update, at a predetermined frequency, the overall score, wherein an updated overall score is calculated for a subsequent predetermined time period.

125. The analyte monitoring system of claim 110, wherein the information related to the overall score comprises a congratulatory message in response to the overall score exceeding a predetermined high threshold overall score value.

126. The analyte monitoring system of claim 125, wherein the predetermined high threshold overall score value is 85, 90, or 95.

127. The analyte monitoring system of claim 110, wherein the information related to the overall score comprises a recommendation on improving the user's glycemic control in response to the overall score falling below a predetermined low threshold overall score value.

128. The analyte monitoring system of claim 127, wherein the predetermined low threshold overall score value is 65, 60, or 55.

129. The analyte monitoring system of claim 110, wherein the information related to the overall score comprises a trend of the user's historical overall scores over a second predetermined time period.
